(19) United States
(12) Patent Application Publication
(10) Pub. No.: US 2025/0154085 A1
(43) Pub. Date: May 15, 2025

(54) Organic Compound And Organic Light-Emitting Device Comprising Same

(71) Applicant: P&H TECH Co., Ltd, Gyeonggi-do (KR)

(72) Inventors: In-Ho LEE, Gyeonggi-do (KR); Seo-Yong HYUN, Gyeonggi-do (KR)

(73) Assignee: P&H TECH Co., Ltd, Gyeonggi-do (KR)

(21) Appl. No.: 18/840,025

(22) PCT Filed: Feb. 16, 2023
(86) PCT No.: PCT/KR2023/002248

§ 371 Date: Aug. 20, 2024

(30) Foreign Application Priority Data
Feb. 21, 2022 (KR) 10-2022-0021980

(51) Int. Cl. C07C 25/13; C07C 22/08; C07F 7/12; H10K 50/858

(52) U.S. Cl. CPC C07C 25/13; C07C 22/08; C07F 7/12; H10K 50/858

(57) ABSTRACT

The present invention relates to an organic compound employed as a light efficiency improving layer (capping layer) material included in an organic light-emitting device. If employed in the light efficiency improving layer included in the organic light-emitting device, the compound according to the present invention can implement low-voltage driving and improved luminescent properties such as excellent luminous efficacy, color purity, and the like, and thus can be effectively used for various lighting devices and display devices.

### TECHNICAL FIELD

The present invention relates to an organic compound that is employed as a material for a light efficiency improving layer (capping layer) provided in an organic light-emitting device, and an organic light-emitting device that employs the same, thus achieving greatly improved luminescent properties such as low-voltage driving of the device and excellent luminous efficiency.

### BACKGROUND ART

The organic light-emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and red, and thus has recently become a subject of intense interest as a next-generation display device.

However, in order for such an organic light-emitting device to exhibit the aforementioned characteristics, the materials constituting an organic layer in the device, such as hole injecting materials, hole transport materials, light-emitting materials, electron transport materials, and electron injecting materials, are prerequisites for the support by stable and efficient materials. However, the development of a stable and efficient organic layer material for an organic light-emitting device has not yet been sufficiently made.

Thus, further improvements in terms of efficiency and life characteristics are required for good stability, high efficiency, long lifetime, and large size of organic light-emitting devices. Particularly, there is a strong need to develop materials constituting each organic layer of organic light-emitting devices.

In addition, recently, research aimed at improving the characteristics of organic light-emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

### DISCLOSURE

### Technical Problem

Thus, the present invention has been made in an effort to provide a novel organic compound which may be employed in a light efficiency improving layer (capping layer) provided in an organic light-emitting device to implement excellent luminescent properties such as low-voltage driving of the device and improved luminous efficiency, and an organic light-emitting device including the same.

### Technical Solution

An aspect of the present invention provides an organic compound represented by Formula I below.

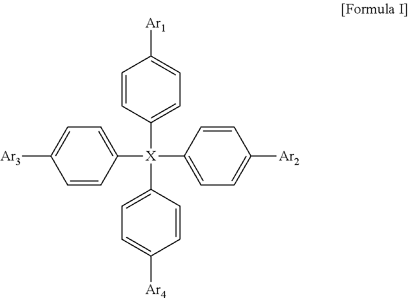

Characteristic structures of Formula I above and specific compounds, X and Ar₁ to Ar₄ implemented thereby will be described below.

Another aspect of the present invention provides an organic light-emitting device including a first electrode, a second electrode, and one or more organic layers arranged between the first and second electrodes, wherein the organic light-emitting device further includes a light efficiency improving layer (capping layer) formed on at least one side opposite to the organic layer among the upper or lower portions of the first electrode and the second electrode, and the light efficiency improving layer includes an organic compound represented by Formula I above.

### Advantageous Effects

According to the present invention, an organic compound is employed as a material for a light efficiency improving layer provided in an organic light-emitting device to implement low-voltage driving of the organic light-emitting device and improved luminescent properties such as excellent luminous efficiency, color purity, etc., and thus can be effectively used in various display devices.

### BEST MODE FOR CARRYING OUT INVENTION

Hereinafter, the present invention will be described in more detail.

The present invention relates to an organic light-emitting compound represented by Formula I below, which is employed as a material for a light efficiency improving layer provided in an organic light-emitting device to achieve low-voltage driving of the device and luminescent properties such as excellent luminous efficiency, color purity, etc.

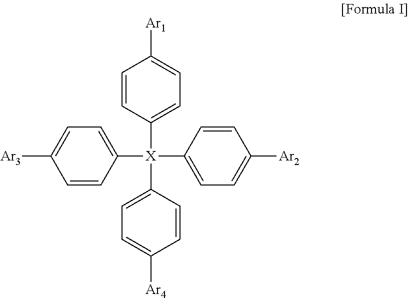

In [Formula I] above,

X is C or Si.

Ar₁ to Ar₄ are the same as or different from each other, and each independently selected from hydrogen, deuterium, a cyano group, a halogen group, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms.

At least one of Ar₁ to Ar₄ is characterized by being any one selected from a halogen group, a substituted or unsubstituted halogenated alkyl group having 1 to 20 carbon atoms, and a substituted or unsubstituted aryl group having 6 to 30 carbon atoms.

According to an embodiment of the present invention, at least one of Ar₁ to Ar₄ is characterized by being an aryl group having 6 to 30 carbon atoms, which is substituted with one or two or more substituents selected from a halogen group and a halogenated alkyl group, or substituted with a substituent to which two or more of the above substituents are linked.

In the definition of Ar₁ to Ar₄, the ‘substituted or unsubstituted’ means substitution of Ar₁ to Ar₄ above with one or at least two substituents selected from the group consisting of deuterium, a halogen group, a cyano group, a nitro group, a hydroxyl group, a silyl group, an amine group, a halogenated alkyl group, a deuterated alkyl group, a cycloalkyl group, a heterocycloalkyl group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy group may be straight-chained or branched. Specific examples thereof include a methoxy group, an ethoxy group, an n-propoxy group, an isopropoxy group, an i-propyloxy group, an n-butoxy group, an isobutoxy group, a tert-butoxy group, a sec-butoxy group, an n-pentyloxy group, an neopentyloxy group, an isopentyloxy group, an n-hexyloxy group, a 3,3-dimethylbutyloxy group, a 2-ethylbutyloxy group, an n-octyloxy group, an n-nonyloxy group, an n-decyloxy group, a benzyloxy group, a p-methylbenzyloxy group, and the like, but are not limited thereto.

In an embodiment of the present invention, the alkoxy groups may be straight or branched. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the amine group may be —NH₂, an alkylamine group, an arylamine group, a heteroaryl amino group, an arylheteroarylamine group, etc., the aryl (heteroaryl)amine group means an amine substituted with an aryl group and/or heteroaryl group, and the alkylamine group means an amine substituted with an alkyl group. Examples of the aryl (heteroaryl)amine group include a substituted or unsubstituted mono aryl (heteroaryl)amine group, a substituted or unsubstituted diaryl (heteroaryl)amine group, or a substituted or unsubstituted triaryl (heteroaryl)amine group, wherein the aryl group and the heteroaryl group in the aryl (heteroaryl)amine group are the same as the definition of the aryl group and the heteroaryl group, and the alkyl group in the alkylamine group is also the same as the definition of the alkyl group.

For example, the arylamine group includes a phenylamine group, a naphthylamine group, a biphenylamine group, an anthracenylamine group, a 3-methyl-phenylamine group, a 4-methyl-naphthylamine group, and a 2-methyl-biphenyl amine group, a 9-methyl-anthracenylamine group, a diphenyl amine group, a phenylnaphthylamine group, a ditolylamine group, a phenyltolylamine group, a triphenylamine group, etc., but is not limited thereto.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (C1), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound according to the present invention represented by Formula I may be used as a material for the light efficiency improving layer (capping layer) provided in the organic light-emitting device due to its structural specificity. Preferred specific examples of the organic compound represented by Formula I according to the present invention include the following compounds but are not limited thereto.

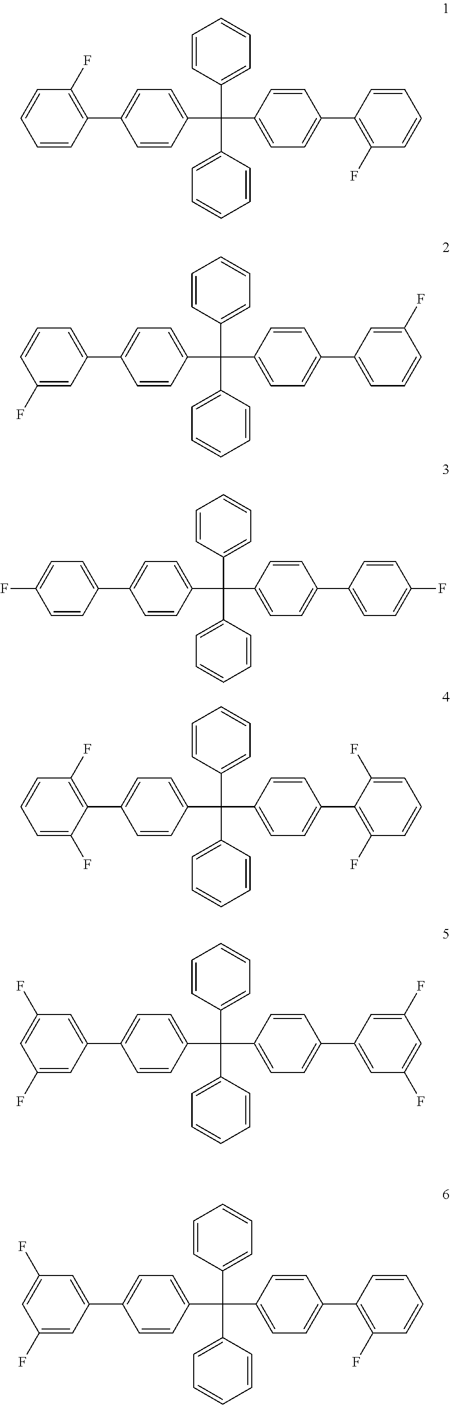

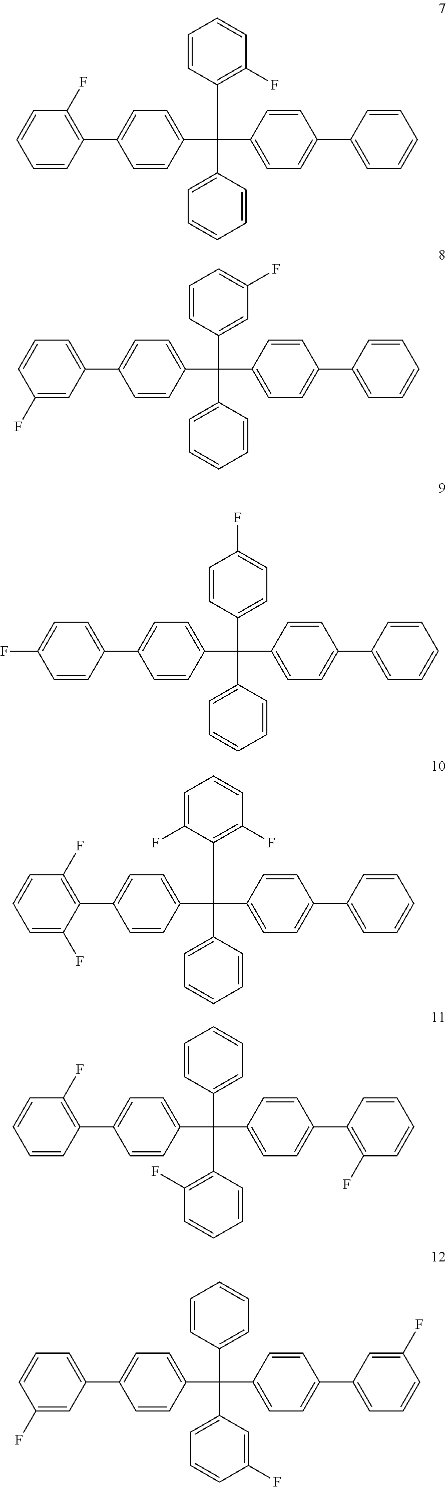

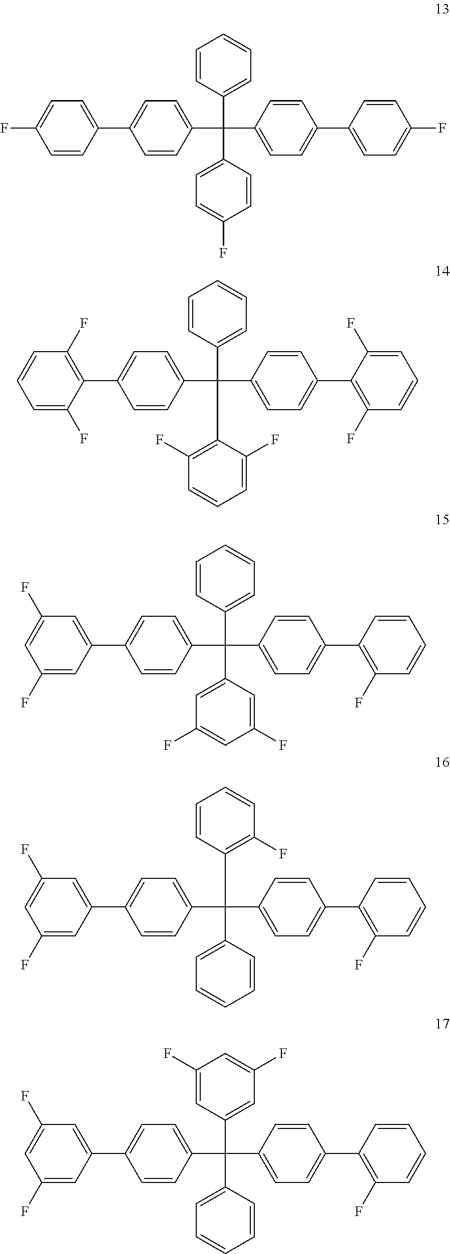

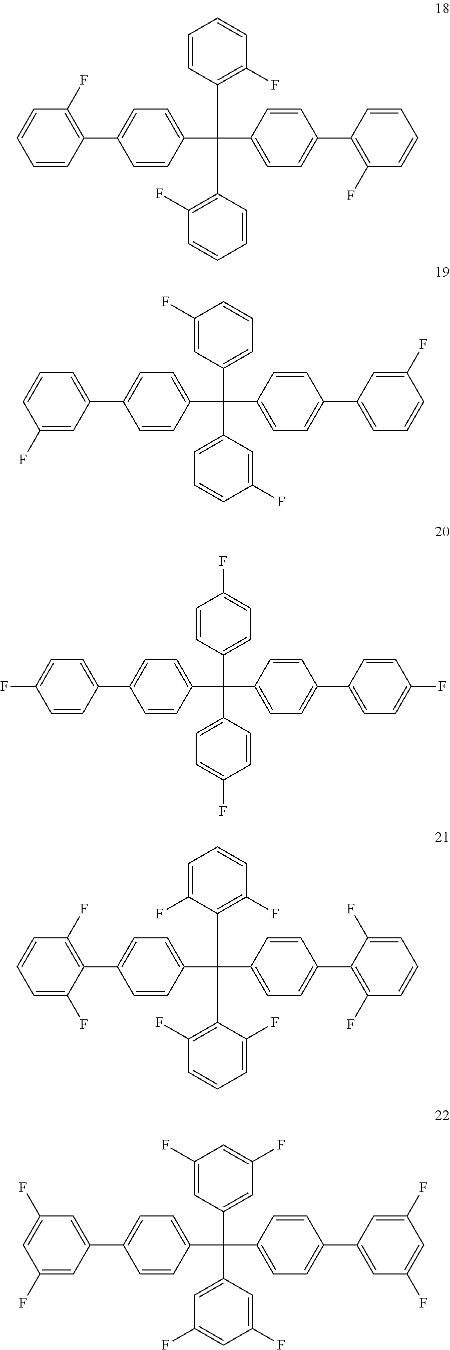

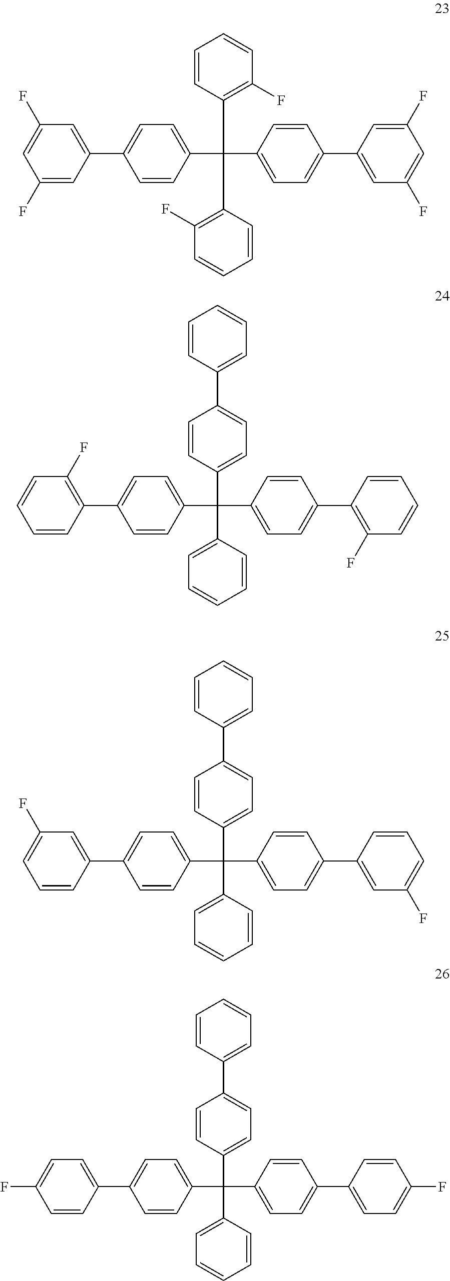

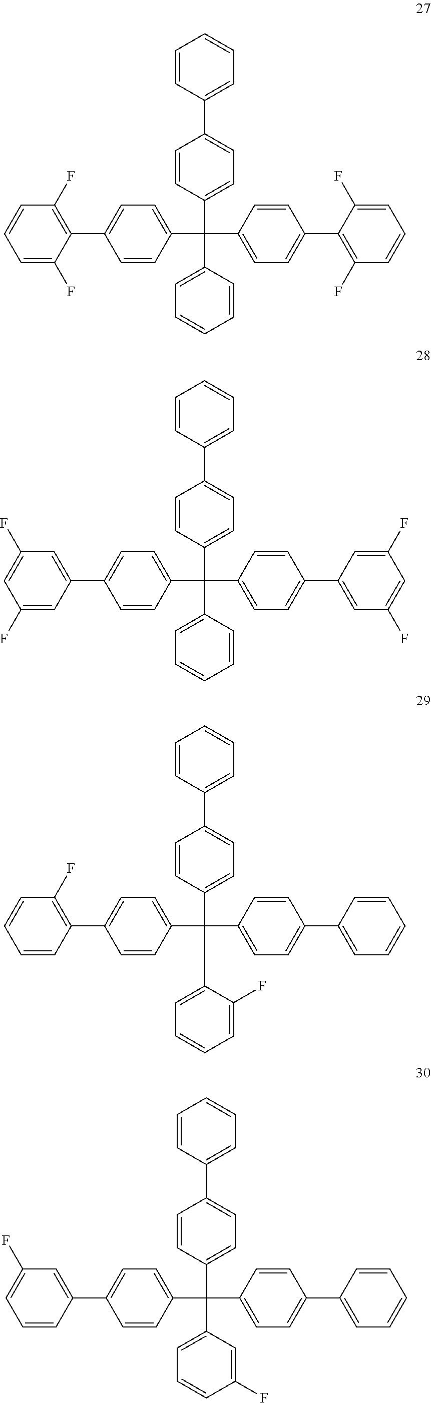

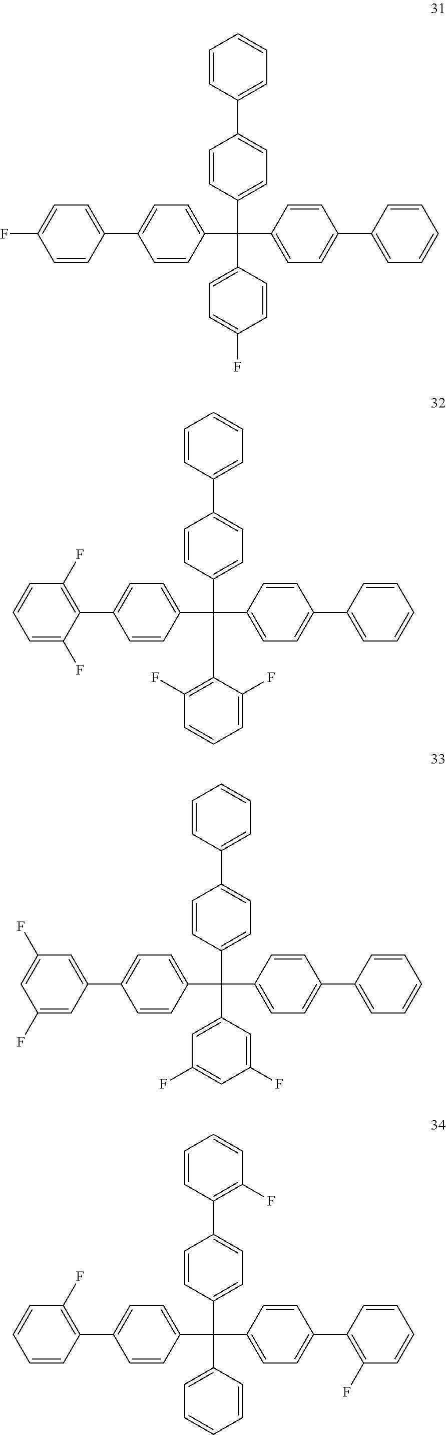

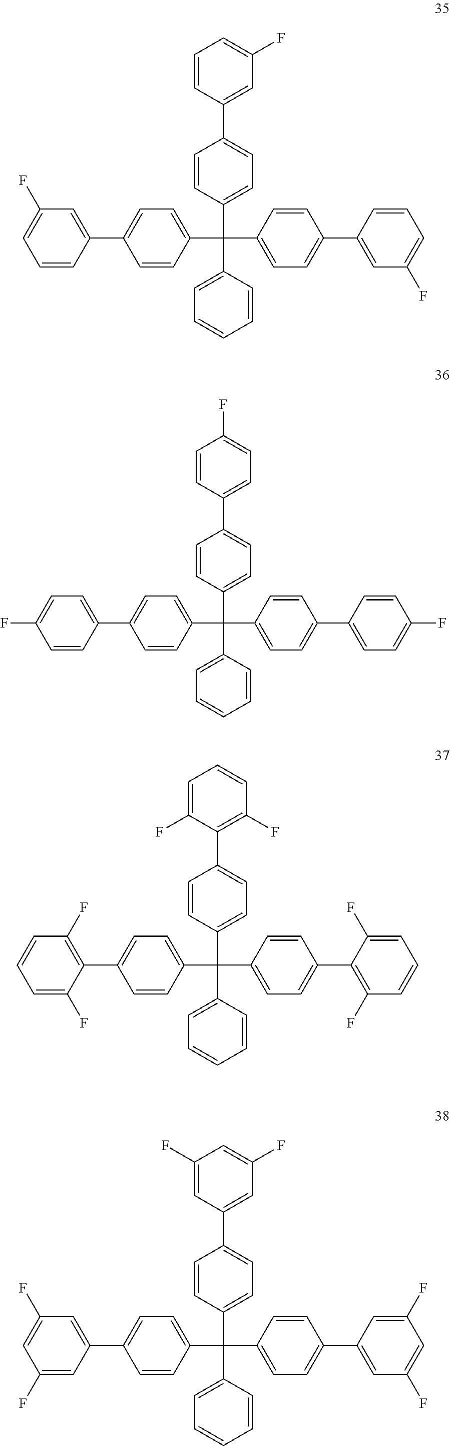 [SMILES: Fc1cccc(-c2ccc(C(c3ccccc3)(c3ccc(-c4cccc(F)c4)cc3)c3ccc(-c4cccc(F)c4)cc3)cc2)c1 ; Fc1ccc(-c2ccc(C(c3ccccc3)(c3ccc(-c4ccc(F)cc4)cc3)c3ccc(-c4ccc(F)cc4)cc3)cc2)cc1 ; Fc1cccc(F)c1-c1ccc(C(c2ccccc2)(c2ccc(-c3c(F)cccc3F)cc2)c2ccc(-c3c(F)cccc3F)cc2)cc1 ; Fc1cc(F)cc(-c2ccc(C(c3ccccc3)(c3ccc(-c4cc(F)cc(F)c4)cc3)c3ccc(-c4cc(F)cc(F)c4)cc3)cc2)c1]

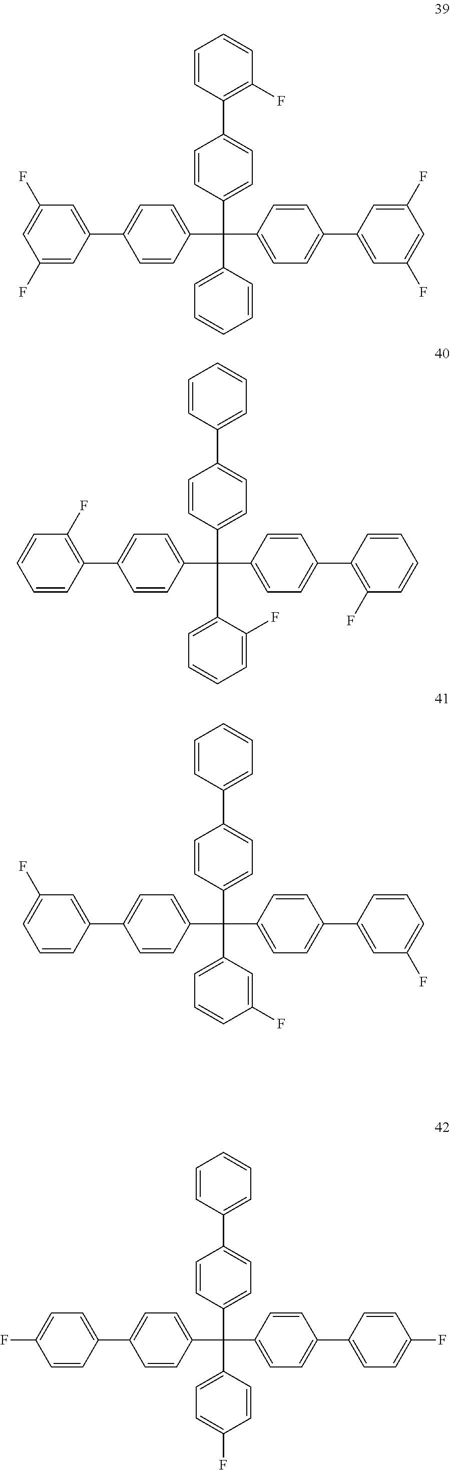

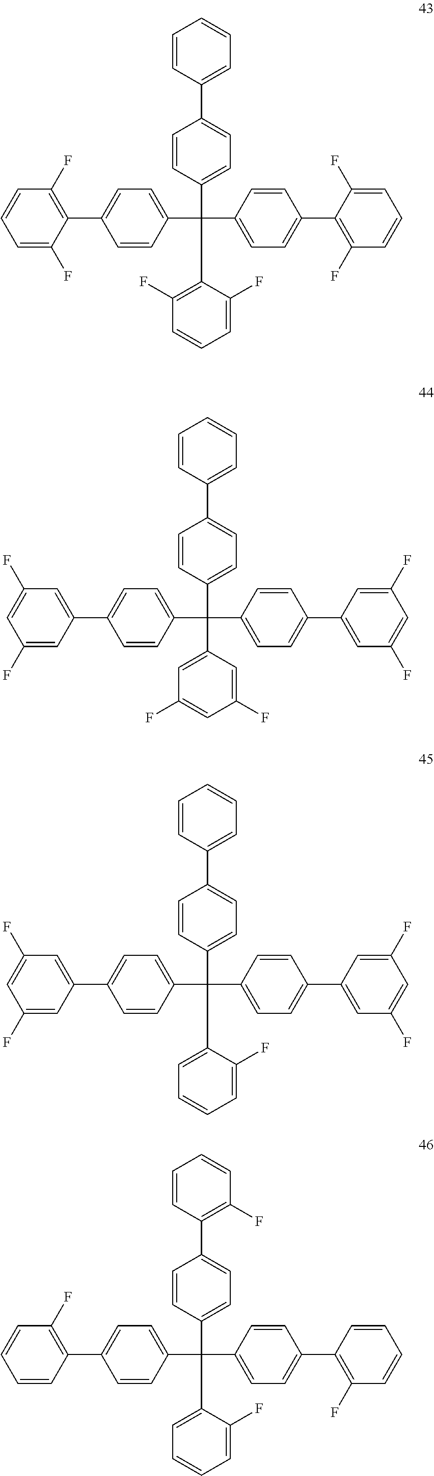

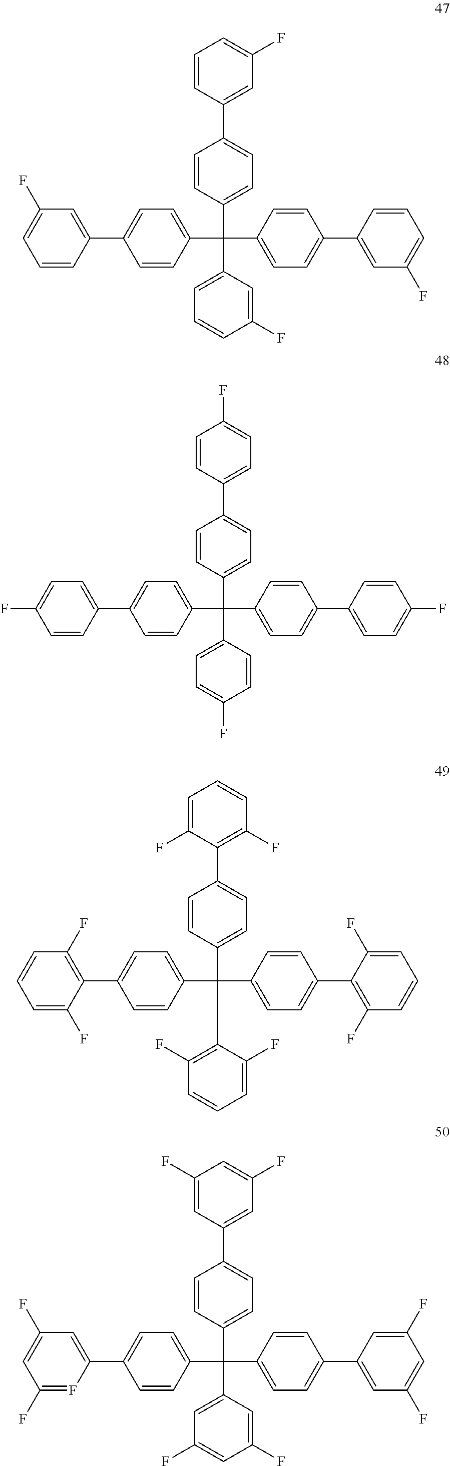

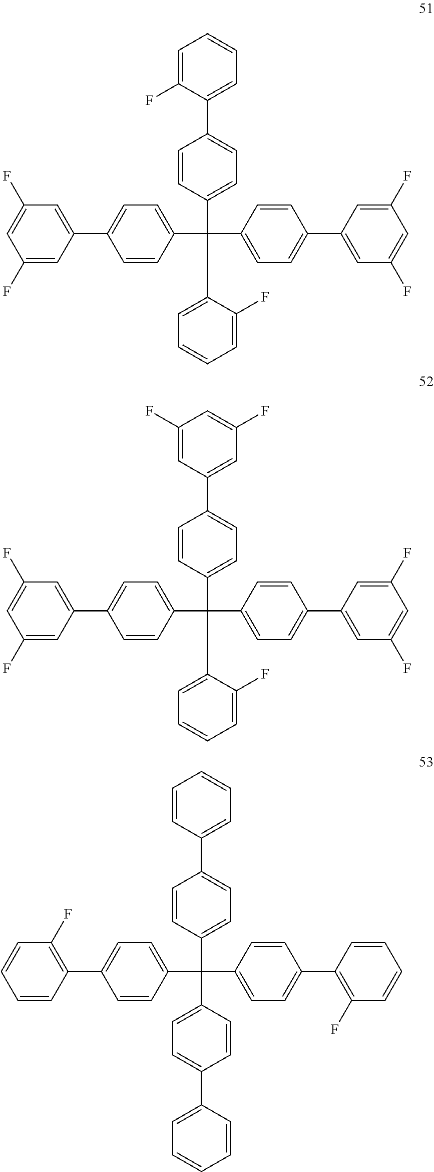

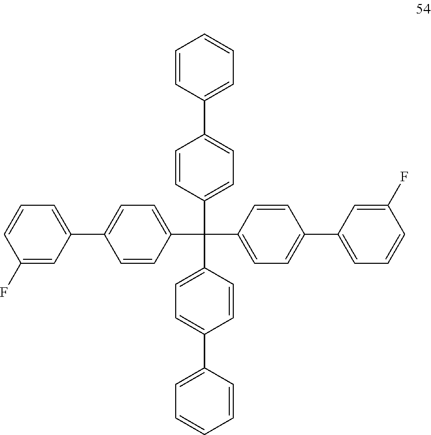 [SMILES: Fc1cccc(-c2ccc(C(c3ccc(-c4ccccc4)cc3)(c3ccc(-c4ccccc4)cc3)c3ccc(-c4cccc(F)c4)cc3)cc2)c1]

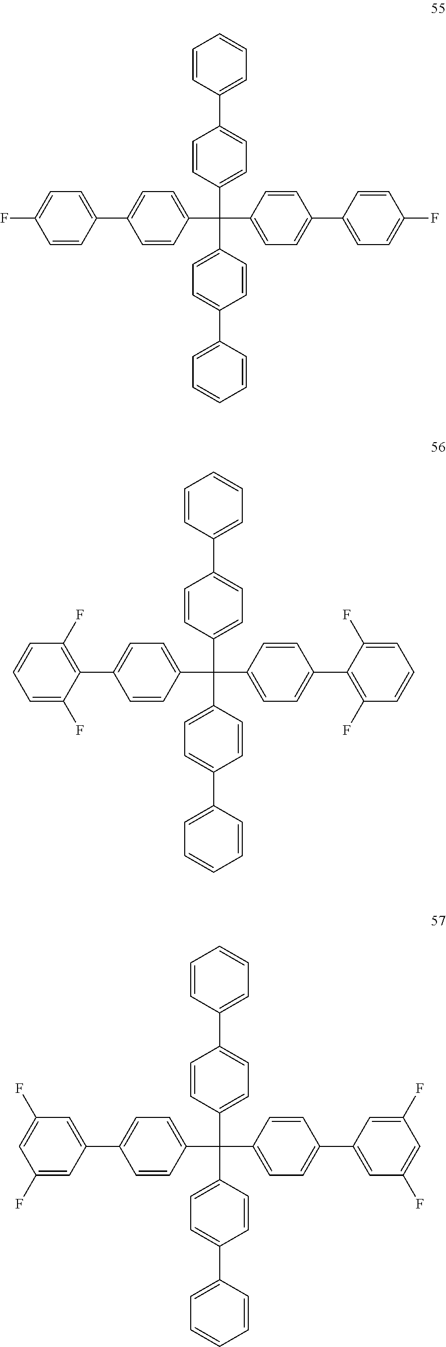 [SMILES: Fc1ccc(-c2ccc(C(c3ccc(-c4ccccc4)cc3)(c3ccc(-c4ccccc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3ccccc3)cc2)c2ccc(-c3c(F)cccc3F)cc2)cc1 ; Fc1cc(F)cc(-c2ccc(C(c3ccc(-c4ccccc4)cc3)(c3ccc(-c4ccccc4)cc3)c3ccc(-c4cc(F)cc(F)c4)cc3)cc2)c1]

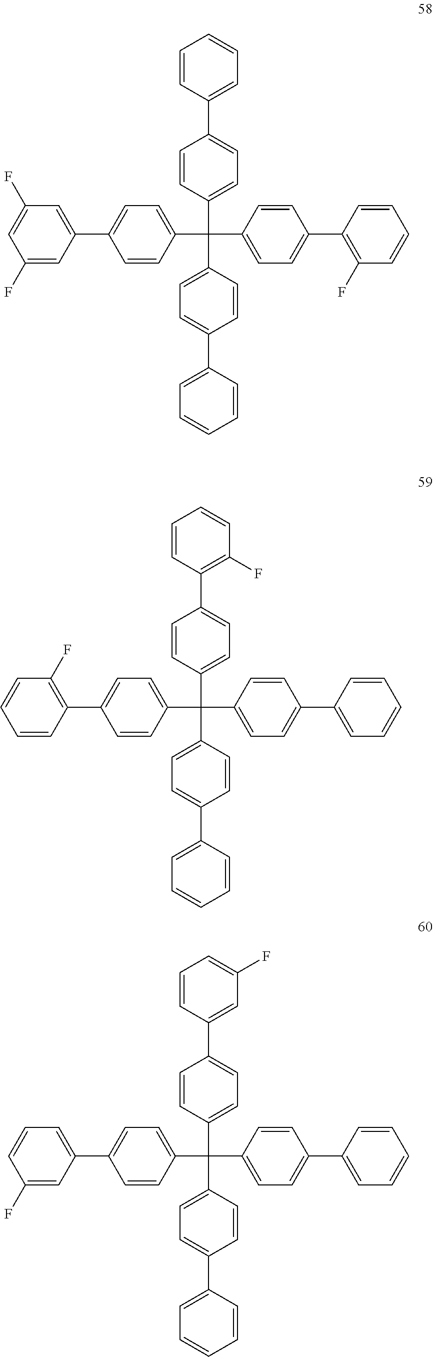 [SMILES: Fc1cc(F)cc(-c2ccc(C(c3ccc(-c4ccccc4)cc3)(c3ccc(-c4ccccc4)cc3)c3ccc(-c4ccccc4F)cc3)cc2)c1 ; Fc1ccccc1-c1ccc(C(c2ccc(-c3ccccc3)cc2)(c2ccc(-c3ccccc3)cc2)c2ccc(-c3ccccc3F)cc2)cc1 ; Fc1cccc(-c2ccc(C(c3ccc(-c4ccccc4)cc3)(c3ccc(-c4ccccc4)cc3)c3ccc(-c4cccc(F)c4)cc3)cc2)c1]

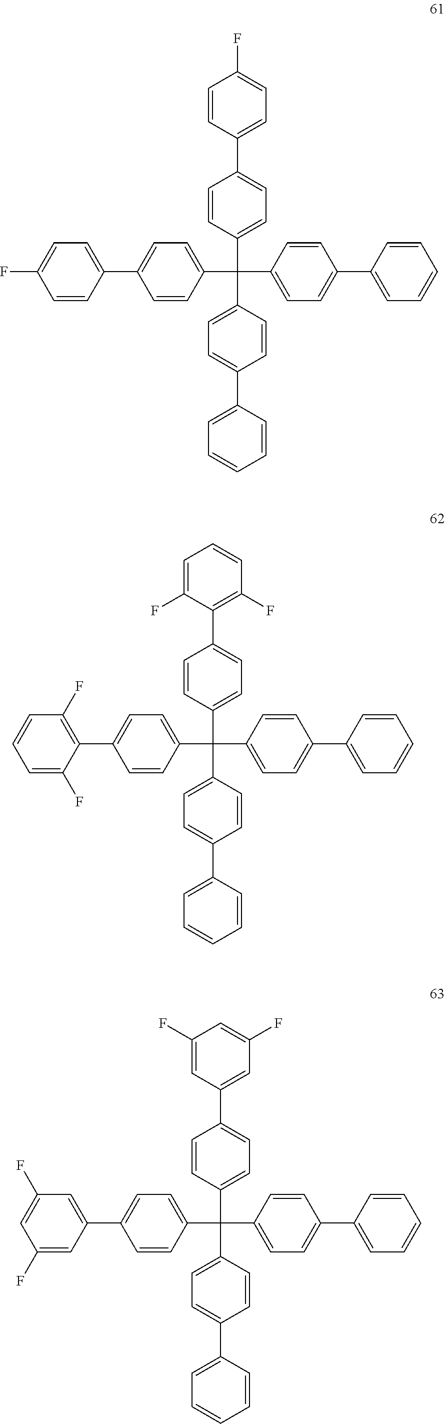 [SMILES: Fc1ccc(-c2ccc(C(c3ccc(-c4ccccc4)cc3)(c3ccc(-c4ccccc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3ccccc3)cc2)c2ccc(-c3c(F)cccc3F)cc2)cc1 ; Fc1cc(F)cc(-c2ccc(C(c3ccc(-c4ccccc4)cc3)(c3ccc(-c4ccccc4)cc3)c3ccc(-c4cc(F)cc(F)c4)cc3)cc2)c1]

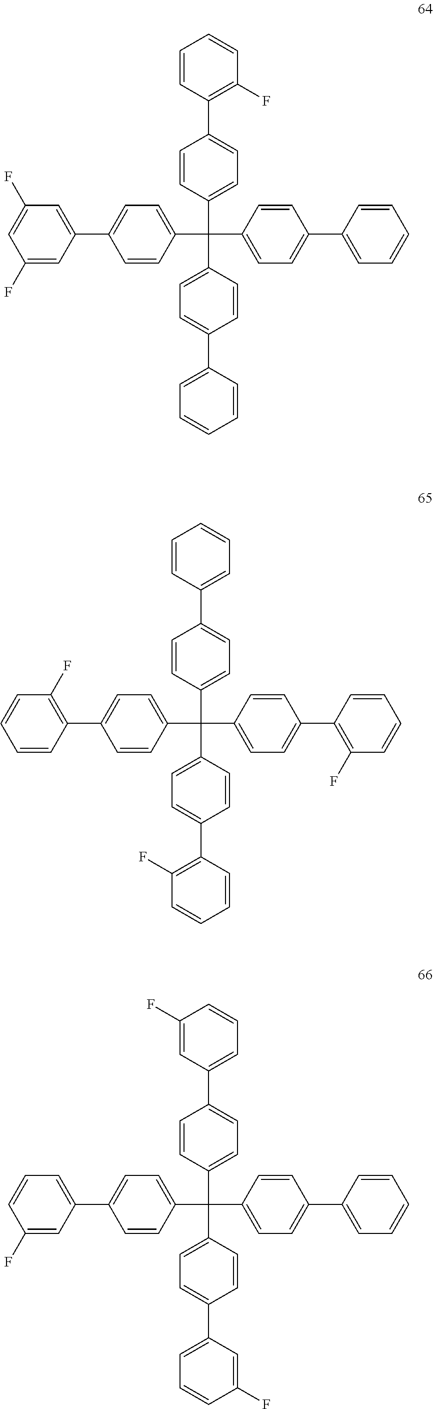 [SMILES: Fc1cc(F)cc(-c2ccc(C(c3ccc(-c4ccccc4)cc3)(c3ccc(-c4ccccc4)cc3)c3ccc(-c4ccccc4F)cc3)cc2)c1 ; Fc1ccccc1-c1ccc(C(c2ccc(-c3ccccc3)cc2)(c2ccc(-c3ccccc3F)cc2)c2ccc(-c3ccccc3F)cc2)cc1 ; Fc1cccc(-c2ccc(C(c3ccc(-c4ccccc4)cc3)(c3ccc(-c4cccc(F)c4)cc3)c3ccc(-c4cccc(F)c4)cc3)cc2)c1]

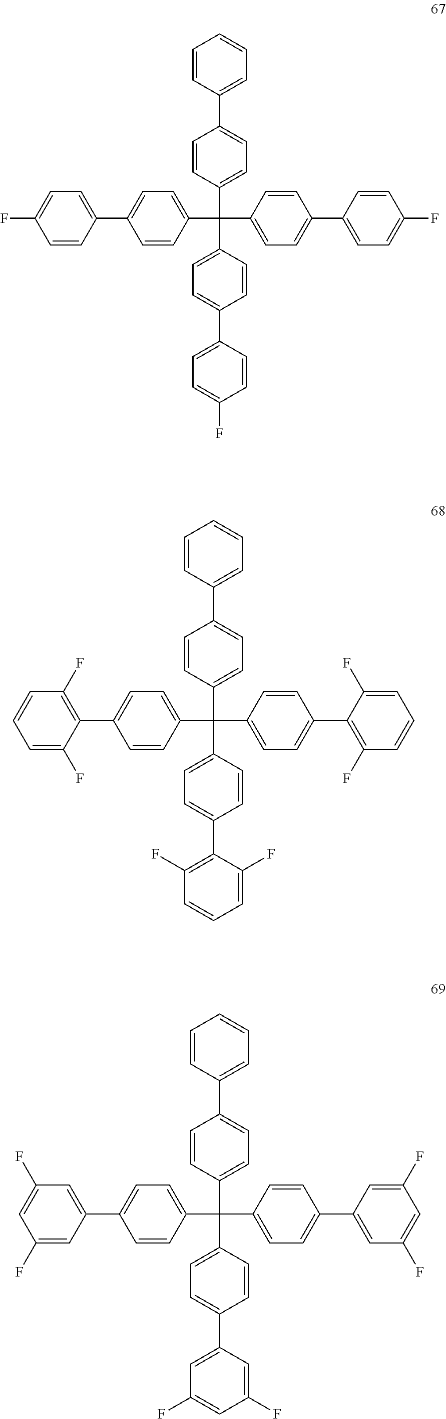 [SMILES: Fc1ccc(-c2ccc(C(c3ccc(-c4ccccc4)cc3)(c3ccc(-c4ccc(F)cc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3c(F)cccc3F)cc2)c2ccc(-c3c(F)cccc3F)cc2)cc1 ; Fc1cc(F)cc(-c2ccc(C(c3ccc(-c4ccccc4)cc3)(c3ccc(-c4cc(F)cc(F)c4)cc3)c3ccc(-c4cc(F)cc(F)c4)cc3)cc2)c1]

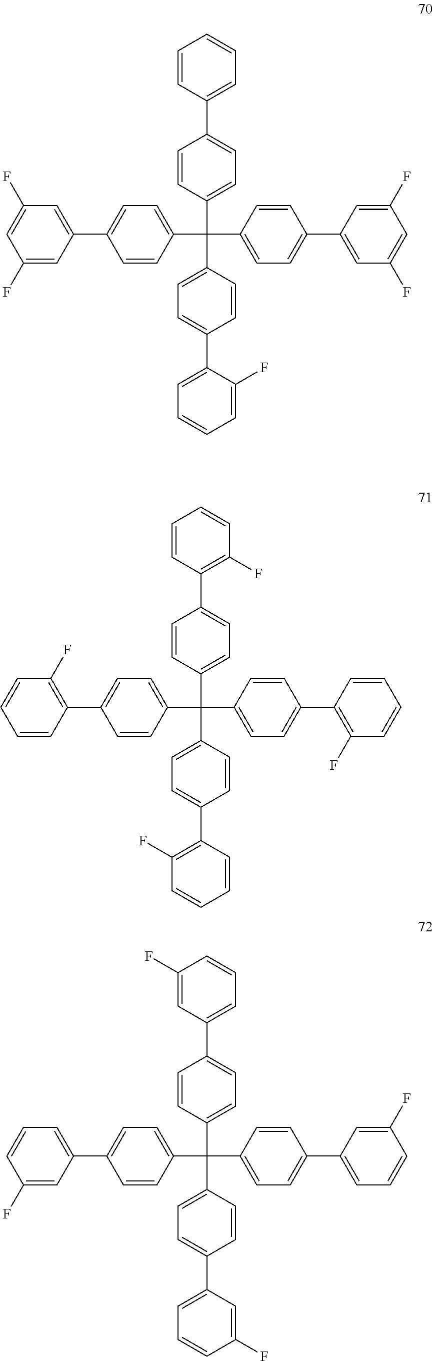 [SMILES: Fc1cc(F)cc(-c2ccc(C(c3ccc(-c4ccccc4)cc3)(c3ccc(-c4cc(F)cc(F)c4)cc3)c3ccc(-c4ccccc4F)cc3)cc2)c1 ; Fc1ccccc1-c1ccc(C(c2ccc(-c3ccccc3F)cc2)(c2ccc(-c3ccccc3F)cc2)c2ccc(-c3ccccc3F)cc2)cc1 ; Fc1cccc(-c2ccc(C(c3ccc(-c4cccc(F)c4)cc3)(c3ccc(-c4cccc(F)c4)cc3)c3ccc(-c4cccc(F)c4)cc3)cc2)c1]

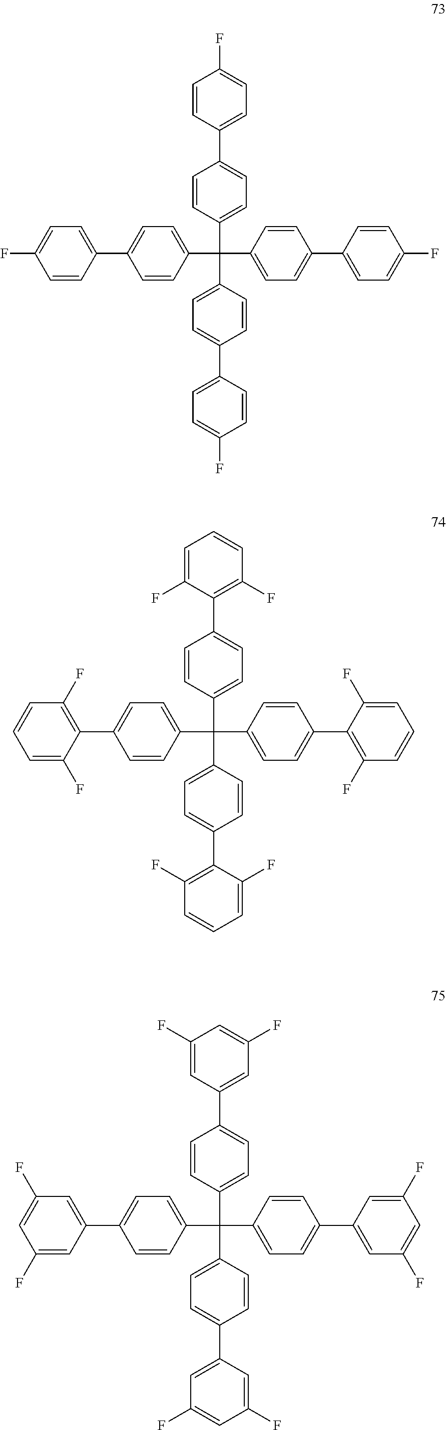 [SMILES: Fc1ccc(-c2ccc(C(c3ccc(-c4ccc(F)cc4)cc3)(c3ccc(-c4ccc(F)cc4)cc3)c3ccc(-c4ccc(F)cc4)cc3)cc2)cc1 ; Fc1cccc(F)c1-c1ccc(C(c2ccc(-c3c(F)cccc3F)cc2)(c2ccc(-c3c(F)cccc3F)cc2)c2ccc(-c3c(F)cccc3F)cc2)cc1 ; Fc1cc(F)cc(-c2ccc(C(c3ccc(-c4cc(F)cc(F)c4)cc3)(c3ccc(-c4cc(F)cc(F)c4)cc3)c3ccc(-c4cc(F)cc(F)c4)cc3)cc2)c1]

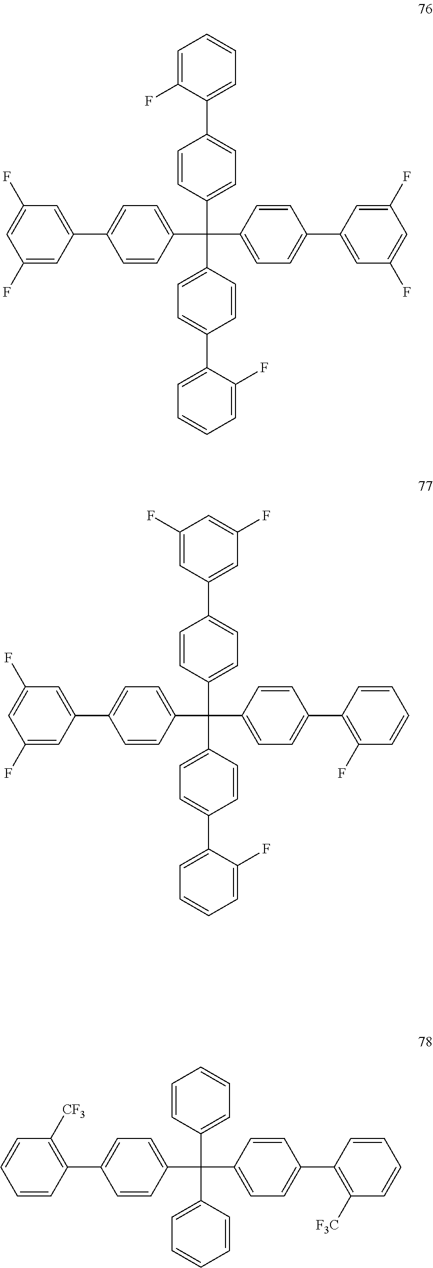 [SMILES: Fc1cc(F)cc(-c2ccc(C(c3ccc(-c4cc(F)cc(F)c4)cc3)(c3ccc(-c4ccccc4F)cc3)c3ccc(-c4ccccc4F)cc3)cc2)c1 ; Fc1cc(F)cc(-c2ccc(C(c3ccc(-c4cc(F)cc(F)c4)cc3)(c3ccc(-c4ccccc4F)cc3)c3ccc(-c4ccccc4F)cc3)cc2)c1 ; FC(F)(F)c1ccccc1-c1ccc(C(c2ccccc2)(c2ccccc2)c2ccc(-c3ccccc3C(F)(F)F)cc2)cc1]

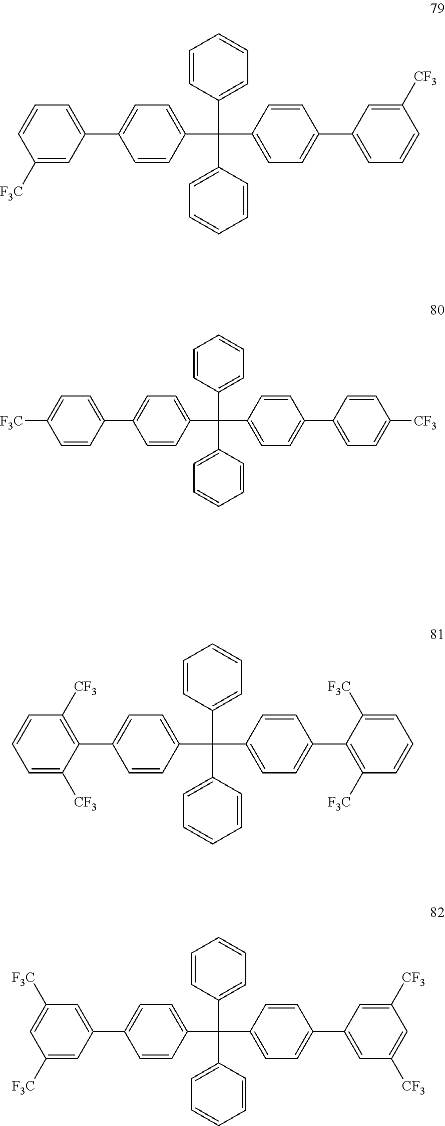 [SMILES: FC(F)(F)c1cccc(-c2ccc(C(c3ccccc3)(c3ccccc3)c3ccc(-c4cccc(C(F)(F)F)c4)cc3)cc2)c1 ; FC(F)(F)c1ccc(-c2ccc(C(c3ccccc3)(c3ccccc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccccc2)c2ccc(-c3c(C(F)(F)F)cccc3C(F)(F)F)cc2)cc1 ; FC(F)(F)c1cc(-c2ccc(C(c3ccccc3)(c3ccccc3)c3ccc(-c4cc(C(F)(F)F)cc(C(F)(F)F)c4)cc3)cc2)cc(C(F)(F)F)c1]

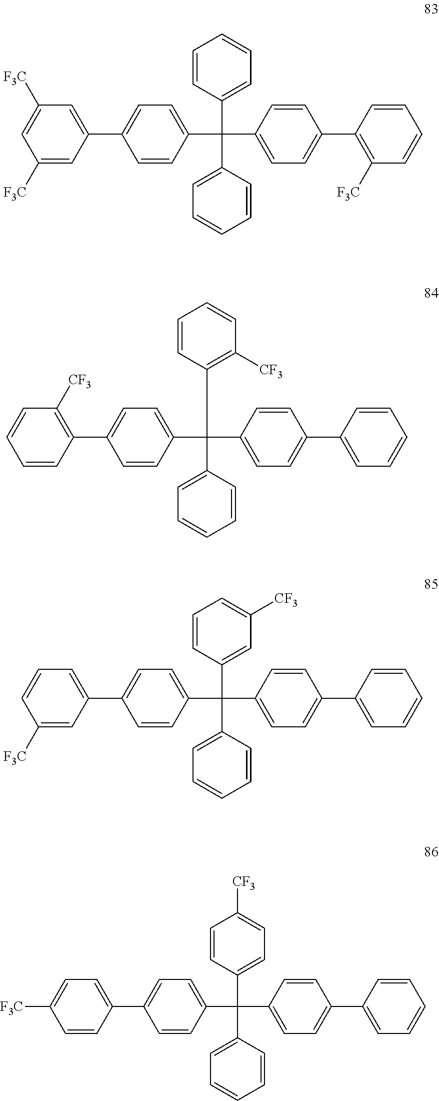 [SMILES: FC(F)(F)c1cc(-c2ccc(C(c3ccccc3)(c3ccccc3)c3ccc(-c4ccccc4C(F)(F)F)cc3)cc2)cc(C(F)(F)F)c1 ; FC(F)(F)c1ccccc1-c1ccc(C(c2ccccc2)(c2ccc(-c3ccccc3)cc2)c2ccccc2C(F)(F)F)cc1 ; FC(F)(F)c1cccc(-c2ccc(C(c3ccccc3)(c3ccc(-c4ccccc4)cc3)c3cccc(C(F)(F)F)c3)cc2)c1 ; FC(F)(F)c1ccc(-c2ccc(C(c3ccccc3)(c3ccc(-c4ccccc4)cc3)c3ccc(C(F)(F)F)cc3)cc2)cc1]

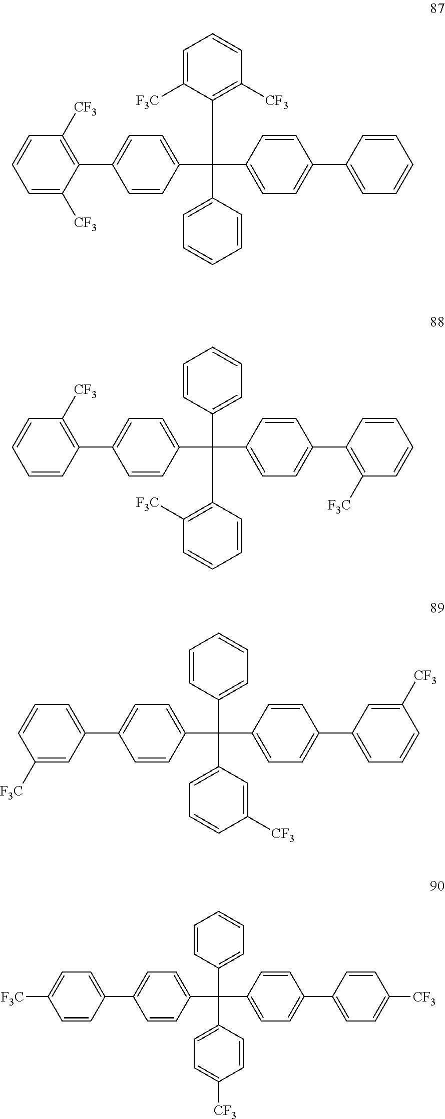 [SMILES: FC(F)(F)c1cccc(C(F)(F)F)c1-c1ccc(C(c2ccccc2)(c2ccc(-c3ccccc3)cc2)c2c(C(F)(F)F)cccc2C(F)(F)F)cc1 ; FC(F)(F)c1ccccc1-c1ccc(C(c2ccccc2)(c2ccc(-c3ccccc3C(F)(F)F)cc2)c2ccccc2C(F)(F)F)cc1 ; FC(F)(F)c1cccc(-c2ccc(C(c3ccccc3)(c3ccc(-c4cccc(C(F)(F)F)c4)cc3)c3cccc(C(F)(F)F)c3)cc2)c1 ; FC(F)(F)c1ccc(-c2ccc(C(c3ccccc3)(c3ccc(-c4ccc(C(F)(F)F)cc4)cc3)c3ccc(C(F)(F)F)cc3)cc2)cc1]

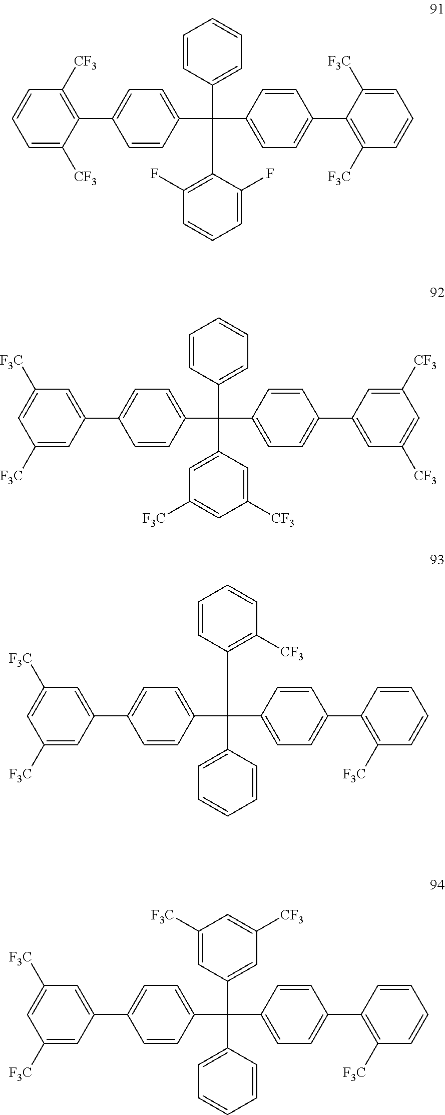 [SMILES: Fc1cccc(F)c1C(c1ccccc1)(c1ccc(-c2c(C(F)(F)F)cccc2C(F)(F)F)cc1)c1ccc(-c2c(C(F)(F)F)cccc2C(F)(F)F)cc1 ; FC(F)(F)c1cc(-c2ccc(C(c3ccccc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc(C(c3ccccc3)(c3ccc(-c4ccccc4C(F)(F)F)cc3)c3ccccc3C(F)(F)F)cc2)cc(C(F)(F)F)c1 ; FC(F)(F)c1cc(-c2ccc(C(c3ccccc3)(c3ccc(-c4ccccc4C(F)(F)F)cc3)c3cc(C(F)(F)F)cc(C(F)(F)F)c3)cc2)cc(C(F)(F)F)c1]

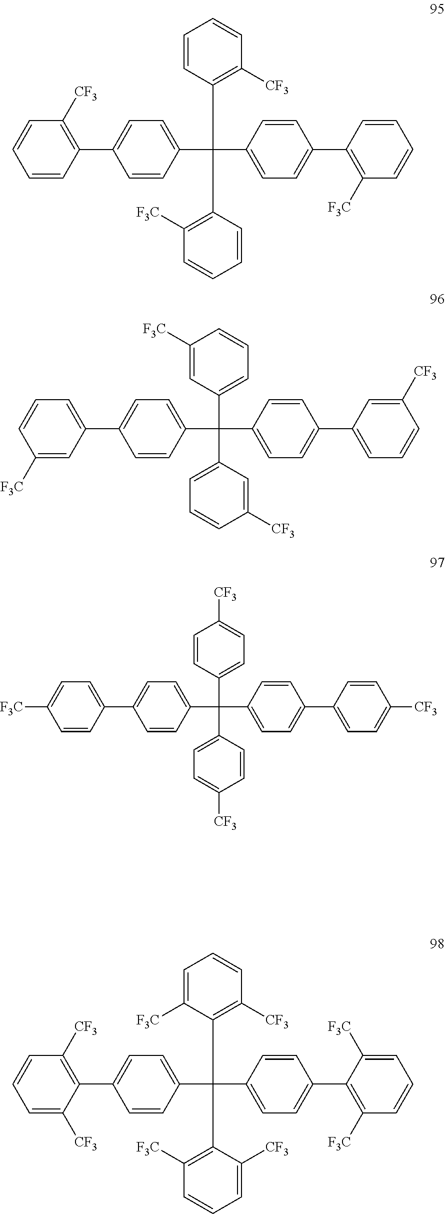 [SMILES: FC(F)(F)c1ccccc1-c1ccc(C(c2ccc(-c3ccccc3C(F)(F)F)cc2)(c2ccccc2C(F)(F)F)c2ccccc2C(F)(F)F)cc1 ; FC(F)(F)c1cccc(-c2ccc(C(c3ccc(-c4cccc(C(F)(F)F)c4)cc3)(c3cccc(C(F)(F)F)c3)c3cccc(C(F)(F)F)c3)cc2)c1 ; FC(F)(F)c1ccc(-c2ccc(C(c3ccc(-c4ccc(C(F)(F)F)cc4)cc3)(c3ccc(C(F)(F)F)cc3)c3ccc(C(F)(F)F)cc3)cc2)cc1 ; FC(F)(F)c1cccc(C(F)(F)F)c1-c1ccc(C(c2ccc(-c3c(C(F)(F)F)cccc3C(F)(F)F)cc2)(c2c(C(F)(F)F)cccc2C(F)(F)F)c2c(C(F)(F)F)cccc2C(F)(F)F)cc1]

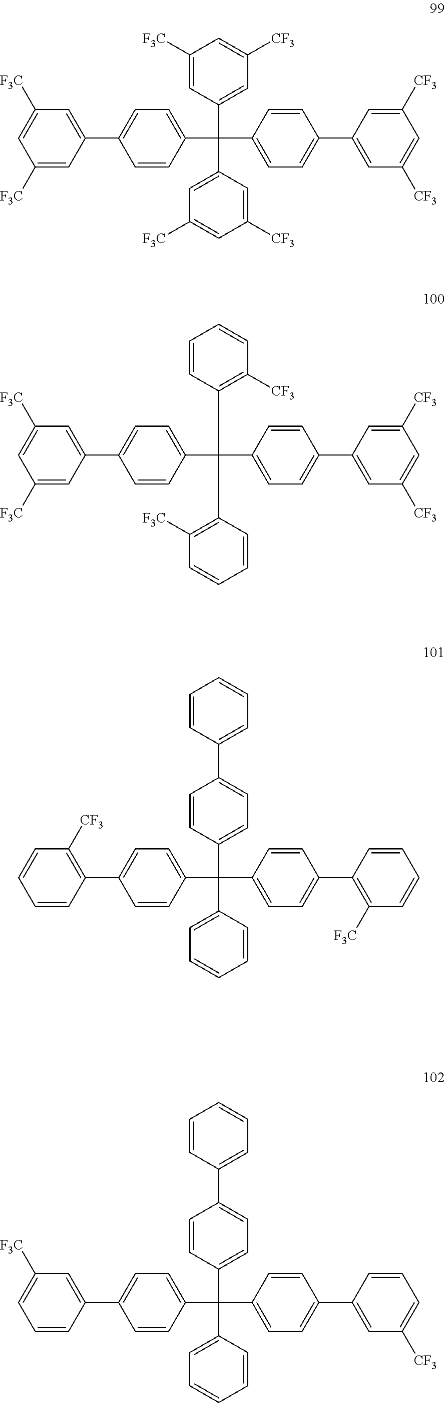 [SMILES: FC(F)(F)c1cc(-c2ccc(C(c3ccc(-c4cc(C(F)(F)F)cc(C(F)(F)F)c4)cc3)(c3cc(C(F)(F)F)cc(C(F)(F)F)c3)c3cc(C(F)(F)F)cc(C(F)(F)F)c3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccccc3C(F)(F)F)c3ccccc3C(F)(F)F)cc2)cc(C(F)(F)F)c1 ; FC(F)(F)c1ccccc1-c1ccc(C(c2ccccc2)(c2ccc(-c3ccccc3)cc2)c2ccc(-c3ccccc3C(F)(F)F)cc2)cc1 ; FC(F)(F)c1cccc(-c2ccc(C(c3ccccc3)(c3ccc(-c4ccccc4)cc3)c3ccc(-c4cccc(C(F)(F)F)c4)cc3)cc2)c1]

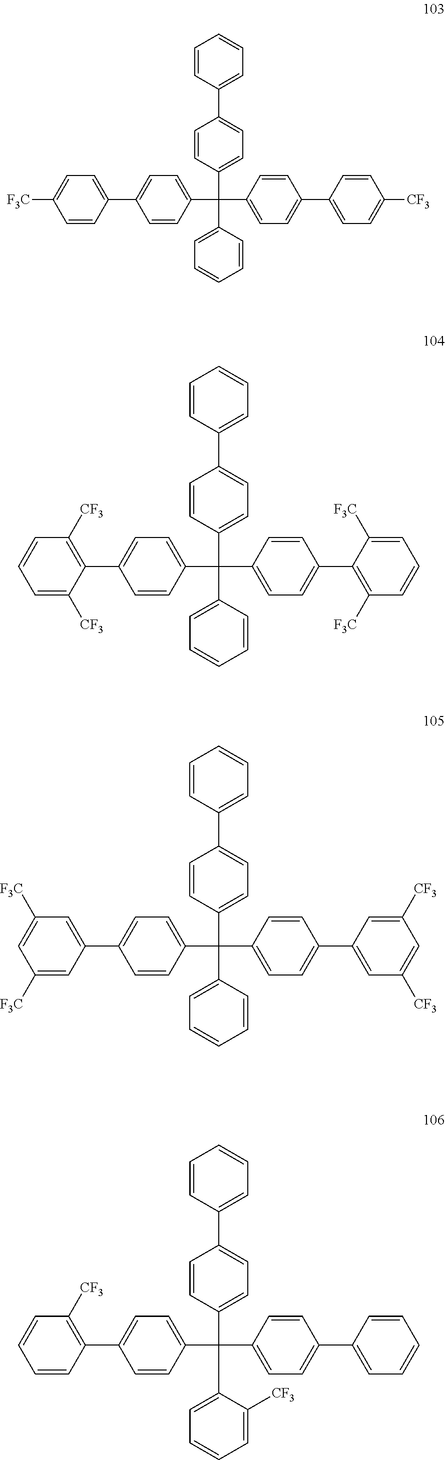 [SMILES: FC(F)(F)c1ccc(-c2ccc(C(c3ccccc3)(c3ccc(-c4ccccc4)cc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccccc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1ccccc1-c1ccc(C(c2ccc(-c3ccccc3)cc2)(c2ccc(-c3ccccc3)cc2)c2ccccc2C(F)(F)F)cc1]

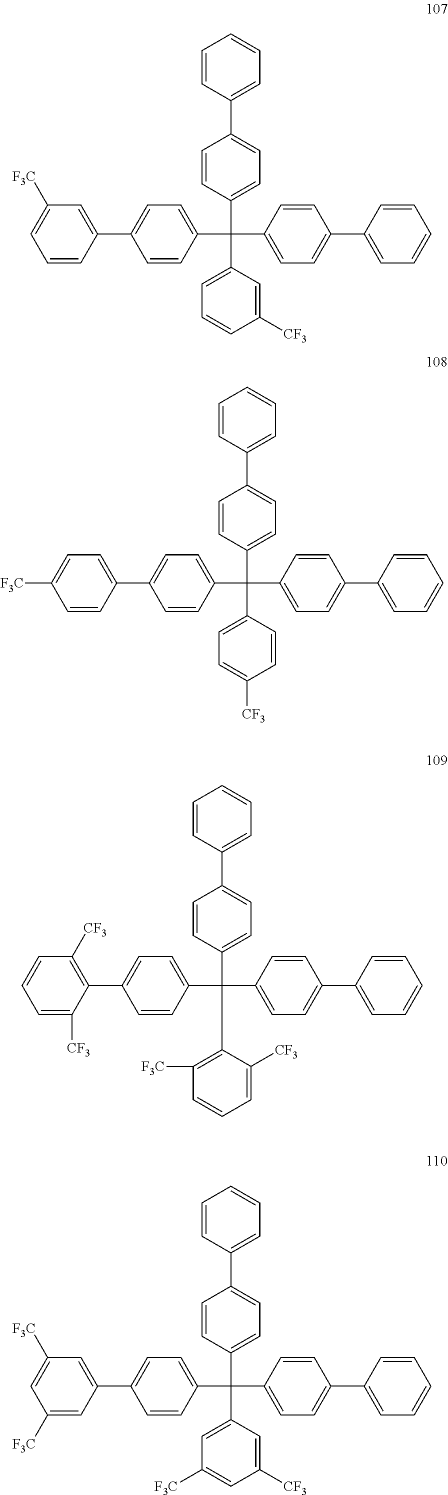 [SMILES: FC(F)(F)c1cccc(-c2ccc(C(c3ccc(-c4ccccc4)cc3)(c3ccc(-c4ccccc4)cc3)c3cccc(C(F)(F)F)c3)cc2)c1 ; FC(F)(F)c1ccc(-c2ccc(C(c3ccc(-c4ccccc4)cc3)(c3ccc(-c4ccccc4)cc3)c3ccc(C(F)(F)F)cc3)cc2)cc1 ; FC(F)(F)c1cccc(C(F)(F)F)c1-c1ccc(C(c2ccc(-c3ccccc3)cc2)(c2ccc(-c3ccccc3)cc2)c2c(C(F)(F)F)cccc2C(F)(F)F)cc1 ; FC(F)(F)c1cc(-c2ccc(C(c3ccc(-c4ccccc4)cc3)(c3ccc(-c4ccccc4)cc3)c3cc(C(F)(F)F)cc(C(F)(F)F)c3)cc2)cc(C(F)(F)F)c1]

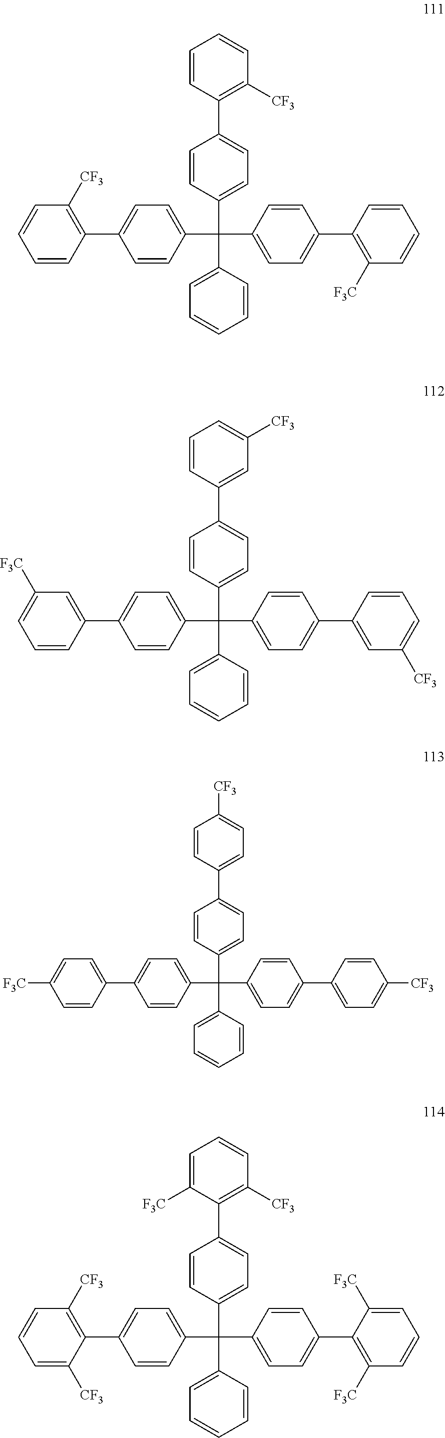 [SMILES: FC(F)(F)c1ccccc1-c1ccc(C(c2ccccc2)(c2ccc(-c3ccccc3C(F)(F)F)cc2)c2ccc(-c3ccccc3C(F)(F)F)cc2)cc1 ; FC(F)(F)c1cccc(-c2ccc(C(c3ccccc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccccc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccc(-c3c(C(F)(F)F)cccc3C(F)(F)F)cc2)c2ccc(-c3c(C(F)(F)F)cccc3C(F)(F)F)cc2)cc1]

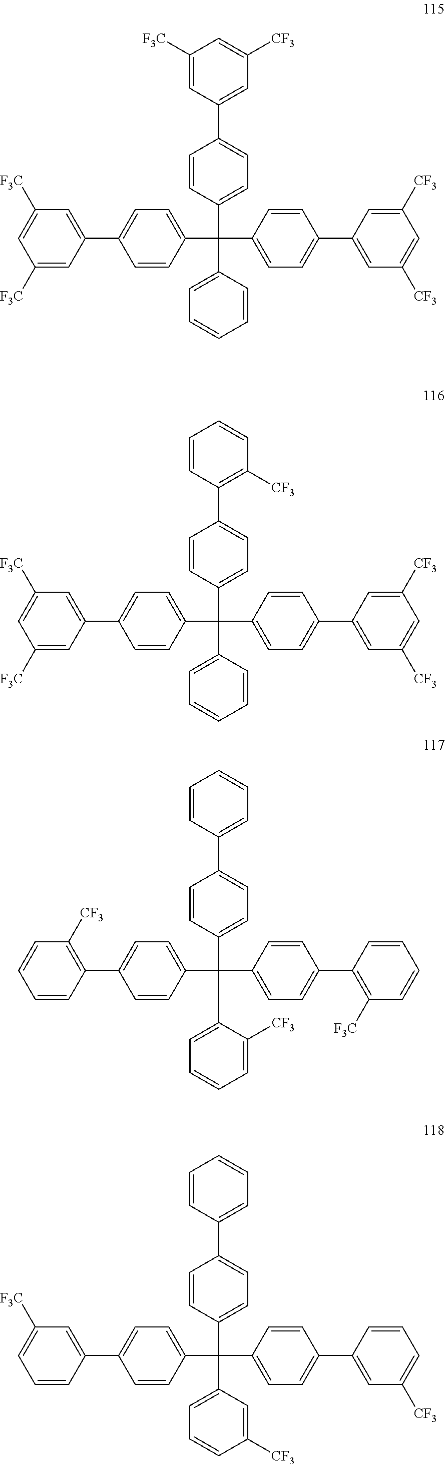 [SMILES: FC(F)(F)c1cc(-c2ccc(C(c3ccccc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccccc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc(C(c2ccc(-c3ccccc3)cc2)(c2ccc(-c3ccccc3C(F)(F)F)cc2)c2ccccc2C(F)(F)F)cc1 ; FC(F)(F)c1cccc(-c2ccc(C(c3ccc(-c4ccccc4)cc3)(c3ccc(-c4cccc(C(F)(F)F)c4)cc3)c3cccc(C(F)(F)F)c3)cc2)c1]

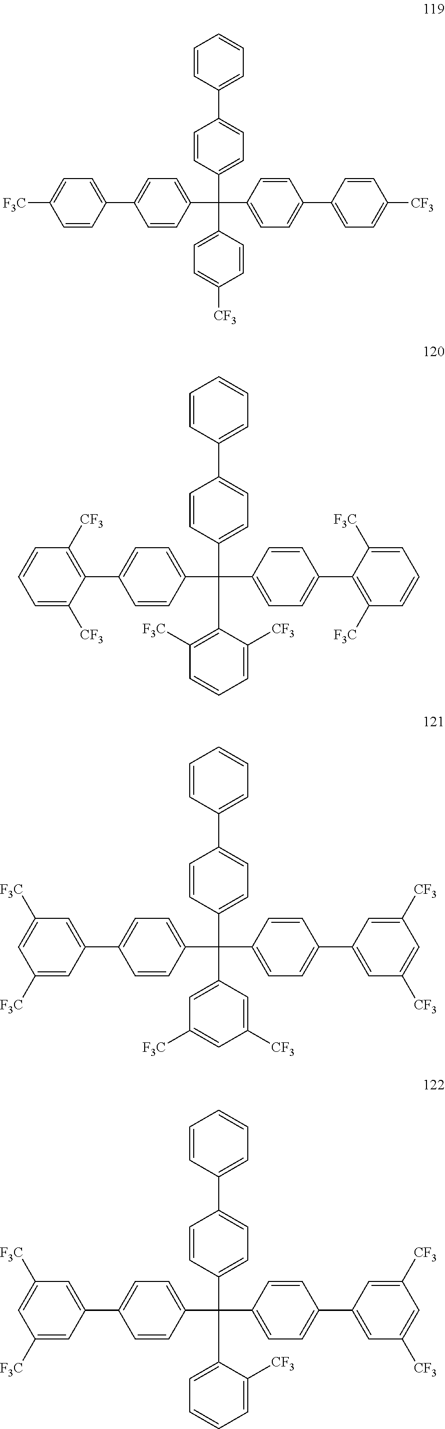 [SMILES: FC(F)(F)c1ccc(-c2ccc(C(c3ccc(-c4ccccc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc(C(c2ccc(-c3ccccc3)cc2)(c2ccc(-c3c(C(F)(F)F)cccc3C(F)(F)F)cc2)c2c(C(F)(F)F)cccc2C(F)(F)F)cc1 ; FC(F)(F)c1cc(-c2ccc(C(c3ccc(-c4ccccc4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc(C(c3ccc(-c4ccccc4)cc3)(c3ccc(-c4cc(C(F)(F)F)cc(C(F)(F)F)c4)cc3)c3ccccc3C(F)(F)F)cc2)cc(C(F)(F)F)c1]

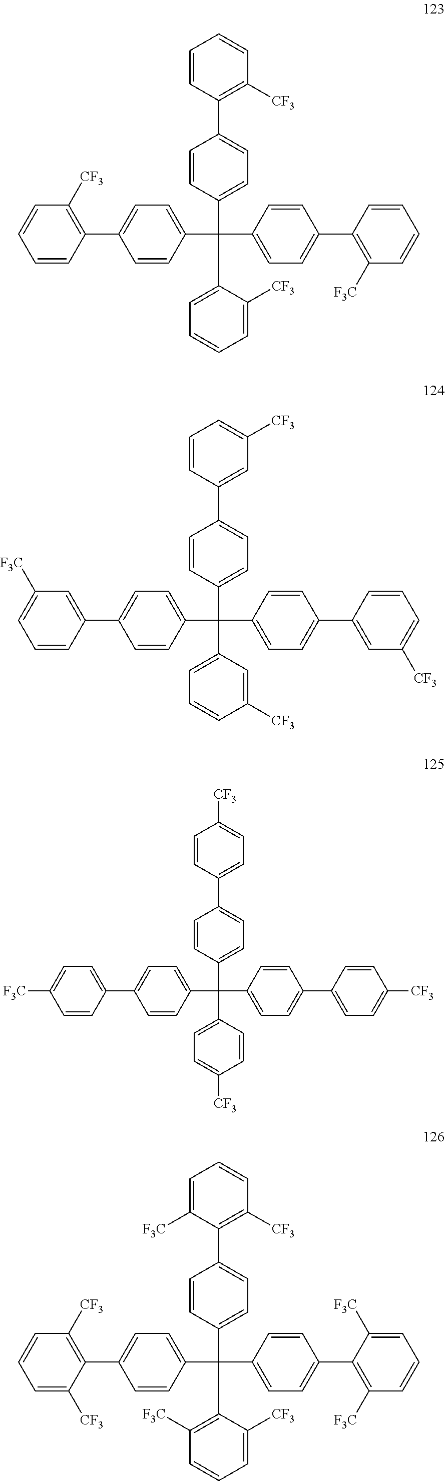 [SMILES: FC(F)(F)c1ccccc1-c1ccc(C(c2ccc(-c3ccccc3C(F)(F)F)cc2)(c2ccc(-c3ccccc3C(F)(F)F)cc2)c2ccccc2C(F)(F)F)cc1 ; FC(F)(F)c1cccc(-c2ccc(C(c3ccc(-c4cccc(C(F)(F)F)c4)cc3)(c3ccc(-c4cccc(C(F)(F)F)c4)cc3)c3cccc(C(F)(F)F)c3)cc2)c1 ; FC(F)(F)c1ccc(-c2ccc(C(c3ccc(-c4ccc(C(F)(F)F)cc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc(C(c2ccc(-c3c(C(F)(F)F)cccc3C(F)(F)F)cc2)(c2ccc(-c3c(C(F)(F)F)cccc3C(F)(F)F)cc2)c2c(C(F)(F)F)cccc2C(F)(F)F)cc1]

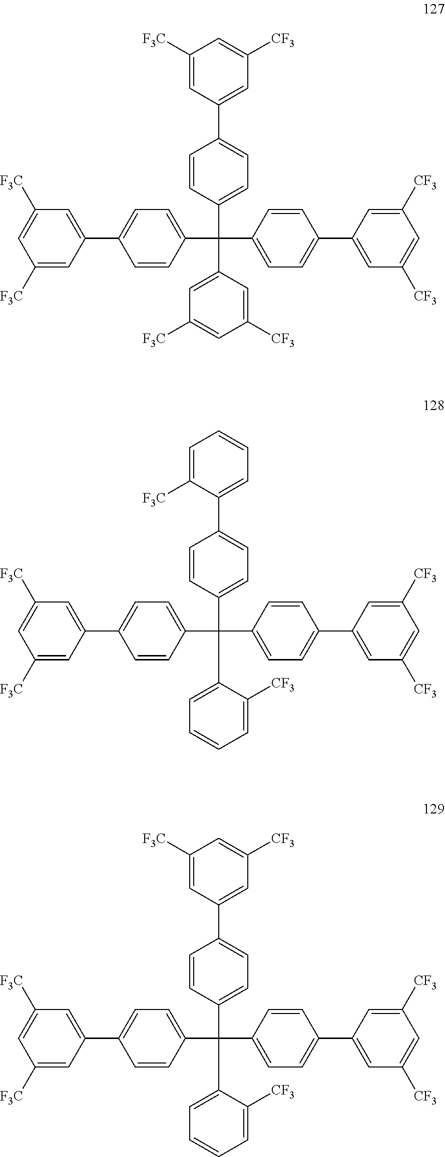 [SMILES: FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccccc3C(F)(F)F)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3ccccc3C(F)(F)F)cc2)cc(C(F)(F)F)c1]

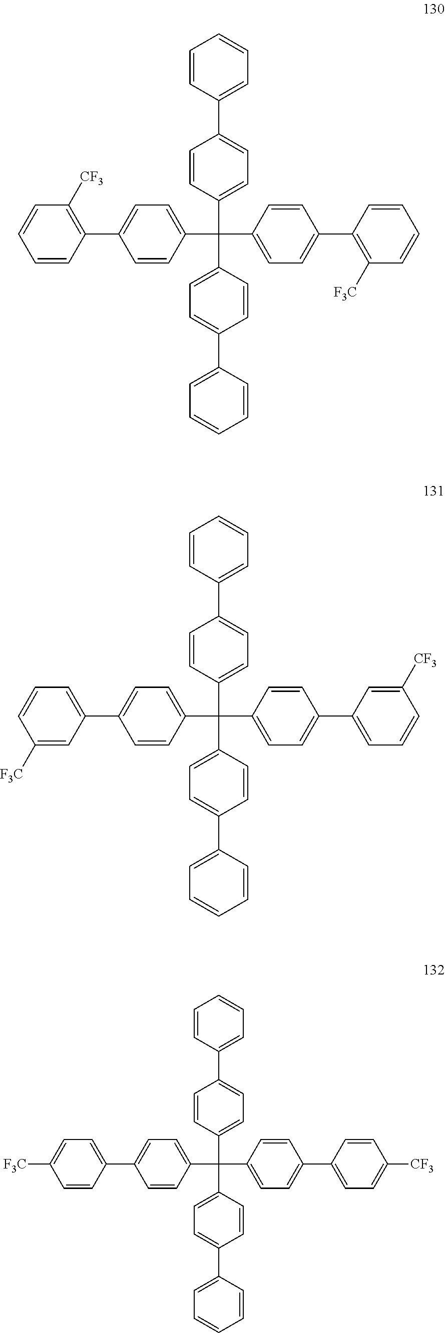 [SMILES: FC(F)(F)c1ccccc1-c1ccc(C(c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc(C(c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccccc4)cc3)c3ccc(-c4ccc(C(F)(F)F)cc4)cc3)cc2)cc1]

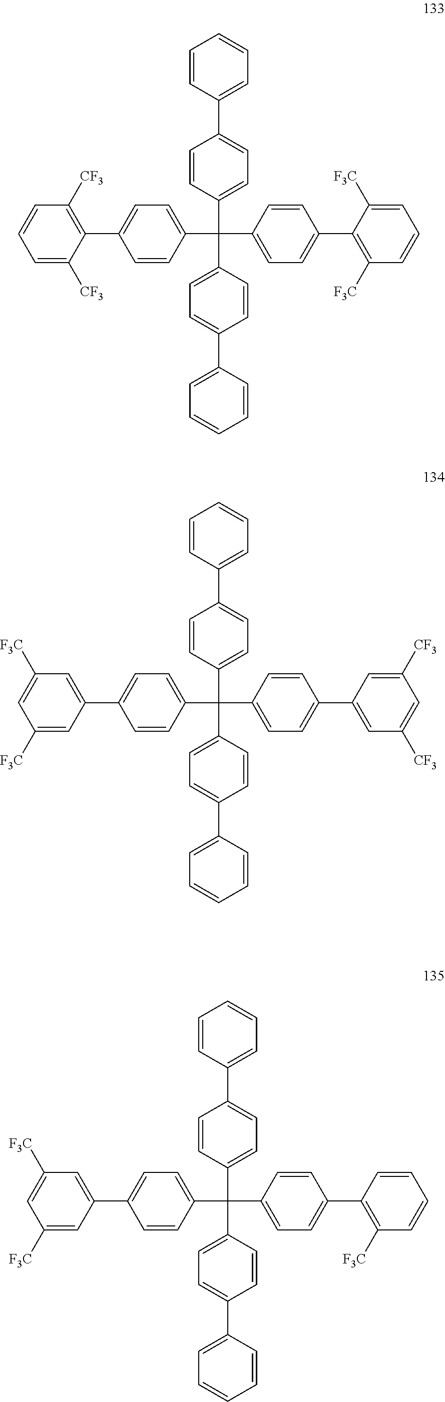 [SMILES: FC(F)(F)c1cccc(C(F)(F)F)c1-c1ccc(C(c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4ccccc4)cc3)c3ccc(-c4ccccc4C(F)(F)F)cc3)cc2)cc(C(F)(F)F)c1]

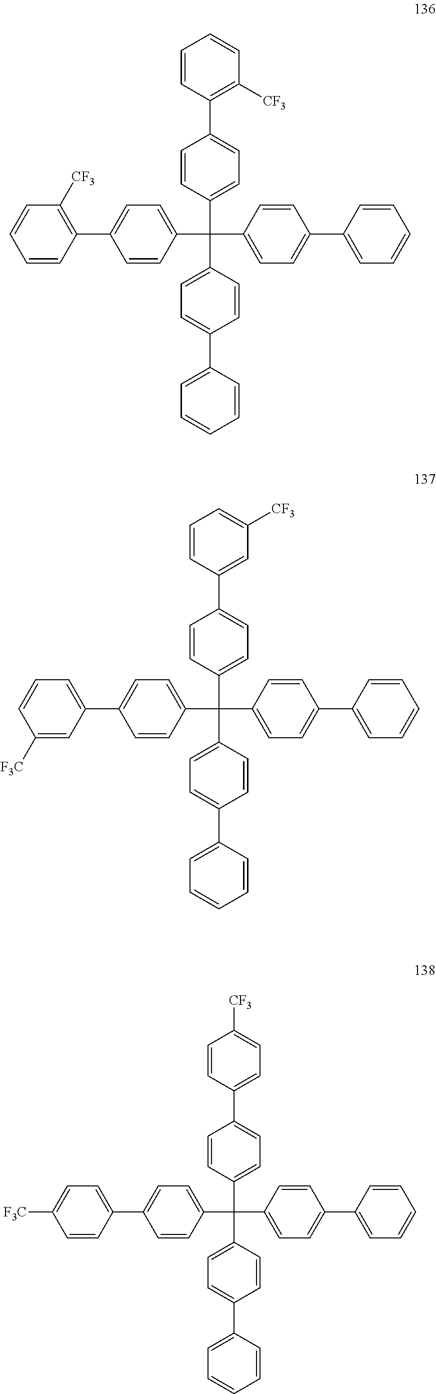 [SMILES: FC(F)(F)c1ccccc1-c1ccc(C(c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc(C(c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccccc4)cc3)c3ccc(-c4ccc(C(F)(F)F)cc4)cc3)cc2)cc1]

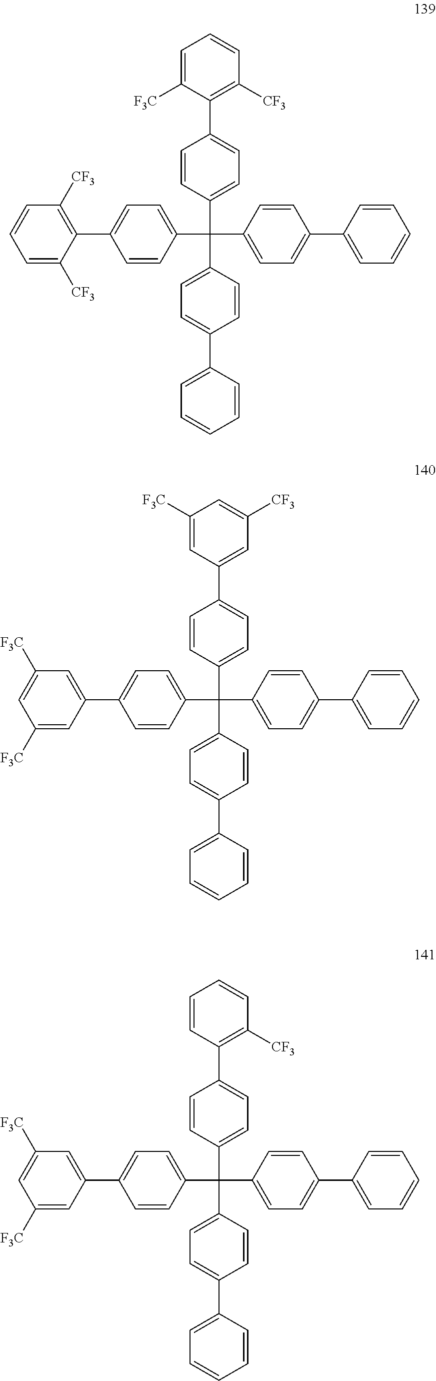 [SMILES: FC(F)(F)c1cccc(C(F)(F)F)c1-c1ccc(C(c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4ccccc4)cc3)c3ccc(-c4ccccc4C(F)(F)F)cc3)cc2)cc(C(F)(F)F)c1]

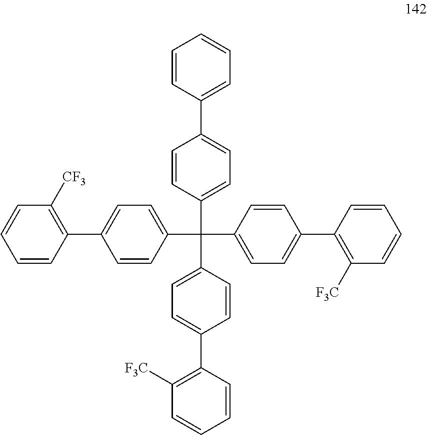 [SMILES: FC(F)(F)c1ccccc1-c1ccc(C(c2ccc(-c3ccccc3)cc2)(c2ccc(-c3ccccc3C(F)(F)F)cc2)c2ccc(-c3ccccc3C(F)(F)F)cc2)cc1]

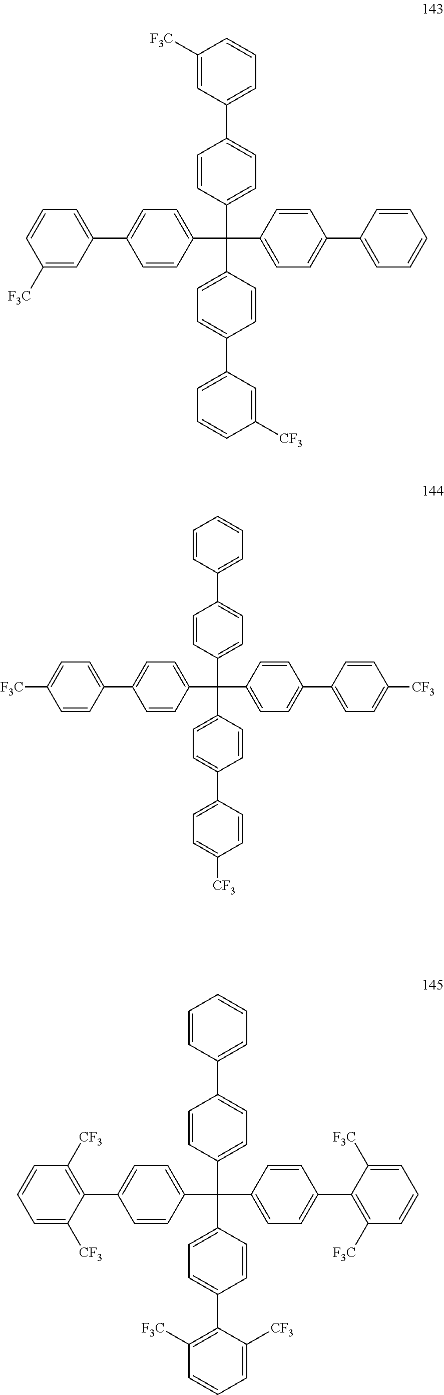 [SMILES: FC(F)(F)c1cccc(-c2ccc(C(c3ccc(-c4ccccc4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccc(-c3ccccc3)cc2)(c2ccc(-c3c(C(F)(F)F)cccc3C(F)(F)F)cc2)c2ccc(-c3c(C(F)(F)F)cccc3C(F)(F)F)cc2)cc1]

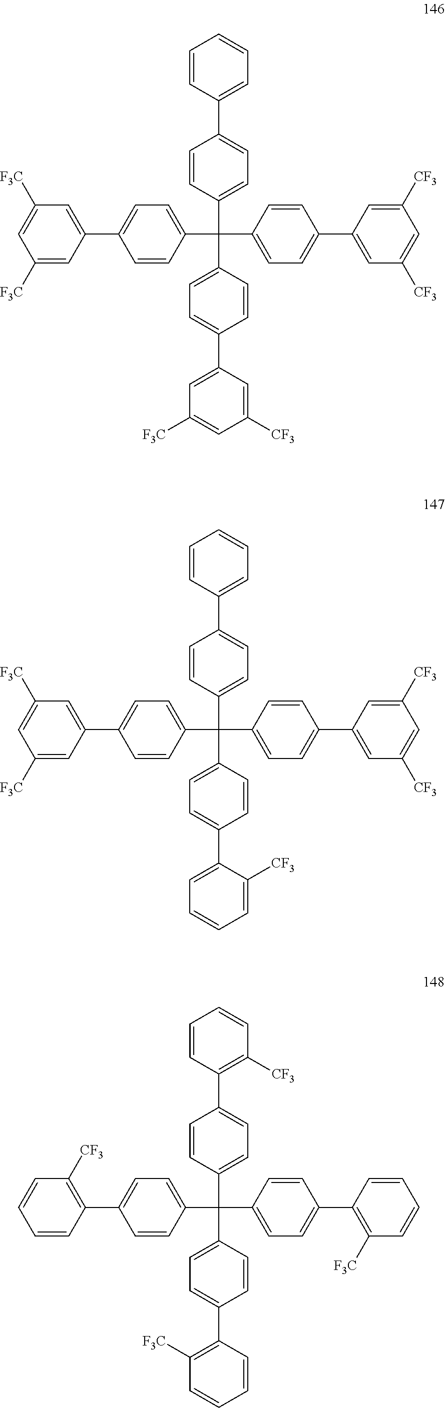 [SMILES: FC(F)(F)c1cc(-c2ccc(C(c3ccc(-c4ccccc4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc(C(c2ccc(-c3ccccc3C(F)(F)F)cc2)(c2ccc(-c3ccccc3C(F)(F)F)cc2)c2ccc(-c3ccccc3C(F)(F)F)cc2)cc1]

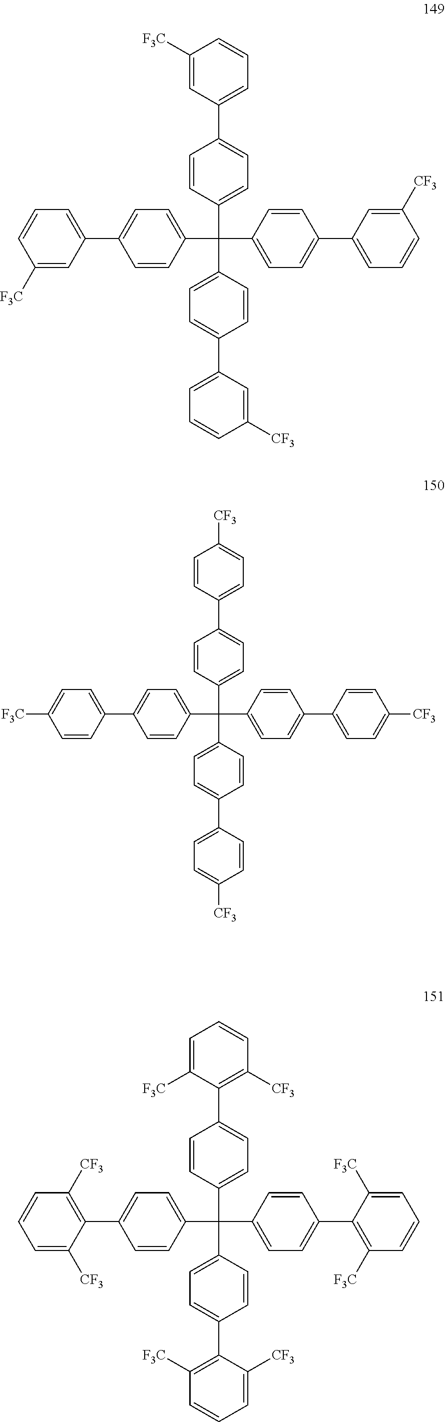 [SMILES: FC(F)(F)c1cccc(-c2ccc(C(c3ccc(-c4cccc(C(F)(F)F)c4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccc(-c4ccc(C(F)(F)F)cc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccc(-c3c(C(F)(F)F)cccc3C(F)(F)F)cc2)(c2ccc(-c3c(C(F)(F)F)cccc3C(F)(F)F)cc2)c2ccc(-c3c(C(F)(F)F)cccc3C(F)(F)F)cc2)cc1]

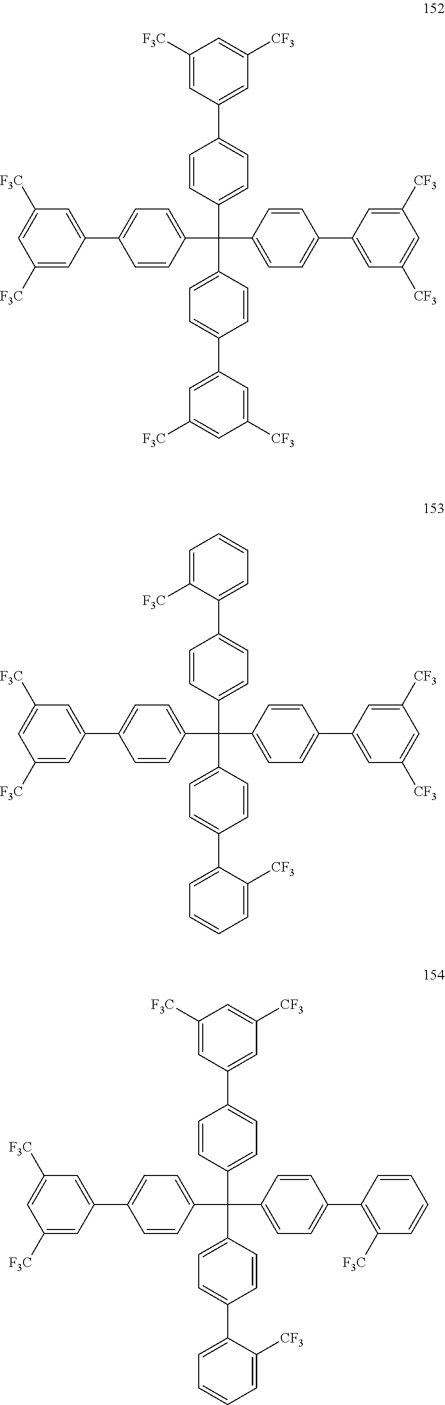 [SMILES: FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1]

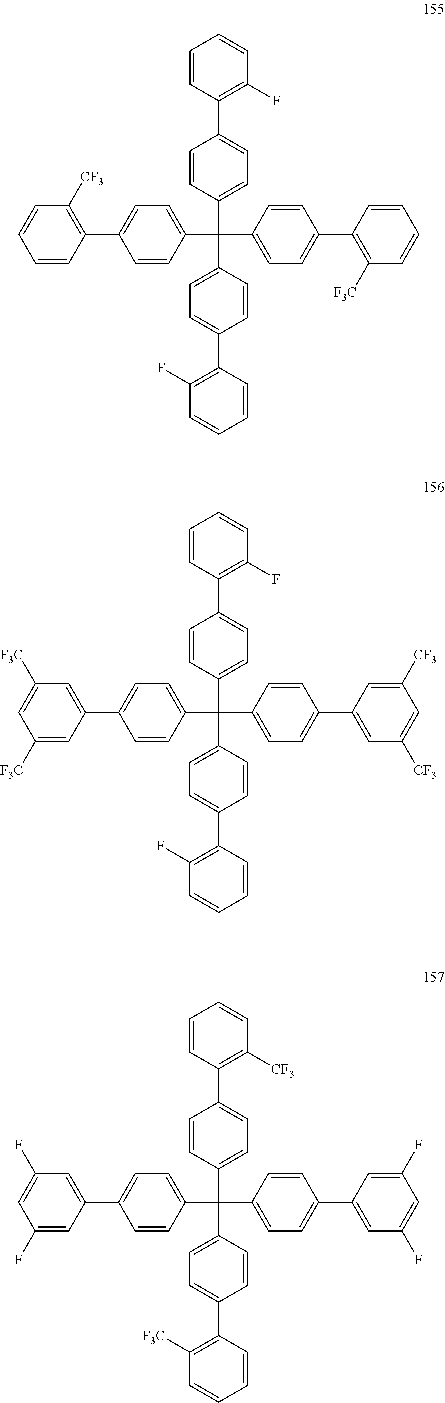 [SMILES: Fc1ccccc1-c1ccc(C(c2ccc(-c3ccccc3F)cc2)(c2ccc(-c3ccccc3C(F)(F)F)cc2)c2ccc(-c3ccccc3C(F)(F)F)cc2)cc1 ; Fc1ccccc1-c1ccc(C(c2ccc(-c3cc(C(F)(F)F)cc(C(F)(F)F)c3)cc2)(c2ccc(-c3cc(C(F)(F)F)cc(C(F)(F)F)c3)cc2)c2ccc(-c3ccccc3F)cc2)cc1 ; Fc1cc(F)cc(-c2ccc(C(c3ccc(-c4cc(F)cc(F)c4)cc3)(c3ccc(-c4ccccc4C(F)(F)F)cc3)c3ccc(-c4ccccc4C(F)(F)F)cc3)cc2)c1]

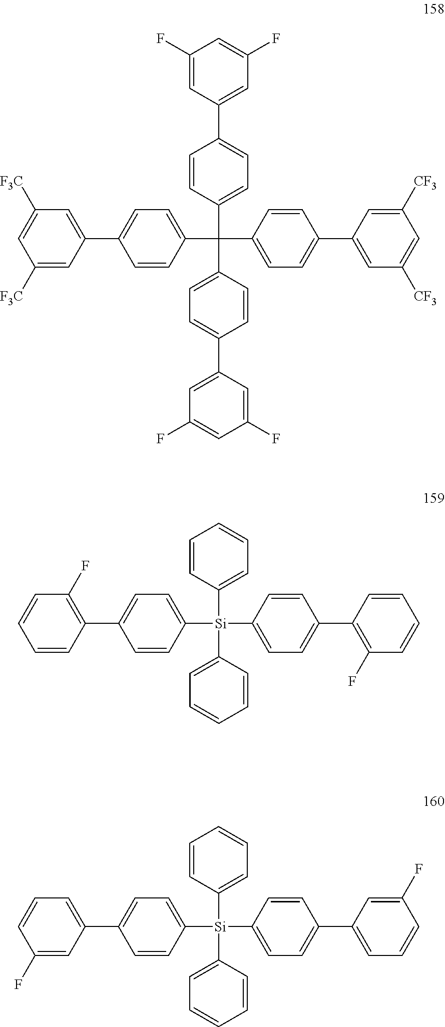 [SMILES: Fc1cc(F)cc(-c2ccc(C(c3ccc(-c4cc(F)cc(F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)c1 ; Fc1ccccc1-c1ccc([Si](c2ccccc2)(c2ccccc2)c2ccc(-c3ccccc3F)cc2)cc1 ; Fc1cccc(-c2ccc([Si](c3ccccc3)(c3ccccc3)c3ccc(-c4cccc(F)c4)cc3)cc2)c1]

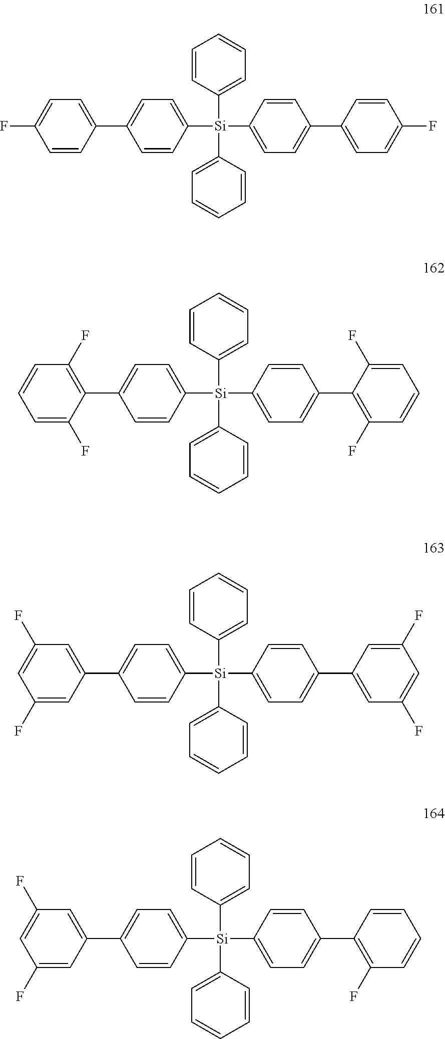 [SMILES: Fc1ccc(-c2ccc([Si](c3ccccc3)(c3ccccc3)c3ccc(-c4ccc(F)cc4)cc3)cc2)cc1 ; Fc1cccc(F)c1-c1ccc([Si](c2ccccc2)(c2ccccc2)c2ccc(-c3c(F)cccc3F)cc2)cc1 ; Fc1cc(F)cc(-c2ccc([Si](c3ccccc3)(c3ccccc3)c3ccc(-c4cc(F)cc(F)c4)cc3)cc2)c1 ; Fc1cc(F)cc(-c2ccc([Si](c3ccccc3)(c3ccccc3)c3ccc(-c4ccccc4F)cc3)cc2)c1]

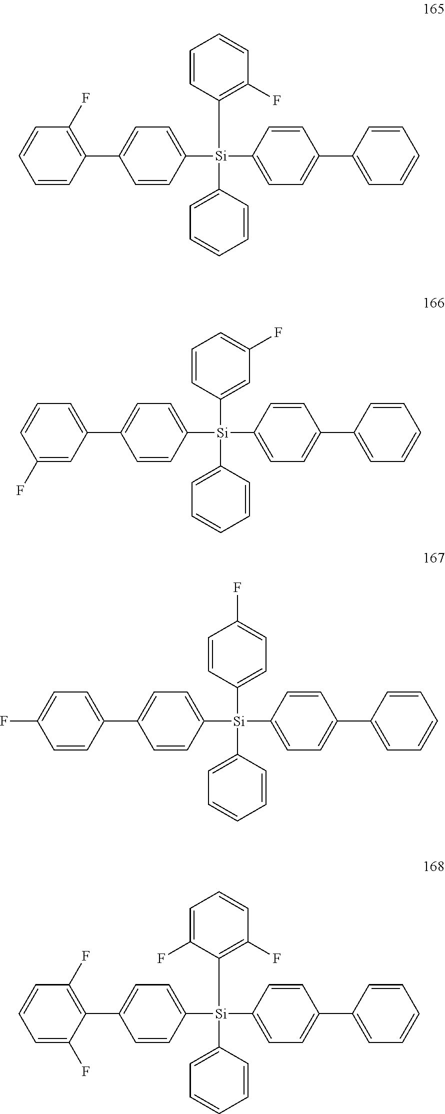 [SMILES: Fc1ccccc1-c1ccc([Si](c2ccccc2)(c2ccc(-c3ccccc3)cc2)c2ccccc2F)cc1 ; Fc1cccc(-c2ccc([Si](c3ccccc3)(c3ccc(-c4ccccc4)cc3)c3cccc(F)c3)cc2)c1 ; Fc1ccc(-c2ccc([Si](c3ccccc3)(c3ccc(F)cc3)c3ccc(-c4ccccc4)cc3)cc2)cc1 ; Fc1cccc(F)c1-c1ccc([Si](c2ccccc2)(c2ccc(-c3ccccc3)cc2)c2c(F)cccc2F)cc1]

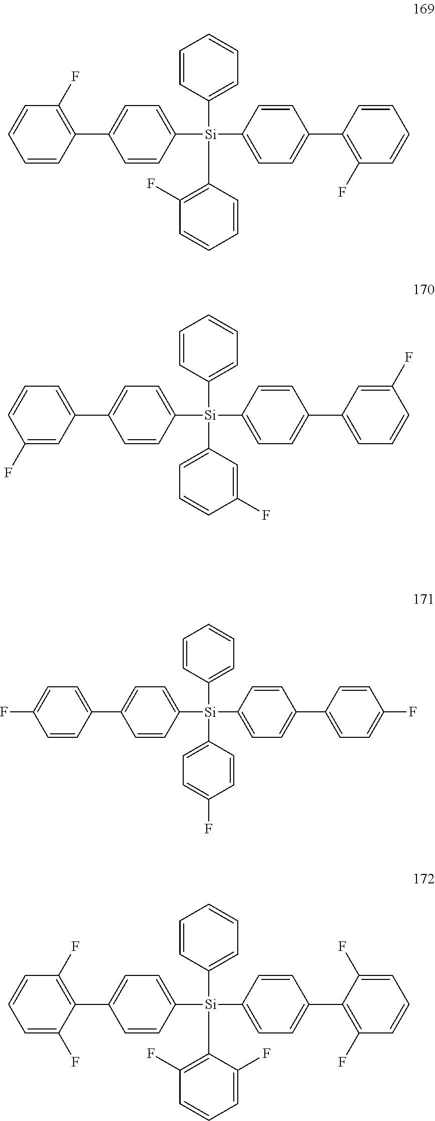 [SMILES: Fc1ccccc1-c1ccc([Si](c2ccccc2)(c2ccc(-c3ccccc3F)cc2)c2ccccc2F)cc1 ; Fc1cccc(-c2ccc([Si](c3ccccc3)(c3ccc(-c4cccc(F)c4)cc3)c3cccc(F)c3)cc2)c1 ; Fc1ccc(-c2ccc([Si](c3ccccc3)(c3ccc(F)cc3)c3ccc(-c4ccc(F)cc4)cc3)cc2)cc1 ; Fc1cccc(F)c1-c1ccc([Si](c2ccccc2)(c2ccc(-c3c(F)cccc3F)cc2)c2c(F)cccc2F)cc1]

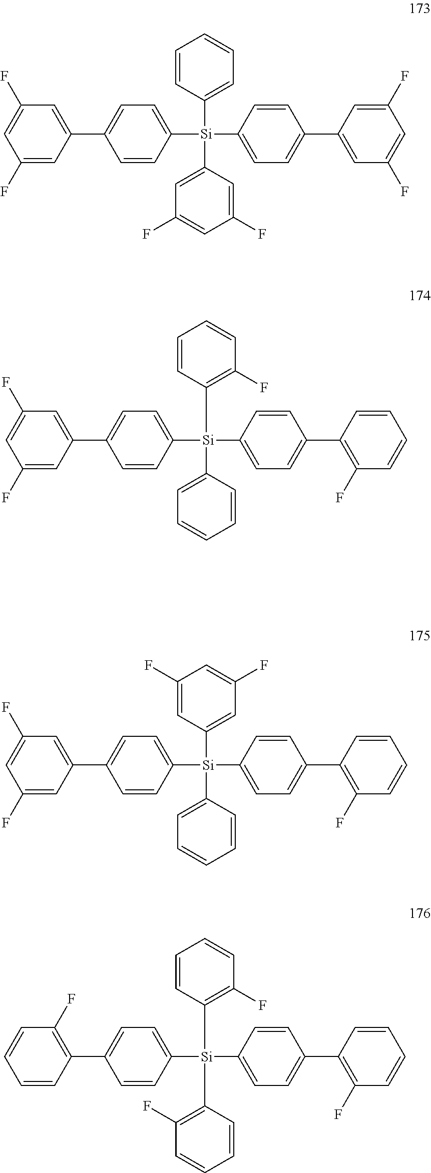 [SMILES: Fc1cc(F)cc(-c2ccc([Si](c3ccccc3)(c3ccc(-c4cc(F)cc(F)c4)cc3)c3cc(F)cc(F)c3)cc2)c1 ; Fc1cc(F)cc(-c2ccc([Si](c3ccccc3)(c3ccc(-c4ccccc4F)cc3)c3ccccc3F)cc2)c1 ; Fc1cc(F)cc(-c2ccc([Si](c3ccccc3)(c3ccc(-c4ccccc4F)cc3)c3cc(F)cc(F)c3)cc2)c1 ; Fc1ccccc1-c1ccc([Si](c2ccc(-c3ccccc3F)cc2)(c2ccccc2F)c2ccccc2F)cc1]

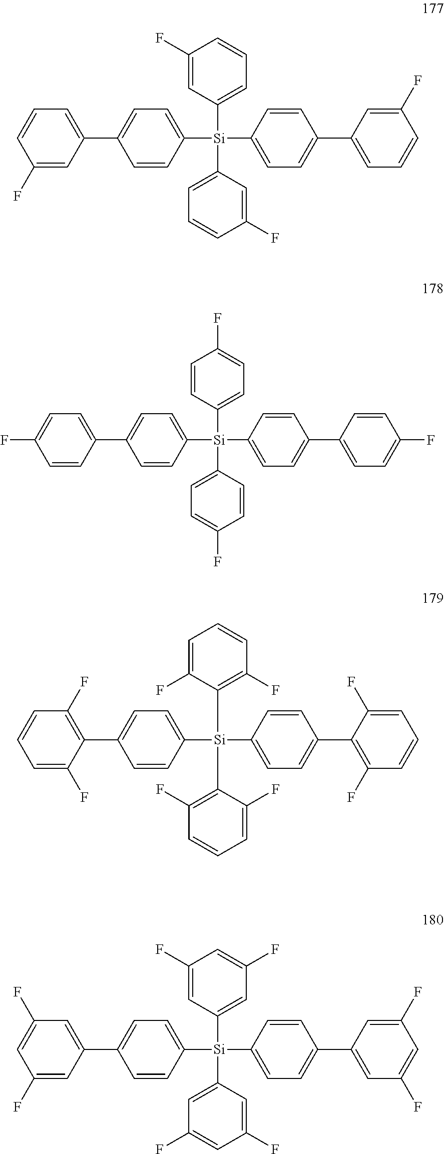 [SMILES: Fc1cccc(-c2ccc([Si](c3ccc(-c4cccc(F)c4)cc3)(c3cccc(F)c3)c3cccc(F)c3)cc2)c1 ; Fc1ccc(-c2ccc([Si](c3ccc(F)cc3)(c3ccc(F)cc3)c3ccc(-c4ccc(F)cc4)cc3)cc2)cc1 ; Fc1cccc(F)c1-c1ccc([Si](c2ccc(-c3c(F)cccc3F)cc2)(c2c(F)cccc2F)c2c(F)cccc2F)cc1 ; Fc1cc(F)cc(-c2ccc([Si](c3ccc(-c4cc(F)cc(F)c4)cc3)(c3cc(F)cc(F)c3)c3cc(F)cc(F)c3)cc2)c1]

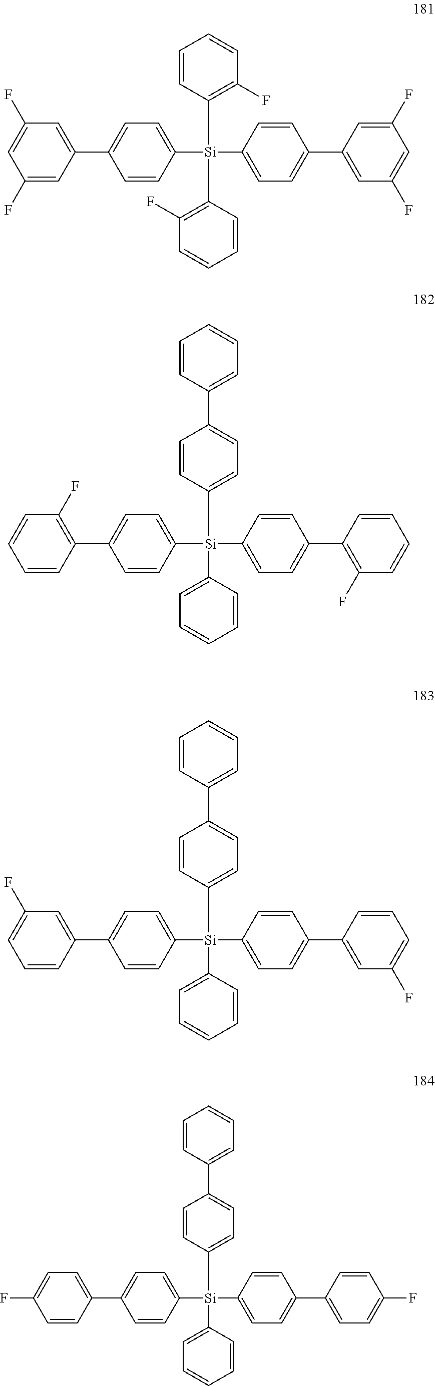 [SMILES: Fc1cc(F)cc(-c2ccc([Si](c3ccc(-c4cc(F)cc(F)c4)cc3)(c3ccccc3F)c3ccccc3F)cc2)c1 ; Fc1ccccc1-c1ccc([Si](c2ccccc2)(c2ccc(-c3ccccc3)cc2)c2ccc(-c3ccccc3F)cc2)cc1 ; Fc1cccc(-c2ccc([Si](c3ccccc3)(c3ccc(-c4ccccc4)cc3)c3ccc(-c4cccc(F)c4)cc3)cc2)c1 ; Fc1ccc(-c2ccc([Si](c3ccccc3)(c3ccc(-c4ccccc4)cc3)c3ccc(-c4ccc(F)cc4)cc3)cc2)cc1]

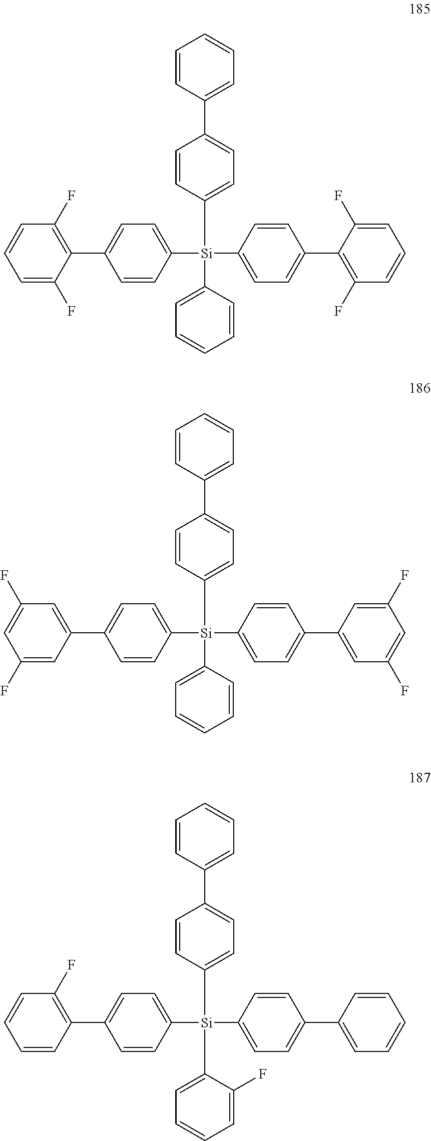 [SMILES: Fc1cccc(F)c1-c1ccc([Si](c2ccccc2)(c2ccc(-c3ccccc3)cc2)c2ccc(-c3c(F)cccc3F)cc2)cc1 ; Fc1cc(F)cc(-c2ccc([Si](c3ccccc3)(c3ccc(-c4ccccc4)cc3)c3ccc(-c4cc(F)cc(F)c4)cc3)cc2)c1 ; Fc1ccccc1-c1ccc([Si](c2ccc(-c3ccccc3)cc2)(c2ccc(-c3ccccc3)cc2)c2ccccc2F)cc1]

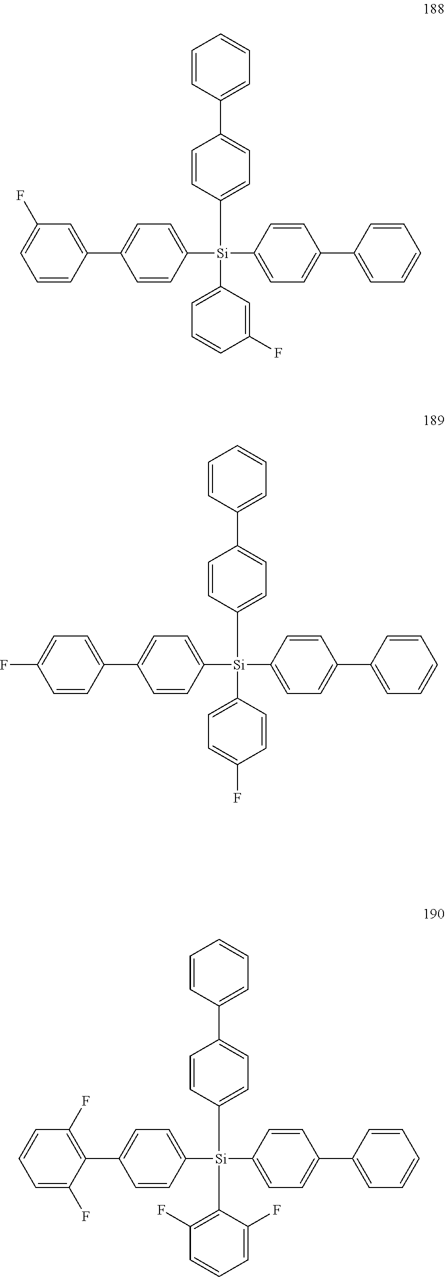 [SMILES: Fc1cccc(-c2ccc([Si](c3ccc(-c4ccccc4)cc3)(c3ccc(-c4ccccc4)cc3)c3cccc(F)c3)cc2)c1 ; Fc1ccc(-c2ccc([Si](c3ccc(F)cc3)(c3ccc(-c4ccccc4)cc3)c3ccc(-c4ccccc4)cc3)cc2)cc1 ; Fc1cccc(F)c1-c1ccc([Si](c2ccc(-c3ccccc3)cc2)(c2ccc(-c3ccccc3)cc2)c2c(F)cccc2F)cc1]

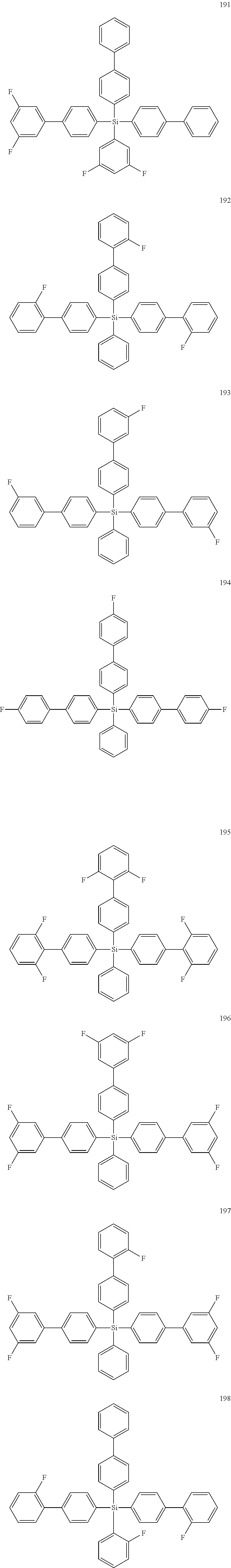

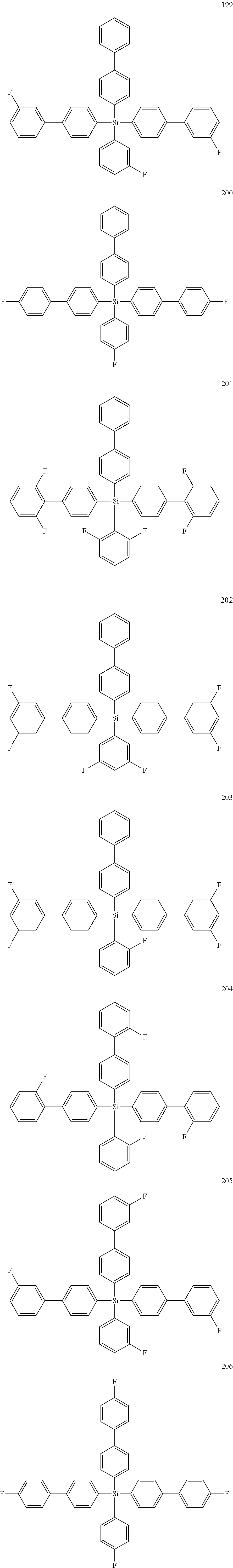

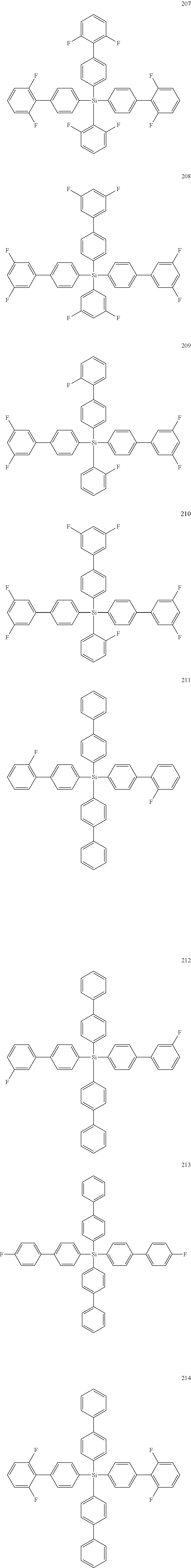

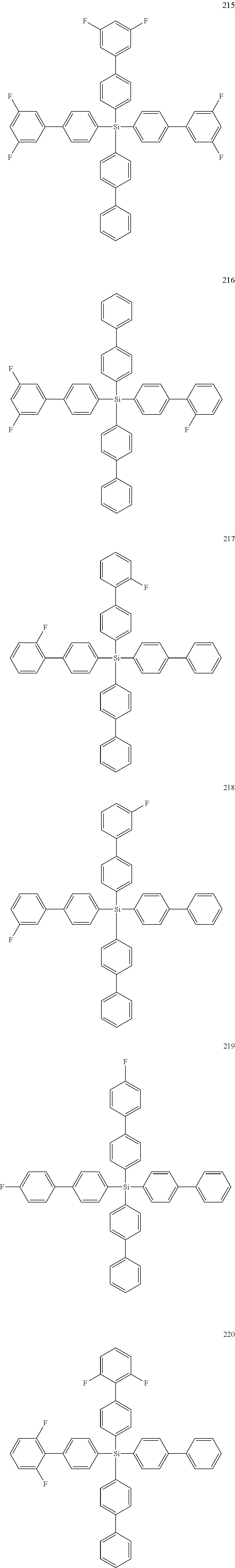

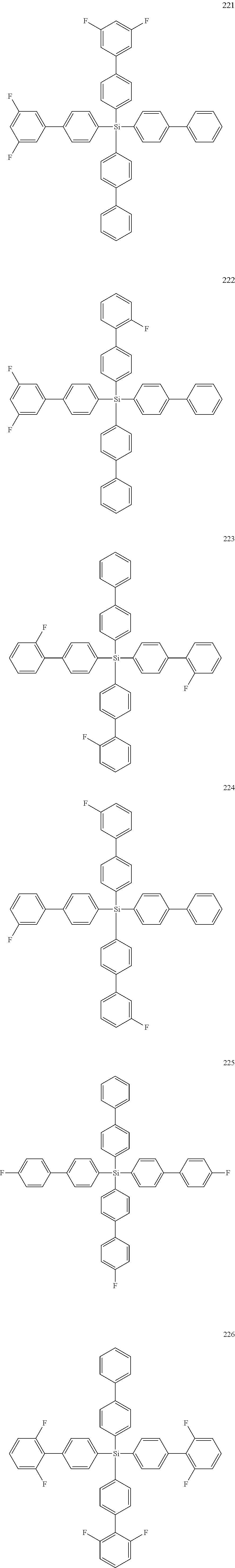

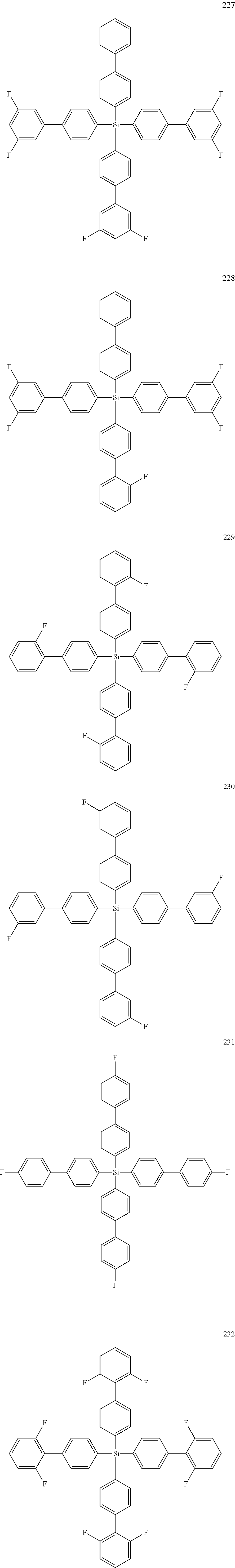

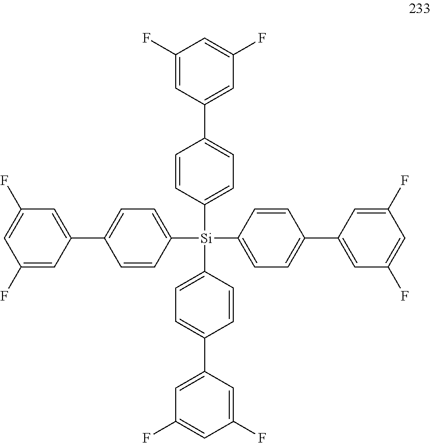 [SMILES: Fc1cc(F)cc(-c2ccc([Si](c3ccc(-c4cc(F)cc(F)c4)cc3)(c3ccc(-c4cc(F)cc(F)c4)cc3)c3ccc(-c4cc(F)cc(F)c4)cc3)cc2)c1]

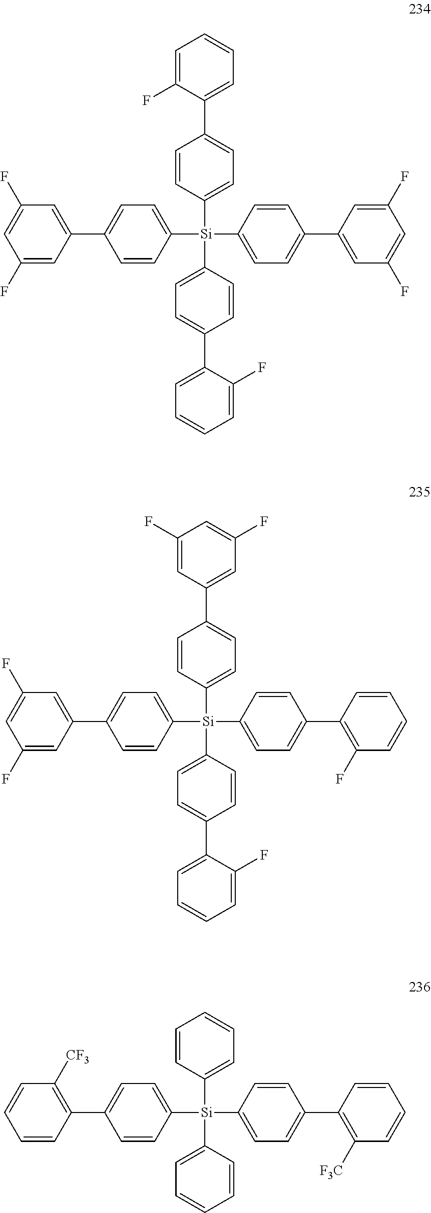 [SMILES: Fc1cc(F)cc(-c2ccc([Si](c3ccc(-c4cc(F)cc(F)c4)cc3)(c3ccc(-c4ccccc4F)cc3)c3ccc(-c4ccccc4F)cc3)cc2)c1 ; Fc1cc(F)cc(-c2ccc([Si](c3ccc(-c4cc(F)cc(F)c4)cc3)(c3ccc(-c4ccccc4F)cc3)c3ccc(-c4ccccc4F)cc3)cc2)c1 ; FC(F)(F)c1ccccc1-c1ccc([Si](c2ccccc2)(c2ccccc2)c2ccc(-c3ccccc3C(F)(F)F)cc2)cc1]

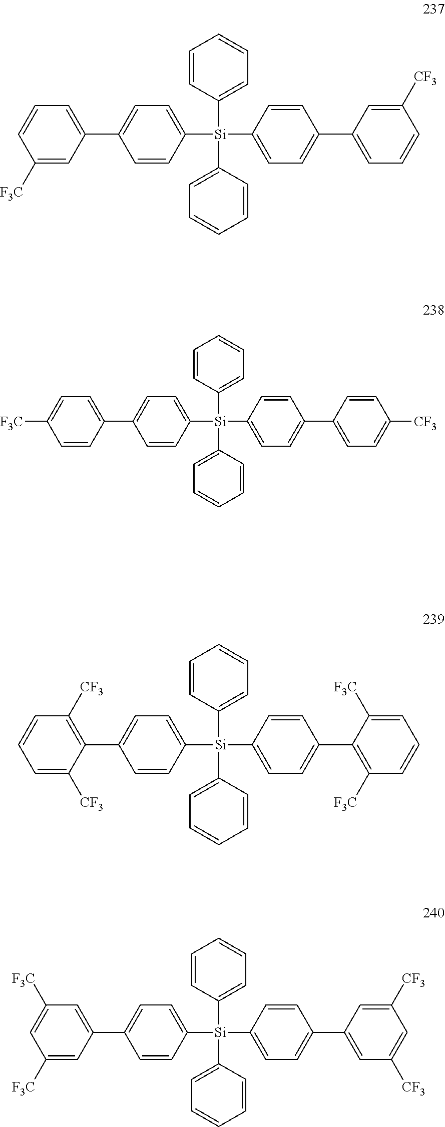 [SMILES: FC(F)(F)c1cccc(-c2ccc([Si](c3ccccc3)(c3ccccc3)c3ccc(-c4cccc(C(F)(F)F)c4)cc3)cc2)c1 ; FC(F)(F)c1ccc(-c2ccc([Si](c3ccccc3)(c3ccccc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccccc2)c2ccc(-c3c(C(F)(F)F)cccc3C(F)(F)F)cc2)cc1 ; FC(F)(F)c1cc(-c2ccc([Si](c3ccccc3)(c3ccccc3)c3ccc(-c4cc(C(F)(F)F)cc(C(F)(F)F)c4)cc3)cc2)cc(C(F)(F)F)c1]

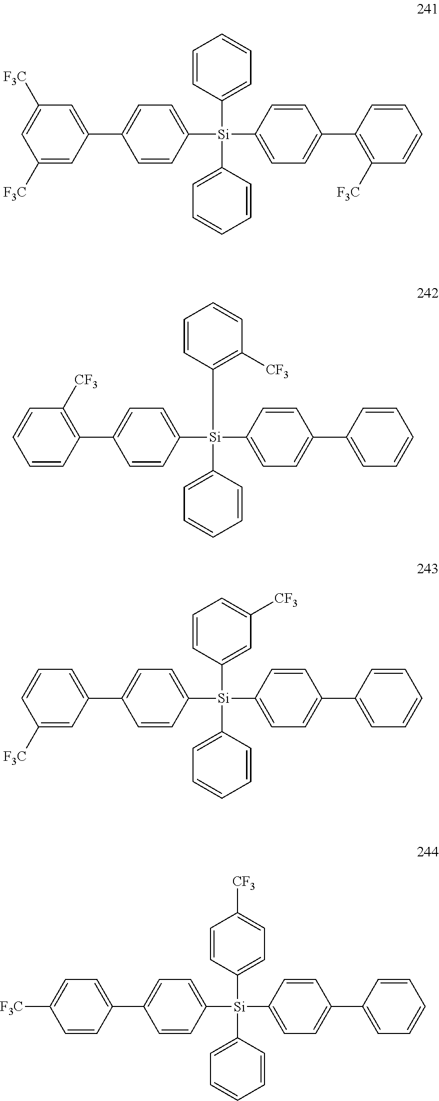 [SMILES: FC(F)(F)c1cc(-c2ccc([Si](c3ccccc3)(c3ccccc3)c3ccc(-c4ccccc4C(F)(F)F)cc3)cc2)cc(C(F)(F)F)c1 ; FC(F)(F)c1ccccc1-c1ccc([Si](c2ccccc2)(c2ccc(-c3ccccc3)cc2)c2ccccc2C(F)(F)F)cc1 ; FC(F)(F)c1cccc(-c2ccc([Si](c3ccccc3)(c3ccc(-c4ccccc4)cc3)c3cccc(C(F)(F)F)c3)cc2)c1 ; FC(F)(F)c1ccc(-c2ccc([Si](c3ccccc3)(c3ccc(-c4ccccc4)cc3)c3ccc(C(F)(F)F)cc3)cc2)cc1]

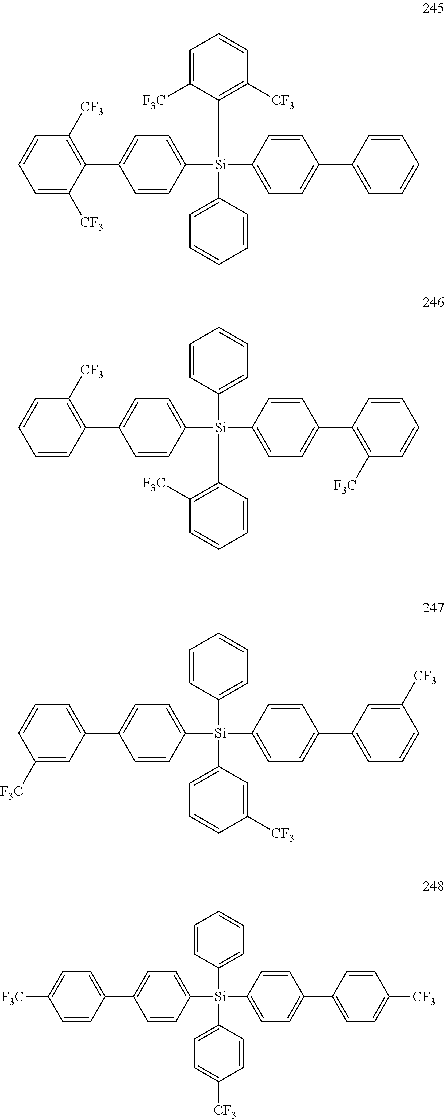 [SMILES: FC(F)(F)c1cccc(C(F)(F)F)c1-c1ccc([Si](c2ccccc2)(c2ccc(-c3ccccc3)cc2)c2c(C(F)(F)F)cccc2C(F)(F)F)cc1 ; FC(F)(F)c1ccccc1-c1ccc([Si](c2ccccc2)(c2ccc(-c3ccccc3C(F)(F)F)cc2)c2ccccc2C(F)(F)F)cc1 ; FC(F)(F)c1cccc(-c2ccc([Si](c3ccccc3)(c3ccc(-c4cccc(C(F)(F)F)c4)cc3)c3cccc(C(F)(F)F)c3)cc2)c1 ; FC(F)(F)c1ccc(-c2ccc([Si](c3ccccc3)(c3ccc(-c4ccc(C(F)(F)F)cc4)cc3)c3ccc(C(F)(F)F)cc3)cc2)cc1]

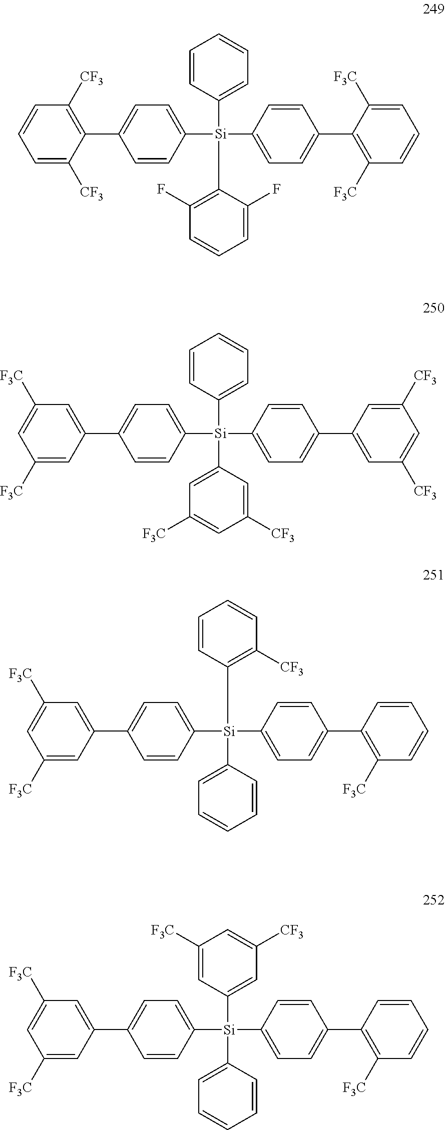 [SMILES: Fc1cccc(F)c1[Si](c1ccccc1)(c1ccc(-c2c(C(F)(F)F)cccc2C(F)(F)F)cc1)c1ccc(-c2c(C(F)(F)F)cccc2C(F)(F)F)cc1 ; FC(F)(F)c1cc(-c2ccc([Si](c3ccccc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccccc3)(c3ccc(-c4ccccc4C(F)(F)F)cc3)c3ccccc3C(F)(F)F)cc2)cc(C(F)(F)F)c1 ; FC(F)(F)c1cc(-c2ccc([Si](c3ccccc3)(c3ccc(-c4ccccc4C(F)(F)F)cc3)c3cc(C(F)(F)F)cc(C(F)(F)F)c3)cc2)cc(C(F)(F)F)c1]

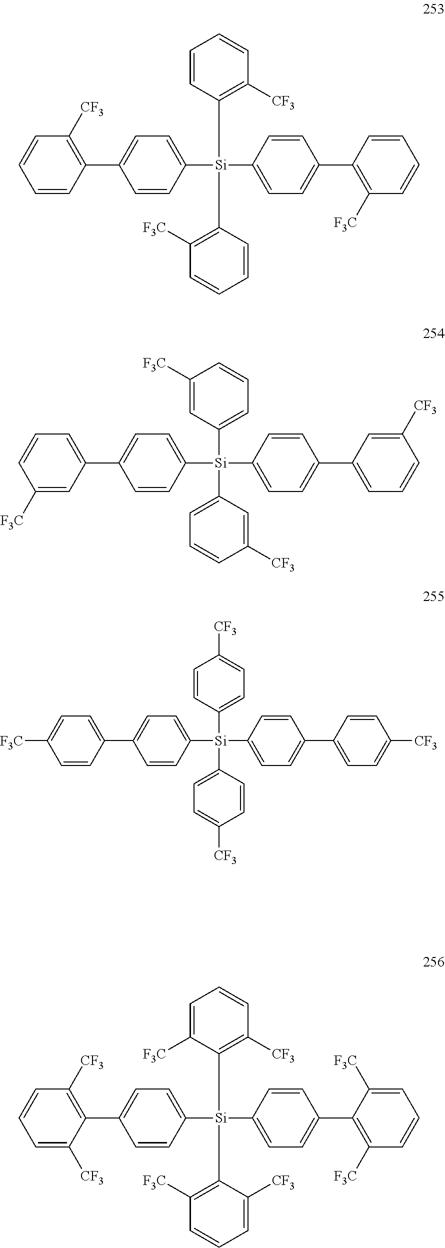 [SMILES: FC(F)(F)c1ccccc1-c1ccc([Si](c2ccc(-c3ccccc3C(F)(F)F)cc2)(c2ccccc2C(F)(F)F)c2ccccc2C(F)(F)F)cc1 ; FC(F)(F)c1cccc(-c2ccc([Si](c3ccc(-c4cccc(C(F)(F)F)c4)cc3)(c3cccc(C(F)(F)F)c3)c3cccc(C(F)(F)F)c3)cc2)c1 ; FC(F)(F)c1ccc(-c2ccc([Si](c3ccc(-c4ccc(C(F)(F)F)cc4)cc3)(c3ccc(C(F)(F)F)cc3)c3ccc(C(F)(F)F)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2c(C(F)(F)F)cccc2C(F)(F)F)c2c(C(F)(F)F)cccc2C(F)(F)F)cc1]

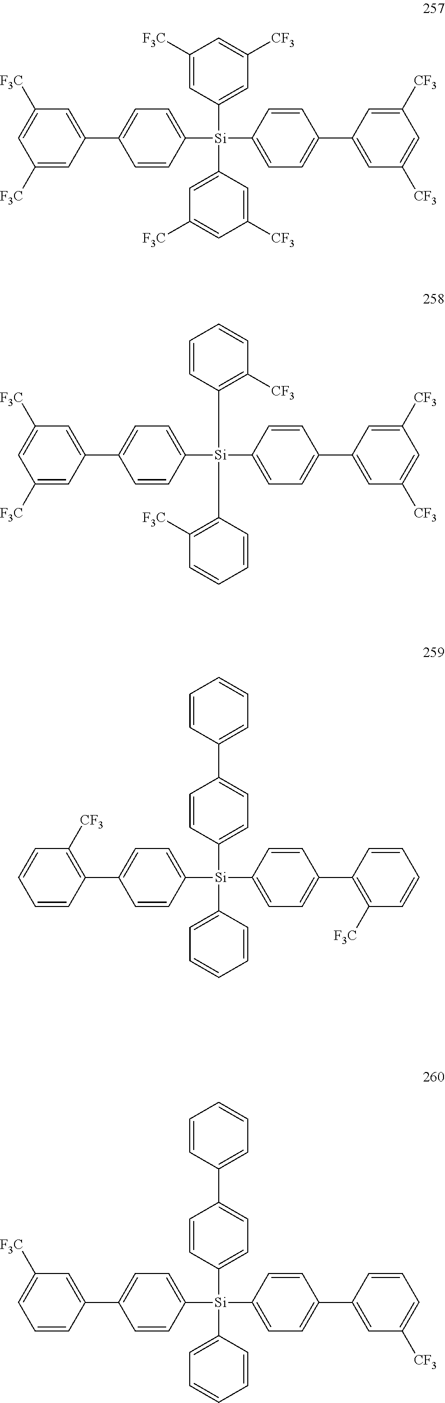 [SMILES: FC(F)(F)c1cc(-c2ccc([Si](c3ccc(-c4cc(C(F)(F)F)cc(C(F)(F)F)c4)cc3)(c3cc(C(F)(F)F)cc(C(F)(F)F)c3)c3cc(C(F)(F)F)cc(C(F)(F)F)c3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccccc3C(F)(F)F)c3ccccc3C(F)(F)F)cc2)cc(C(F)(F)F)c1 ; FC(F)(F)c1ccccc1-c1ccc([Si](c2ccccc2)(c2ccc(-c3ccccc3)cc2)c2ccc(-c3ccccc3C(F)(F)F)cc2)cc1 ; FC(F)(F)c1cccc(-c2ccc([Si](c3ccccc3)(c3ccc(-c4ccccc4)cc3)c3ccc(-c4cccc(C(F)(F)F)c4)cc3)cc2)c1]

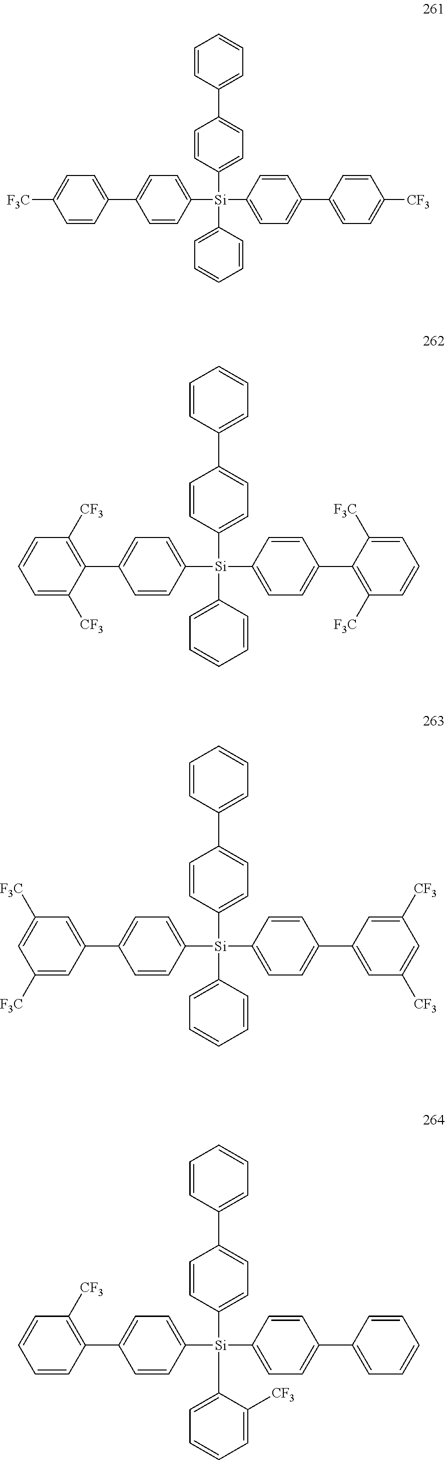 [SMILES: FC(F)(F)c1ccc(-c2ccc([Si](c3ccccc3)(c3ccc(-c4ccccc4)cc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccccc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1ccccc1-c1ccc([Si](c2ccc(-c3ccccc3)cc2)(c2ccc(-c3ccccc3)cc2)c2ccccc2C(F)(F)F)cc1]

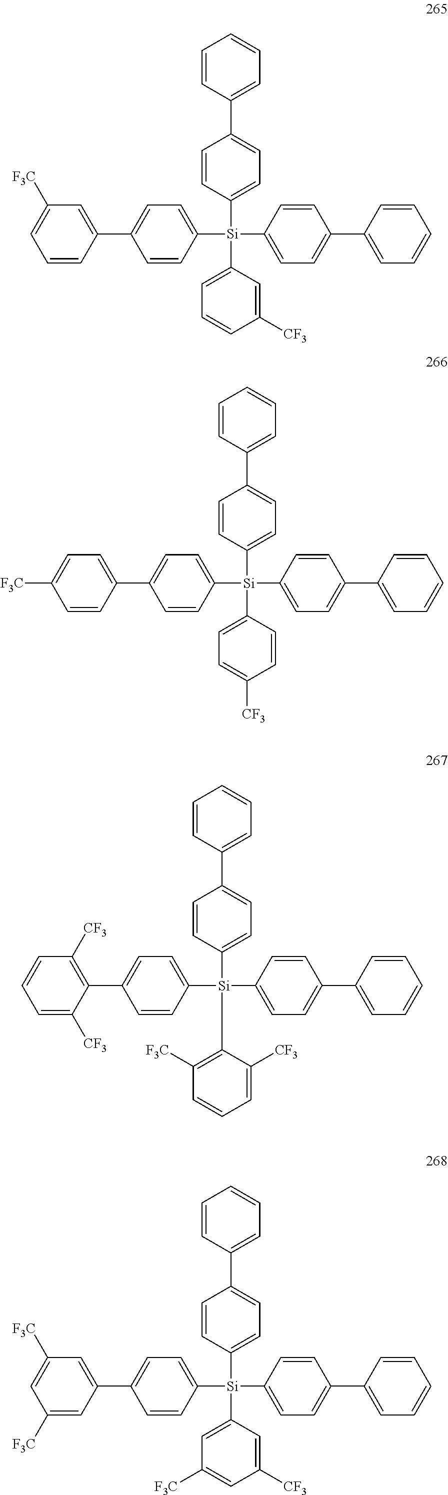 [SMILES: FC(F)(F)c1cccc(-c2ccc([Si](c3ccc(-c4ccccc4)cc3)(c3ccc(-c4ccccc4)cc3)c3cccc(C(F)(F)F)c3)cc2)c1 ; FC(F)(F)c1ccc(-c2ccc([Si](c3ccc(-c4ccccc4)cc3)(c3ccc(-c4ccccc4)cc3)c3ccc(C(F)(F)F)cc3)cc2)cc1 ; FC(F)(F)c1cccc(C(F)(F)F)c1-c1ccc([Si](c2ccc(-c3ccccc3)cc2)(c2ccc(-c3ccccc3)cc2)c2c(C(F)(F)F)cccc2C(F)(F)F)cc1 ; FC(F)(F)c1cc(-c2ccc([Si](c3ccc(-c4ccccc4)cc3)(c3ccc(-c4ccccc4)cc3)c3cc(C(F)(F)F)cc(C(F)(F)F)c3)cc2)cc(C(F)(F)F)c1]

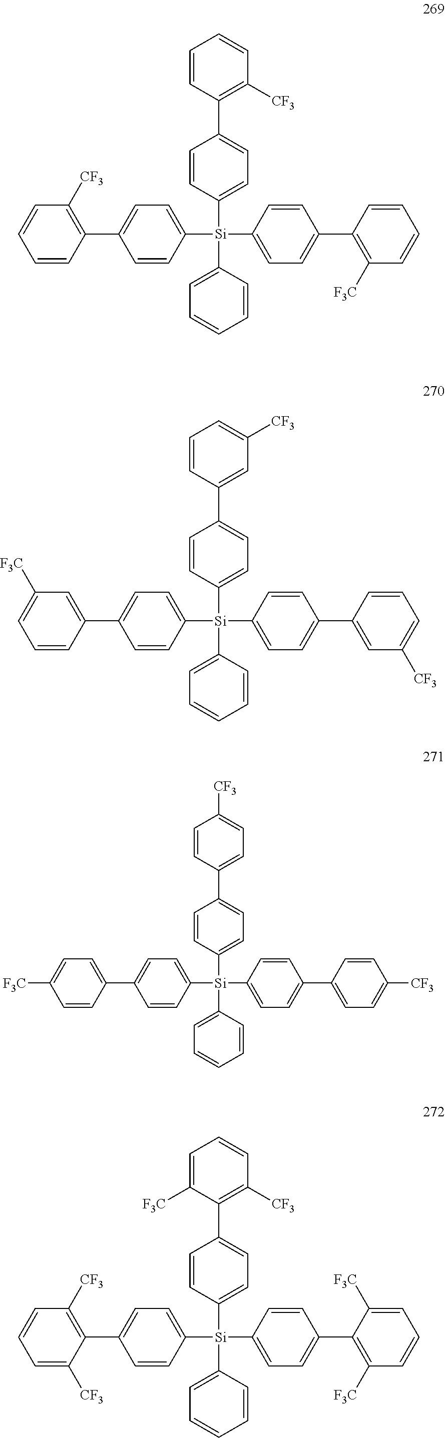 [SMILES: FC(F)(F)c1ccccc1-c1ccc([Si](c2ccccc2)(c2ccc(-c3ccccc3C(F)(F)F)cc2)c2ccc(-c3ccccc3C(F)(F)F)cc2)cc1 ; FC(F)(F)c1cccc(-c2ccc([Si](c3ccccc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccccc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccc(-c3c(C(F)(F)F)cccc3C(F)(F)F)cc2)c2ccc(-c3c(C(F)(F)F)cccc3C(F)(F)F)cc2)cc1]

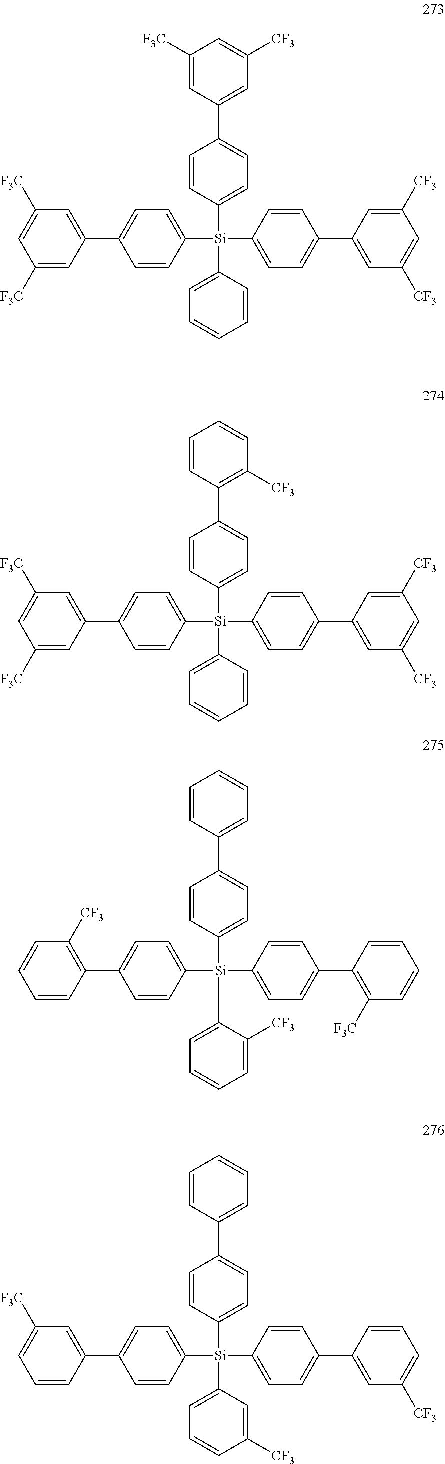 [SMILES: FC(F)(F)c1cc(-c2ccc([Si](c3ccccc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccccc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc([Si](c2ccc(-c3ccccc3)cc2)(c2ccc(-c3ccccc3C(F)(F)F)cc2)c2ccccc2C(F)(F)F)cc1 ; FC(F)(F)c1cccc(-c2ccc([Si](c3ccc(-c4ccccc4)cc3)(c3ccc(-c4cccc(C(F)(F)F)c4)cc3)c3cccc(C(F)(F)F)c3)cc2)c1]

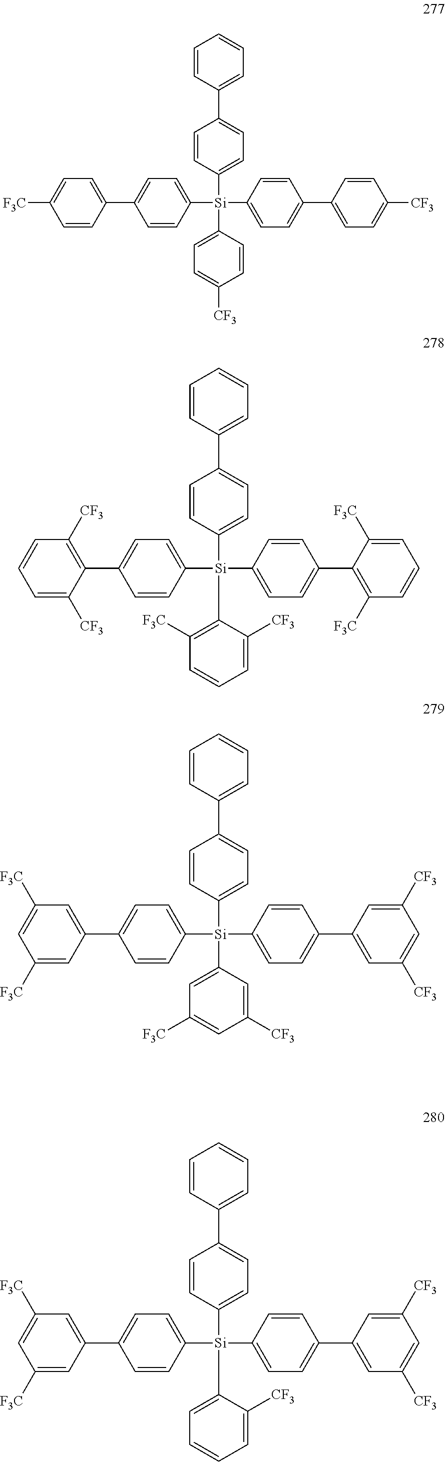 [SMILES: FC(F)(F)c1ccc(-c2ccc([Si](c3ccc(-c4ccccc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc([Si](c2ccc(-c3ccccc3)cc2)(c2ccc(-c3c(C(F)(F)F)cccc3C(F)(F)F)cc2)c2c(C(F)(F)F)cccc2C(F)(F)F)cc1 ; FC(F)(F)c1cc(-c2ccc([Si](c3ccc(-c4ccccc4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccc(-c4ccccc4)cc3)(c3ccc(-c4cc(C(F)(F)F)cc(C(F)(F)F)c4)cc3)c3ccccc3C(F)(F)F)cc2)cc(C(F)(F)F)c1]

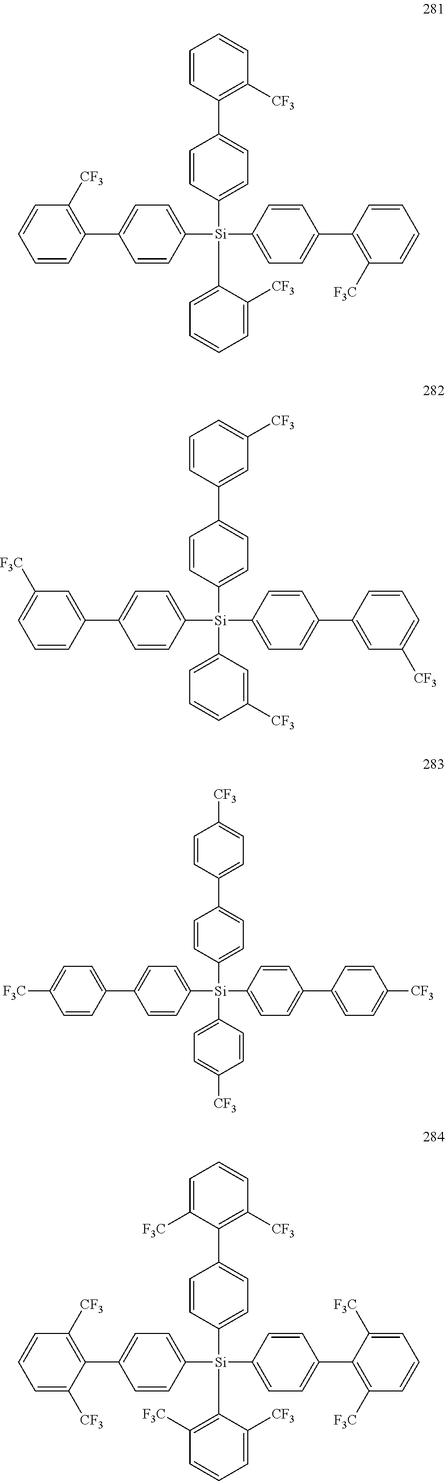 [SMILES: FC(F)(F)c1ccccc1-c1ccc([Si](c2ccc(-c3ccccc3C(F)(F)F)cc2)(c2ccc(-c3ccccc3C(F)(F)F)cc2)c2ccccc2C(F)(F)F)cc1 ; FC(F)(F)c1cccc(-c2ccc([Si](c3ccc(-c4cccc(C(F)(F)F)c4)cc3)(c3ccc(-c4cccc(C(F)(F)F)c4)cc3)c3cccc(C(F)(F)F)c3)cc2)c1 ; FC(F)(F)c1ccc(-c2ccc([Si](c3ccc(-c4ccc(C(F)(F)F)cc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2ccc(-c3c(C(F)(F)F)cccc3C(F)(F)F)cc2)c2c(C(F)(F)F)cccc2C(F)(F)F)cc1]

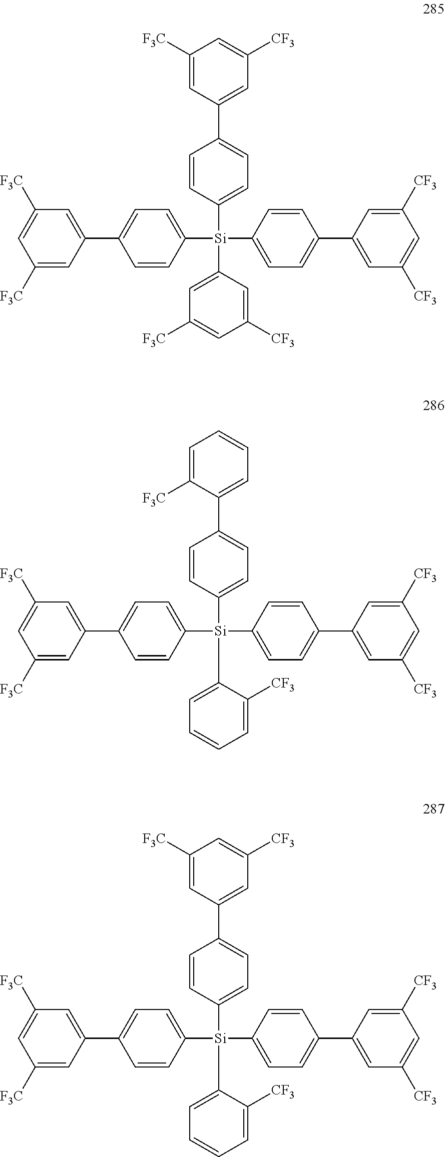 [SMILES: FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccccc3C(F)(F)F)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3ccccc3C(F)(F)F)cc2)cc(C(F)(F)F)c1]

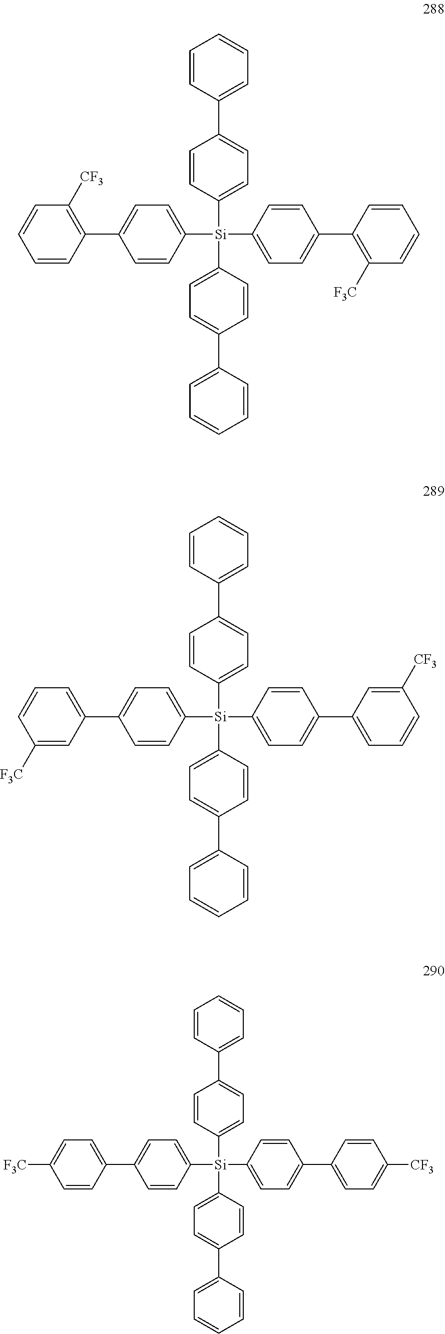 [SMILES: FC(F)(F)c1ccccc1-c1ccc([Si](c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc([Si](c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccccc4)cc3)c3ccc(-c4ccc(C(F)(F)F)cc4)cc3)cc2)cc1]

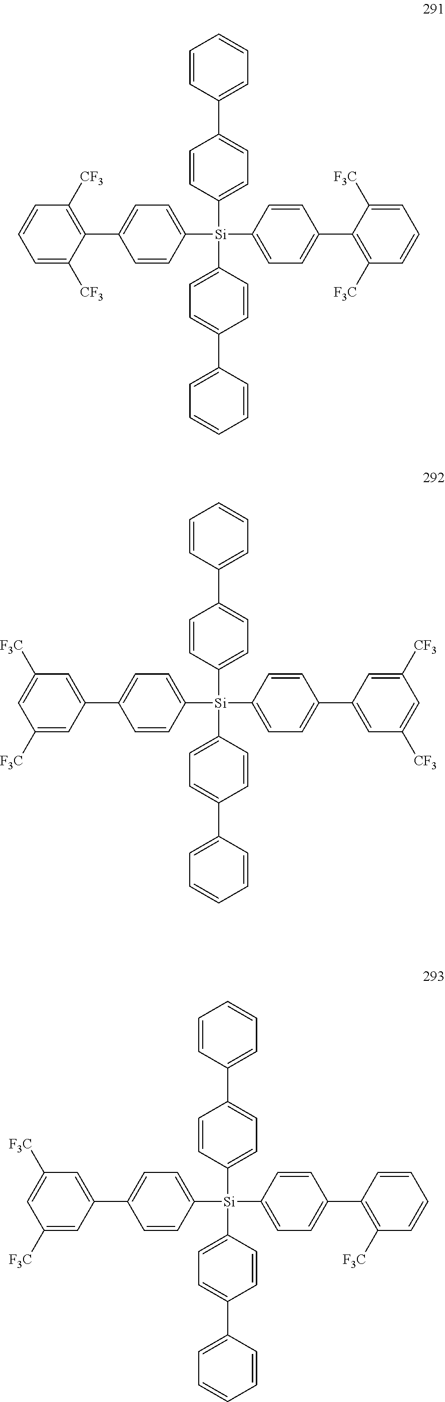 [SMILES: FC(F)(F)c1cccc(C(F)(F)F)c1-c1ccc([Si](c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4ccccc4)cc3)c3ccc(-c4ccccc4C(F)(F)F)cc3)cc2)cc(C(F)(F)F)c1]

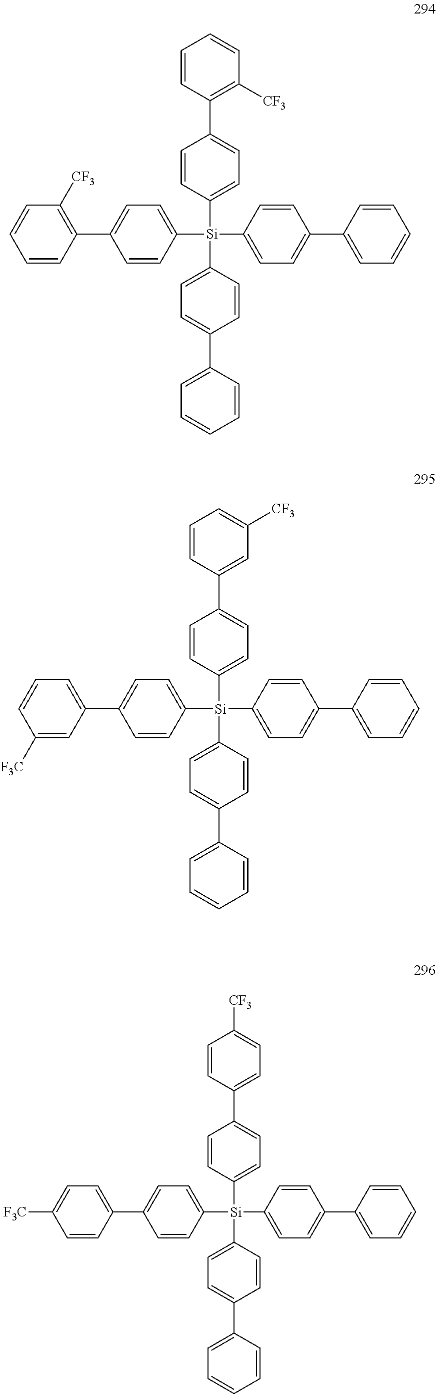 [SMILES: FC(F)(F)c1ccccc1-c1ccc([Si](c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc([Si](c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccccc4)cc3)c3ccc(-c4ccc(C(F)(F)F)cc4)cc3)cc2)cc1]

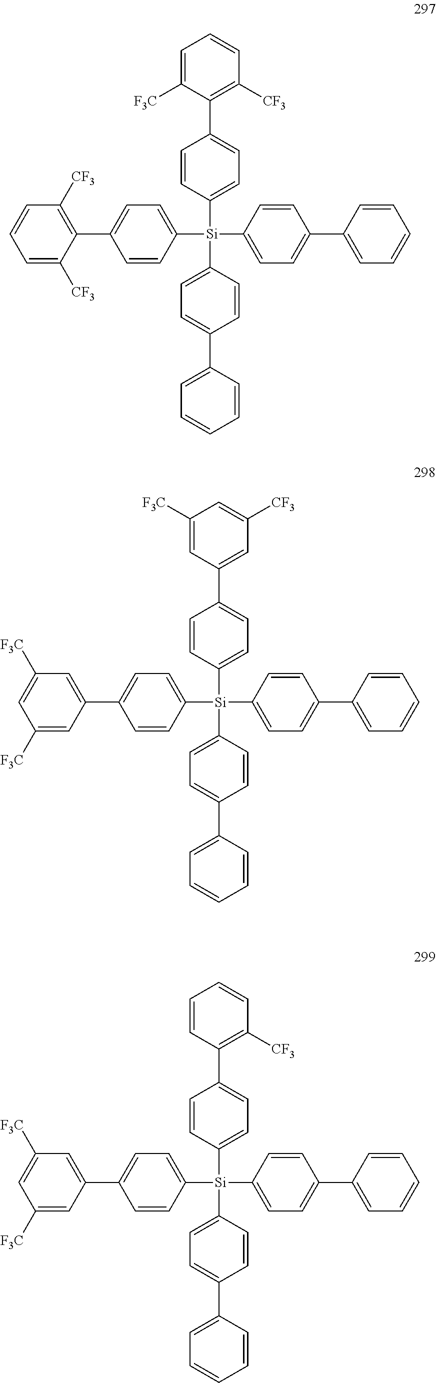 [SMILES: FC(F)(F)c1cccc(C(F)(F)F)c1-c1ccc([Si](c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4ccccc4)cc3)c3ccc(-c4ccccc4C(F)(F)F)cc3)cc2)cc(C(F)(F)F)c1]

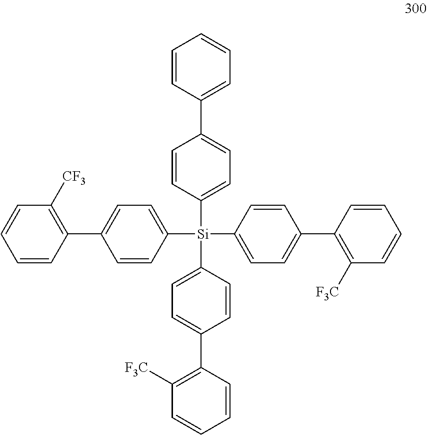 [SMILES: FC(F)(F)c1ccccc1-c1ccc([Si](c2ccc(-c3ccccc3)cc2)(c2ccc(-c3ccccc3C(F)(F)F)cc2)c2ccc(-c3ccccc3C(F)(F)F)cc2)cc1]

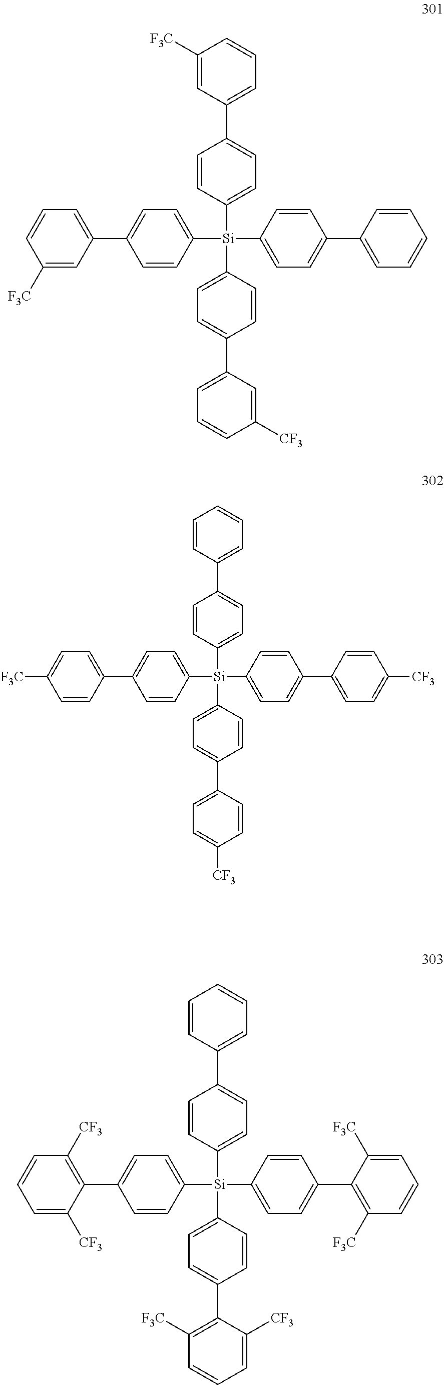 [SMILES: FC(F)(F)c1cccc(-c2ccc([Si](c3ccc(-c4ccccc4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccc(-c3ccccc3)cc2)(c2ccc(-c3c(C(F)(F)F)cccc3C(F)(F)F)cc2)c2ccc(-c3c(C(F)(F)F)cccc3C(F)(F)F)cc2)cc1]

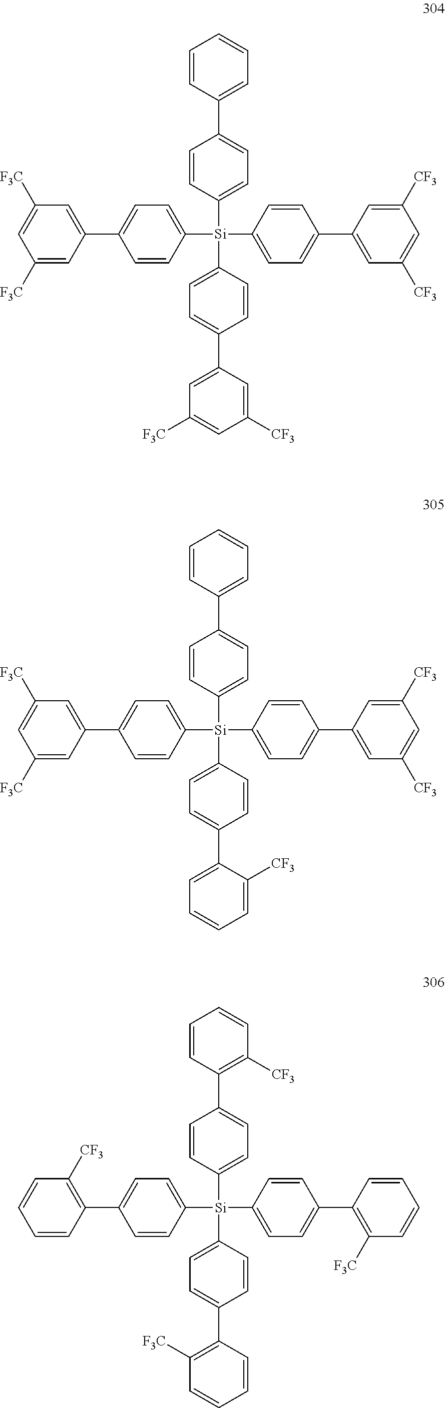 [SMILES: FC(F)(F)c1cc(-c2ccc([Si](c3ccc(-c4ccccc4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc([Si](c2ccc(-c3ccccc3C(F)(F)F)cc2)(c2ccc(-c3ccccc3C(F)(F)F)cc2)c2ccc(-c3ccccc3C(F)(F)F)cc2)cc1]

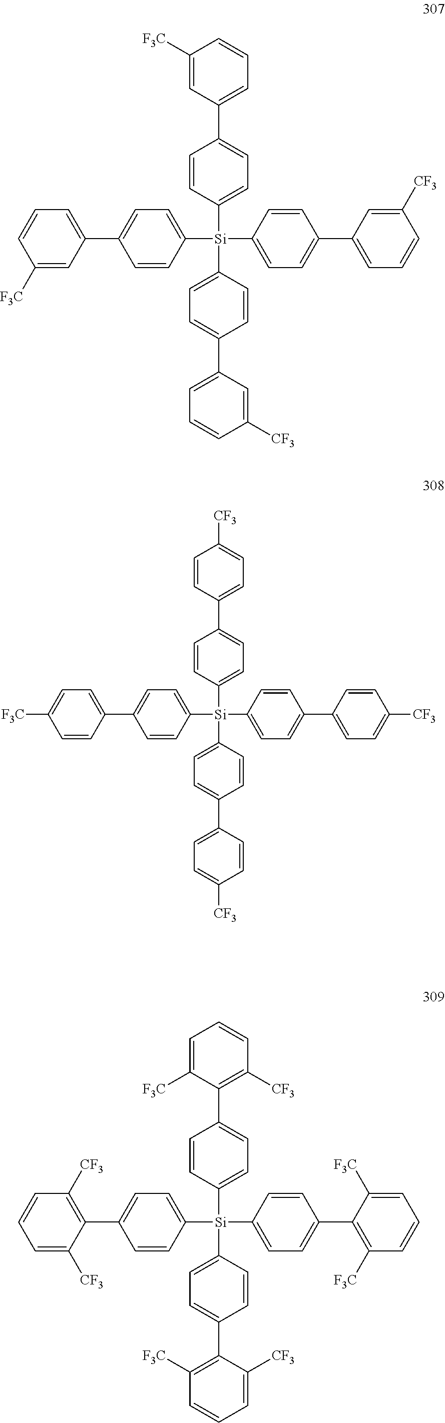 [SMILES: FC(F)(F)c1cccc(-c2ccc([Si](c3ccc(-c4cccc(C(F)(F)F)c4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccc(-c4ccc(C(F)(F)F)cc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2ccc(-c3c(C(F)(F)F)cccc3C(F)(F)F)cc2)c2ccc(-c3c(C(F)(F)F)cccc3C(F)(F)F)cc2)cc1]

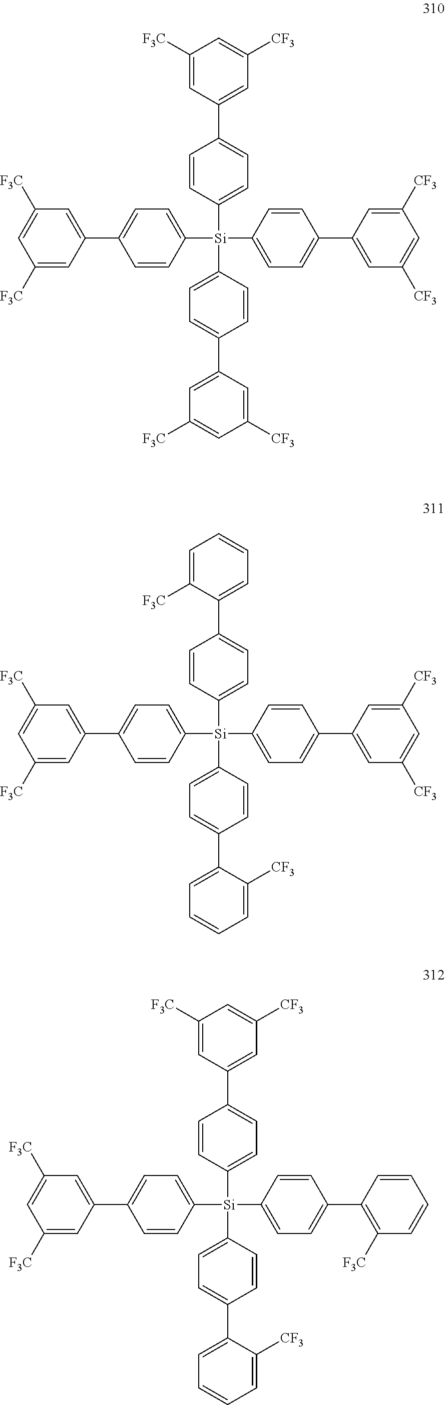 [SMILES: FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1]

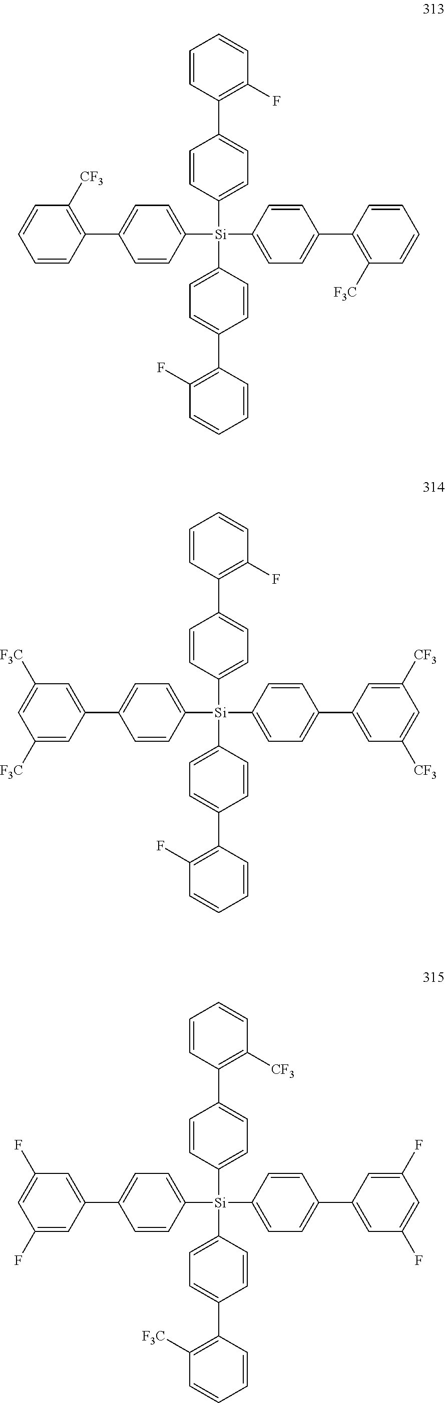 [SMILES: Fc1ccccc1-c1ccc([Si](c2ccc(-c3ccccc3F)cc2)(c2ccc(-c3ccccc3C(F)(F)F)cc2)c2ccc(-c3ccccc3C(F)(F)F)cc2)cc1 ; Fc1ccccc1-c1ccc([Si](c2ccc(-c3cc(C(F)(F)F)cc(C(F)(F)F)c3)cc2)(c2ccc(-c3cc(C(F)(F)F)cc(C(F)(F)F)c3)cc2)c2ccc(-c3ccccc3F)cc2)cc1 ; Fc1cc(F)cc(-c2ccc([Si](c3ccc(-c4cc(F)cc(F)c4)cc3)(c3ccc(-c4ccccc4C(F)(F)F)cc3)c3ccc(-c4ccccc4C(F)(F)F)cc3)cc2)c1]

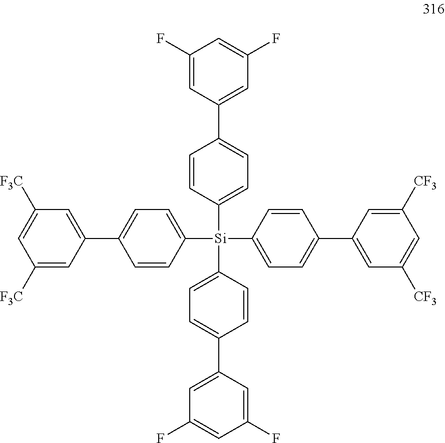 [SMILES: Fc1cc(F)cc(-c2ccc([Si](c3ccc(-c4cc(F)cc(F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)c1]

As such, the organic compound according to the present invention can synthesize organic compounds with various properties using moieties with unique properties. As a result, when the organic compound according to the present invention is applied to the light efficiency improving layer provided in the organic light-emitting device, it is possible to further improve the luminescent properties such as luminous efficiency, etc. of the device.

In addition, the compound of an embodiment of the present invention may be applied to a device according to a general method for manufacturing an organic light-emitting device.

The organic light-emitting device according to an exemplary embodiment of the present invention may be composed of a structure including a first electrode, a second electrode and an organic layer disposed therebetween, and may be manufactured using typical device manufacturing methods and materials, except that the organic light-emitting compound according to the present invention is used in an organic layer of the device.

An organic light-emitting device according to an embodiment of the present invention may include a first electrode, a second electrode, and an organic layer arranged therebetween. The organic light-emitting device may be manufactured using a general device manufacturing method and material, except that the organic compound of an embodiment of the present invention is used to form the organic layer of the device.

The organic layer of the organic light-emitting device according to the present invention may be composed of a single-layered structure, but may also be composed of a multi-layered structure in which two or more organic layers are stacked. For example, the organic layer may have a structure including a hole injection layer, a hole transport layer, a light-emitting layer, an electron transport layer, an electron injection layer, an electron blocking layer, a hole blocking layer, a light efficiency improving layer (capping layer), etc. However, the structure of the organic layer is not limited thereto, and may include a fewer or greater number of organic layers.

The organic layer of the organic light-emitting device according to an embodiment of the present invention may have a monolayer structure or a multilayer structure in which two or more organic layers are stacked. For example, the structure of the organic layers may include a hole injecting layer, a hole transport layer, a light-emitting layer, an electron transport layer, an electron injecting layer, an electron blocking layer, a hole blocking layer, and a light efficiency improving layer (capping layer). The number of the organic layers is not limited and may be increased or decreased.

In addition, the organic electroluminescent device may include a substrate, a first electrode (anode), an organic layer, a second electrode (cathode), and a light efficiency improving layer (capping layer), of which may be formed under the first electrode (bottom emission type) or on the second electrode (top emission type).

When the organic electroluminescent device is of a top emission type, light from the light-emitting layer is emitted to the cathode and passes through the light efficiency improving layer (CPL) formed using the compound according to an embodiment of the present invention having a relatively high refractive index. The wavelength of the light is amplified, resulting in an increase in luminous efficiency.

The organic layer structure of a preferred organic light-emitting device according to the present invention, and the like will be described in more detail in the Examples to be described below.

Preferred structures of the organic layers of the organic light-emitting according to an embodiment of the present invention will be explained in more detail in the examples to be described later.

Further, the organic light-emitting device according to the present invention may be manufactured by depositing a metal or a metal oxide having conductivity, or an alloy thereof on a substrate to form a positive electrode, forming an organic layer including a hole injection layer, a hole transport layer, a light-emitting layer, and an electron transport layer thereon, and then depositing a material, which may be used as a negative electrode, thereon, by using a physical vapor deposition (PVD) method such as sputtering or e-beam evaporation.

In addition, the organic electroluminescent device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light-emitting layer, and an electron transport layer thereon, and depositing a cathode material thereon.

In addition to the above methods, the organic light-emitting device may be fabricated by depositing a cathode material, organic layer materials, and an anode material in this order on a substrate. The organic layers may have a multilayer structure including a hole injecting layer, a hole transport layer, a light-emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene] (PEDT), polypyrrole, and polyaniline.

As the negative electrode material, materials having a low work function are usually preferred so as to facilitate the injection of electrons into an organic layer. Specific examples of the negative electrode material include: a metal such as magnesium, calcium, sodium, potassium, titanium, indium, yttrium, lithium, gadolinium, aluminum, silver, tin, and lead, or an alloy thereof, a multi-layer structured material, such as LiF/Al or LiO₂/Al, and the like, but are not limited thereto.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

As the hole transfer material, materials capable of receiving holes from an anode or a hole injection layer, moving the holes to a light-emitting layer, and having high mobility for the holes are suited. Specific examples thereof include arylamine-based organic materials, conductive polymers, block copolymers having conjugated parts and non-conjugated parts together, and the like, but are not limited thereto.

The hole transport material is a material that may receive holes transported from the anode or the hole injecting layer and may transfer the holes to the light-emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light-emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light-emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light-emitting device according to the present disclosure may be a top-emission type, a bottom-emission type or a dual-emission type depending on the materials used.

The organic light-emitting device according to an embodiment of the present invention may be of a top emission, bottom emission or dual emission type according to the materials used.

Furthermore, the organic light-emitting compound according to the present invention may be operated by a principle which is similar to the principle applied to an organic light-emitting device, even in an organic electroluminescent device including an organic solar cell, an organic photoconductor, an organic transistor, and the like.

In addition, the organic compound according to an embodiment of the present invention may perform its function even in organic electronic devices, including organic solar cells, organic photoconductors, and organic transistors, based on a similar principle to that applied to the organic light-emitting device.

### Mode for Carrying Out Invention

Hereinafter, the present invention will be exemplified in more detail through preferred examples. However, these examples are for more specifically describing the present invention, the scope of the present invention is not limited thereto, and it will be obvious to a person with ordinary skill in the art that various changes and modifications can be made within the scope of the present invention and the scope of the technical spirit.

### Synthesis Example 1: Synthesis of Compound 71

### (1) Preparation Example 1: Synthesis of Compound 71

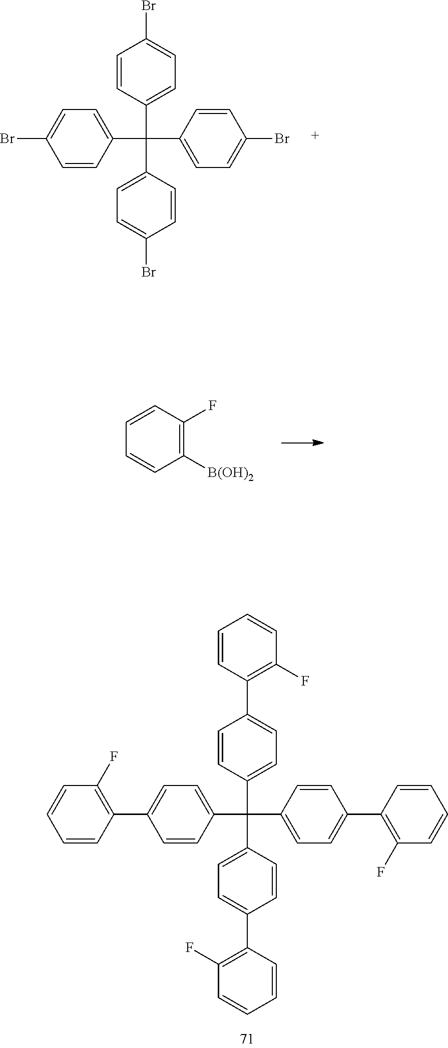 [SMILES: Brc1ccc(C(c2ccc(Br)cc2)(c2ccc(Br)cc2)c2ccc(Br)cc2)cc1 ; OB(O)c1ccccc1F ; Fc1ccccc1-c1ccc(C(c2ccc(-c3ccccc3F)cc2)(c2ccc(-c3ccccc3F)cc2)c2ccc(-c3ccccc3F)cc2)cc1]

200 mL of toluene, 50 mL of ethanol, and 50 mL of H₂O were added to tetrakis(4-bromophenyl) methane (10.0 g, 0.016 mol), 2-fluorophenylboronic acid (10.6 g, 0.076 mol), K₂CO₃ (26.1 g, 0.189 mol), and Pd(PPh₃) 4 (1.5 g, 0.001 mol), and the resulting mixture was stirred at 100° C. for 6 hours and reacted. After completion of the reaction, the resulting product was extracted and concentrated, and then subjected to column chromatography and recrystallization to obtain 7.9 g (yield 72.1%) of <Compound 71>.

LC/MS: m/z=696[(M)⁺]

### Synthesis Example 2: Synthesis of Compound 142

### (1) Preparation Example 1: Synthesis of Intermediate 142-1

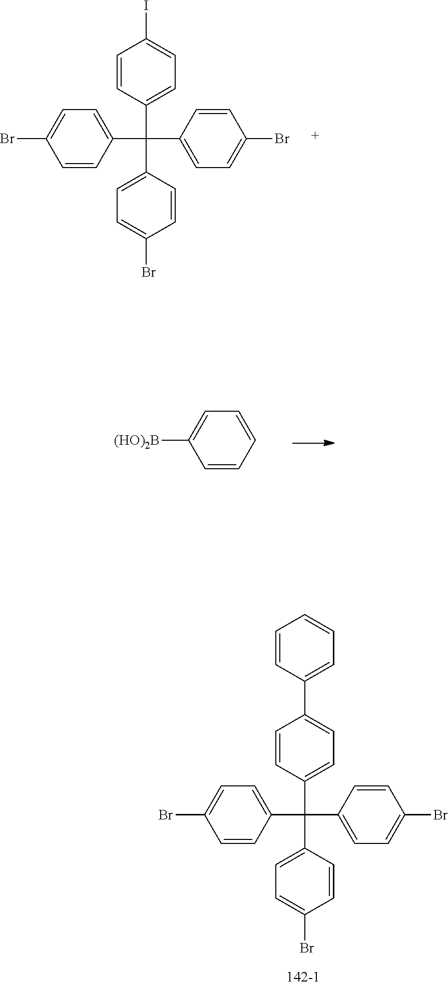 [SMILES: Brc1ccc(C(c2ccc(Br)cc2)(c2ccc(Br)cc2)c2ccc(I)cc2)cc1 ; OB(O)c1ccccc1 ; Brc1ccc(C(c2ccc(Br)cc2)(c2ccc(Br)cc2)c2ccc(-c3ccccc3)cc2)cc1]

200 mL of toluene, 50 mL of ethanol, and 50 mL of H₂O were added to 1,1′,1″-[(4-iodophenyl)methylidyne]tris[4-bromobenzene] (10.0 g, 0.015 mol), phenylboronic acid (2.1 g, 0.018 mol), K₂CO₃ (6.1 g, 0.044 mol), and Pd(PPh₃) 4 (0.3 g, 0.0003 mol), and the resulting mixture was stirred at 100° C. for 6 hours and reacted. After completion of the reaction, the resulting product was extracted and concentrated, and then subjected to column chromatography and recrystallization to obtain 3.8 g (yield 41.0%) of <Intermediate 142-1>.

### (2) Preparation Example 2: Synthesis of Compound 142

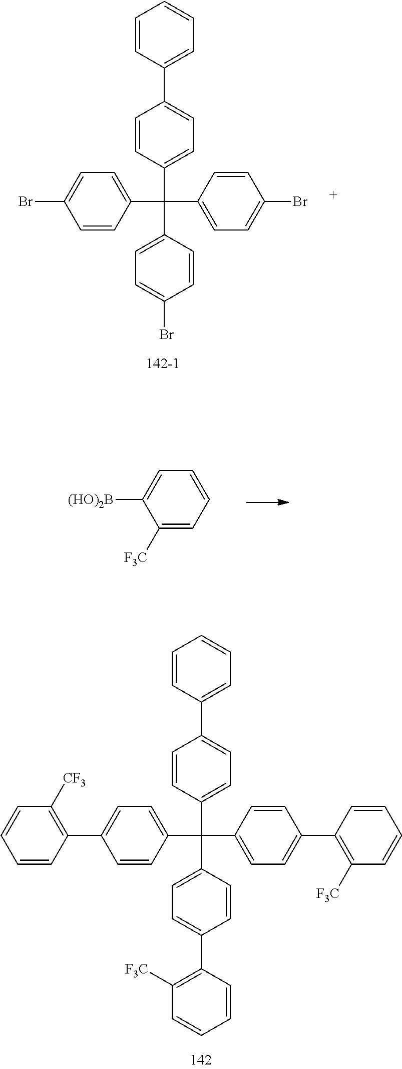 [SMILES: Brc1ccc(C(c2ccc(Br)cc2)(c2ccc(Br)cc2)c2ccc(-c3ccccc3)cc2)cc1 ; OB(O)c1ccccc1C(F)(F)F ; FC(F)(F)c1ccccc1-c1ccc(C(c2ccc(-c3ccccc3)cc2)(c2ccc(-c3ccccc3C(F)(F)F)cc2)c2ccc(-c3ccccc3C(F)(F)F)cc2)cc1]

200 mL of toluene, 50 mL of ethanol, and 50 mL of H₂O were added to Intermediate 142-1 (10.0 g, 0.016 mol), 2-trifluoromethylphenylboronic acid (10.8 g, 0.057 mol), K₂CO₃ (19.6 g, 0.142 mol), and Pd(PPh₃) 4 (0.4 g, 0.0003 mol), and the resulting mixture was stirred at 100° C. for 6 hours and reacted. After completion of the reaction, the resulting product was extracted and concentrated, and then subjected to column chromatography and recrystallization to obtain 8.7 g (yield 66.5%) of <Compound 142>.

LC/MS: m/z=828[(M)⁺]

### Synthesis Example 3: Synthesis of Compound 159

### (1) Preparation Example 1: Synthesis of Compound 159

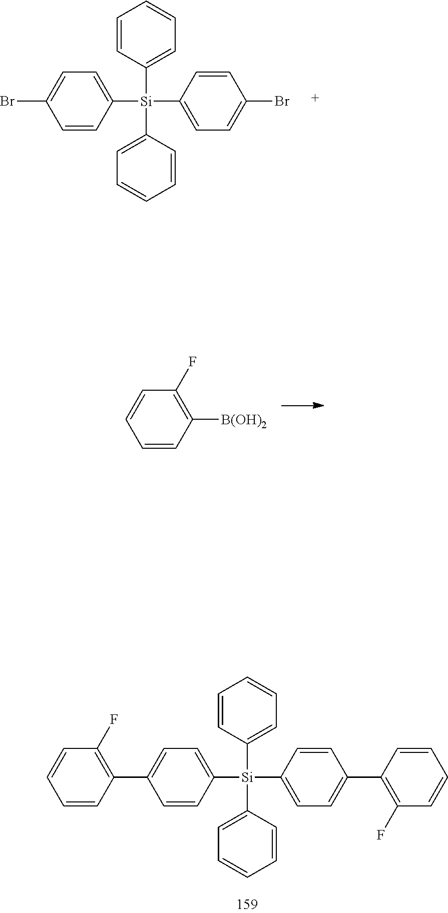 [SMILES: Brc1ccc([Si](c2ccccc2)(c2ccccc2)c2ccc(Br)cc2)cc1 ; OB(O)c1ccccc1F ; Fc1ccccc1-c1ccc([Si](c2ccccc2)(c2ccccc2)c2ccc(-c3ccccc3F)cc2)cc1]

200 mL of toluene, 50 mL of ethanol, and 50 mL of H₂O were added to bis(4-bromophenyl)-diphenyl-silane (10.0 g, 0.020 mol), 2-fluorophenylboronic acid (6.8 g, 0.049 mol), K₂CO₃ (16.8 g, 0.121 mol), and Pd(PPh₃) 4 (0.5 g, 0.0004 mol), and the resulting mixture was stirred at 100° C. for 6 hours and reacted. After completion of the reaction, the resulting product was extracted and concentrated, and then subjected to column chromatography and recrystallization to obtain 6.8 g (yield 64.1%) of <Compound 159>.

LC/MS: m/z=524[(M)⁺]

### Synthesis Example 4: Synthesis of Compound 239

### (1) Preparation Example 1: Synthesis of Compound 239

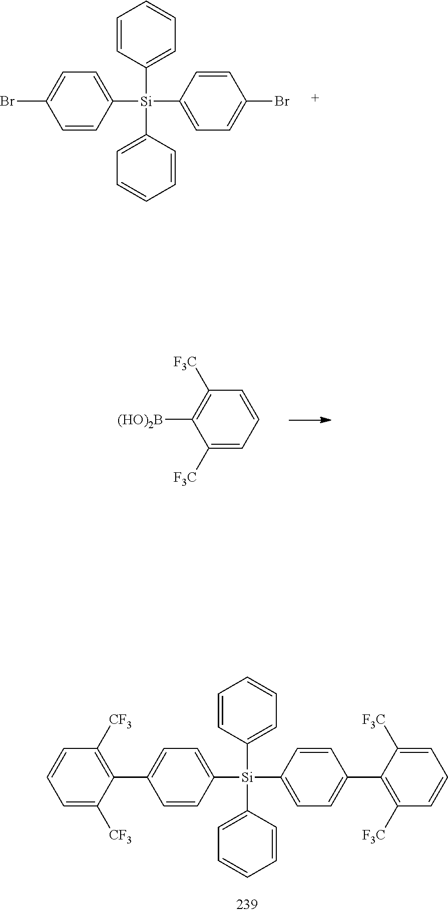 [SMILES: Brc1ccc([Si](c2ccccc2)(c2ccccc2)c2ccc(Br)cc2)cc1 ; OB(O)c1c(C(F)(F)F)cccc1C(F)(F)F ; FC(F)(F)c1cccc(C(F)(F)F)c1-c1ccc([Si](c2ccccc2)(c2ccccc2)c2ccc(-c3c(C(F)(F)F)cccc3C(F)(F)F)cc2)cc1]

200 mL of toluene, 50 mL of ethanol, and 50 mL of H₂O were added to bis(4-bromophenyl)-diphenyl-silane (10.0 g, 0.020 mol), B-[2,6-bis(trifluoromethyl)phenyl]boronic acid (12.5 g, 0.049 mol), K₂CO₃ (16.8 g, 0.121 mol), and Pd(PPh₃) 4 (0.5 g, 0.0004 mol), and the resulting mixture was stirred at 100° C. for 6 hours and reacted. After completion of the reaction, the resulting product was extracted and concentrated, and then subjected to column chromatography and recrystallization to obtain 8.6 g (yield 55.9%) of <Compound 239>.

LC/MS: m/z=760[(M)⁺]

### Synthesis Example 5: Synthesis of Compound 310

### (1) Preparation Example 1: Synthesis of Compound 310

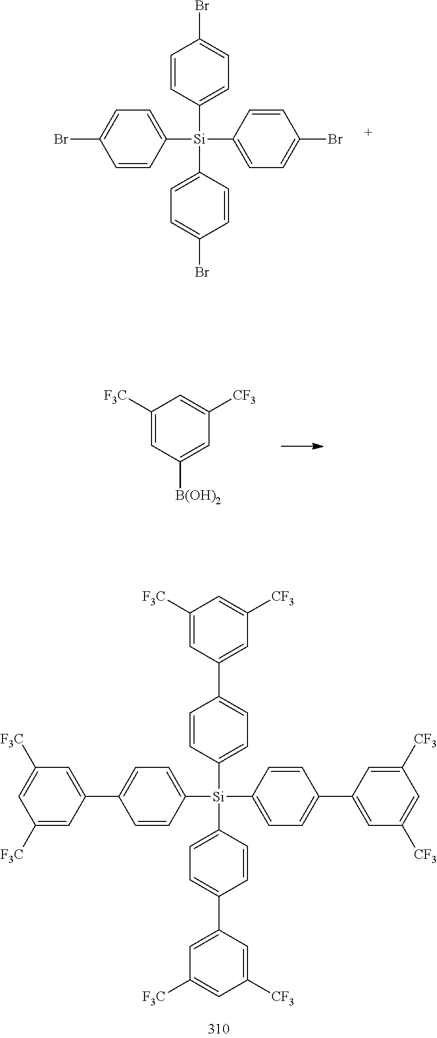 [SMILES: Brc1ccc([Si](c2ccc(Br)cc2)(c2ccc(Br)cc2)c2ccc(Br)cc2)cc1 ; OB(O)c1cc(C(F)(F)F)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1]

200 mL of toluene, 50 mL of ethanol, and 50 mL of H₂O were added to 1,1′,1″,1′″-silanetetrayltetrakis[4-bromobenzene] (10.0 g, 0.015 mol), 3,5-bis(trifluoromethyl)phenylboronic acid (19.0 g, 0.074 mol), K₂CO₃ (25.4 g, 0.184 mol), and Pd(PPh₃) 4 (1.4 g, 0.001 mol), and the resulting mixture was stirred at 100° C. for 6 hours and reacted. After completion of the reaction, the resulting product was extracted and concentrated, and then subjected to column chromatography and recrystallization to obtain 10.3 g (yield 56.7%) of <Compound 310>.

LC/MS: m/z=1184[(M)⁺]

### Device Examples (CPL)

In embodiments according to the present invention, an anode was patterned using an ITO glass substrate including Ag of 25 mm×25 mm×0.7 mm such that a light-emitting area had a size of 2 mm×2 mm, and then washed. After the patterned ITO substrate was mounted in a vacuum chamber, an organic material and a metal were deposited on the substrate at a process pressure of 1×10⁻⁶ torr or more as the following structure.

### Device Examples 1 to 37

After an organic light-emitting device having the following device structure was manufactured by employing a compound implemented by the present invention for a light efficiency improving layer, the light-emitting and driving characteristics of the compound embodied according to the present invention and an organic light-emitting device using the same were measured.

Ag/ITO/hole injection layer (HAT-CN, 5 nm)/hole transport layer (α-NPB, 100 nm)/electron blocking layer (EB1, 10 nm)/light-emitting layer (20 nm)/electron transport layer (ET1:Liq, 30 nm)/LiF (1 nm)/Mg:Ag (15 nm)/light efficiency improving layer (70 nm)

After [HAT-CN] was film-formed to a thickness of 5 nm on an ITO transparent electrode containing Ag on a glass substrate to form a hole injection layer, [α-NPB] was film-formed to 100 nm to form a hole transport layer, and [EB1] was film-formed to a thickness of 10 nm to form an electron blocking layer, [BH1] as a host compound and [BD1] as a dopant compound were used and co-deposited to 20 nm to form a light-emitting layer, an electron transport layer (doped with 50% of the following [ET1] compound Liq) was deposited to a thickness of 30 nm, and then LiF was film-formed to a thickness of 1 nm to form an electron injection layer, and then Mg and Ag were film-formed to a thickness of 15 nm at a ratio of 1:9 to form a cathode. And, a light efficiency improving layer (capping layer) was film-formed to a thickness of 70 nm using the compound embodied by the present invention to manufacture an organic light-emitting device.

### Device Comparative Example 1

An organic light-emitting device for Device Comparative Example 1 was manufactured in the same manner as in the device structures in Examples 1 to 37, except that the light efficiency improving layer was not used.

### Device Comparative Example 2

An organic light-emitting device for Device Comparative Example 2 was manufactured in the same manner as in the device structures in Examples 1 to 37, except that as the light efficiency improving layer compound, Alq₃ was used instead of the compound of the present invention.

### Device Comparative Example 3

An organic light-emitting device for Device Comparative Example 3 was manufactured in the same manner as in the device structures in Examples 1 to 37, except that as the light efficiency improving layer compound, CP1 was used instead of the compound of the present invention.

### Experimental Example 1: Light-Emitting Characteristics of Device Examples 1 to 37

For the organic light-emitting devices manufactured by the Examples and the Comparative Examples, driving voltage, current efficiency and color coordinate were measured using a source meter (Model237, Keithley) and a luminance meter (PR-650, Photo Research), and the result values based on 1,000 nits are shown in the following [Table 1].

TABLE 1

| | Light efficiency | | | | |
|---|---|---|---|---|---|
| Example | improving layer | V | cd/A | CIEx | CIEy |
| 1 | Formula 1 | 3.76 | 8.49 | 0.1319 | 0.0603 |
| 2 | Formula 5 | 3.60 | 8.79 | 0.1353 | 0.0621 |
| 3 | Formula 10 | 3.63 | 8.61 | 0.1309 | 0.0653 |
| 4 | Formula 15 | 3.53 | 8.80 | 0.1323 | 0.0589 |
| 5 | Formula 22 | 3.50 | 8.34 | 0.1328 | 0.0595 |
| 6 | Formula 28 | 3.61 | 8.96 | 0.1378 | 0.0635 |
| 7 | Formula 34 | 3.32 | 8.39 | 0.1321 | 0.0627 |
| 8 | Formula 46 | 3.35 | 8.64 | 0.1340 | 0.0607 |
| 9 | Formula 50 | 3.39 | 8.69 | 0.1360 | 0.0534 |
| 10 | Formula 57 | 3.58 | 8.59 | 0.1379 | 0.0605 |
| 11 | Formula 64 | 3.61 | 8.96 | 0.1387 | 0.0643 |
| 12 | Formula 71 | 3.70 | 8.39 | 0.1317 | 0.0578 |
| 13 | Formula 75 | 3.59 | 8.62 | 0.1314 | 0.0643 |
| 14 | Formula 80 | 3.71 | 8.66 | 0.1318 | 0.0635 |
| 15 | Formula 89 | 3.59 | 8.69 | 0.1389 | 0.0623 |
| 16 | Formula 92 | 3.70 | 8.49 | 0.1319 | 0.0583 |
| 17 | Formula 99 | 3.62 | 8.52 | 0.1349 | 0.0550 |
| 18 | Formula 116 | 3.63 | 8.67 | 0.1333 | 0.0621 |
| 19 | Formula 132 | 3.74 | 8.38 | 0.1349 | 0.0542 |
| 20 | Formula 142 | 3.77 | 8.27 | 0.1350 | 0.0576 |
| 21 | Formula 150 | 3.82 | 8.89 | 0.1353 | 0.0546 |
| 22 | Formula 159 | 3.75 | 8.41 | 0.1372 | 0.0513 |
| 23 | Formula 163 | 3.96 | 8.43 | 0.1380 | 0.0514 |
| 24 | Formula 180 | 3.78 | 9.21 | 0.1389 | 0.0633 |
| 25 | Formula 196 | 3.86 | 8.71 | 0.1399 | 0.0663 |
| 26 | Formula 208 | 3.87 | 8.41 | 0.1340 | 0.0549 |
| 27 | Formula 218 | 3.93 | 8.85 | 0.1365 | 0.0522 |
| 28 | Formula 227 | 3.51 | 8.99 | 0.1359 | 0.0633 |
| 29 | Formula 233 | 3.59 | 8.69 | 0.1389 | 0.0623 |
| 30 | Formula 239 | 3.63 | 8.68 | 0.1321 | 0.0615 |
| 31 | Formula 257 | 3.76 | 8.35 | 0.1365 | 0.0593 |
| 32 | Formula 289 | 3.79 | 8.66 | 0.1374 | 0.0600 |
| 33 | Formula 298 | 3.77 | 8.39 | 0.1363 | 0.0591 |
| 34 | Formula 304 | 3.59 | 8.62 | 0.1314 | 0.0643 |
| 35 | Formula 308 | 3.72 | 8.47 | 0.1358 | 0.0510 |
| 36 | Formula 310 | 3.73 | 8.56 | 0.1322 | 0.0592 |
| 37 | Formula 314 | 3.77 | 8.47 | 0.1328 | 0.0540 |
| Comparative | Not used | 4.68 | 7.03 | 0.1502 | 0.1412 |
| Example 1 | | | | | |
| Comparative | Alq₃ | 4.33 | 7.84 | 0.1471 | 0.0583 |
| Example 2 | | | | | |
| Comparative | CP1 | 4.19 | 8.04 | 0.1391 | 0.0683 |
| Example 3 | | | | | |

Referring to the results shown in [Table 1], it can be confirmed that the organic light-emitting device in which the compound according to the present invention is applied to a light efficiency improving layer has a reduced driving voltage and an improved current efficiency compared to devices in the related art (Comparative Examples 1 to 3).

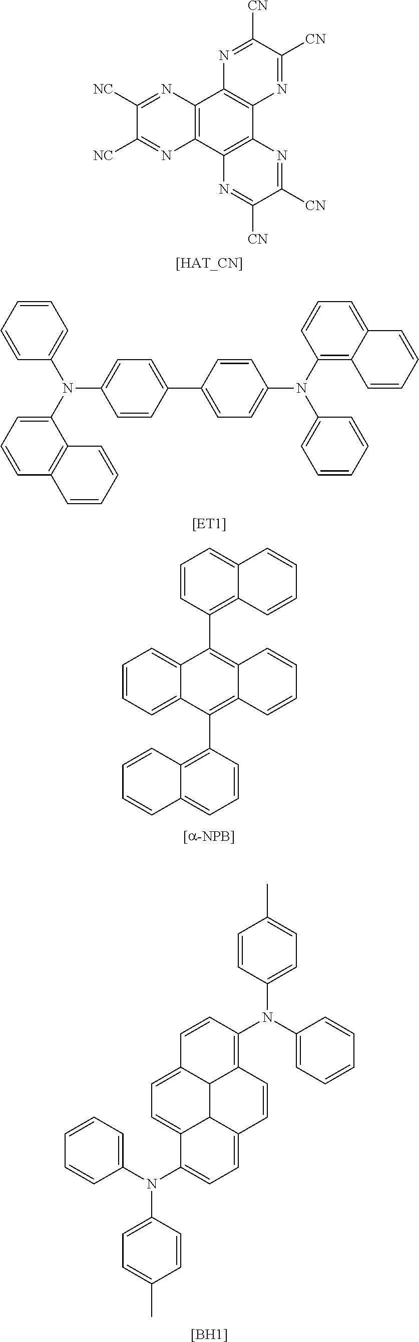

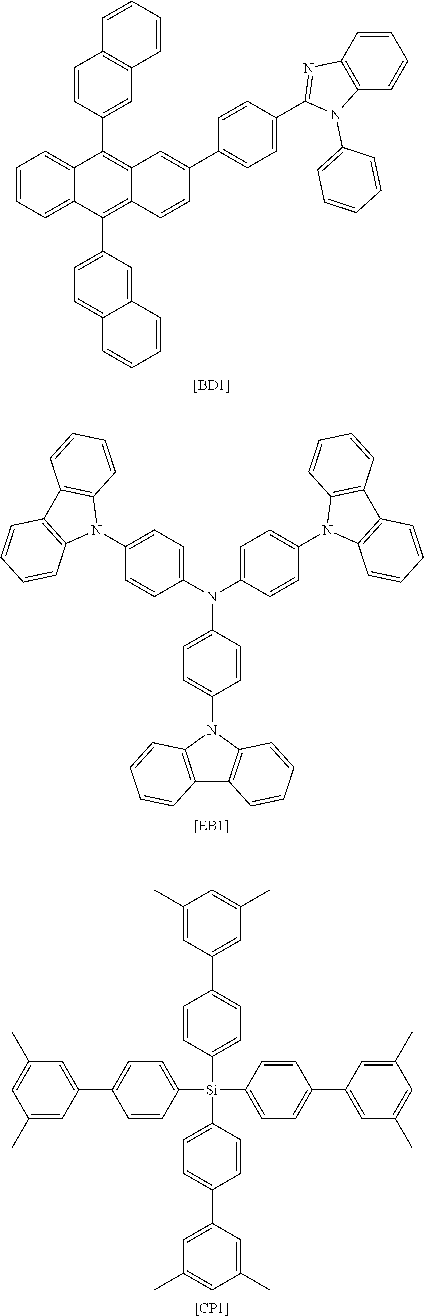

### INDUSTRIAL APPLICABILITY

The present invention relates to a compound for a light efficiency improving layer material provided in an organic light-emitting device, and when the compound according to the present invention is adopted for a light efficiency improving layer provided in an organic light-emitting device, the organic light-emitting compound can be industrially usefully used for various lighting devices and display devices because it is possible to implement improved light-emitting characteristics such as low voltage driving and excellent light-emitting efficiency and color purity.

1. An organic compound represented by Formula I below: 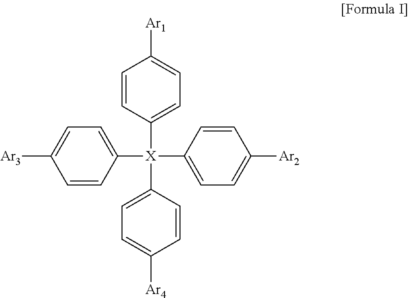
wherein, in Formula I above,
X is C or Si,
Ar₁ to Ar₄ are the same as or different from each other, and each independently any one selected from hydrogen, deuterium, a cyano group, a halogen group, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and
at least one of Ar₁ to Ar₄ is any one selected from a halogen group, a substituted or unsubstituted halogenated alkyl group having 1 to 20 carbon atoms, and a substituted or unsubstituted aryl group having 6 to 30 carbon atoms.

2. The organic compound of claim 1, wherein in the definitions of Ar₁ to Ar₄, ‘substituted or unsubstituted’ means that each of Ar₁ to Ar₄ is substituted with one or two or more substituents selected from the group consisting of deuterium, a halogen group, a cyano group, a nitro group, a hydroxyl group, a silyl group, an amine group, a halogenated alkyl group, a deuterated alkyl group, a cycloalkyl group, a heterocycloalkyl group, a halogenated alkoxy group, a deuterated alkoxy group, an aryl group, a heteroaryl group, an alkylsilyl group, and an arylsilyl group, is substituted with a substituent to which two or more substituent among the above substituents are linked, or has no substituent.

3. The organic compound of claim 1, wherein at least one of Ar₁ to Ar₄ is an aryl group having 6 to 30 carbon atoms, which is substituted with one or two or more substituents selected from a halogen group and a halogenated alkyl group, or substituted with a substituent to which two or more of the above substituents are linked.

4. The organic compound of claim 1, wherein Formula I above is any one selected from the following [Compound 1] to [Compound 316]:

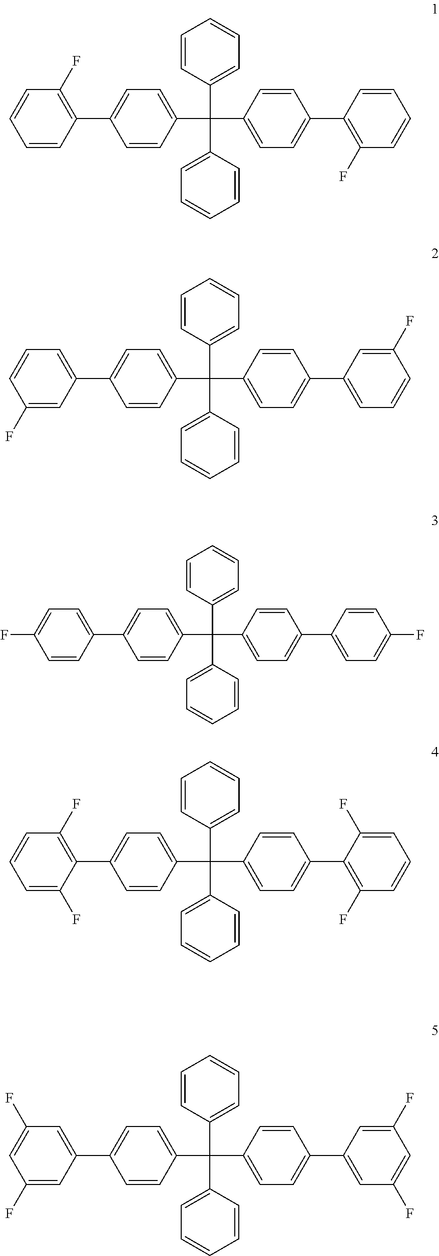

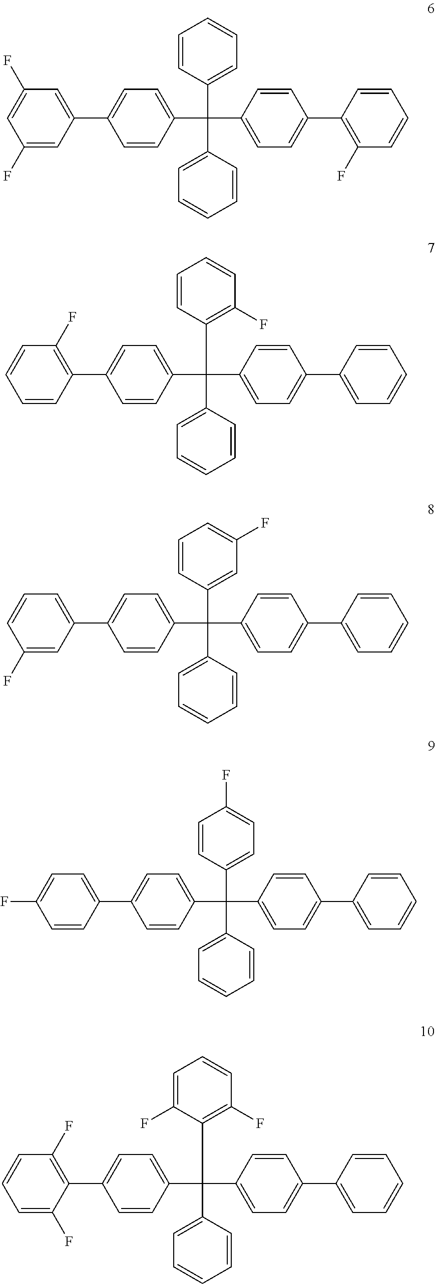

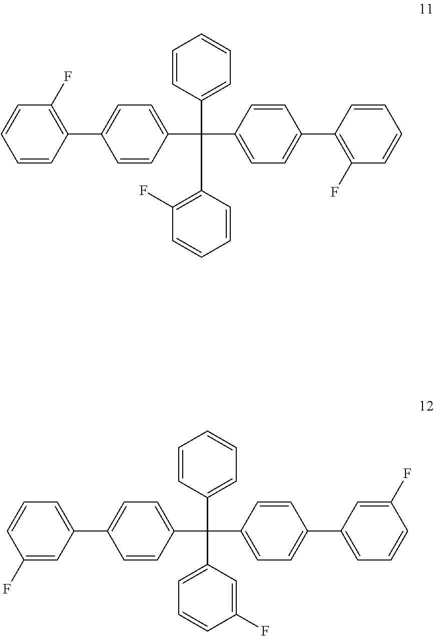 [SMILES: Fc1ccccc1-c1ccc(C(c2ccccc2)(c2ccc(-c3ccccc3F)cc2)c2ccccc2F)cc1 ; Fc1cccc(-c2ccc(C(c3ccccc3)(c3ccc(-c4cccc(F)c4)cc3)c3cccc(F)c3)cc2)c1]

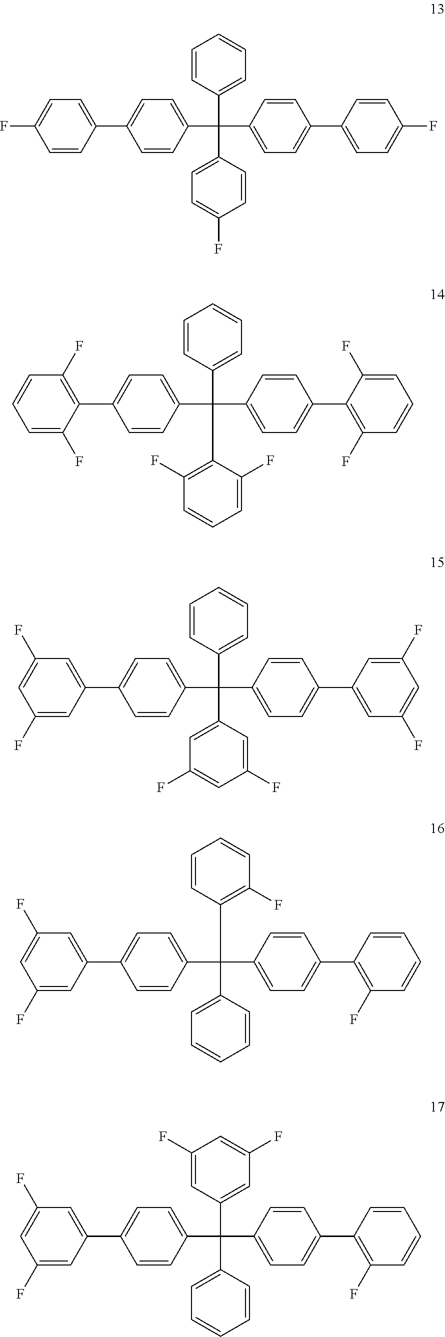 [SMILES: Fc1ccc(-c2ccc(C(c3ccccc3)(c3ccc(F)cc3)c3ccc(-c4ccc(F)cc4)cc3)cc2)cc1 ; Fc1cccc(F)c1-c1ccc(C(c2ccccc2)(c2ccc(-c3c(F)cccc3F)cc2)c2c(F)cccc2F)cc1 ; Fc1cc(F)cc(-c2ccc(C(c3ccccc3)(c3ccc(-c4cc(F)cc(F)c4)cc3)c3cc(F)cc(F)c3)cc2)c1 ; Fc1cc(F)cc(-c2ccc(C(c3ccccc3)(c3ccc(-c4ccccc4F)cc3)c3ccccc3F)cc2)c1 ; Fc1cc(F)cc(-c2ccc(C(c3ccccc3)(c3ccc(-c4ccccc4F)cc3)c3cc(F)cc(F)c3)cc2)c1]

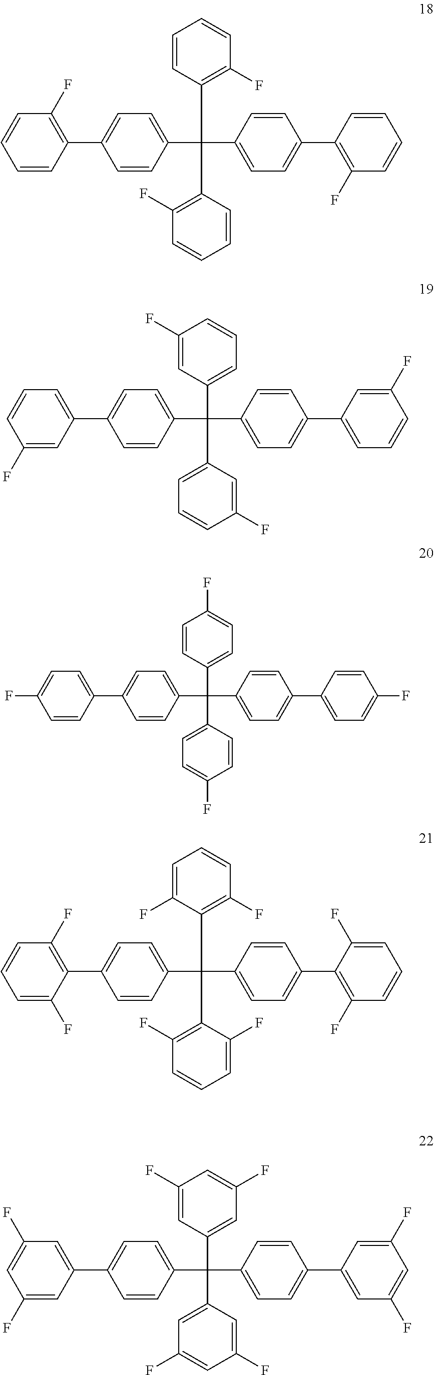

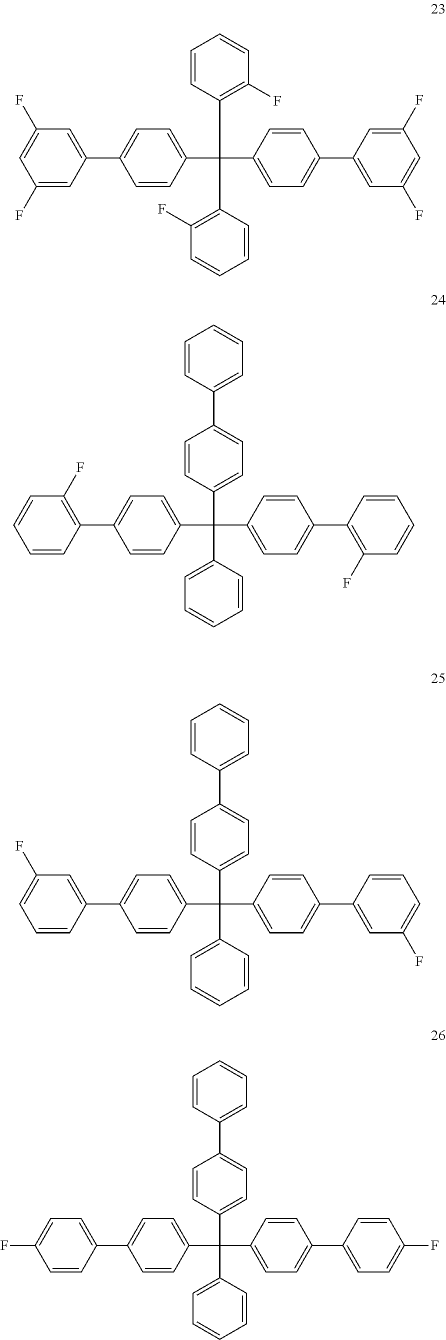 [SMILES: Fc1cc(F)cc(-c2ccc(C(c3ccc(-c4cc(F)cc(F)c4)cc3)(c3ccccc3F)c3ccccc3F)cc2)c1 ; Fc1ccccc1-c1ccc(C(c2ccccc2)(c2ccc(-c3ccccc3)cc2)c2ccc(-c3ccccc3F)cc2)cc1 ; Fc1cccc(-c2ccc(C(c3ccccc3)(c3ccc(-c4ccccc4)cc3)c3ccc(-c4cccc(F)c4)cc3)cc2)c1 ; Fc1ccc(-c2ccc(C(c3ccccc3)(c3ccc(-c4ccccc4)cc3)c3ccc(-c4ccc(F)cc4)cc3)cc2)cc1]

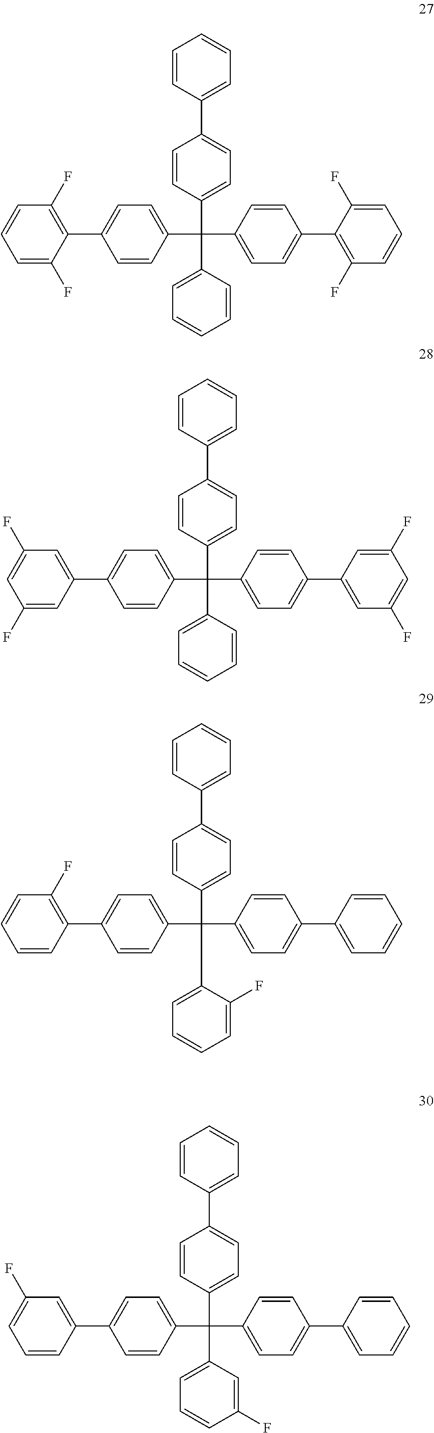

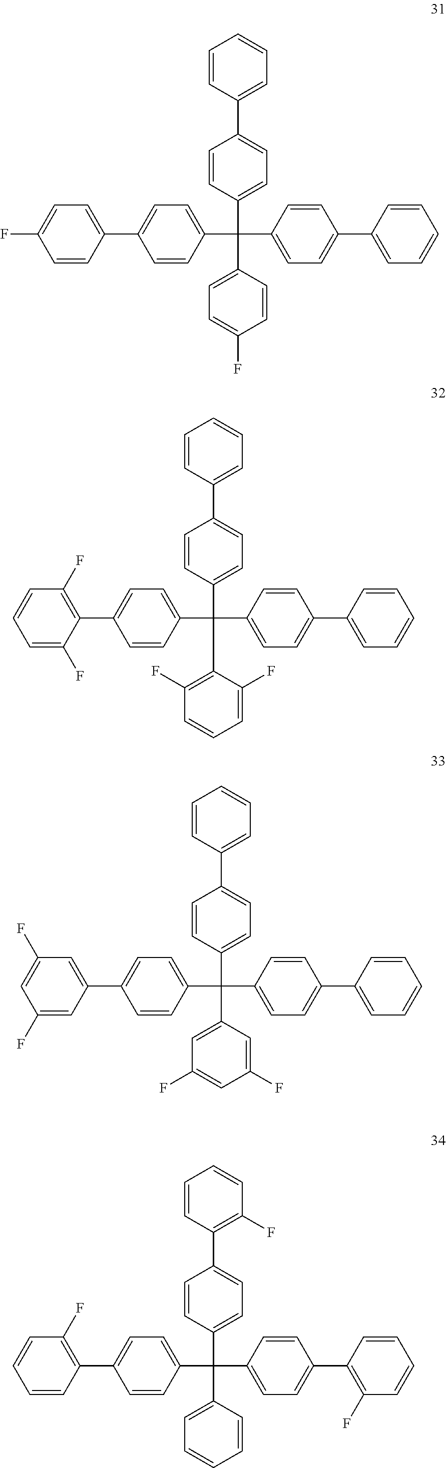 [SMILES: Fc1ccc(-c2ccc(C(c3ccc(F)cc3)(c3ccc(-c4ccccc4)cc3)c3ccc(-c4ccccc4)cc3)cc2)cc1 ; Fc1cccc(F)c1-c1ccc(C(c2ccc(-c3ccccc3)cc2)(c2ccc(-c3ccccc3)cc2)c2c(F)cccc2F)cc1 ; Fc1cc(F)cc(-c2ccc(C(c3ccc(-c4ccccc4)cc3)(c3ccc(-c4ccccc4)cc3)c3cc(F)cc(F)c3)cc2)c1 ; Fc1ccccc1-c1ccc(C(c2ccccc2)(c2ccc(-c3ccccc3F)cc2)c2ccc(-c3ccccc3F)cc2)cc1]

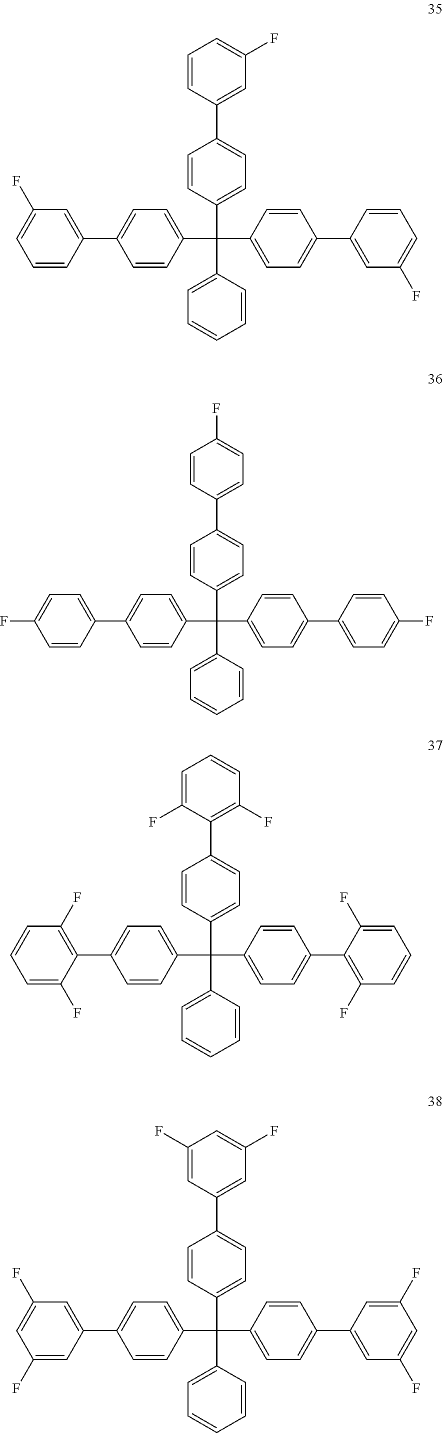

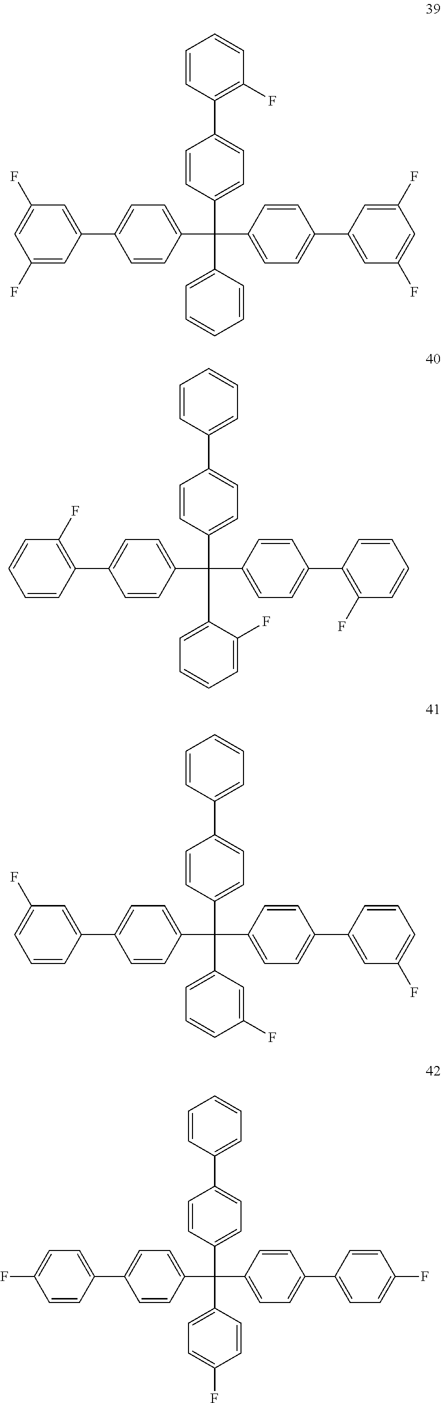

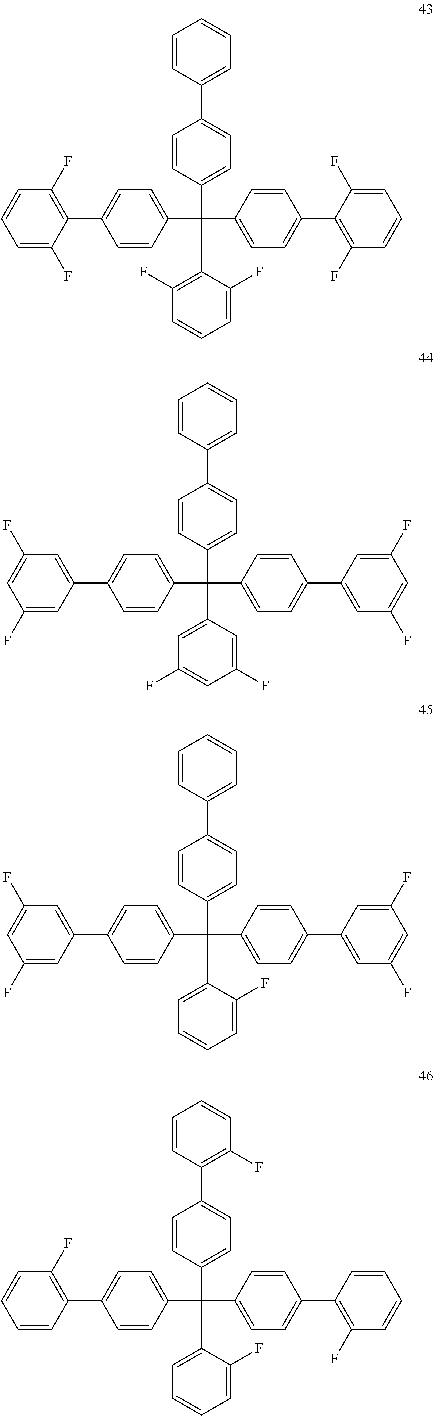

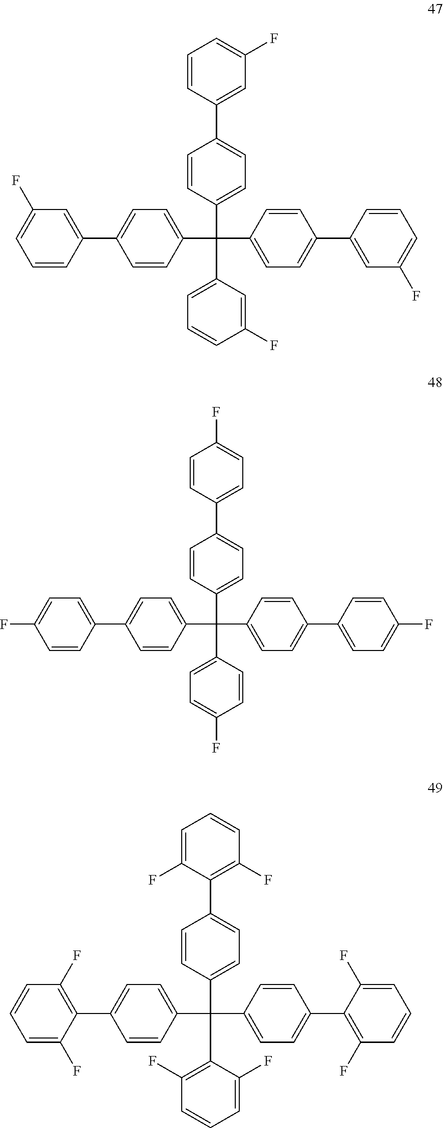 [SMILES: Fc1cccc(-c2ccc(C(c3ccc(-c4cccc(F)c4)cc3)(c3ccc(-c4cccc(F)c4)cc3)c3cccc(F)c3)cc2)c1 ; Fc1ccc(-c2ccc(C(c3ccc(F)cc3)(c3ccc(-c4ccc(F)cc4)cc3)c3ccc(-c4ccc(F)cc4)cc3)cc2)cc1 ; Fc1cccc(F)c1-c1ccc(C(c2ccc(-c3c(F)cccc3F)cc2)(c2ccc(-c3c(F)cccc3F)cc2)c2c(F)cccc2F)cc1]

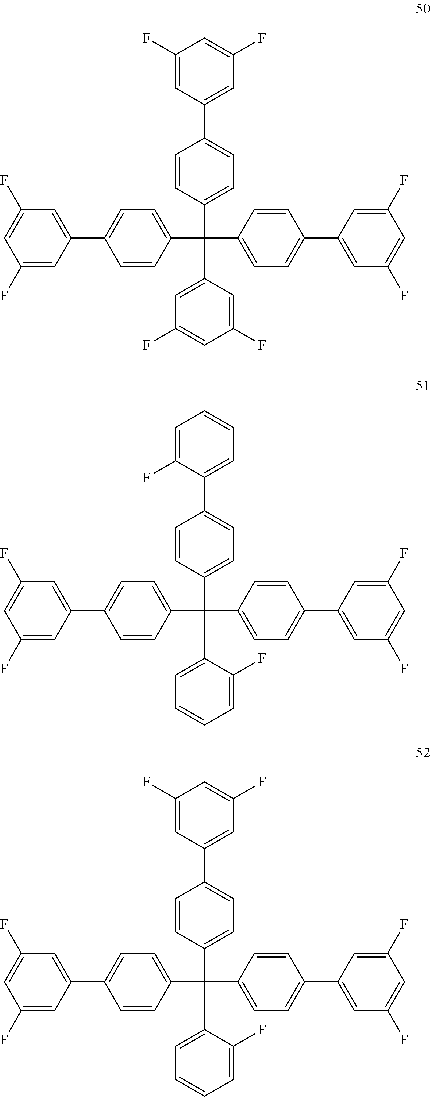 [SMILES: Fc1cc(F)cc(-c2ccc(C(c3ccc(-c4cc(F)cc(F)c4)cc3)(c3ccc(-c4cc(F)cc(F)c4)cc3)c3cc(F)cc(F)c3)cc2)c1 ; Fc1cc(F)cc(-c2ccc(C(c3ccc(-c4cc(F)cc(F)c4)cc3)(c3ccc(-c4ccccc4F)cc3)c3ccccc3F)cc2)c1 ; Fc1cc(F)cc(-c2ccc(C(c3ccc(-c4cc(F)cc(F)c4)cc3)(c3ccc(-c4cc(F)cc(F)c4)cc3)c3ccccc3F)cc2)c1]

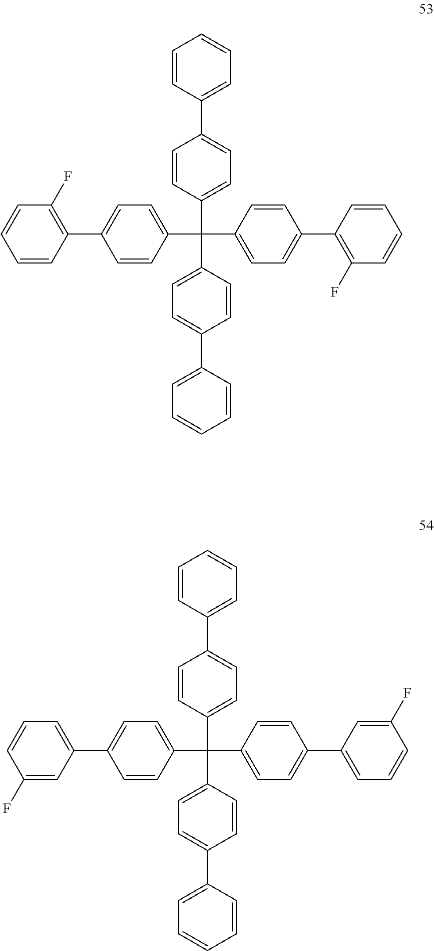 [SMILES: Fc1ccccc1-c1ccc(C(c2ccc(-c3ccccc3)cc2)(c2ccc(-c3ccccc3)cc2)c2ccc(-c3ccccc3F)cc2)cc1 ; Fc1cccc(-c2ccc(C(c3ccc(-c4ccccc4)cc3)(c3ccc(-c4ccccc4)cc3)c3ccc(-c4cccc(F)c4)cc3)cc2)c1]

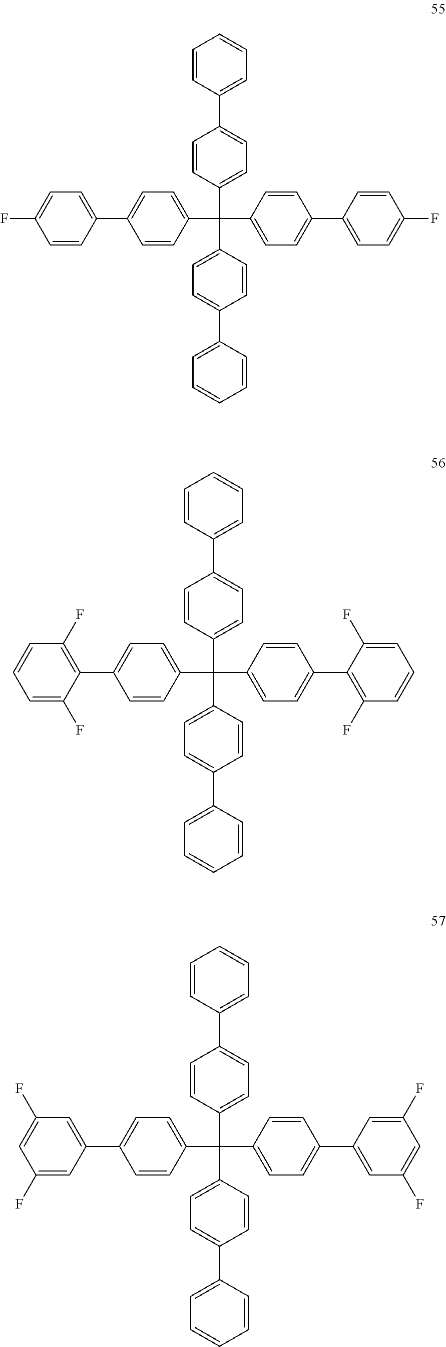 [SMILES: Fc1ccc(-c2ccc(C(c3ccc(-c4ccccc4)cc3)(c3ccc(-c4ccccc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3ccccc3)cc2)c2ccc(-c3c(F)cccc3F)cc2)cc1 ; Fc1cc(F)cc(-c2ccc(C(c3ccc(-c4ccccc4)cc3)(c3ccc(-c4ccccc4)cc3)c3ccc(-c4cc(F)cc(F)c4)cc3)cc2)c1]

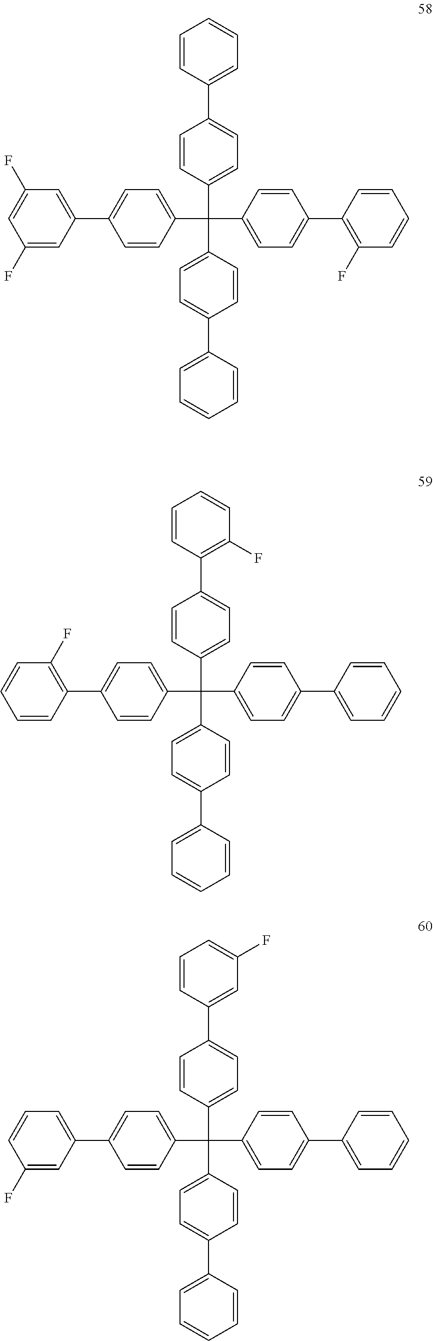 [SMILES: Fc1cc(F)cc(-c2ccc(C(c3ccc(-c4ccccc4)cc3)(c3ccc(-c4ccccc4)cc3)c3ccc(-c4ccccc4F)cc3)cc2)c1 ; Fc1ccccc1-c1ccc(C(c2ccc(-c3ccccc3)cc2)(c2ccc(-c3ccccc3)cc2)c2ccc(-c3ccccc3F)cc2)cc1 ; Fc1cccc(-c2ccc(C(c3ccc(-c4ccccc4)cc3)(c3ccc(-c4ccccc4)cc3)c3ccc(-c4cccc(F)c4)cc3)cc2)c1]

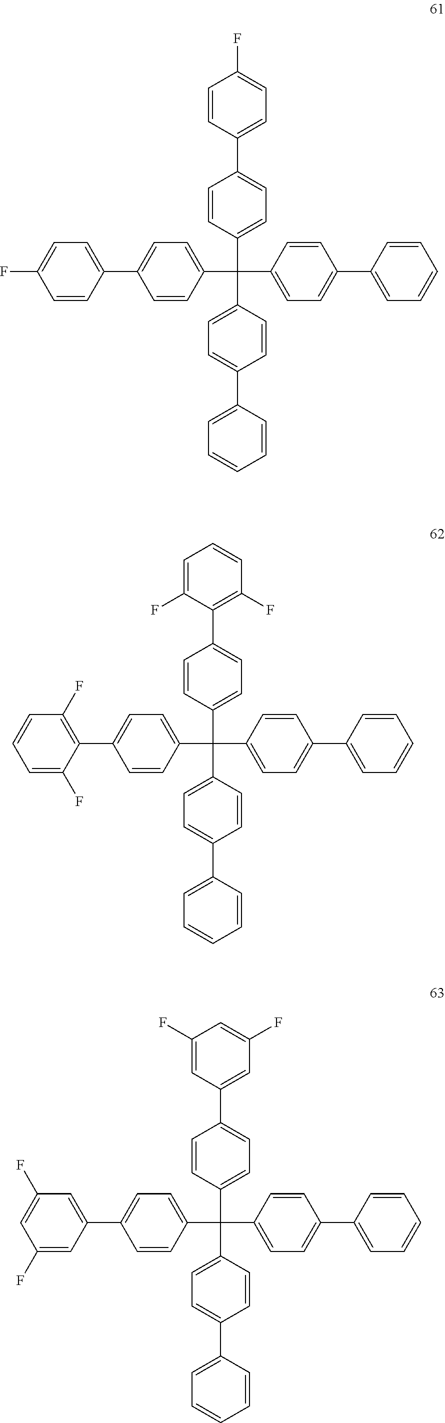 [SMILES: Fc1ccc(-c2ccc(C(c3ccc(-c4ccccc4)cc3)(c3ccc(-c4ccccc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3ccccc3)cc2)c2ccc(-c3c(F)cccc3F)cc2)cc1 ; Fc1cc(F)cc(-c2ccc(C(c3ccc(-c4ccccc4)cc3)(c3ccc(-c4ccccc4)cc3)c3ccc(-c4cc(F)cc(F)c4)cc3)cc2)c1]

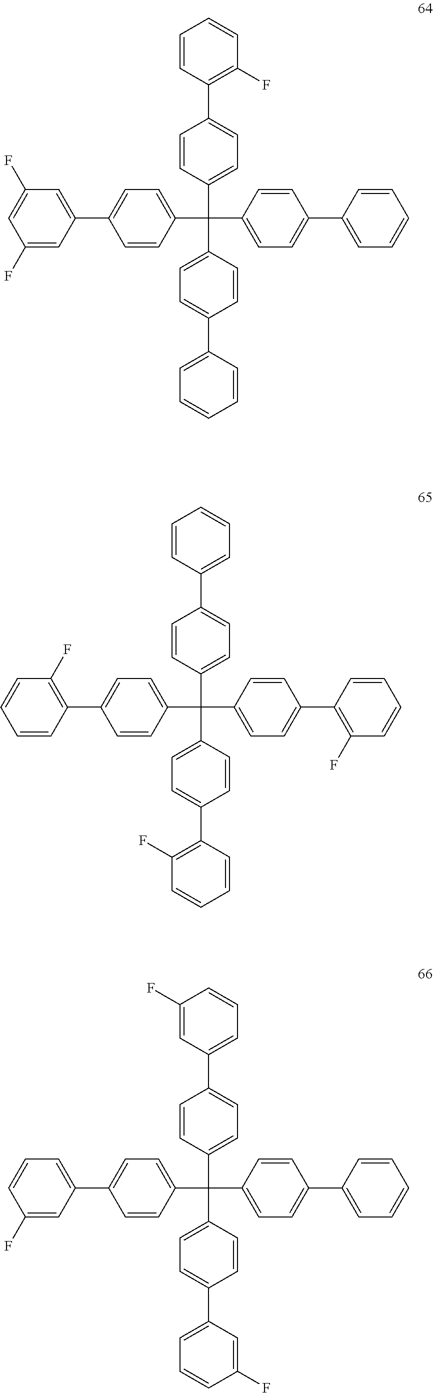 [SMILES: Fc1cc(F)cc(-c2ccc(C(c3ccc(-c4ccccc4)cc3)(c3ccc(-c4ccccc4)cc3)c3ccc(-c4ccccc4F)cc3)cc2)c1 ; Fc1ccccc1-c1ccc(C(c2ccc(-c3ccccc3)cc2)(c2ccc(-c3ccccc3F)cc2)c2ccc(-c3ccccc3F)cc2)cc1 ; Fc1cccc(-c2ccc(C(c3ccc(-c4ccccc4)cc3)(c3ccc(-c4cccc(F)c4)cc3)c3ccc(-c4cccc(F)c4)cc3)cc2)c1]

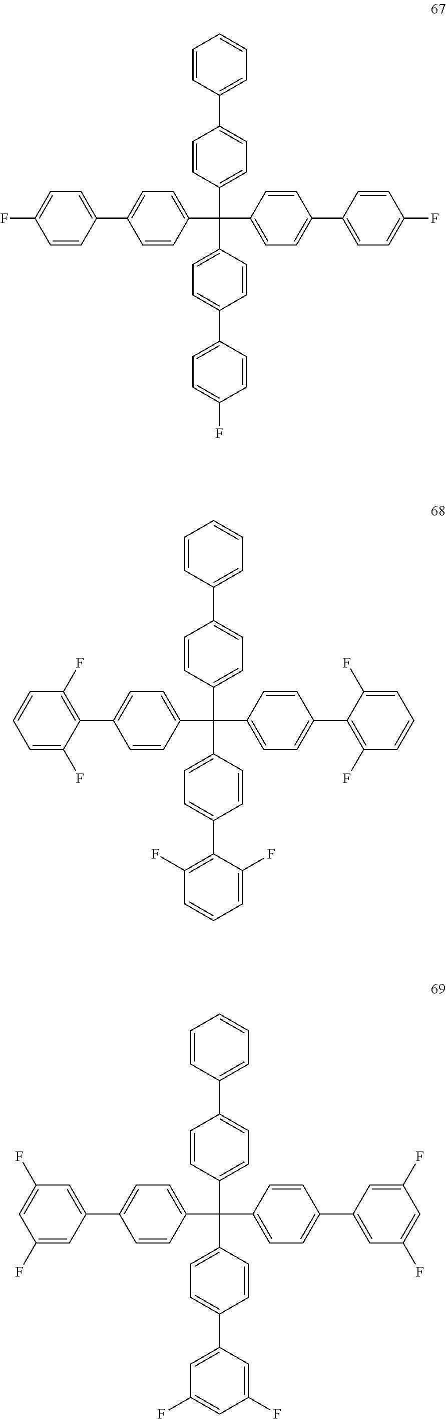 [SMILES: Fc1ccc(-c2ccc(C(c3ccc(-c4ccccc4)cc3)(c3ccc(-c4ccc(F)cc4)cc3)c3ccc(-c4ccc(F)cc4)cc3)cc2)cc1 ; Fc1cccc(F)c1-c1ccc(C(c2ccc(-c3ccccc3)cc2)(c2ccc(-c3c(F)cccc3F)cc2)c2ccc(-c3c(F)cccc3F)cc2)cc1 ; Fc1cc(F)cc(-c2ccc(C(c3ccc(-c4ccccc4)cc3)(c3ccc(-c4cc(F)cc(F)c4)cc3)c3ccc(-c4cc(F)cc(F)c4)cc3)cc2)c1]

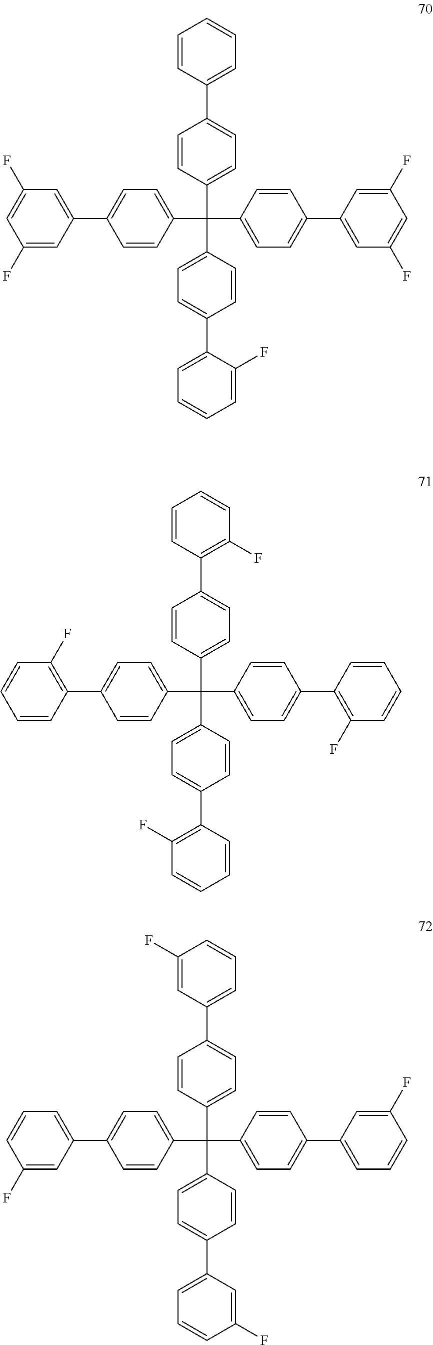 [SMILES: Fc1cc(F)cc(-c2ccc(C(c3ccc(-c4ccccc4)cc3)(c3ccc(-c4cc(F)cc(F)c4)cc3)c3ccc(-c4ccccc4F)cc3)cc2)c1 ; Fc1ccccc1-c1ccc(C(c2ccc(-c3ccccc3F)cc2)(c2ccc(-c3ccccc3F)cc2)c2ccc(-c3ccccc3F)cc2)cc1 ; Fc1cccc(-c2ccc(C(c3ccc(-c4cccc(F)c4)cc3)(c3ccc(-c4cccc(F)c4)cc3)c3ccc(-c4cccc(F)c4)cc3)cc2)c1]

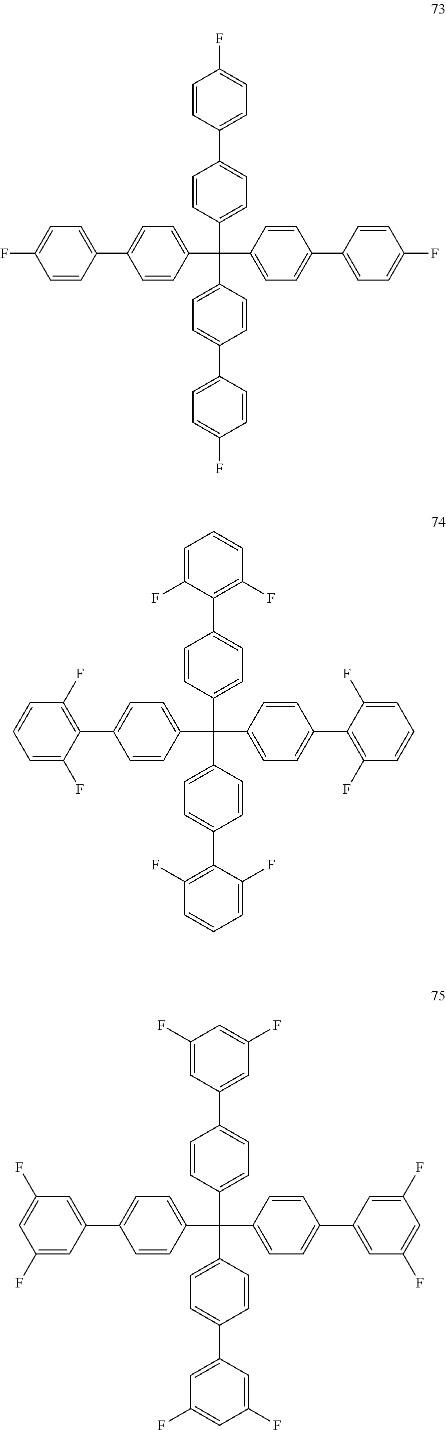 [SMILES: Fc1ccc(-c2ccc(C(c3ccc(-c4ccc(F)cc4)cc3)(c3ccc(-c4ccc(F)cc4)cc3)c3ccc(-c4ccc(F)cc4)cc3)cc2)cc1 ; Fc1cccc(F)c1-c1ccc(C(c2ccc(-c3c(F)cccc3F)cc2)(c2ccc(-c3c(F)cccc3F)cc2)c2ccc(-c3c(F)cccc3F)cc2)cc1 ; Fc1cc(F)cc(-c2ccc(C(c3ccc(-c4cc(F)cc(F)c4)cc3)(c3ccc(-c4cc(F)cc(F)c4)cc3)c3ccc(-c4cc(F)cc(F)c4)cc3)cc2)c1]

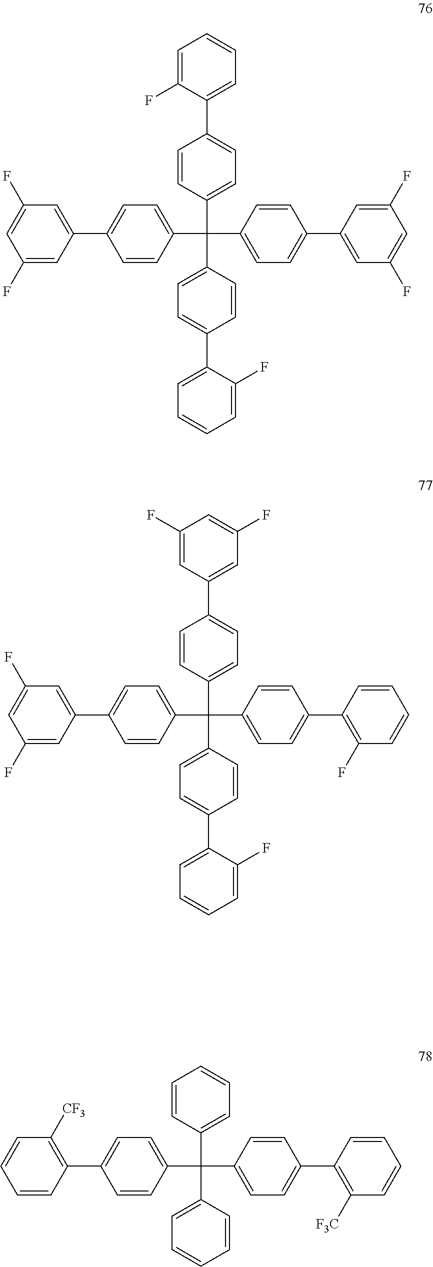 [SMILES: Fc1cc(F)cc(-c2ccc(C(c3ccc(-c4cc(F)cc(F)c4)cc3)(c3ccc(-c4ccccc4F)cc3)c3ccc(-c4ccccc4F)cc3)cc2)c1 ; Fc1cc(F)cc(-c2ccc(C(c3ccc(-c4cc(F)cc(F)c4)cc3)(c3ccc(-c4ccccc4F)cc3)c3ccc(-c4ccccc4F)cc3)cc2)c1 ; FC(F)(F)c1ccccc1-c1ccc(C(c2ccccc2)(c2ccccc2)c2ccc(-c3ccccc3C(F)(F)F)cc2)cc1]

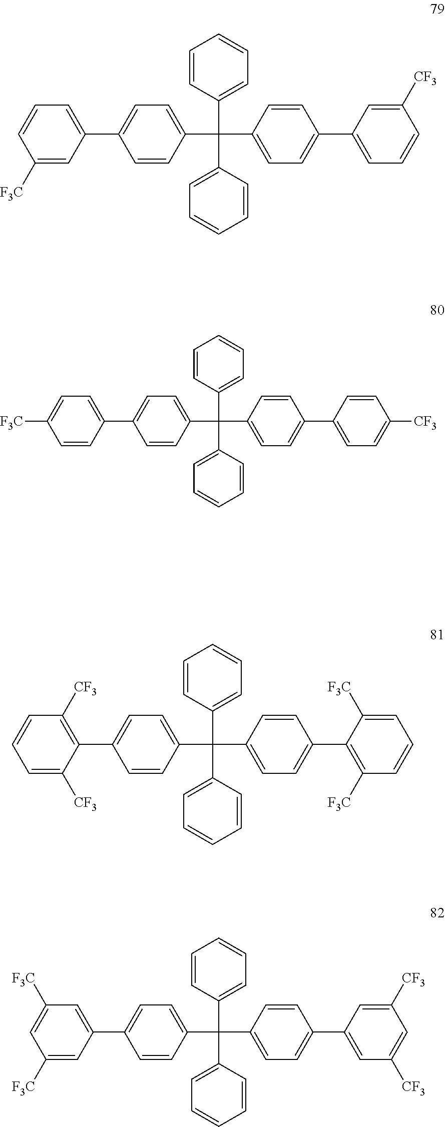 [SMILES: FC(F)(F)c1cccc(-c2ccc(C(c3ccccc3)(c3ccccc3)c3ccc(-c4cccc(C(F)(F)F)c4)cc3)cc2)c1 ; FC(F)(F)c1ccc(-c2ccc(C(c3ccccc3)(c3ccccc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccccc2)c2ccc(-c3c(C(F)(F)F)cccc3C(F)(F)F)cc2)cc1 ; FC(F)(F)c1cc(-c2ccc(C(c3ccccc3)(c3ccccc3)c3ccc(-c4cc(C(F)(F)F)cc(C(F)(F)F)c4)cc3)cc2)cc(C(F)(F)F)c1]

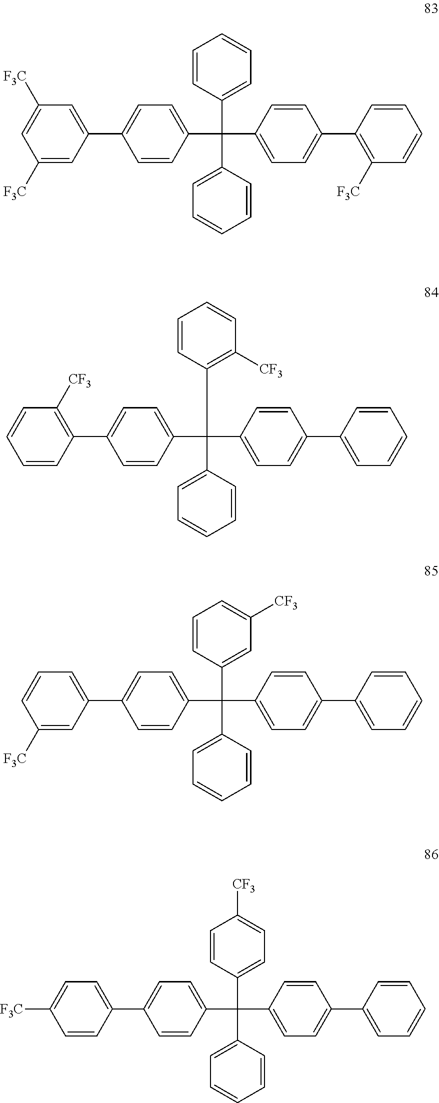 [SMILES: FC(F)(F)c1cc(-c2ccc(C(c3ccccc3)(c3ccccc3)c3ccc(-c4ccccc4C(F)(F)F)cc3)cc2)cc(C(F)(F)F)c1 ; FC(F)(F)c1ccccc1-c1ccc(C(c2ccccc2)(c2ccc(-c3ccccc3)cc2)c2ccccc2C(F)(F)F)cc1 ; FC(F)(F)c1cccc(-c2ccc(C(c3ccccc3)(c3ccc(-c4ccccc4)cc3)c3cccc(C(F)(F)F)c3)cc2)c1 ; FC(F)(F)c1ccc(-c2ccc(C(c3ccccc3)(c3ccc(-c4ccccc4)cc3)c3ccc(C(F)(F)F)cc3)cc2)cc1]

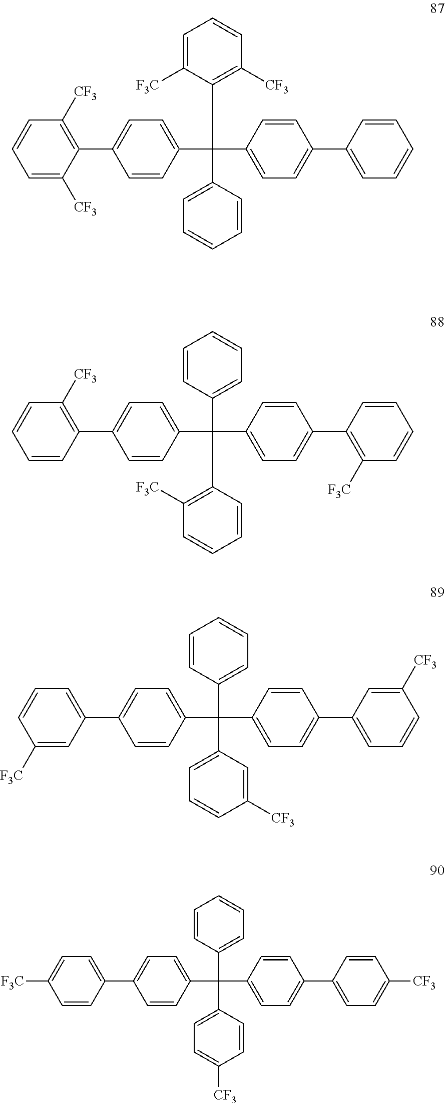 [SMILES: FC(F)(F)c1cccc(C(F)(F)F)c1-c1ccc(C(c2ccccc2)(c2ccc(-c3ccccc3)cc2)c2c(C(F)(F)F)cccc2C(F)(F)F)cc1 ; FC(F)(F)c1ccccc1-c1ccc(C(c2ccccc2)(c2ccc(-c3ccccc3C(F)(F)F)cc2)c2ccccc2C(F)(F)F)cc1 ; FC(F)(F)c1cccc(-c2ccc(C(c3ccccc3)(c3ccc(-c4cccc(C(F)(F)F)c4)cc3)c3cccc(C(F)(F)F)c3)cc2)c1 ; FC(F)(F)c1ccc(-c2ccc(C(c3ccccc3)(c3ccc(-c4ccc(C(F)(F)F)cc4)cc3)c3ccc(C(F)(F)F)cc3)cc2)cc1]

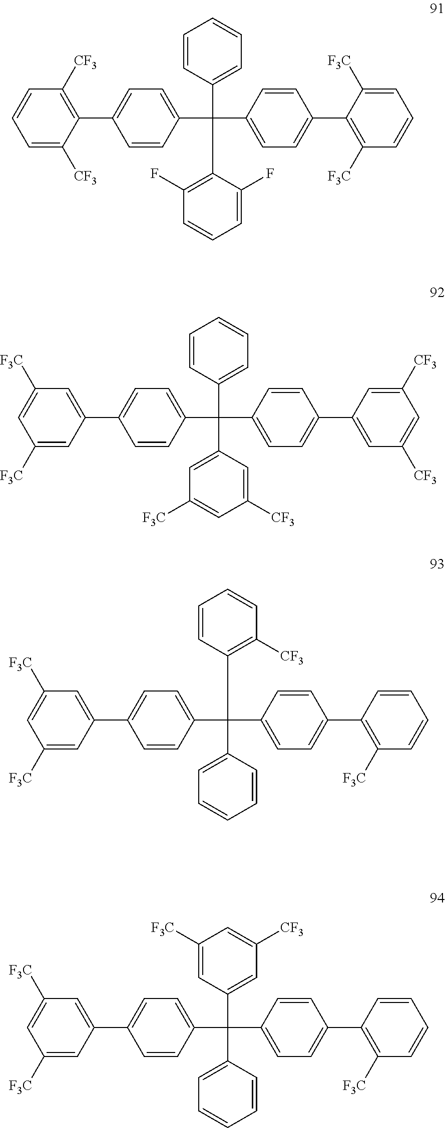 [SMILES: Fc1cccc(F)c1C(c1ccccc1)(c1ccc(-c2c(C(F)(F)F)cccc2C(F)(F)F)cc1)c1ccc(-c2c(C(F)(F)F)cccc2C(F)(F)F)cc1 ; FC(F)(F)c1cc(-c2ccc(C(c3ccccc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc(C(c3ccccc3)(c3ccc(-c4ccccc4C(F)(F)F)cc3)c3ccccc3C(F)(F)F)cc2)cc(C(F)(F)F)c1 ; FC(F)(F)c1cc(-c2ccc(C(c3ccccc3)(c3ccc(-c4ccccc4C(F)(F)F)cc3)c3cc(C(F)(F)F)cc(C(F)(F)F)c3)cc2)cc(C(F)(F)F)c1]

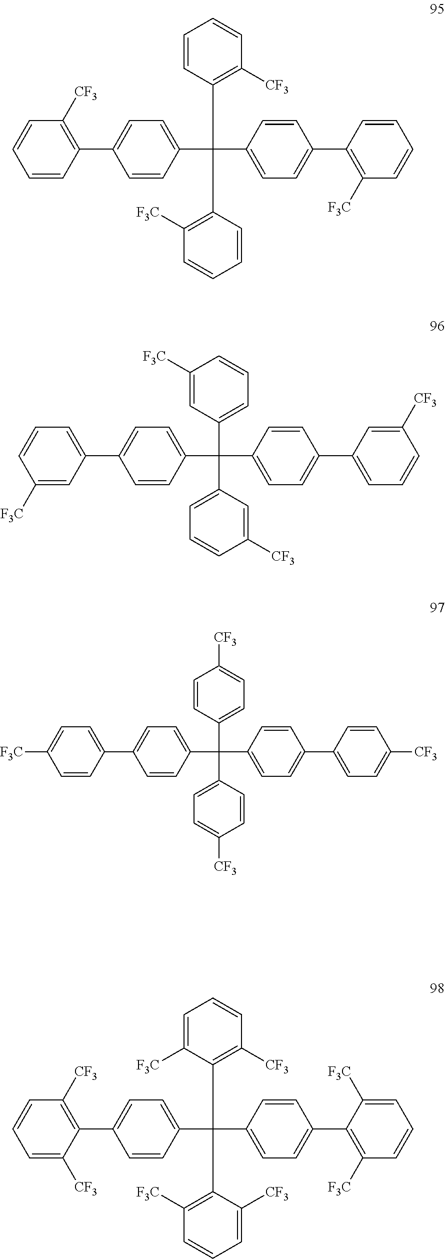 [SMILES: FC(F)(F)c1ccccc1-c1ccc(C(c2ccc(-c3ccccc3C(F)(F)F)cc2)(c2ccccc2C(F)(F)F)c2ccccc2C(F)(F)F)cc1 ; FC(F)(F)c1cccc(-c2ccc(C(c3ccc(-c4cccc(C(F)(F)F)c4)cc3)(c3cccc(C(F)(F)F)c3)c3cccc(C(F)(F)F)c3)cc2)c1 ; FC(F)(F)c1ccc(-c2ccc(C(c3ccc(-c4ccc(C(F)(F)F)cc4)cc3)(c3ccc(C(F)(F)F)cc3)c3ccc(C(F)(F)F)cc3)cc2)cc1 ; FC(F)(F)c1cccc(C(F)(F)F)c1-c1ccc(C(c2ccc(-c3c(C(F)(F)F)cccc3C(F)(F)F)cc2)(c2c(C(F)(F)F)cccc2C(F)(F)F)c2c(C(F)(F)F)cccc2C(F)(F)F)cc1]

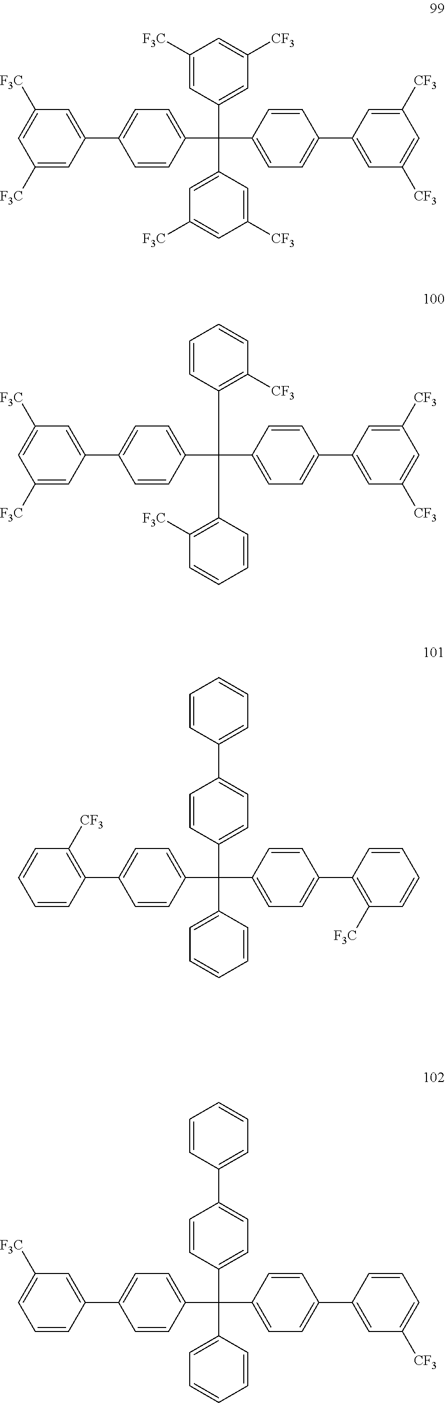 [SMILES: FC(F)(F)c1cc(-c2ccc(C(c3ccc(-c4cc(C(F)(F)F)cc(C(F)(F)F)c4)cc3)(c3cc(C(F)(F)F)cc(C(F)(F)F)c3)c3cc(C(F)(F)F)cc(C(F)(F)F)c3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccccc3C(F)(F)F)c3ccccc3C(F)(F)F)cc2)cc(C(F)(F)F)c1 ; FC(F)(F)c1ccccc1-c1ccc(C(c2ccccc2)(c2ccc(-c3ccccc3)cc2)c2ccc(-c3ccccc3C(F)(F)F)cc2)cc1 ; FC(F)(F)c1cccc(-c2ccc(C(c3ccccc3)(c3ccc(-c4ccccc4)cc3)c3ccc(-c4cccc(C(F)(F)F)c4)cc3)cc2)c1]

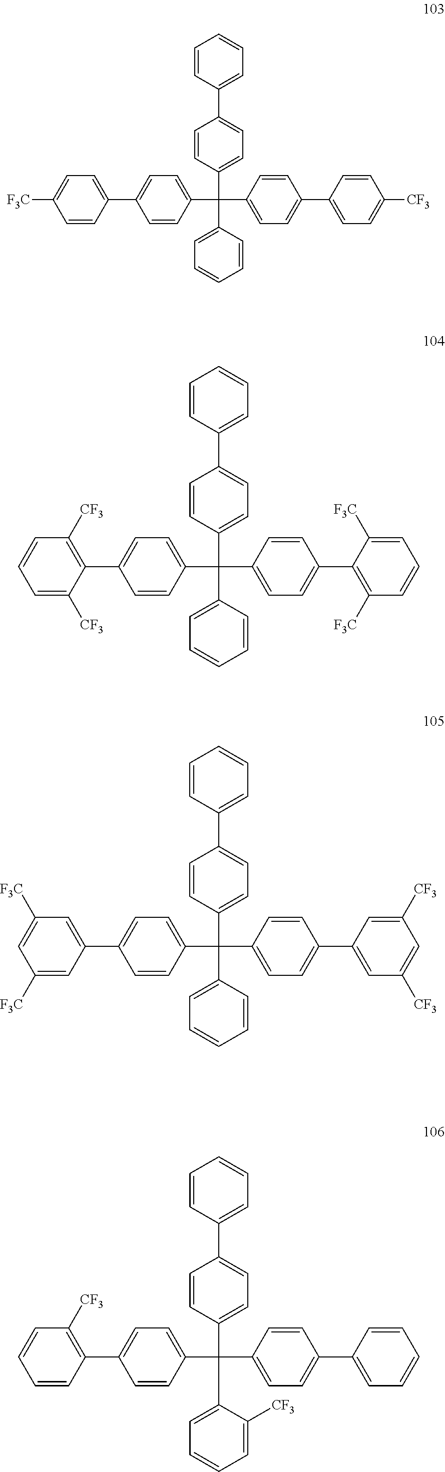 [SMILES: FC(F)(F)c1ccc(-c2ccc(C(c3ccccc3)(c3ccc(-c4ccccc4)cc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccccc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1ccccc1-c1ccc(C(c2ccc(-c3ccccc3)cc2)(c2ccc(-c3ccccc3)cc2)c2ccccc2C(F)(F)F)cc1]

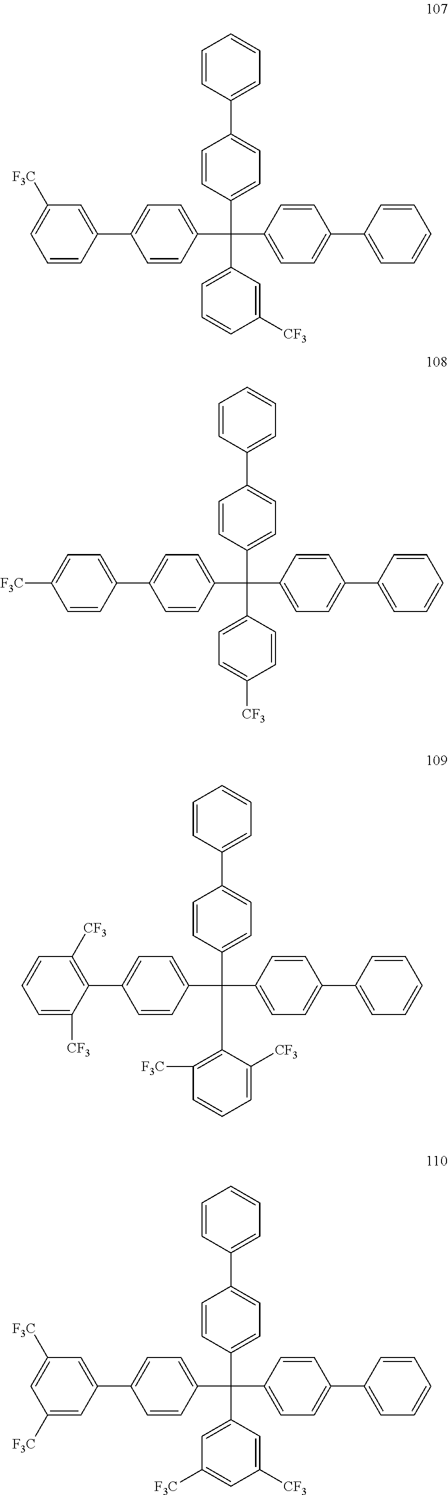 [SMILES: FC(F)(F)c1cccc(-c2ccc(C(c3ccc(-c4ccccc4)cc3)(c3ccc(-c4ccccc4)cc3)c3cccc(C(F)(F)F)c3)cc2)c1 ; FC(F)(F)c1ccc(-c2ccc(C(c3ccc(-c4ccccc4)cc3)(c3ccc(-c4ccccc4)cc3)c3ccc(C(F)(F)F)cc3)cc2)cc1 ; FC(F)(F)c1cccc(C(F)(F)F)c1-c1ccc(C(c2ccc(-c3ccccc3)cc2)(c2ccc(-c3ccccc3)cc2)c2c(C(F)(F)F)cccc2C(F)(F)F)cc1 ; FC(F)(F)c1cc(-c2ccc(C(c3ccc(-c4ccccc4)cc3)(c3ccc(-c4ccccc4)cc3)c3cc(C(F)(F)F)cc(C(F)(F)F)c3)cc2)cc(C(F)(F)F)c1]

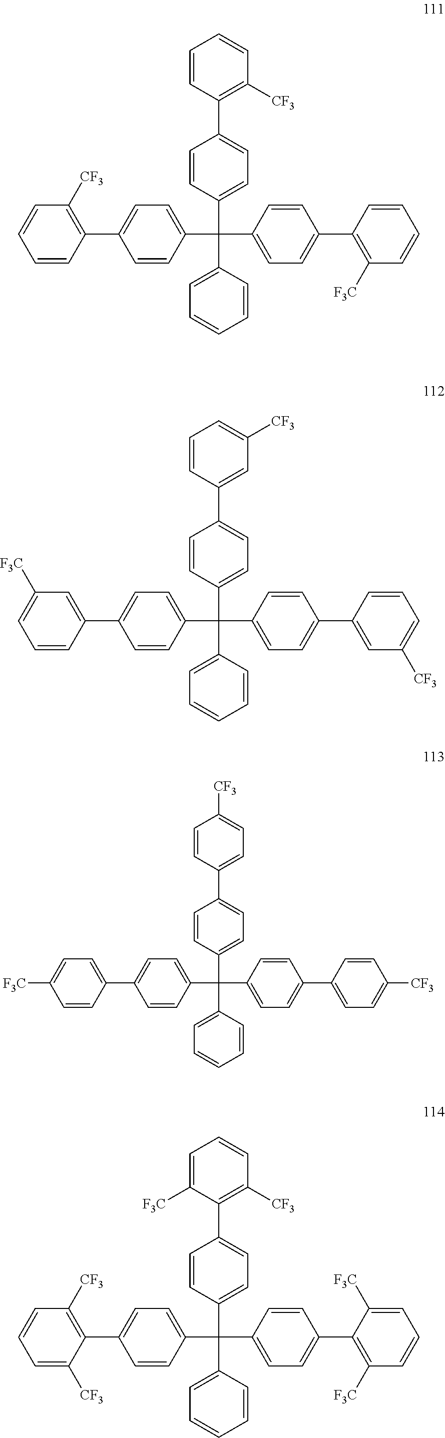 [SMILES: FC(F)(F)c1ccccc1-c1ccc(C(c2ccccc2)(c2ccc(-c3ccccc3C(F)(F)F)cc2)c2ccc(-c3ccccc3C(F)(F)F)cc2)cc1 ; FC(F)(F)c1cccc(-c2ccc(C(c3ccccc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccccc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccccc2)(c2ccc(-c3c(C(F)(F)F)cccc3C(F)(F)F)cc2)c2ccc(-c3c(C(F)(F)F)cccc3C(F)(F)F)cc2)cc1]

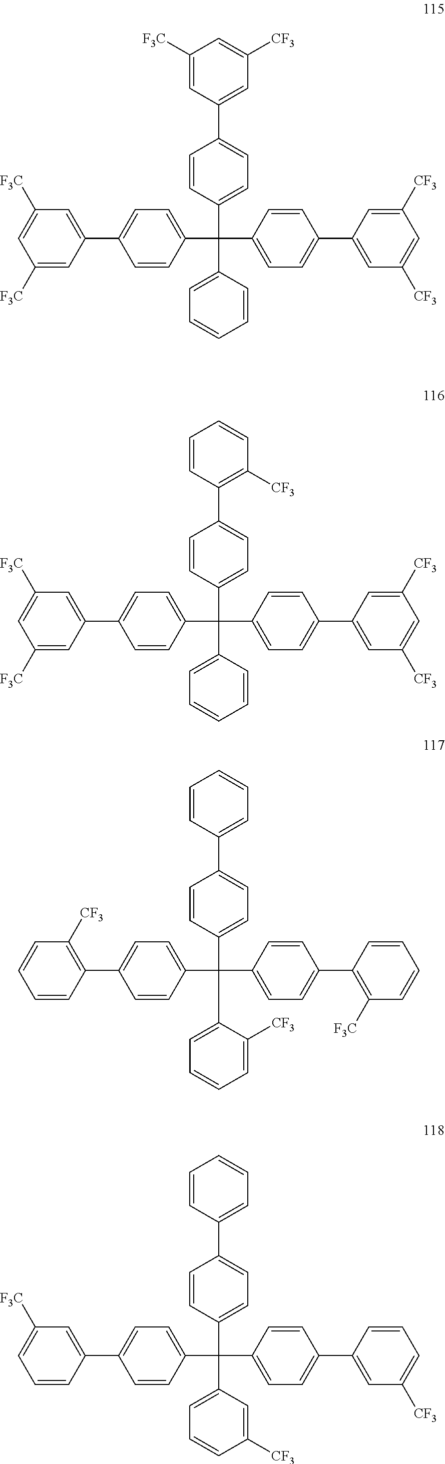 [SMILES: FC(F)(F)c1cc(-c2ccc(C(c3ccccc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccccc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc(C(c2ccc(-c3ccccc3)cc2)(c2ccc(-c3ccccc3C(F)(F)F)cc2)c2ccccc2C(F)(F)F)cc1 ; FC(F)(F)c1cccc(-c2ccc(C(c3ccc(-c4ccccc4)cc3)(c3ccc(-c4cccc(C(F)(F)F)c4)cc3)c3cccc(C(F)(F)F)c3)cc2)c1]

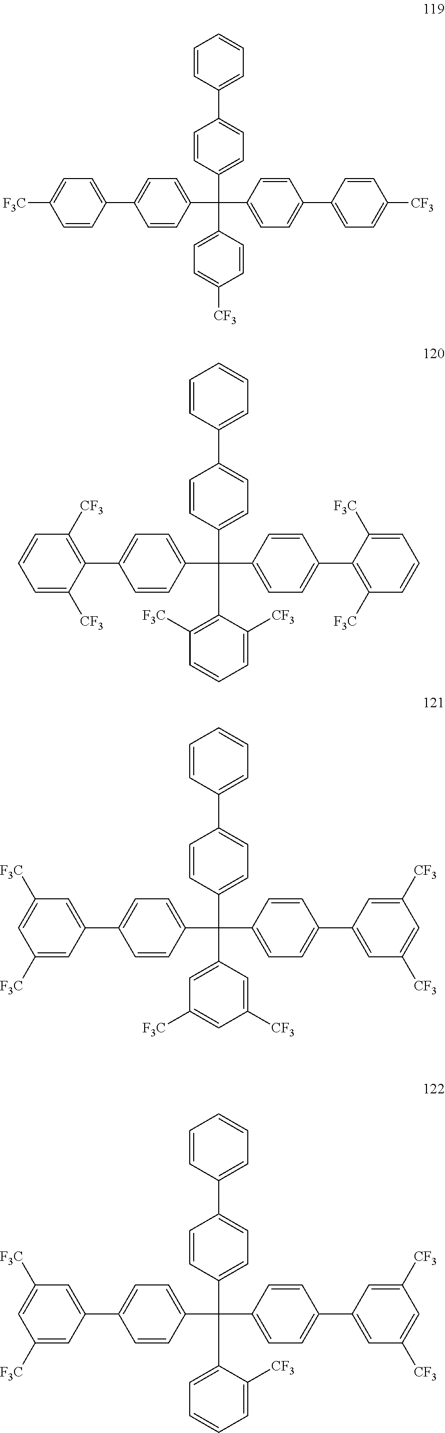

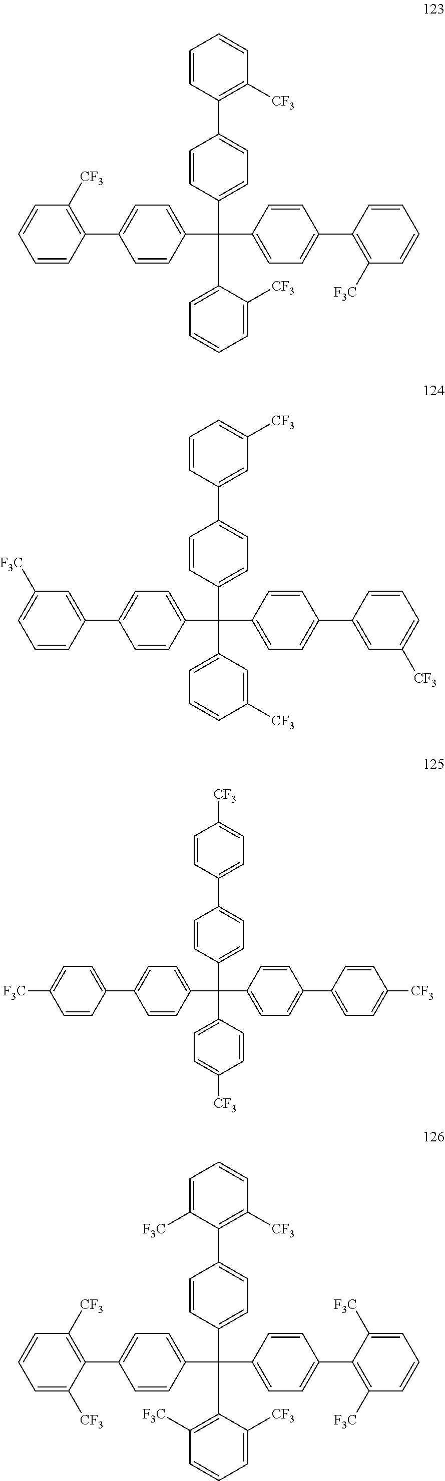 [SMILES: FC(F)(F)c1ccccc1-c1ccc(C(c2ccc(-c3ccccc3C(F)(F)F)cc2)(c2ccc(-c3ccccc3C(F)(F)F)cc2)c2ccccc2C(F)(F)F)cc1 ; FC(F)(F)c1cccc(-c2ccc(C(c3ccc(-c4cccc(C(F)(F)F)c4)cc3)(c3ccc(-c4cccc(C(F)(F)F)c4)cc3)c3cccc(C(F)(F)F)c3)cc2)c1 ; FC(F)(F)c1ccc(-c2ccc(C(c3ccc(-c4ccc(C(F)(F)F)cc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc(C(c2ccc(-c3c(C(F)(F)F)cccc3C(F)(F)F)cc2)(c2ccc(-c3c(C(F)(F)F)cccc3C(F)(F)F)cc2)c2c(C(F)(F)F)cccc2C(F)(F)F)cc1]

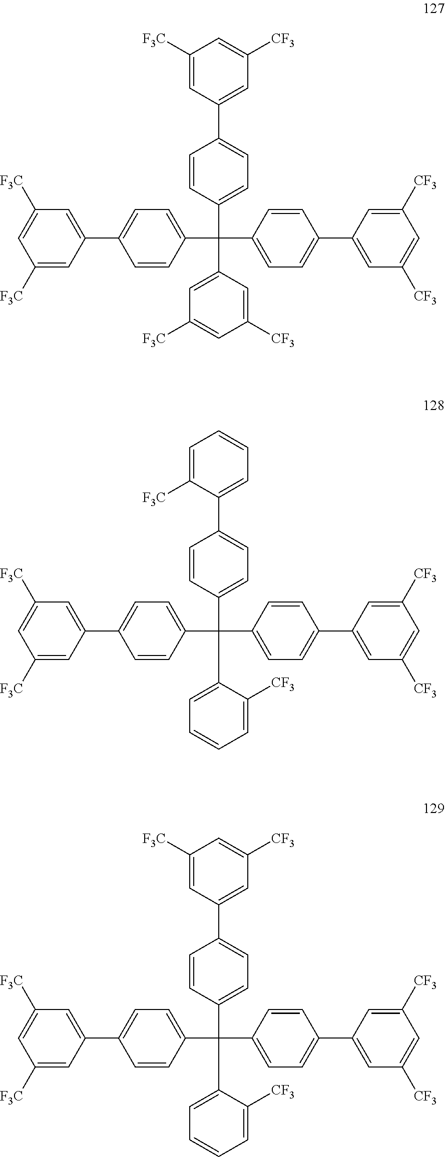 [SMILES: FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccccc3C(F)(F)F)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3ccccc3C(F)(F)F)cc2)cc(C(F)(F)F)c1]

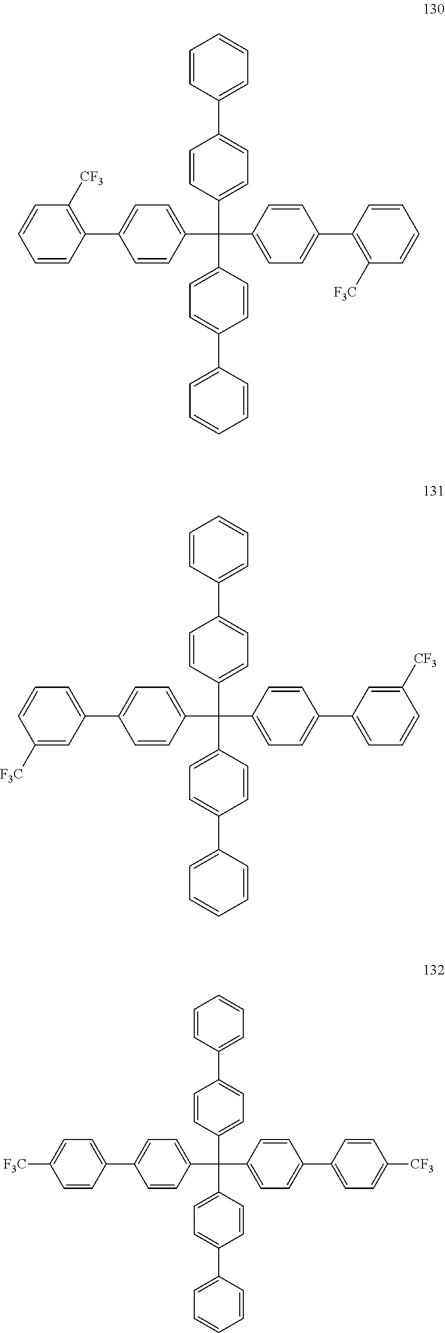 [SMILES: FC(F)(F)c1ccccc1-c1ccc(C(c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc(C(c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccccc4)cc3)c3ccc(-c4ccc(C(F)(F)F)cc4)cc3)cc2)cc1]

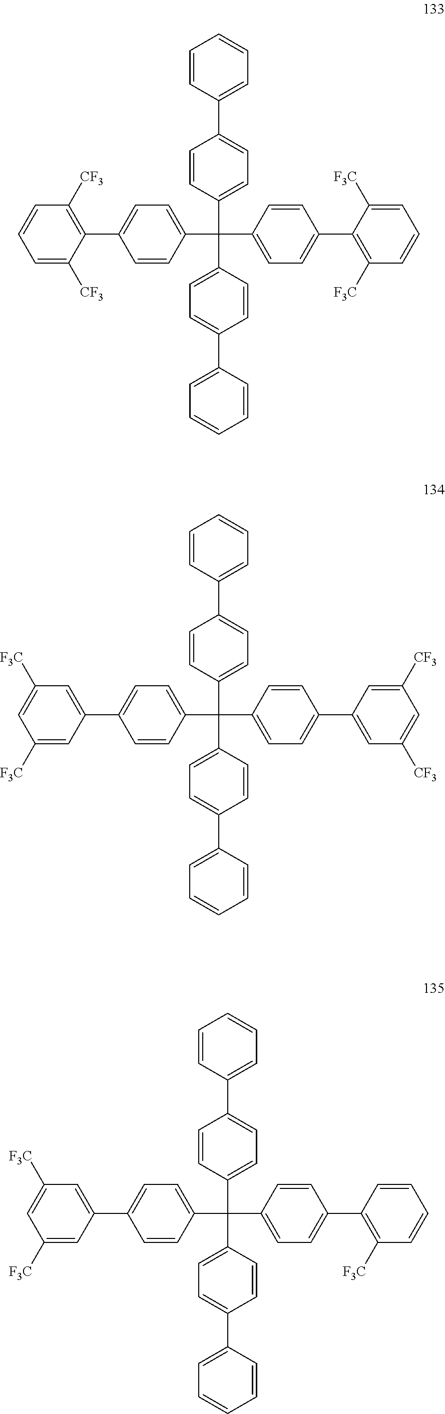 [SMILES: FC(F)(F)c1cccc(C(F)(F)F)c1-c1ccc(C(c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4ccccc4)cc3)c3ccc(-c4ccccc4C(F)(F)F)cc3)cc2)cc(C(F)(F)F)c1]

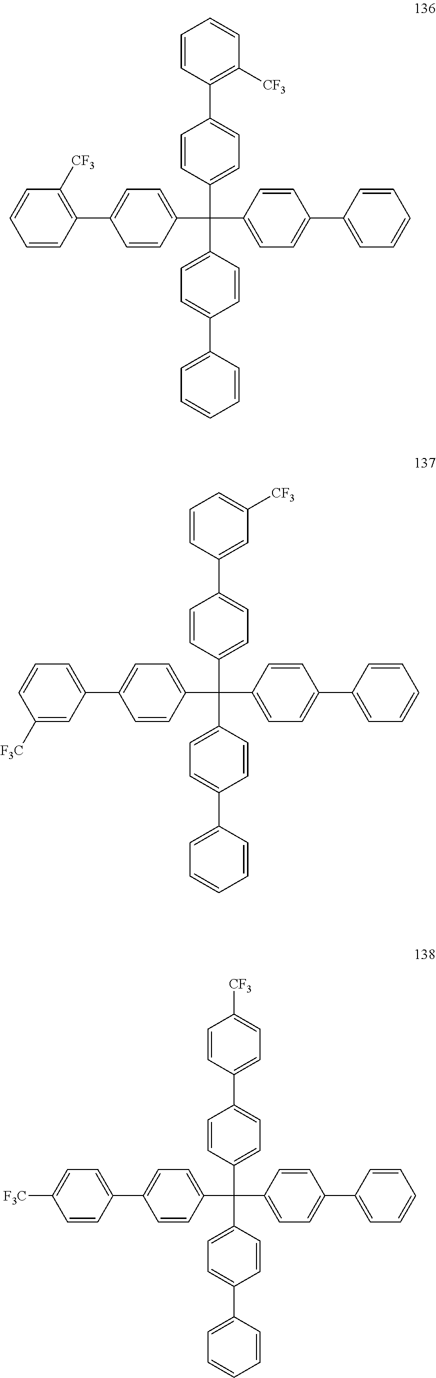 [SMILES: FC(F)(F)c1ccccc1-c1ccc(C(c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc(C(c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccccc4)cc3)c3ccc(-c4ccc(C(F)(F)F)cc4)cc3)cc2)cc1]

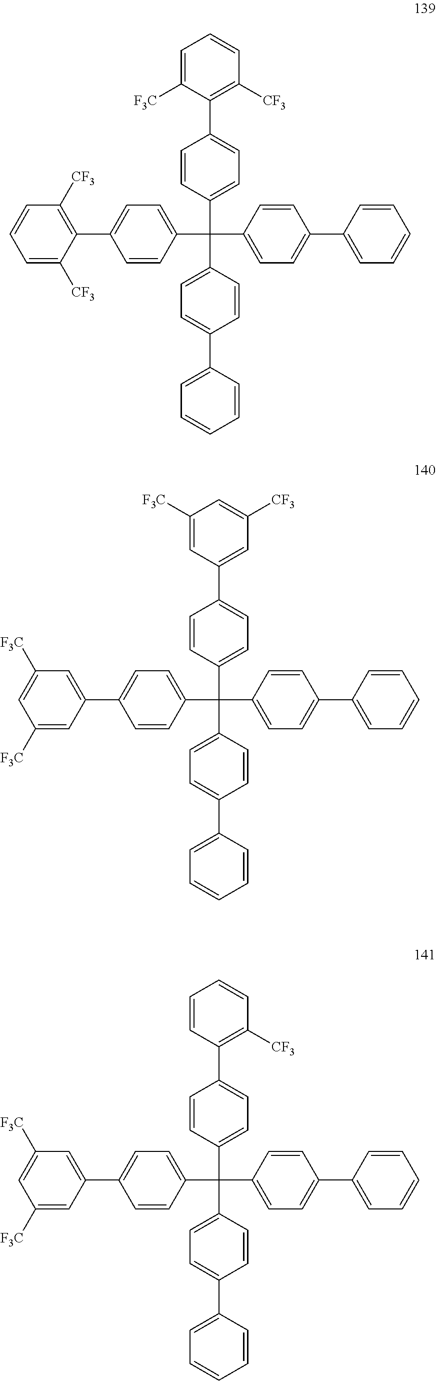 [SMILES: FC(F)(F)c1cccc(C(F)(F)F)c1-c1ccc(C(c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc(C(c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4ccccc4)cc3)c3ccc(-c4ccccc4C(F)(F)F)cc3)cc2)cc(C(F)(F)F)c1]

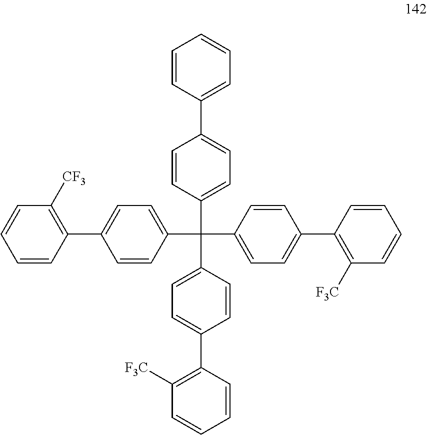 [SMILES: FC(F)(F)c1ccccc1-c1ccc(C(c2ccc(-c3ccccc3)cc2)(c2ccc(-c3ccccc3C(F)(F)F)cc2)c2ccc(-c3ccccc3C(F)(F)F)cc2)cc1]

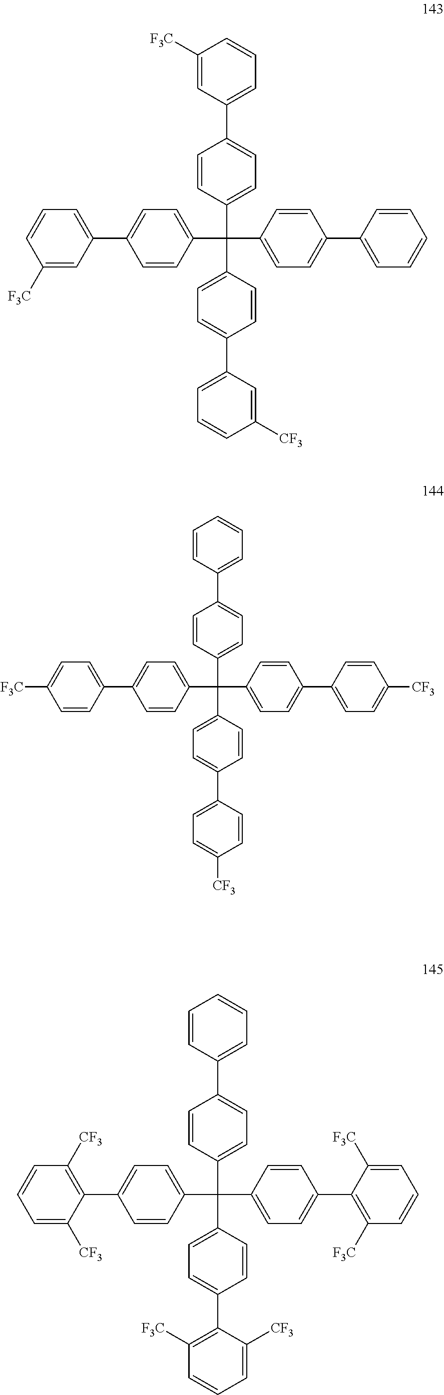 [SMILES: FC(F)(F)c1cccc(-c2ccc(C(c3ccc(-c4ccccc4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccc(-c4ccccc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccc(-c3ccccc3)cc2)(c2ccc(-c3c(C(F)(F)F)cccc3C(F)(F)F)cc2)c2ccc(-c3c(C(F)(F)F)cccc3C(F)(F)F)cc2)cc1]

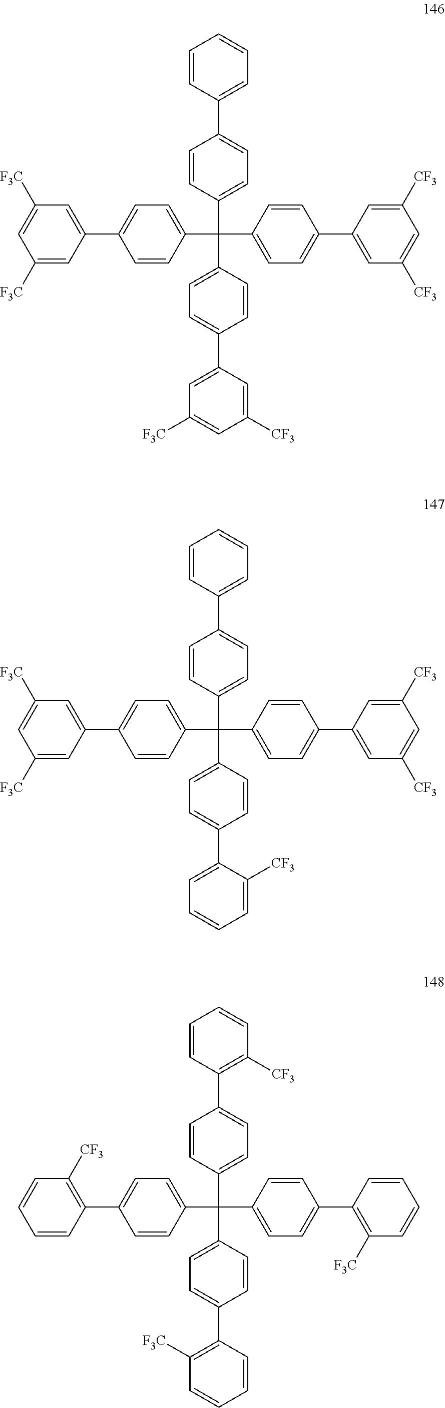 [SMILES: FC(F)(F)c1cc(-c2ccc(C(c3ccc(-c4ccccc4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4ccccc4)cc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc(C(c2ccc(-c3ccccc3C(F)(F)F)cc2)(c2ccc(-c3ccccc3C(F)(F)F)cc2)c2ccc(-c3ccccc3C(F)(F)F)cc2)cc1]

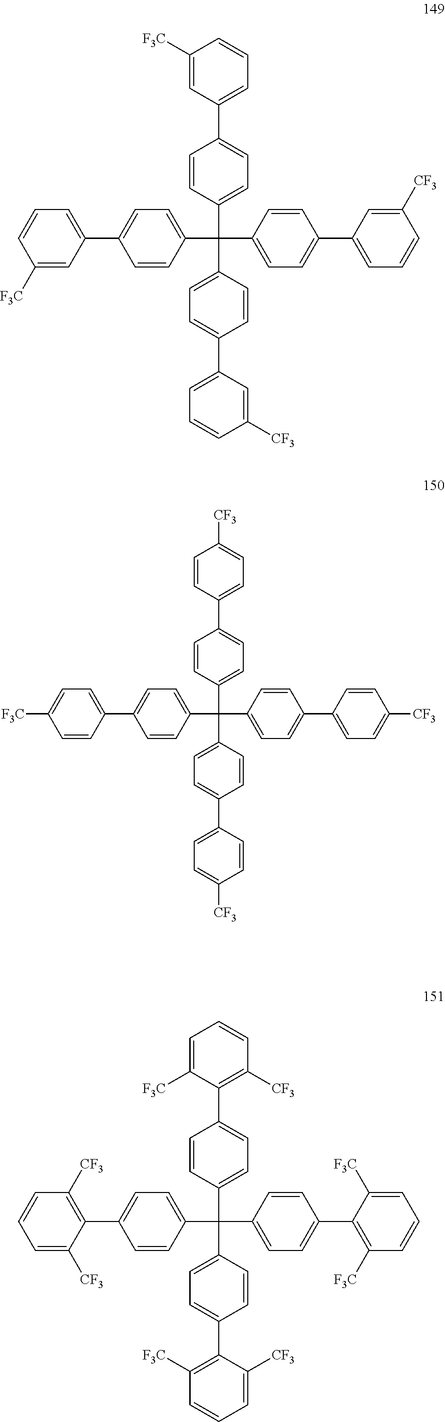 [SMILES: FC(F)(F)c1cccc(-c2ccc(C(c3ccc(-c4cccc(C(F)(F)F)c4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc(C(c3ccc(-c4ccc(C(F)(F)F)cc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc(C(c2ccc(-c3c(C(F)(F)F)cccc3C(F)(F)F)cc2)(c2ccc(-c3c(C(F)(F)F)cccc3C(F)(F)F)cc2)c2ccc(-c3c(C(F)(F)F)cccc3C(F)(F)F)cc2)cc1]

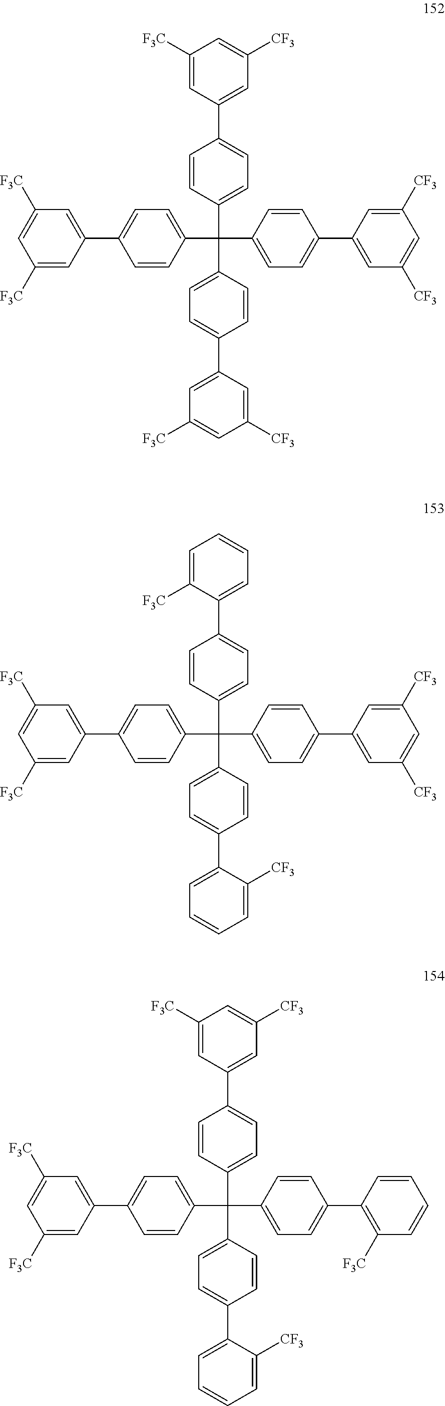 [SMILES: FC(F)(F)c1cc(-c2ccc(C(c3ccc(-c4cc(C(F)(F)F)cc(C(F)(F)F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1 ; FC(F)(F)c1cc(-c2ccc(C(c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1]

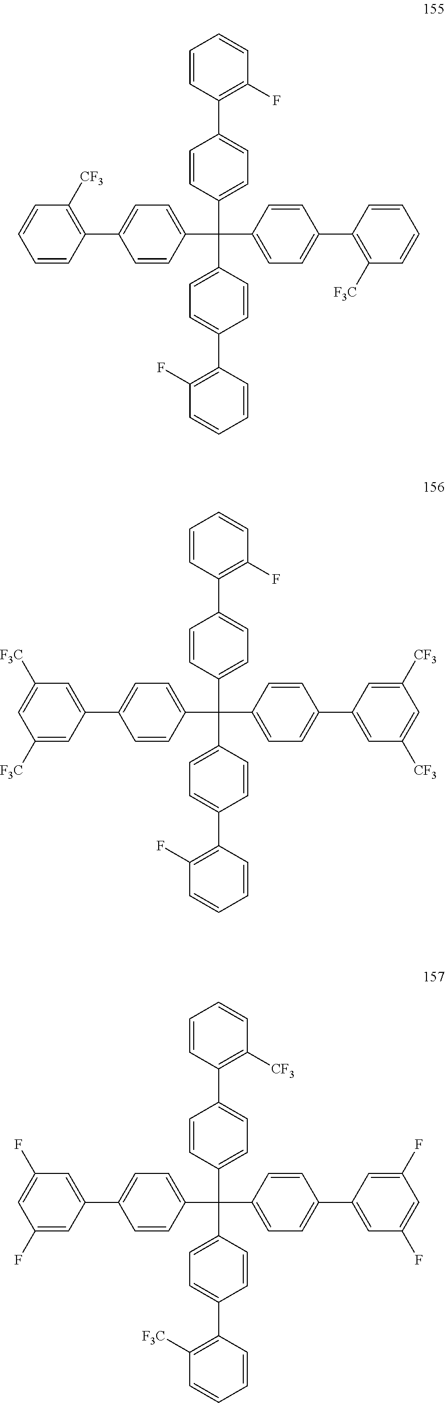 [SMILES: Fc1ccccc1-c1ccc(C(c2ccc(-c3ccccc3F)cc2)(c2ccc(-c3ccccc3C(F)(F)F)cc2)c2ccc(-c3ccccc3C(F)(F)F)cc2)cc1 ; Fc1ccccc1-c1ccc(C(c2ccc(-c3cc(C(F)(F)F)cc(C(F)(F)F)c3)cc2)(c2ccc(-c3cc(C(F)(F)F)cc(C(F)(F)F)c3)cc2)c2ccc(-c3ccccc3F)cc2)cc1 ; Fc1cc(F)cc(-c2ccc(C(c3ccc(-c4cc(F)cc(F)c4)cc3)(c3ccc(-c4ccccc4C(F)(F)F)cc3)c3ccc(-c4ccccc4C(F)(F)F)cc3)cc2)c1]

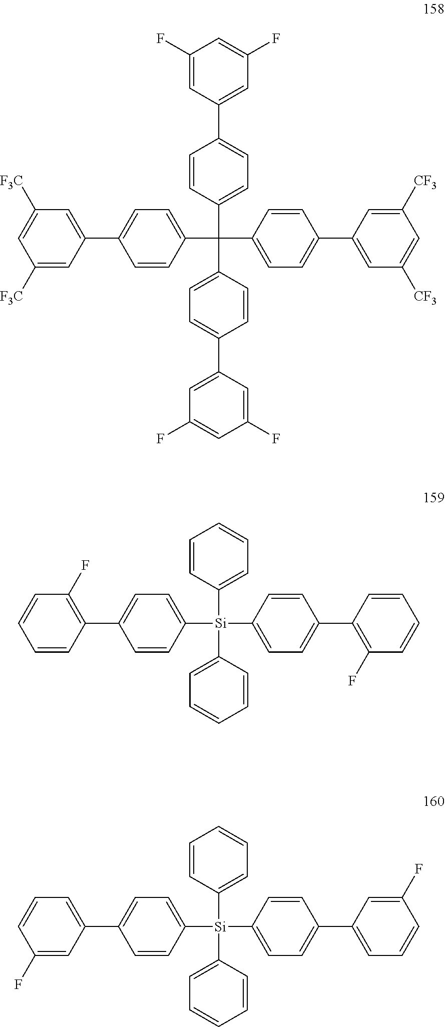 [SMILES: Fc1cc(F)cc(-c2ccc(C(c3ccc(-c4cc(F)cc(F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)c1 ; Fc1ccccc1-c1ccc([Si](c2ccccc2)(c2ccccc2)c2ccc(-c3ccccc3F)cc2)cc1 ; Fc1cccc(-c2ccc([Si](c3ccccc3)(c3ccccc3)c3ccc(-c4cccc(F)c4)cc3)cc2)c1]

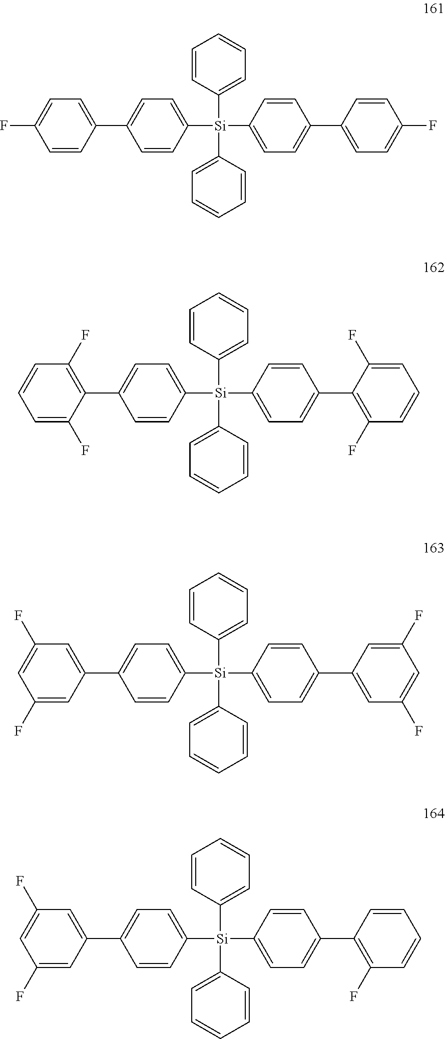 [SMILES: Fc1ccc(-c2ccc([Si](c3ccccc3)(c3ccccc3)c3ccc(-c4ccc(F)cc4)cc3)cc2)cc1 ; Fc1cccc(F)c1-c1ccc([Si](c2ccccc2)(c2ccccc2)c2ccc(-c3c(F)cccc3F)cc2)cc1 ; Fc1cc(F)cc(-c2ccc([Si](c3ccccc3)(c3ccccc3)c3ccc(-c4cc(F)cc(F)c4)cc3)cc2)c1 ; Fc1cc(F)cc(-c2ccc([Si](c3ccccc3)(c3ccccc3)c3ccc(-c4ccccc4F)cc3)cc2)c1]

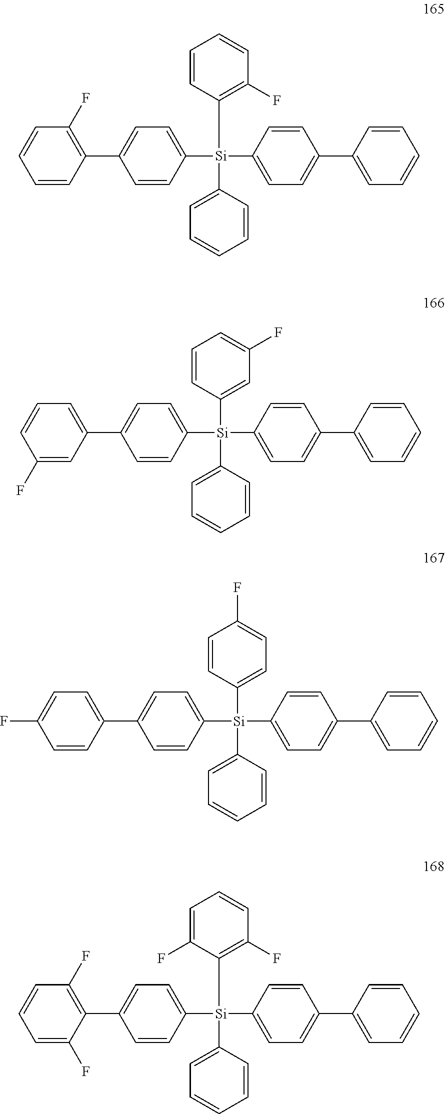 [SMILES: Fc1ccccc1-c1ccc([Si](c2ccccc2)(c2ccc(-c3ccccc3)cc2)c2ccccc2F)cc1 ; Fc1cccc(-c2ccc([Si](c3ccccc3)(c3ccc(-c4ccccc4)cc3)c3cccc(F)c3)cc2)c1 ; Fc1ccc(-c2ccc([Si](c3ccccc3)(c3ccc(F)cc3)c3ccc(-c4ccccc4)cc3)cc2)cc1 ; Fc1cccc(F)c1-c1ccc([Si](c2ccccc2)(c2ccc(-c3ccccc3)cc2)c2c(F)cccc2F)cc1]

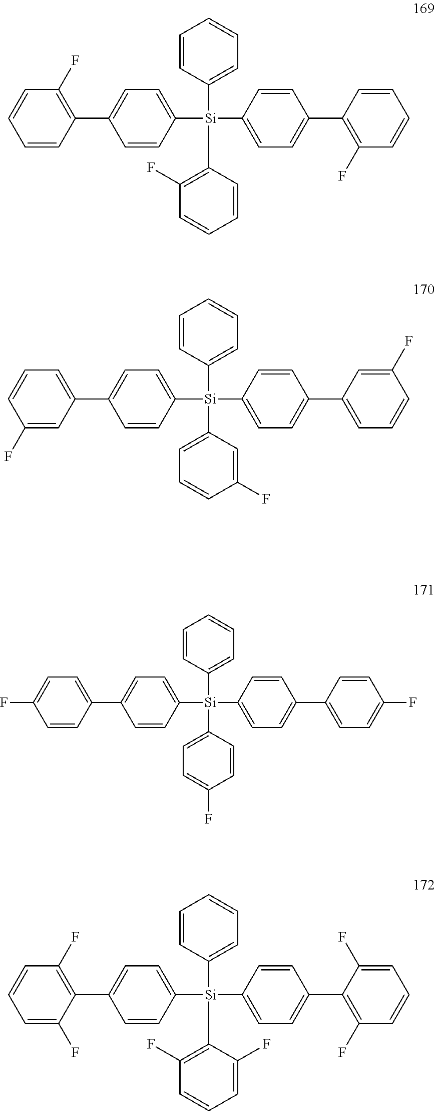 [SMILES: Fc1ccccc1-c1ccc([Si](c2ccccc2)(c2ccc(-c3ccccc3F)cc2)c2ccccc2F)cc1 ; Fc1cccc(-c2ccc([Si](c3ccccc3)(c3ccc(-c4cccc(F)c4)cc3)c3cccc(F)c3)cc2)c1 ; Fc1ccc(-c2ccc([Si](c3ccccc3)(c3ccc(F)cc3)c3ccc(-c4ccc(F)cc4)cc3)cc2)cc1 ; Fc1cccc(F)c1-c1ccc([Si](c2ccccc2)(c2ccc(-c3c(F)cccc3F)cc2)c2c(F)cccc2F)cc1]

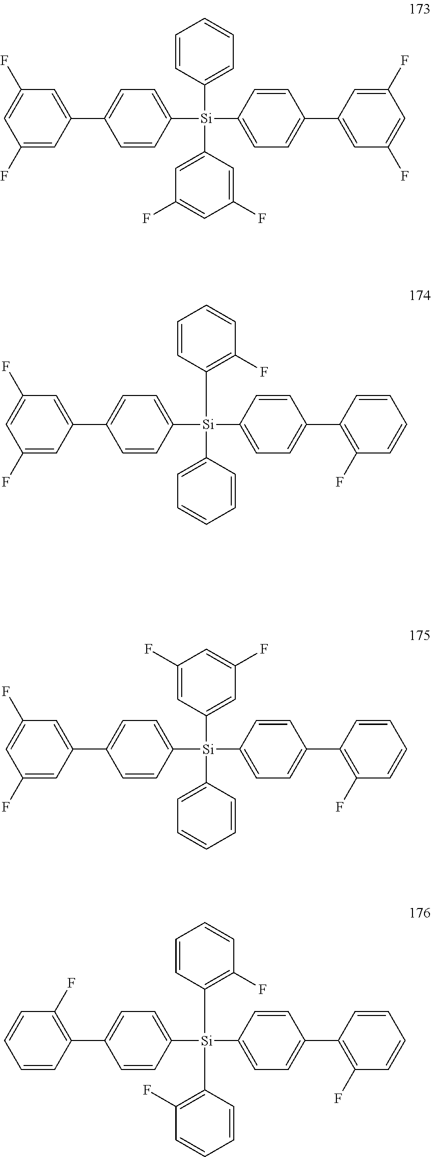 [SMILES: Fc1cc(F)cc(-c2ccc([Si](c3ccccc3)(c3ccc(-c4cc(F)cc(F)c4)cc3)c3cc(F)cc(F)c3)cc2)c1 ; Fc1cc(F)cc(-c2ccc([Si](c3ccccc3)(c3ccc(-c4ccccc4F)cc3)c3ccccc3F)cc2)c1 ; Fc1cc(F)cc(-c2ccc([Si](c3ccccc3)(c3ccc(-c4ccccc4F)cc3)c3cc(F)cc(F)c3)cc2)c1 ; Fc1ccccc1-c1ccc([Si](c2ccc(-c3ccccc3F)cc2)(c2ccccc2F)c2ccccc2F)cc1]

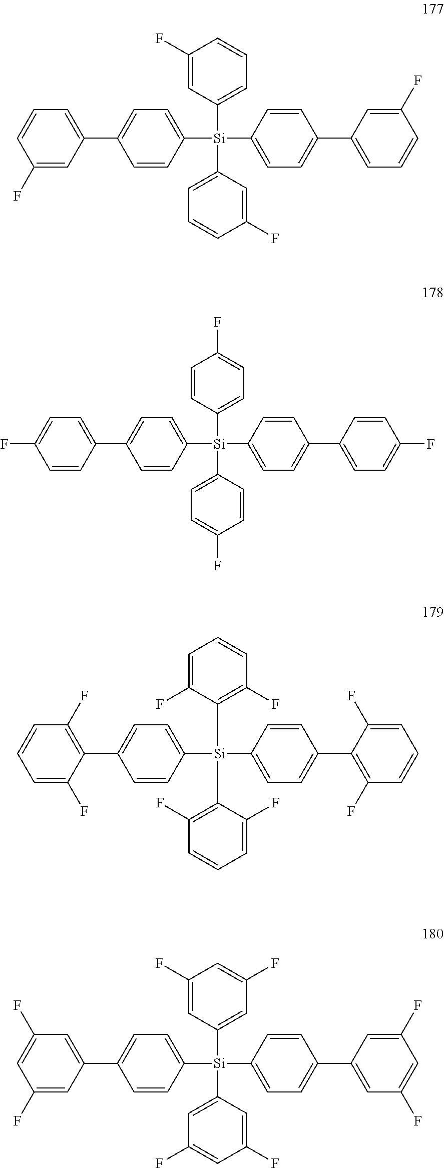 [SMILES: Fc1cccc(-c2ccc([Si](c3ccc(-c4cccc(F)c4)cc3)(c3cccc(F)c3)c3cccc(F)c3)cc2)c1 ; Fc1ccc(-c2ccc([Si](c3ccc(F)cc3)(c3ccc(F)cc3)c3ccc(-c4ccc(F)cc4)cc3)cc2)cc1 ; Fc1cccc(F)c1-c1ccc([Si](c2ccc(-c3c(F)cccc3F)cc2)(c2c(F)cccc2F)c2c(F)cccc2F)cc1 ; Fc1cc(F)cc(-c2ccc([Si](c3ccc(-c4cc(F)cc(F)c4)cc3)(c3cc(F)cc(F)c3)c3cc(F)cc(F)c3)cc2)c1]

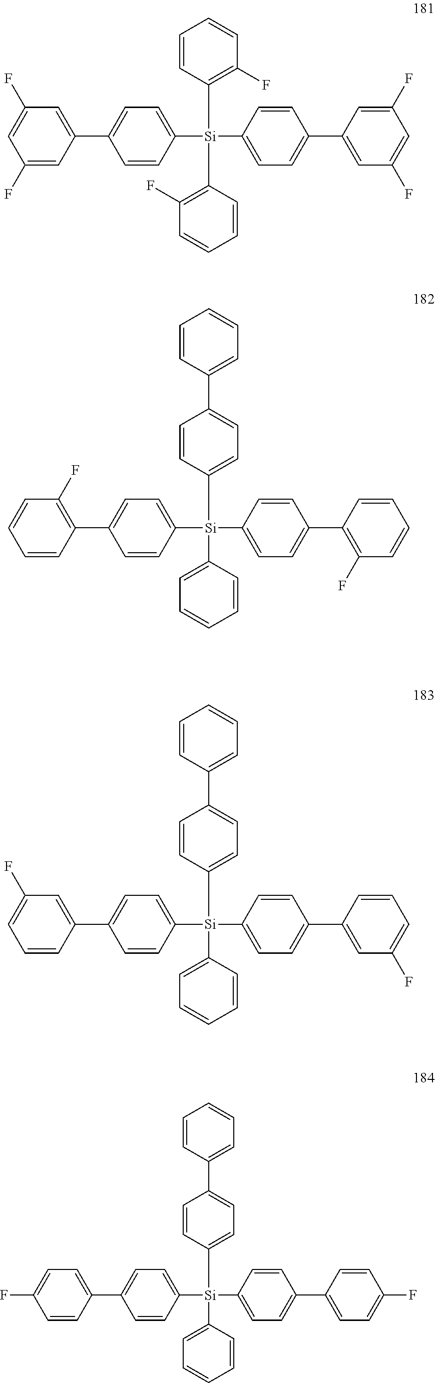 [SMILES: Fc1cc(F)cc(-c2ccc([Si](c3ccc(-c4cc(F)cc(F)c4)cc3)(c3ccccc3F)c3ccccc3F)cc2)c1 ; Fc1ccccc1-c1ccc([Si](c2ccccc2)(c2ccc(-c3ccccc3)cc2)c2ccc(-c3ccccc3F)cc2)cc1 ; Fc1cccc(-c2ccc([Si](c3ccccc3)(c3ccc(-c4ccccc4)cc3)c3ccc(-c4cccc(F)c4)cc3)cc2)c1 ; Fc1ccc(-c2ccc([Si](c3ccccc3)(c3ccc(-c4ccccc4)cc3)c3ccc(-c4ccc(F)cc4)cc3)cc2)cc1]

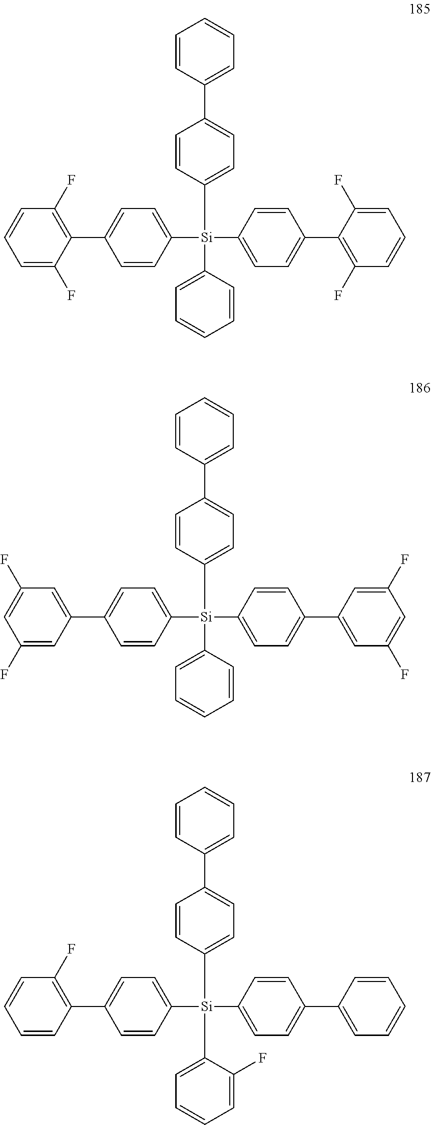 [SMILES: Fc1cccc(F)c1-c1ccc([Si](c2ccccc2)(c2ccc(-c3ccccc3)cc2)c2ccc(-c3c(F)cccc3F)cc2)cc1 ; Fc1cc(F)cc(-c2ccc([Si](c3ccccc3)(c3ccc(-c4ccccc4)cc3)c3ccc(-c4cc(F)cc(F)c4)cc3)cc2)c1 ; Fc1ccccc1-c1ccc([Si](c2ccc(-c3ccccc3)cc2)(c2ccc(-c3ccccc3)cc2)c2ccccc2F)cc1]

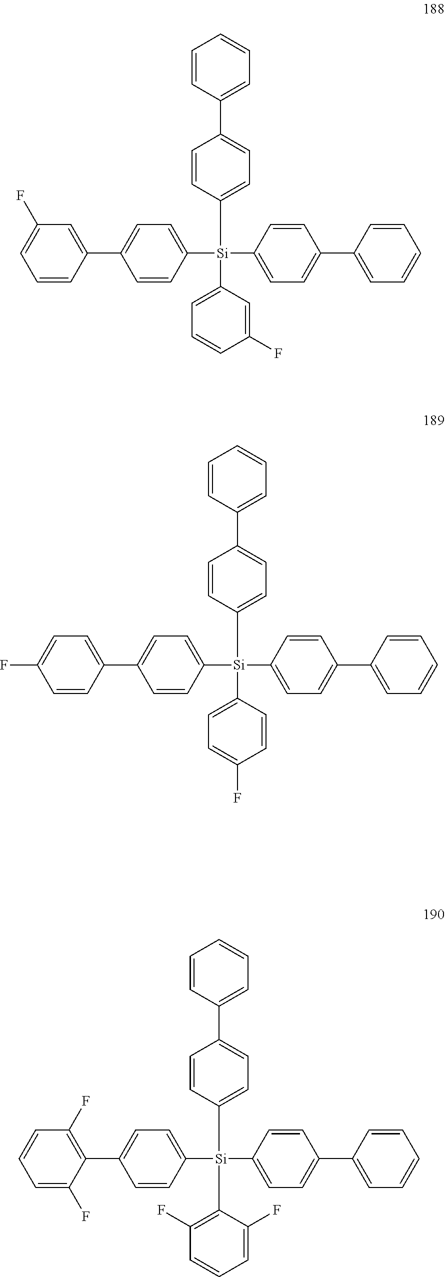 [SMILES: Fc1cccc(-c2ccc([Si](c3ccc(-c4ccccc4)cc3)(c3ccc(-c4ccccc4)cc3)c3cccc(F)c3)cc2)c1 ; Fc1ccc(-c2ccc([Si](c3ccc(F)cc3)(c3ccc(-c4ccccc4)cc3)c3ccc(-c4ccccc4)cc3)cc2)cc1 ; Fc1cccc(F)c1-c1ccc([Si](c2ccc(-c3ccccc3)cc2)(c2ccc(-c3ccccc3)cc2)c2c(F)cccc2F)cc1]

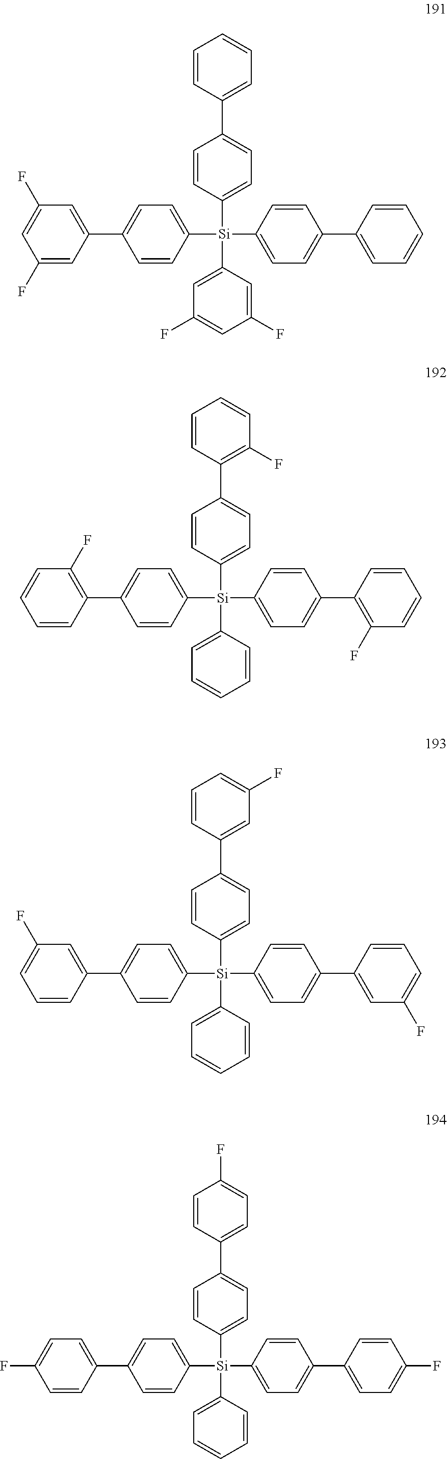 [SMILES: Fc1cc(F)cc(-c2ccc([Si](c3ccc(-c4ccccc4)cc3)(c3ccc(-c4ccccc4)cc3)c3cc(F)cc(F)c3)cc2)c1 ; Fc1ccccc1-c1ccc([Si](c2ccccc2)(c2ccc(-c3ccccc3F)cc2)c2ccc(-c3ccccc3F)cc2)cc1 ; Fc1cccc(-c2ccc([Si](c3ccccc3)(c3ccc(-c4cccc(F)c4)cc3)c3ccc(-c4cccc(F)c4)cc3)cc2)c1 ; Fc1ccc(-c2ccc([Si](c3ccccc3)(c3ccc(-c4ccc(F)cc4)cc3)c3ccc(-c4ccc(F)cc4)cc3)cc2)cc1]

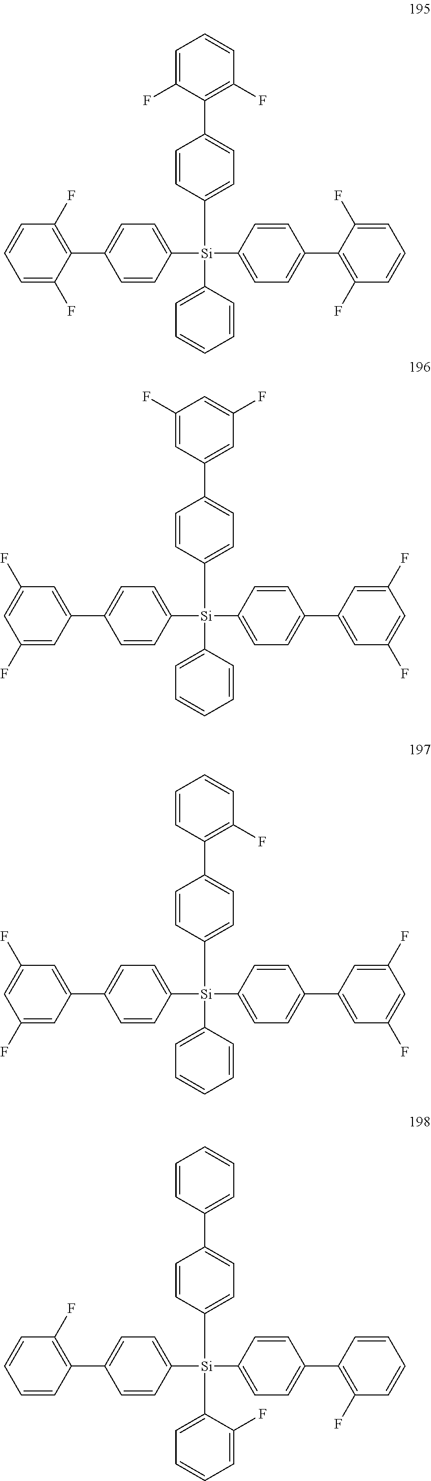

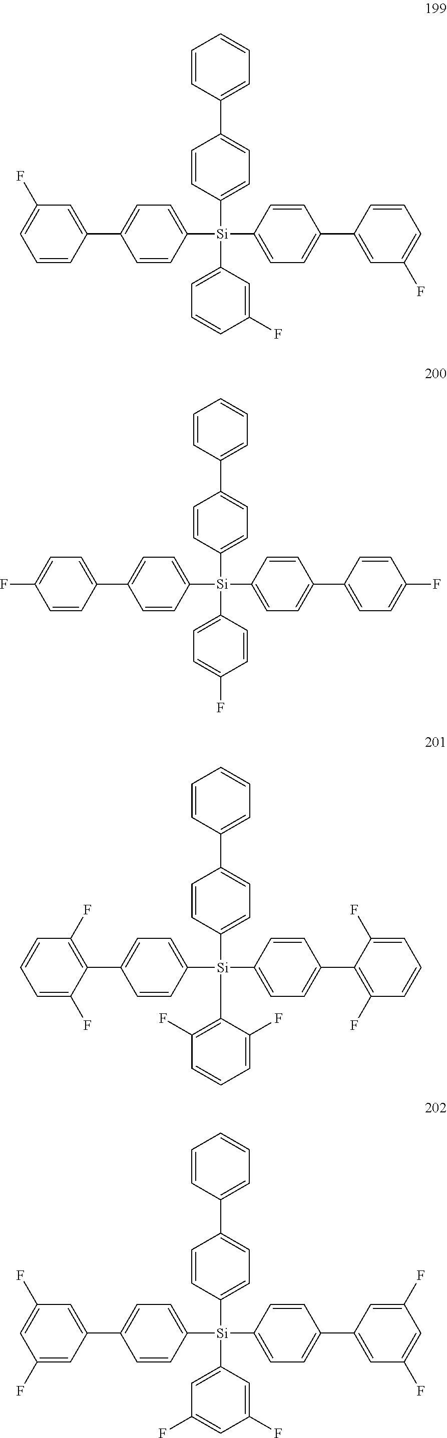 [SMILES: Fc1cccc(-c2ccc([Si](c3ccc(-c4ccccc4)cc3)(c3ccc(-c4cccc(F)c4)cc3)c3cccc(F)c3)cc2)c1 ; Fc1ccc(-c2ccc([Si](c3ccc(F)cc3)(c3ccc(-c4ccccc4)cc3)c3ccc(-c4ccc(F)cc4)cc3)cc2)cc1 ; Fc1cccc(F)c1-c1ccc([Si](c2ccc(-c3ccccc3)cc2)(c2ccc(-c3c(F)cccc3F)cc2)c2c(F)cccc2F)cc1 ; Fc1cc(F)cc(-c2ccc([Si](c3ccc(-c4ccccc4)cc3)(c3ccc(-c4cc(F)cc(F)c4)cc3)c3cc(F)cc(F)c3)cc2)c1]

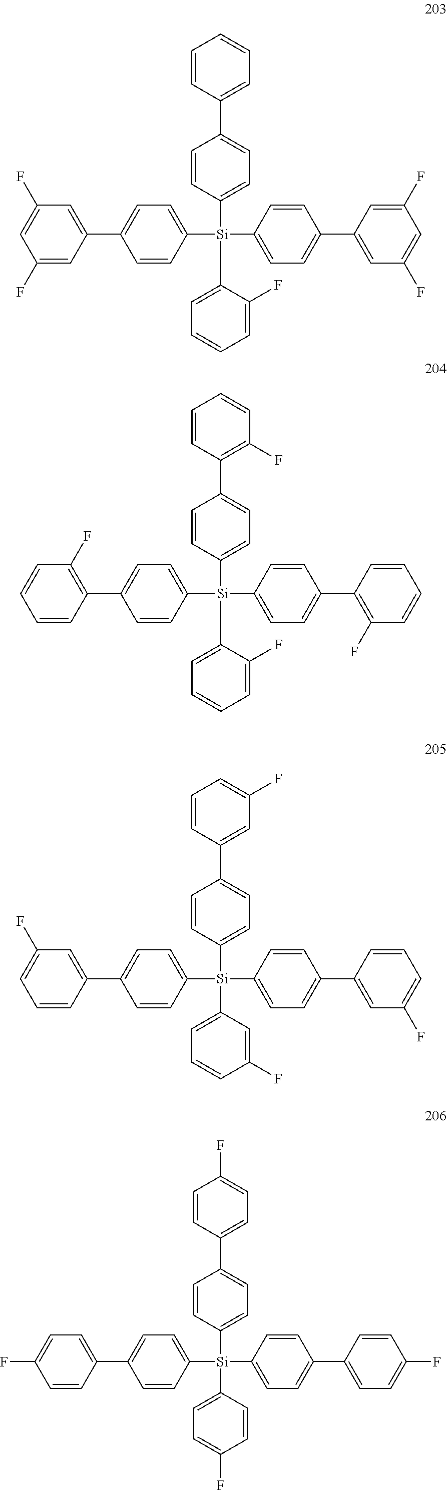

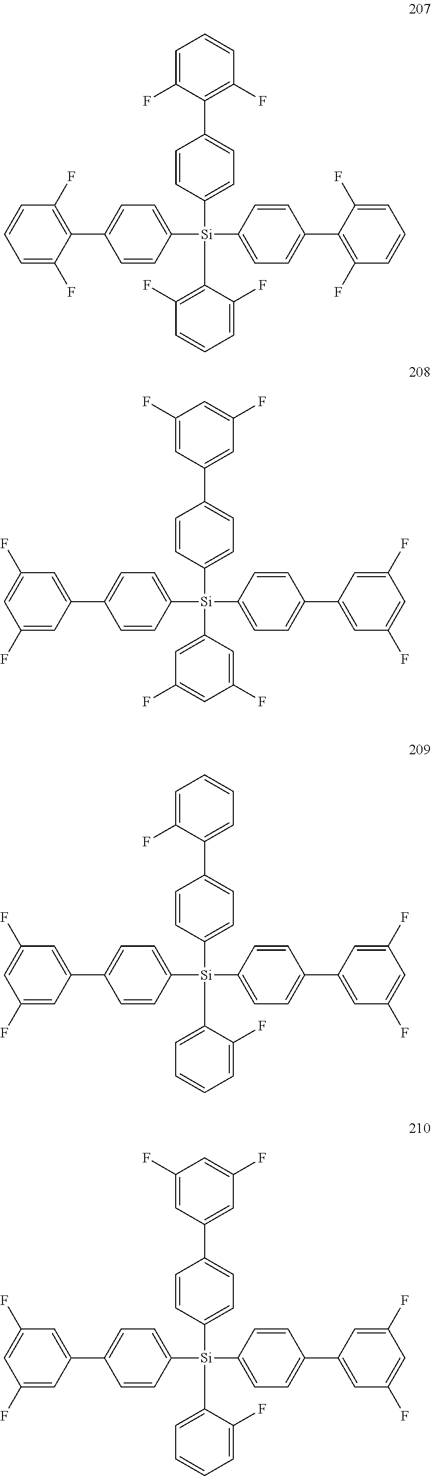 [SMILES: Fc1cccc(F)c1-c1ccc([Si](c2ccc(-c3c(F)cccc3F)cc2)(c2ccc(-c3c(F)cccc3F)cc2)c2c(F)cccc2F)cc1 ; Fc1cc(F)cc(-c2ccc([Si](c3ccc(-c4cc(F)cc(F)c4)cc3)(c3ccc(-c4cc(F)cc(F)c4)cc3)c3cc(F)cc(F)c3)cc2)c1 ; Fc1cc(F)cc(-c2ccc([Si](c3ccc(-c4cc(F)cc(F)c4)cc3)(c3ccc(-c4ccccc4F)cc3)c3ccccc3F)cc2)c1 ; Fc1cc(F)cc(-c2ccc([Si](c3ccc(-c4cc(F)cc(F)c4)cc3)(c3ccc(-c4cc(F)cc(F)c4)cc3)c3ccccc3F)cc2)c1]

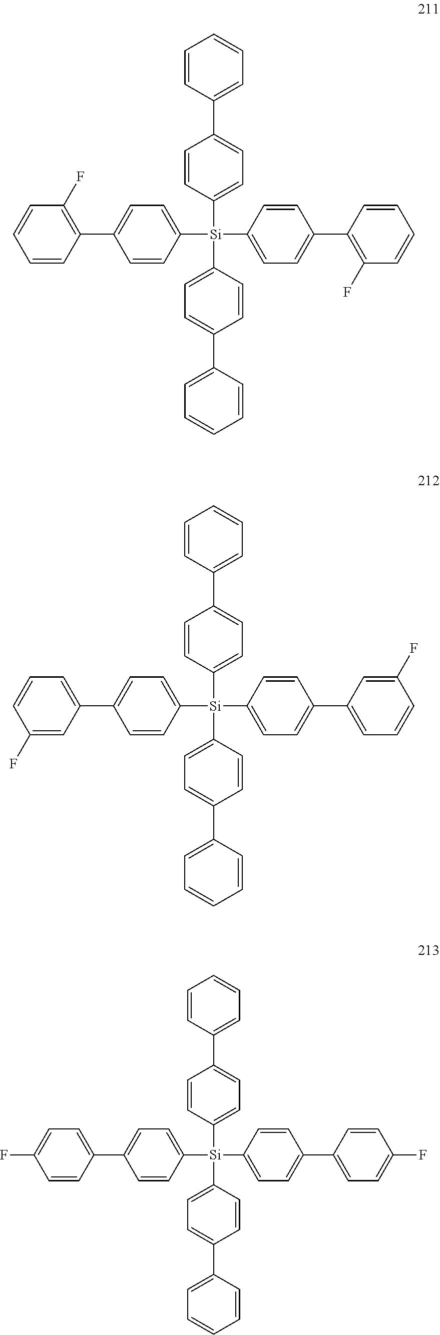 [SMILES: Fc1ccccc1-c1ccc([Si](c2ccc(-c3ccccc3)cc2)(c2ccc(-c3ccccc3)cc2)c2ccc(-c3ccccc3F)cc2)cc1 ; Fc1cccc(-c2ccc([Si](c3ccc(-c4ccccc4)cc3)(c3ccc(-c4ccccc4)cc3)c3ccc(-c4cccc(F)c4)cc3)cc2)c1 ; Fc1ccc(-c2ccc([Si](c3ccc(-c4ccccc4)cc3)(c3ccc(-c4ccccc4)cc3)c3ccc(-c4ccc(F)cc4)cc3)cc2)cc1]

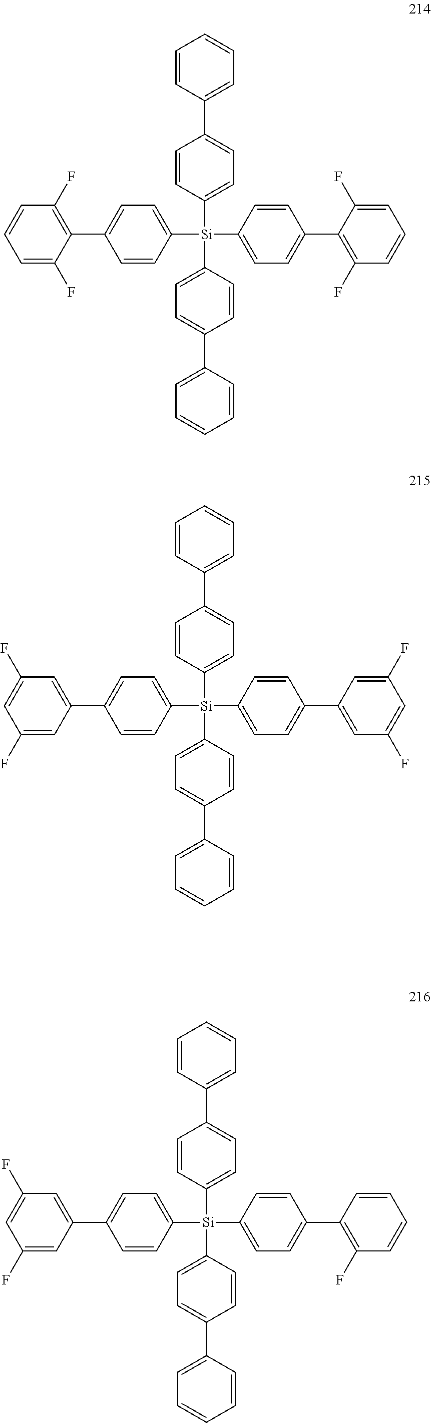 [SMILES: Fc1cccc(F)c1-c1ccc([Si](c2ccc(-c3ccccc3)cc2)(c2ccc(-c3ccccc3)cc2)c2ccc(-c3c(F)cccc3F)cc2)cc1 ; Fc1cc(F)cc(-c2ccc([Si](c3ccc(-c4ccccc4)cc3)(c3ccc(-c4ccccc4)cc3)c3ccc(-c4cc(F)cc(F)c4)cc3)cc2)c1 ; Fc1cc(F)cc(-c2ccc([Si](c3ccc(-c4ccccc4)cc3)(c3ccc(-c4ccccc4)cc3)c3ccc(-c4ccccc4F)cc3)cc2)c1]

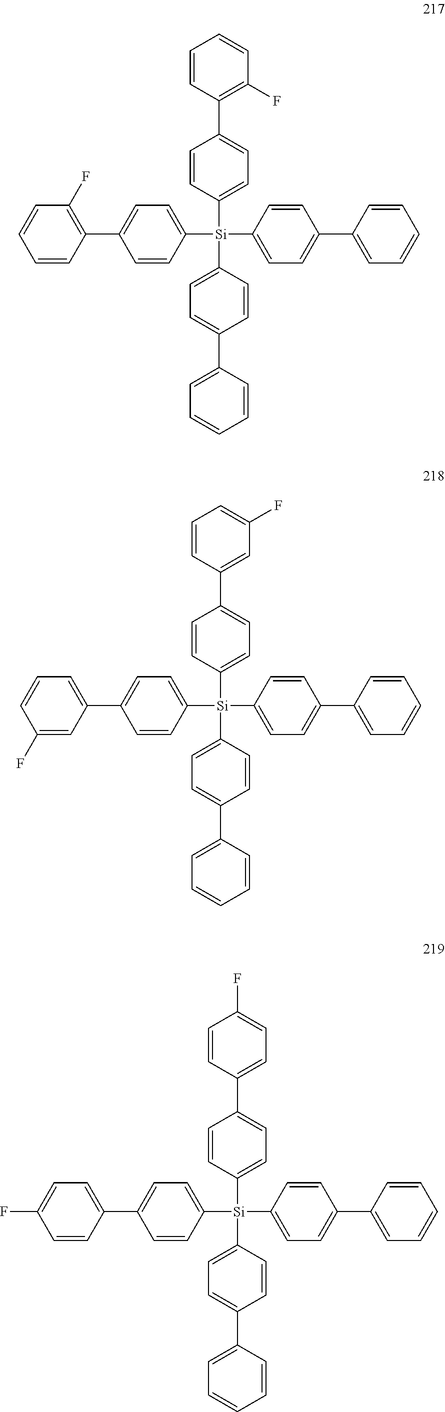 [SMILES: Fc1ccccc1-c1ccc([Si](c2ccc(-c3ccccc3)cc2)(c2ccc(-c3ccccc3)cc2)c2ccc(-c3ccccc3F)cc2)cc1 ; Fc1cccc(-c2ccc([Si](c3ccc(-c4ccccc4)cc3)(c3ccc(-c4ccccc4)cc3)c3ccc(-c4cccc(F)c4)cc3)cc2)c1 ; Fc1ccc(-c2ccc([Si](c3ccc(-c4ccccc4)cc3)(c3ccc(-c4ccccc4)cc3)c3ccc(-c4ccc(F)cc4)cc3)cc2)cc1]

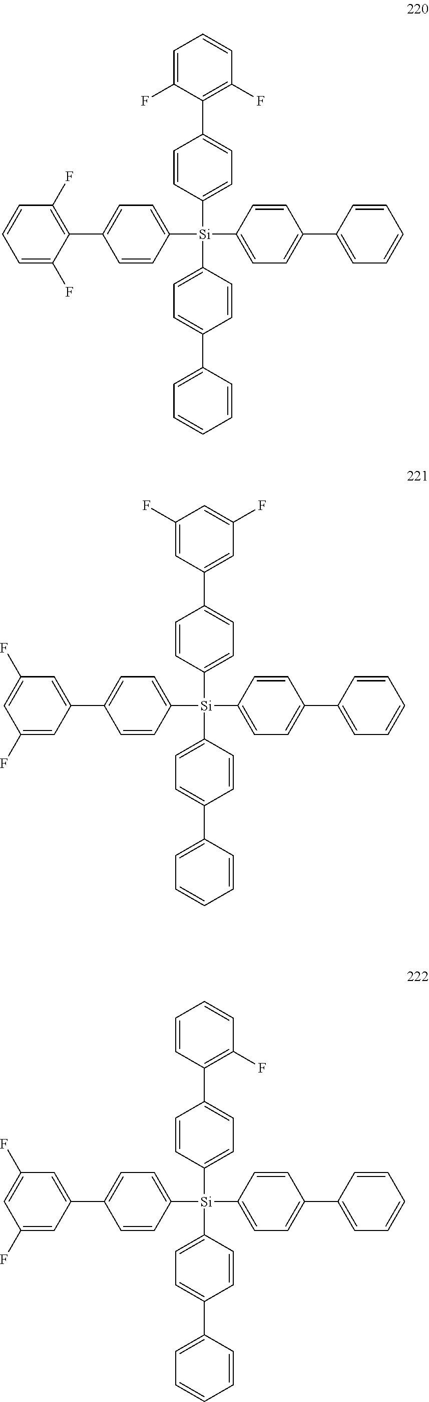 [SMILES: Fc1cccc(F)c1-c1ccc([Si](c2ccc(-c3ccccc3)cc2)(c2ccc(-c3ccccc3)cc2)c2ccc(-c3c(F)cccc3F)cc2)cc1 ; Fc1cc(F)cc(-c2ccc([Si](c3ccc(-c4ccccc4)cc3)(c3ccc(-c4ccccc4)cc3)c3ccc(-c4cc(F)cc(F)c4)cc3)cc2)c1 ; Fc1cc(F)cc(-c2ccc([Si](c3ccc(-c4ccccc4)cc3)(c3ccc(-c4ccccc4)cc3)c3ccc(-c4ccccc4F)cc3)cc2)c1]

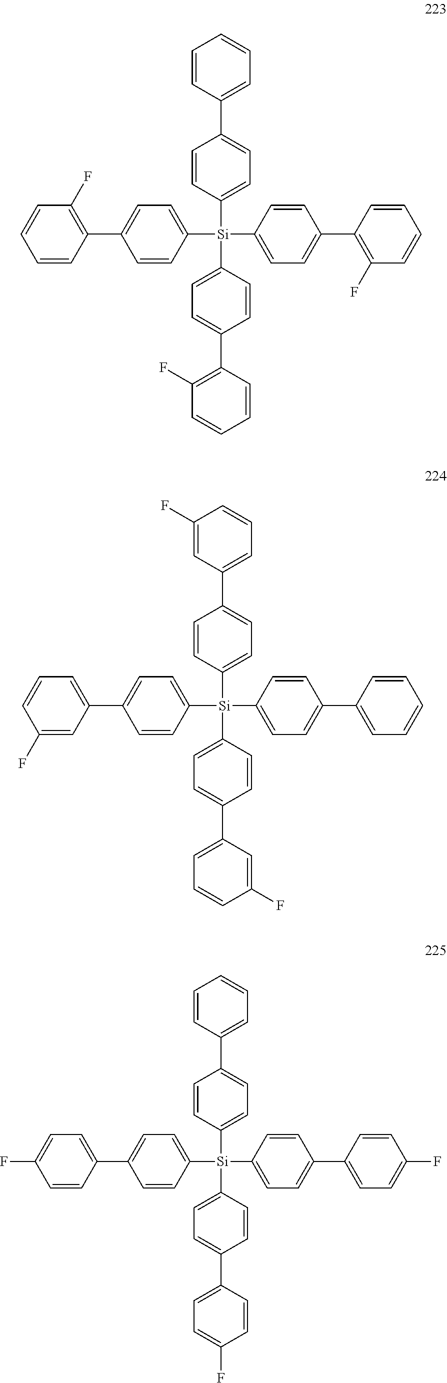 [SMILES: Fc1ccccc1-c1ccc([Si](c2ccc(-c3ccccc3)cc2)(c2ccc(-c3ccccc3F)cc2)c2ccc(-c3ccccc3F)cc2)cc1 ; Fc1cccc(-c2ccc([Si](c3ccc(-c4ccccc4)cc3)(c3ccc(-c4cccc(F)c4)cc3)c3ccc(-c4cccc(F)c4)cc3)cc2)c1 ; Fc1ccc(-c2ccc([Si](c3ccc(-c4ccccc4)cc3)(c3ccc(-c4ccc(F)cc4)cc3)c3ccc(-c4ccc(F)cc4)cc3)cc2)cc1]

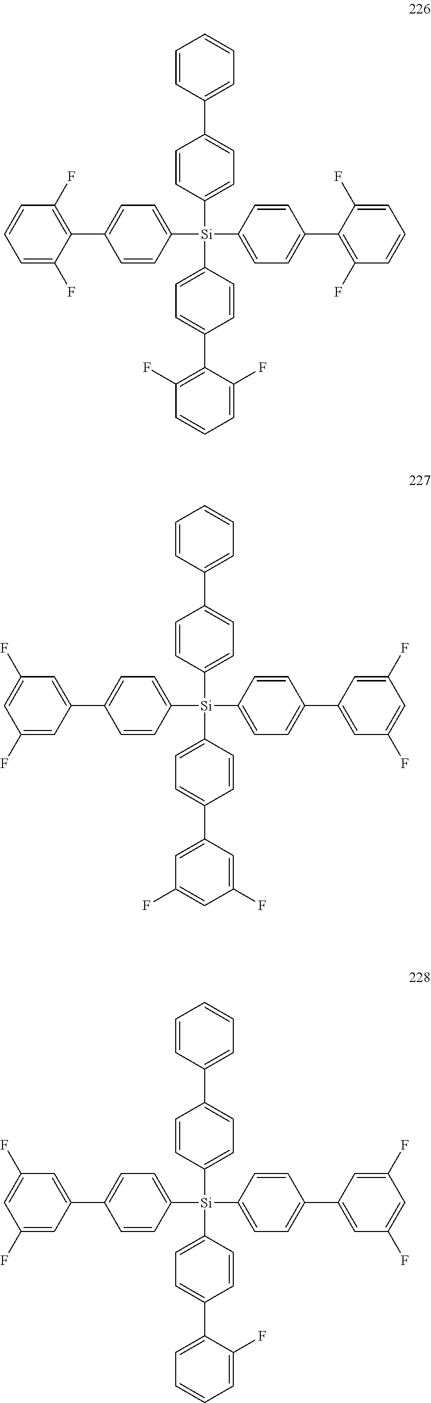 [SMILES: Fc1cccc(F)c1-c1ccc([Si](c2ccc(-c3ccccc3)cc2)(c2ccc(-c3c(F)cccc3F)cc2)c2ccc(-c3c(F)cccc3F)cc2)cc1 ; Fc1cc(F)cc(-c2ccc([Si](c3ccc(-c4ccccc4)cc3)(c3ccc(-c4cc(F)cc(F)c4)cc3)c3ccc(-c4cc(F)cc(F)c4)cc3)cc2)c1 ; Fc1cc(F)cc(-c2ccc([Si](c3ccc(-c4ccccc4)cc3)(c3ccc(-c4cc(F)cc(F)c4)cc3)c3ccc(-c4ccccc4F)cc3)cc2)c1]

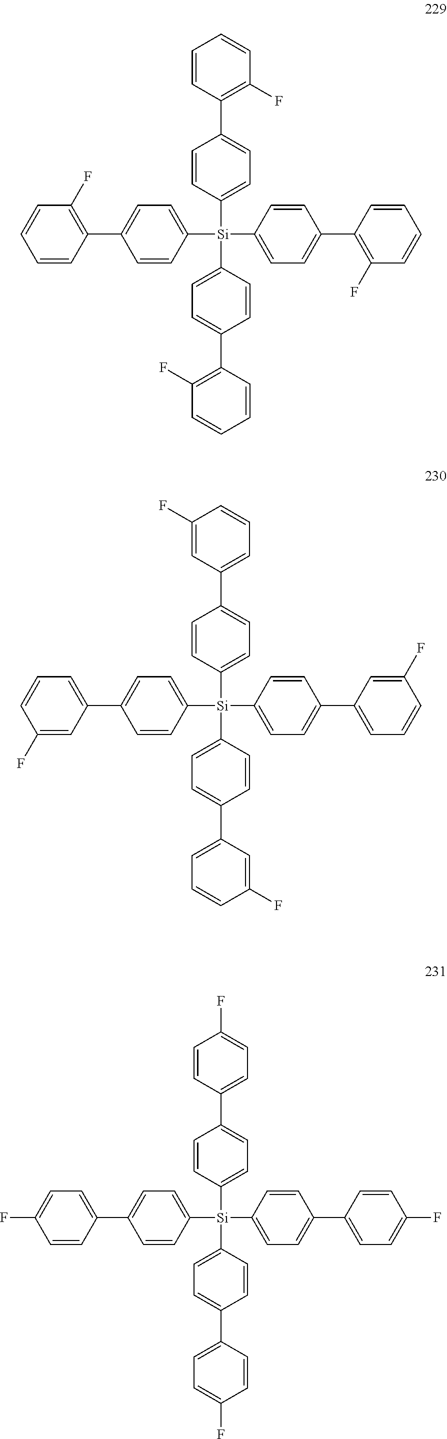 [SMILES: Fc1ccccc1-c1ccc([Si](c2ccc(-c3ccccc3F)cc2)(c2ccc(-c3ccccc3F)cc2)c2ccc(-c3ccccc3F)cc2)cc1 ; Fc1cccc(-c2ccc([Si](c3ccc(-c4cccc(F)c4)cc3)(c3ccc(-c4cccc(F)c4)cc3)c3ccc(-c4cccc(F)c4)cc3)cc2)c1 ; Fc1ccc(-c2ccc([Si](c3ccc(-c4ccc(F)cc4)cc3)(c3ccc(-c4ccc(F)cc4)cc3)c3ccc(-c4ccc(F)cc4)cc3)cc2)cc1]

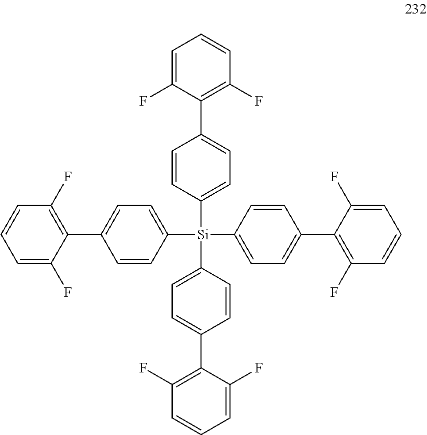 [SMILES: Fc1cccc(F)c1-c1ccc([Si](c2ccc(-c3c(F)cccc3F)cc2)(c2ccc(-c3c(F)cccc3F)cc2)c2ccc(-c3c(F)cccc3F)cc2)cc1]

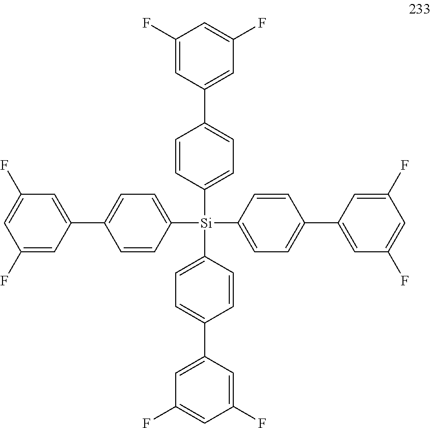 [SMILES: Fc1cc(F)cc(-c2ccc([Si](c3ccc(-c4cc(F)cc(F)c4)cc3)(c3ccc(-c4cc(F)cc(F)c4)cc3)c3ccc(-c4cc(F)cc(F)c4)cc3)cc2)c1]

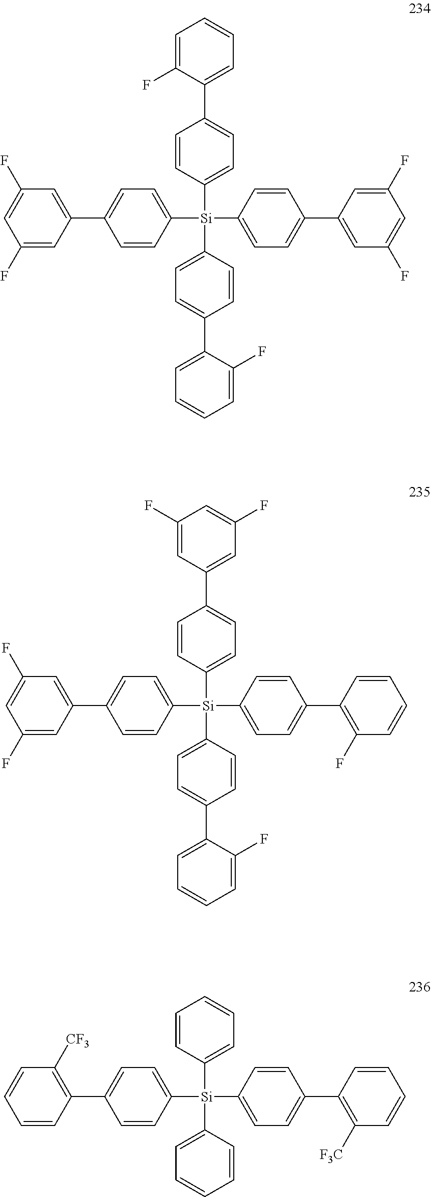 [SMILES: Fc1cc(F)cc(-c2ccc([Si](c3ccc(-c4cc(F)cc(F)c4)cc3)(c3ccc(-c4ccccc4F)cc3)c3ccc(-c4ccccc4F)cc3)cc2)c1 ; Fc1cc(F)cc(-c2ccc([Si](c3ccc(-c4cc(F)cc(F)c4)cc3)(c3ccc(-c4ccccc4F)cc3)c3ccc(-c4ccccc4F)cc3)cc2)c1 ; FC(F)(F)c1ccccc1-c1ccc([Si](c2ccccc2)(c2ccccc2)c2ccc(-c3ccccc3C(F)(F)F)cc2)cc1]

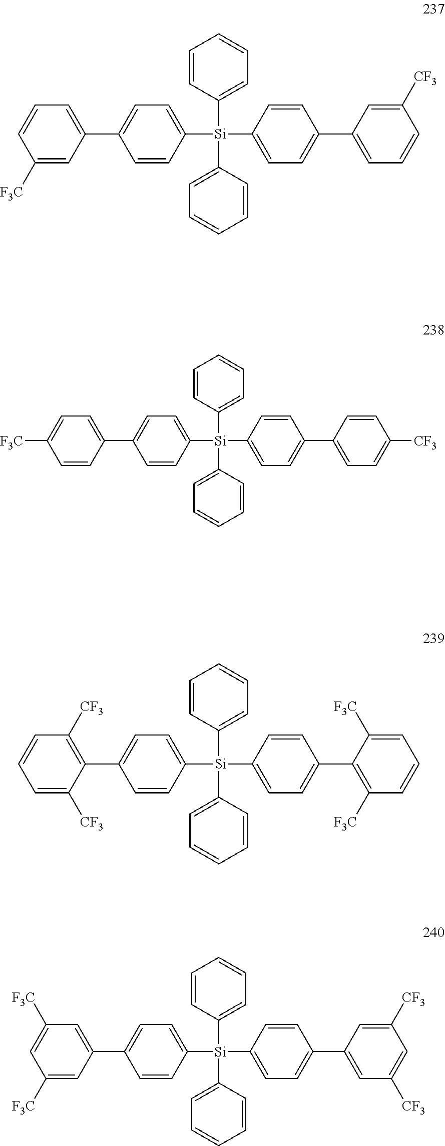 [SMILES: FC(F)(F)c1cccc(-c2ccc([Si](c3ccccc3)(c3ccccc3)c3ccc(-c4cccc(C(F)(F)F)c4)cc3)cc2)c1 ; FC(F)(F)c1ccc(-c2ccc([Si](c3ccccc3)(c3ccccc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccccc2)c2ccc(-c3c(C(F)(F)F)cccc3C(F)(F)F)cc2)cc1 ; FC(F)(F)c1cc(-c2ccc([Si](c3ccccc3)(c3ccccc3)c3ccc(-c4cc(C(F)(F)F)cc(C(F)(F)F)c4)cc3)cc2)cc(C(F)(F)F)c1]

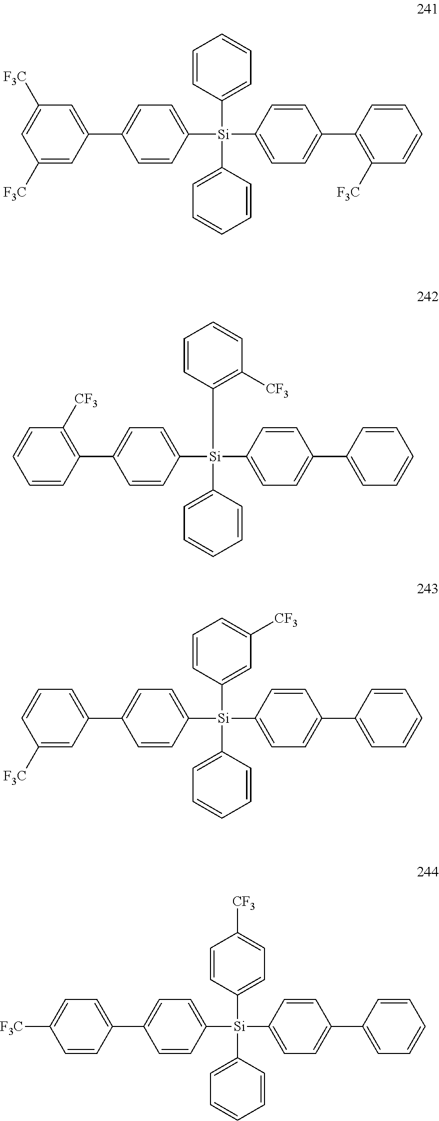 [SMILES: FC(F)(F)c1cc(-c2ccc([Si](c3ccccc3)(c3ccccc3)c3ccc(-c4ccccc4C(F)(F)F)cc3)cc2)cc(C(F)(F)F)c1 ; FC(F)(F)c1ccccc1-c1ccc([Si](c2ccccc2)(c2ccc(-c3ccccc3)cc2)c2ccccc2C(F)(F)F)cc1 ; FC(F)(F)c1cccc(-c2ccc([Si](c3ccccc3)(c3ccc(-c4ccccc4)cc3)c3cccc(C(F)(F)F)c3)cc2)c1 ; FC(F)(F)c1ccc(-c2ccc([Si](c3ccccc3)(c3ccc(-c4ccccc4)cc3)c3ccc(C(F)(F)F)cc3)cc2)cc1]

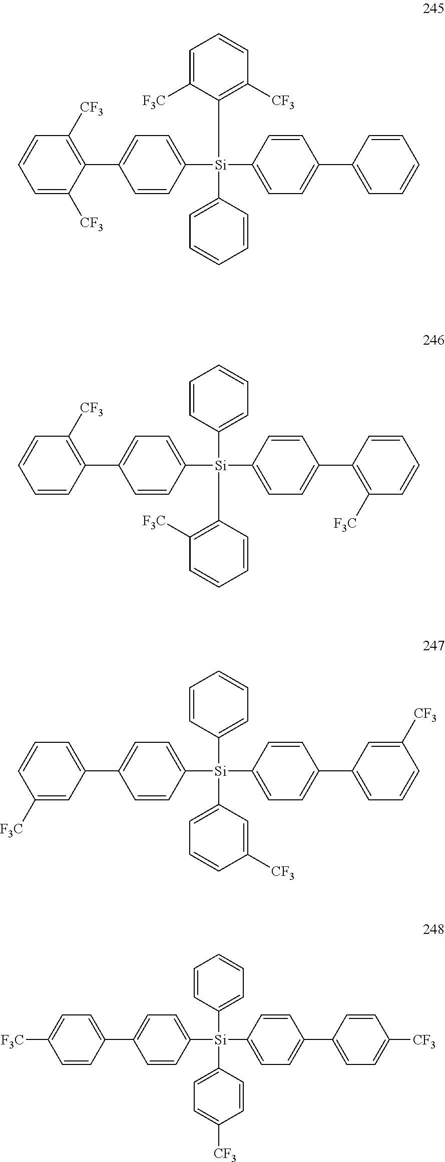 [SMILES: FC(F)(F)c1cccc(C(F)(F)F)c1-c1ccc([Si](c2ccccc2)(c2ccc(-c3ccccc3)cc2)c2c(C(F)(F)F)cccc2C(F)(F)F)cc1 ; FC(F)(F)c1ccccc1-c1ccc([Si](c2ccccc2)(c2ccc(-c3ccccc3C(F)(F)F)cc2)c2ccccc2C(F)(F)F)cc1 ; FC(F)(F)c1cccc(-c2ccc([Si](c3ccccc3)(c3ccc(-c4cccc(C(F)(F)F)c4)cc3)c3cccc(C(F)(F)F)c3)cc2)c1 ; FC(F)(F)c1ccc(-c2ccc([Si](c3ccccc3)(c3ccc(-c4ccc(C(F)(F)F)cc4)cc3)c3ccc(C(F)(F)F)cc3)cc2)cc1]

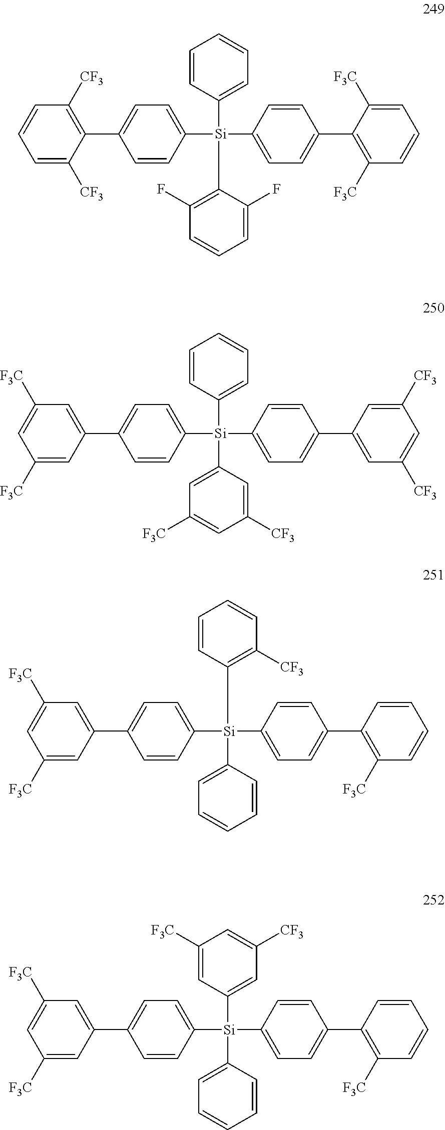 [SMILES: Fc1cccc(F)c1[Si](c1ccccc1)(c1ccc(-c2c(C(F)(F)F)cccc2C(F)(F)F)cc1)c1ccc(-c2c(C(F)(F)F)cccc2C(F)(F)F)cc1 ; FC(F)(F)c1cc(-c2ccc([Si](c3ccccc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccccc3)(c3ccc(-c4ccccc4C(F)(F)F)cc3)c3ccccc3C(F)(F)F)cc2)cc(C(F)(F)F)c1 ; FC(F)(F)c1cc(-c2ccc([Si](c3ccccc3)(c3ccc(-c4ccccc4C(F)(F)F)cc3)c3cc(C(F)(F)F)cc(C(F)(F)F)c3)cc2)cc(C(F)(F)F)c1]

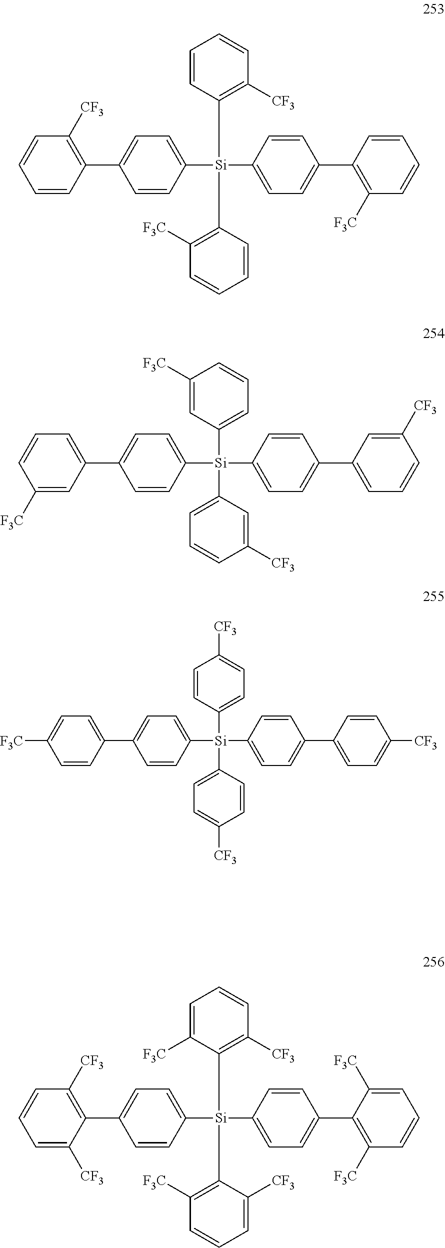 [SMILES: FC(F)(F)c1ccccc1-c1ccc([Si](c2ccc(-c3ccccc3C(F)(F)F)cc2)(c2ccccc2C(F)(F)F)c2ccccc2C(F)(F)F)cc1 ; FC(F)(F)c1cccc(-c2ccc([Si](c3ccc(-c4cccc(C(F)(F)F)c4)cc3)(c3cccc(C(F)(F)F)c3)c3cccc(C(F)(F)F)c3)cc2)c1 ; FC(F)(F)c1ccc(-c2ccc([Si](c3ccc(-c4ccc(C(F)(F)F)cc4)cc3)(c3ccc(C(F)(F)F)cc3)c3ccc(C(F)(F)F)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2c(C(F)(F)F)cccc2C(F)(F)F)c2c(C(F)(F)F)cccc2C(F)(F)F)cc1]

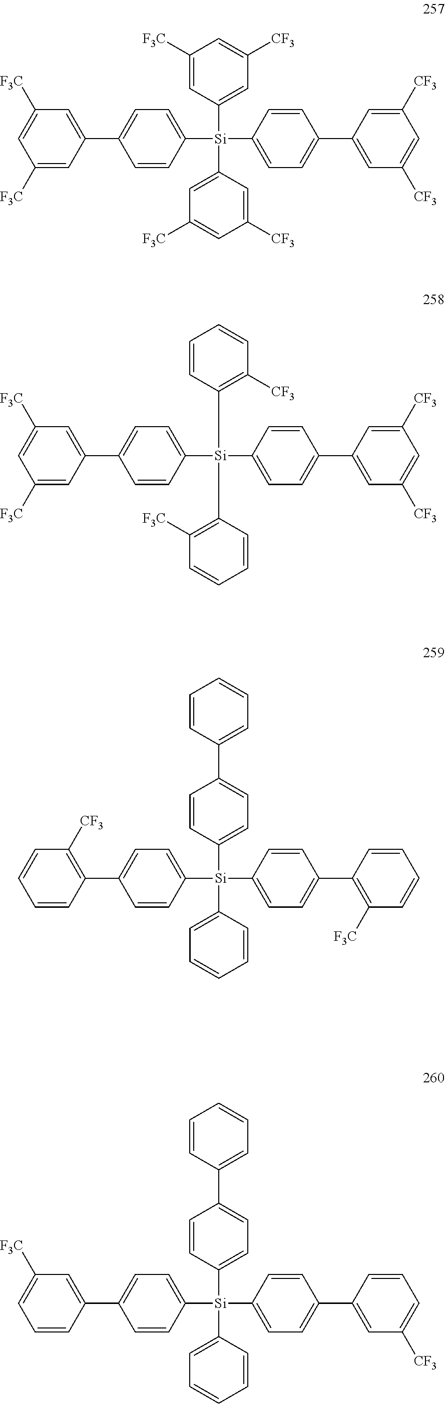 [SMILES: FC(F)(F)c1cc(-c2ccc([Si](c3ccc(-c4cc(C(F)(F)F)cc(C(F)(F)F)c4)cc3)(c3cc(C(F)(F)F)cc(C(F)(F)F)c3)c3cc(C(F)(F)F)cc(C(F)(F)F)c3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccccc3C(F)(F)F)c3ccccc3C(F)(F)F)cc2)cc(C(F)(F)F)c1 ; FC(F)(F)c1ccccc1-c1ccc([Si](c2ccccc2)(c2ccc(-c3ccccc3)cc2)c2ccc(-c3ccccc3C(F)(F)F)cc2)cc1 ; FC(F)(F)c1cccc(-c2ccc([Si](c3ccccc3)(c3ccc(-c4ccccc4)cc3)c3ccc(-c4cccc(C(F)(F)F)c4)cc3)cc2)c1]

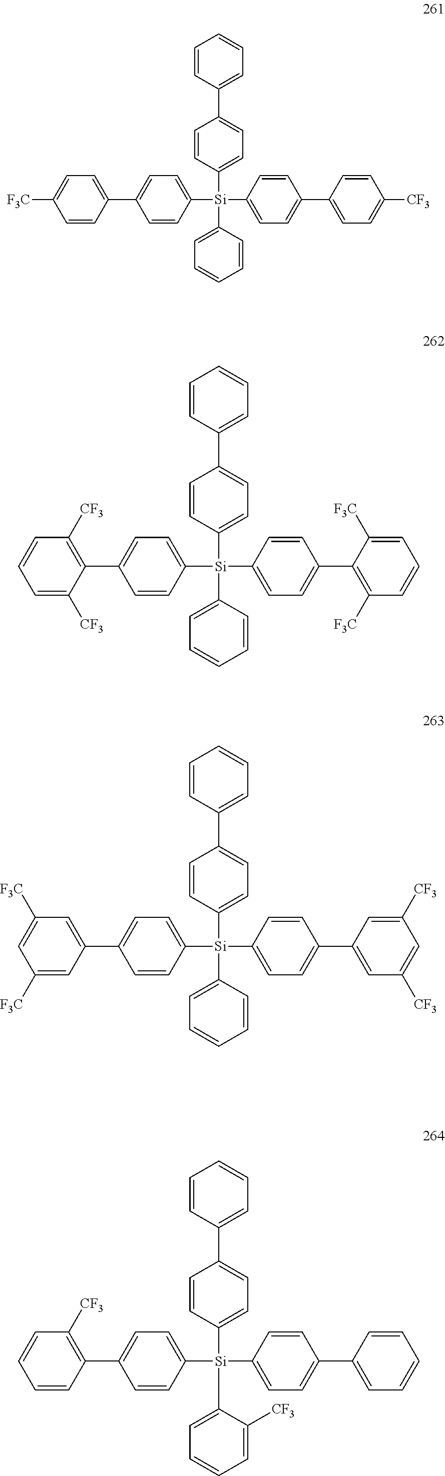 [SMILES: FC(F)(F)c1ccc(-c2ccc([Si](c3ccccc3)(c3ccc(-c4ccccc4)cc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccccc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1ccccc1-c1ccc([Si](c2ccc(-c3ccccc3)cc2)(c2ccc(-c3ccccc3)cc2)c2ccccc2C(F)(F)F)cc1]

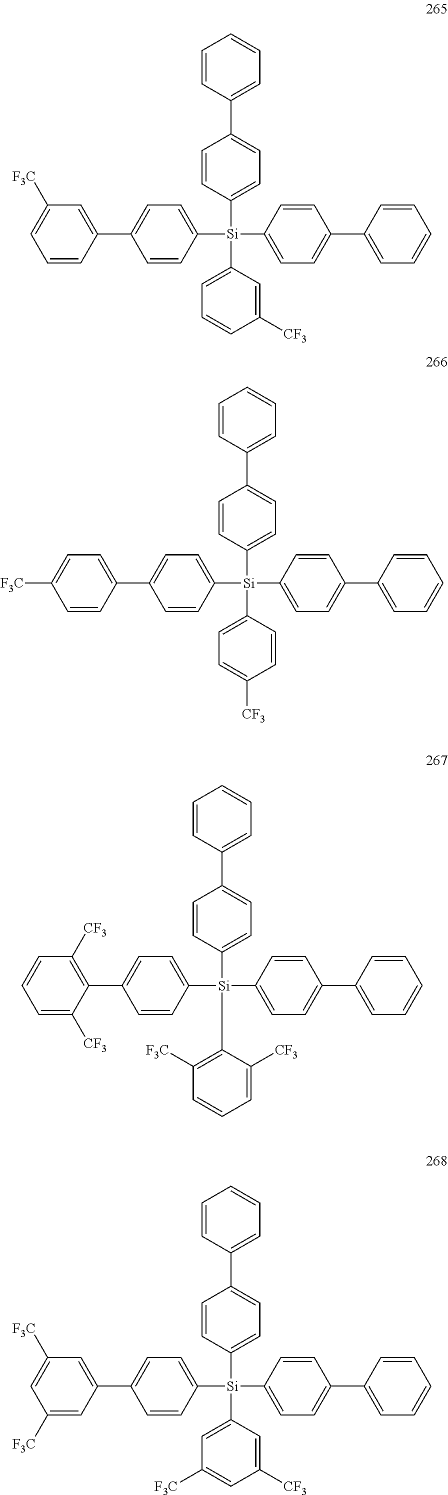 [SMILES: FC(F)(F)c1cccc(-c2ccc([Si](c3ccc(-c4ccccc4)cc3)(c3ccc(-c4ccccc4)cc3)c3cccc(C(F)(F)F)c3)cc2)c1 ; FC(F)(F)c1ccc(-c2ccc([Si](c3ccc(-c4ccccc4)cc3)(c3ccc(-c4ccccc4)cc3)c3ccc(C(F)(F)F)cc3)cc2)cc1 ; FC(F)(F)c1cccc(C(F)(F)F)c1-c1ccc([Si](c2ccc(-c3ccccc3)cc2)(c2ccc(-c3ccccc3)cc2)c2c(C(F)(F)F)cccc2C(F)(F)F)cc1 ; FC(F)(F)c1cc(-c2ccc([Si](c3ccc(-c4ccccc4)cc3)(c3ccc(-c4ccccc4)cc3)c3cc(C(F)(F)F)cc(C(F)(F)F)c3)cc2)cc(C(F)(F)F)c1]

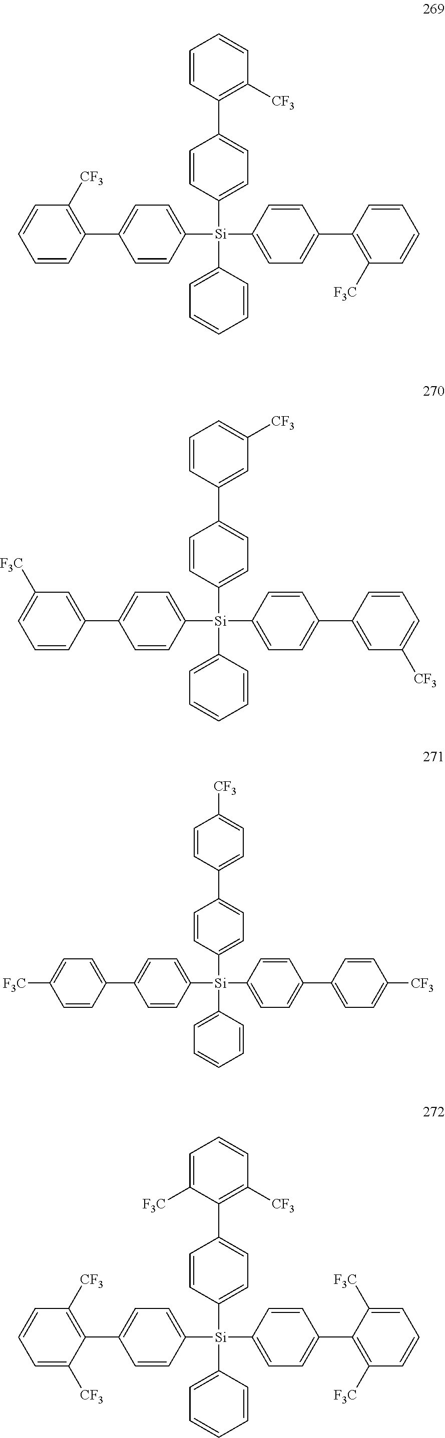 [SMILES: FC(F)(F)c1ccccc1-c1ccc([Si](c2ccccc2)(c2ccc(-c3ccccc3C(F)(F)F)cc2)c2ccc(-c3ccccc3C(F)(F)F)cc2)cc1 ; FC(F)(F)c1cccc(-c2ccc([Si](c3ccccc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccccc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccccc2)(c2ccc(-c3c(C(F)(F)F)cccc3C(F)(F)F)cc2)c2ccc(-c3c(C(F)(F)F)cccc3C(F)(F)F)cc2)cc1]

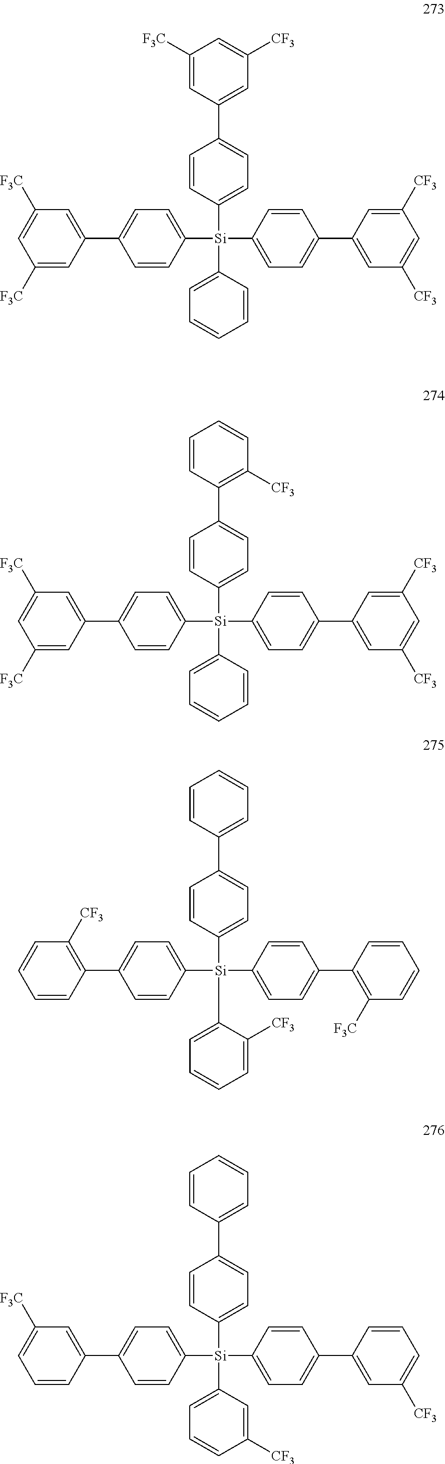 [SMILES: FC(F)(F)c1cc(-c2ccc([Si](c3ccccc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccccc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc([Si](c2ccc(-c3ccccc3)cc2)(c2ccc(-c3ccccc3C(F)(F)F)cc2)c2ccccc2C(F)(F)F)cc1 ; FC(F)(F)c1cccc(-c2ccc([Si](c3ccc(-c4ccccc4)cc3)(c3ccc(-c4cccc(C(F)(F)F)c4)cc3)c3cccc(C(F)(F)F)c3)cc2)c1]

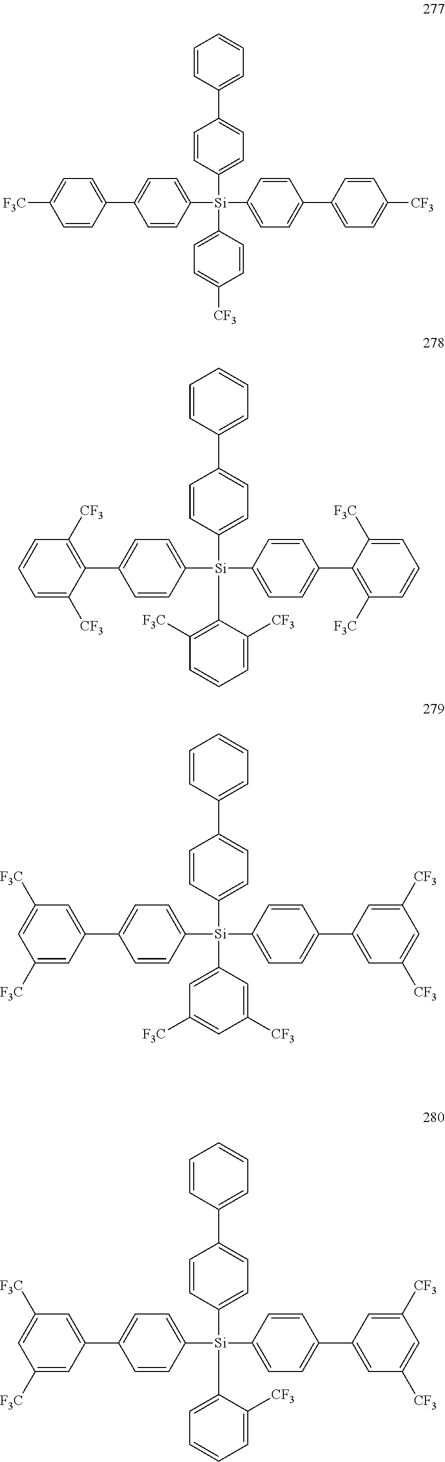 [SMILES: FC(F)(F)c1ccc(-c2ccc([Si](c3ccc(-c4ccccc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc([Si](c2ccc(-c3ccccc3)cc2)(c2ccc(-c3c(C(F)(F)F)cccc3C(F)(F)F)cc2)c2c(C(F)(F)F)cccc2C(F)(F)F)cc1 ; FC(F)(F)c1cc(-c2ccc([Si](c3ccc(-c4ccccc4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccc(-c4ccccc4)cc3)(c3ccc(-c4cc(C(F)(F)F)cc(C(F)(F)F)c4)cc3)c3ccccc3C(F)(F)F)cc2)cc(C(F)(F)F)c1]

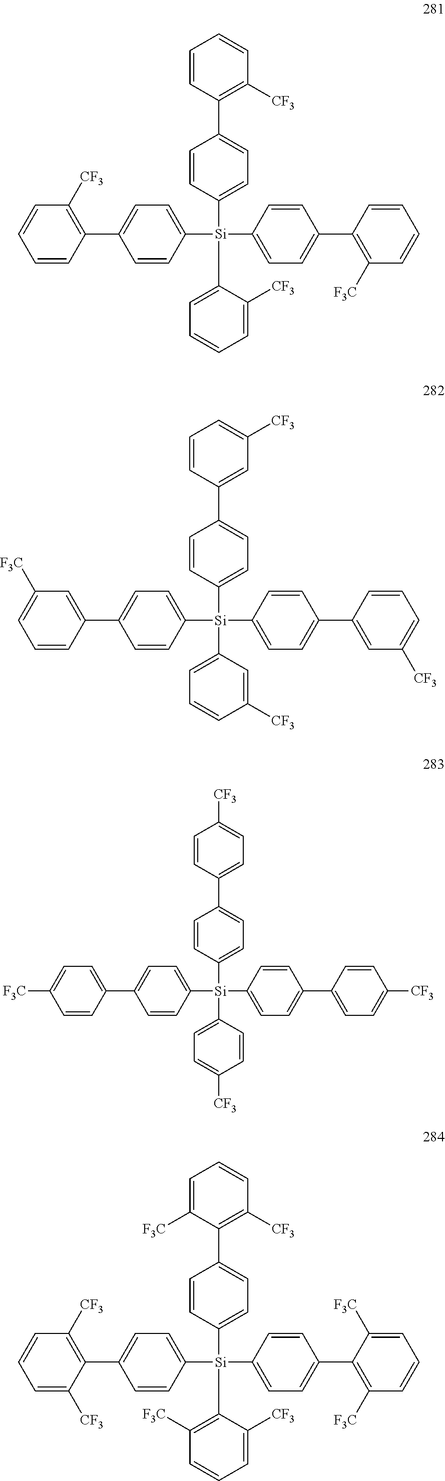 [SMILES: FC(F)(F)c1ccccc1-c1ccc([Si](c2ccc(-c3ccccc3C(F)(F)F)cc2)(c2ccc(-c3ccccc3C(F)(F)F)cc2)c2ccccc2C(F)(F)F)cc1 ; FC(F)(F)c1cccc(-c2ccc([Si](c3ccc(-c4cccc(C(F)(F)F)c4)cc3)(c3ccc(-c4cccc(C(F)(F)F)c4)cc3)c3cccc(C(F)(F)F)c3)cc2)c1 ; FC(F)(F)c1ccc(-c2ccc([Si](c3ccc(-c4ccc(C(F)(F)F)cc4)cc3)(c3ccc(-c4ccc(C(F)(F)F)cc4)cc3)c3ccc(C(F)(F)F)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2ccc(-c3c(C(F)(F)F)cccc3C(F)(F)F)cc2)c2c(C(F)(F)F)cccc2C(F)(F)F)cc1]

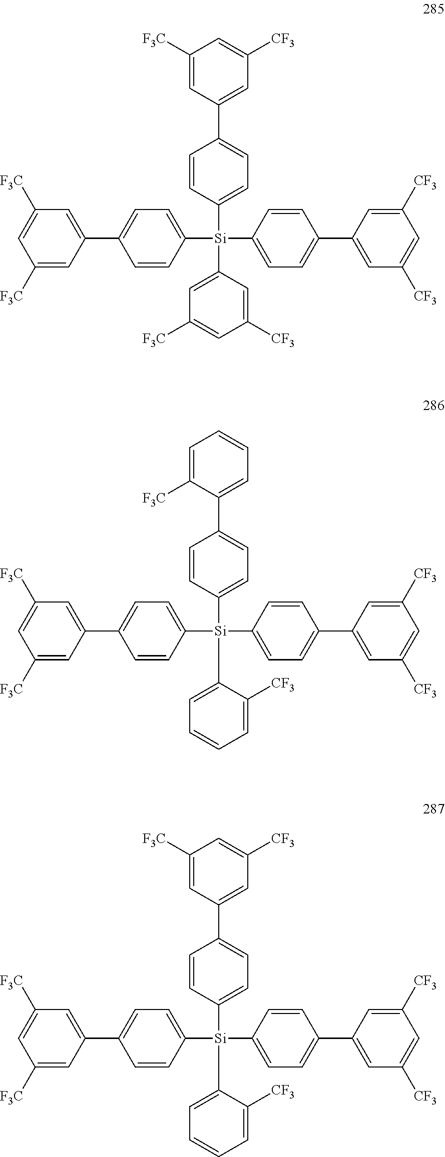 [SMILES: FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3cc(C(F)(F)F)cc(C(F)(F)F)c3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccccc3C(F)(F)F)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3ccccc3C(F)(F)F)cc2)cc(C(F)(F)F)c1]

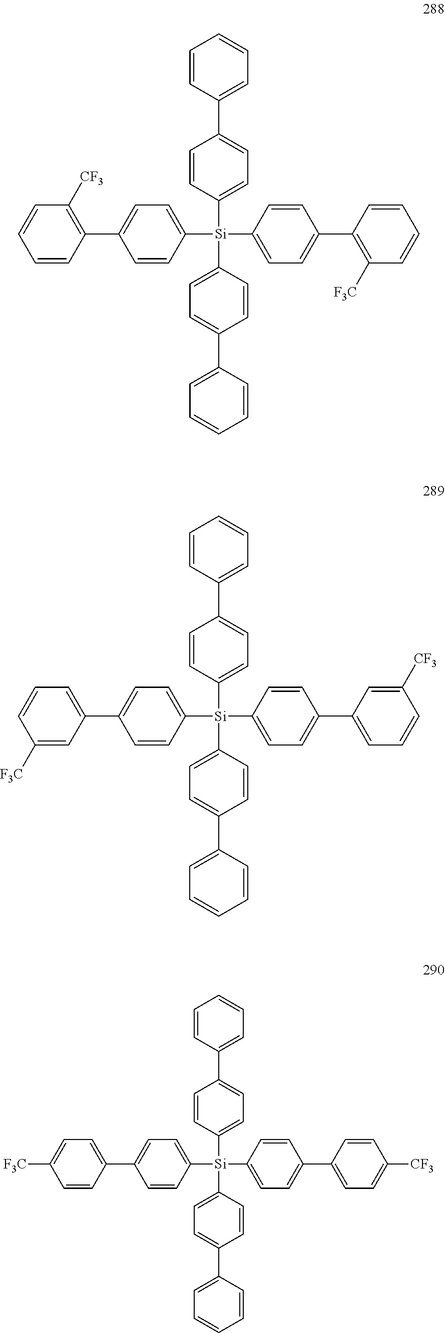 [SMILES: FC(F)(F)c1ccccc1-c1ccc([Si](c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc([Si](c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccccc4)cc3)c3ccc(-c4ccc(C(F)(F)F)cc4)cc3)cc2)cc1]

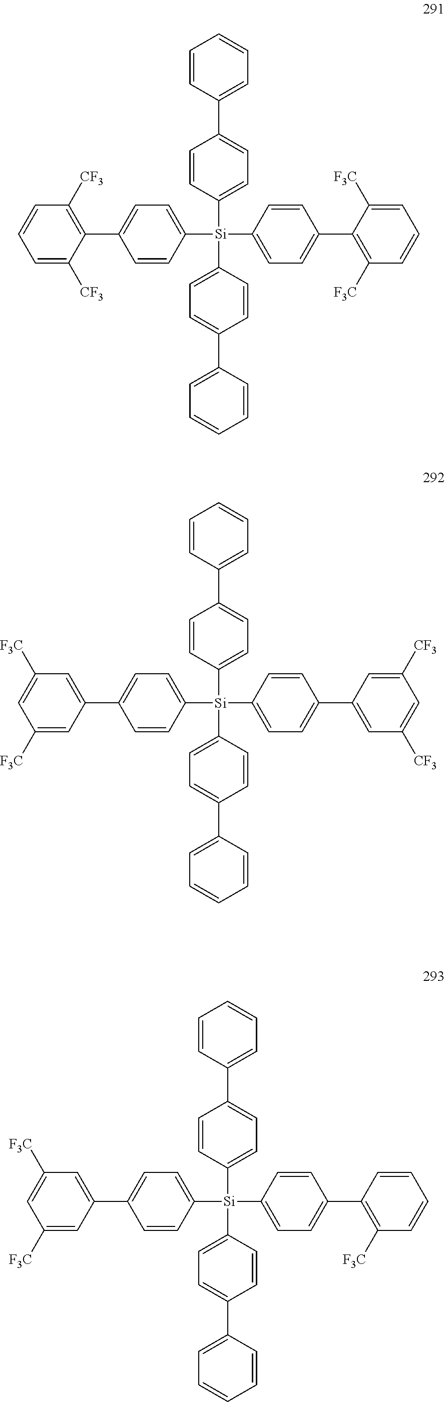 [SMILES: FC(F)(F)c1cccc(C(F)(F)F)c1-c1ccc([Si](c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4ccccc4)cc3)c3ccc(-c4ccccc4C(F)(F)F)cc3)cc2)cc(C(F)(F)F)c1]

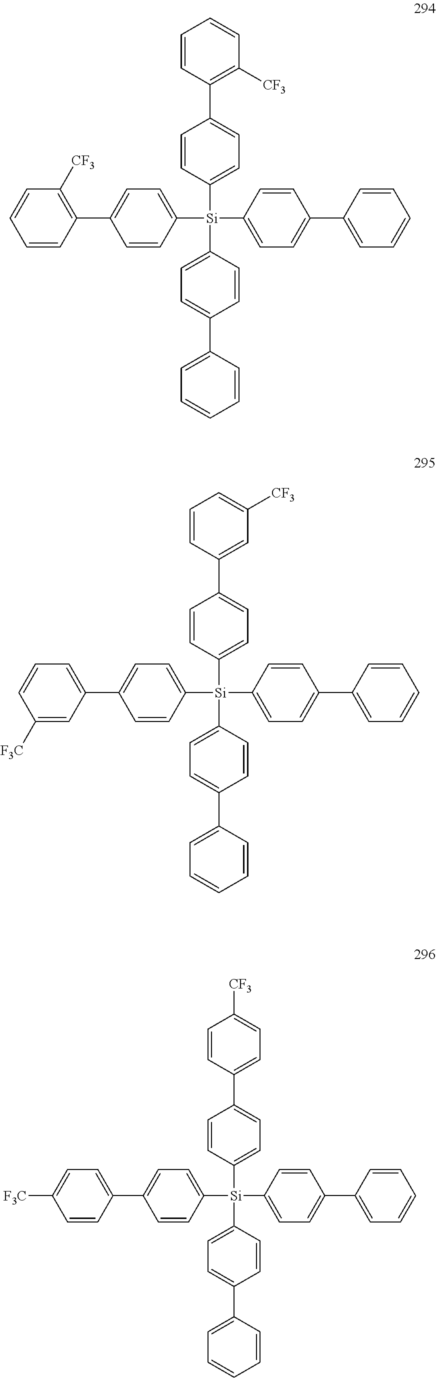 [SMILES: FC(F)(F)c1ccccc1-c1ccc([Si](c2ccc(-c3ccccc3)cc2)(c2ccc(-c3ccccc3)cc2)c2ccc(-c3ccccc3C(F)(F)F)cc2)cc1 ; FC(F)(F)c1cccc(-c2ccc([Si](c3ccc(-c4ccccc4)cc3)(c3ccc(-c4ccccc4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccccc4)cc3)c3ccc(-c4ccc(C(F)(F)F)cc4)cc3)cc2)cc1]

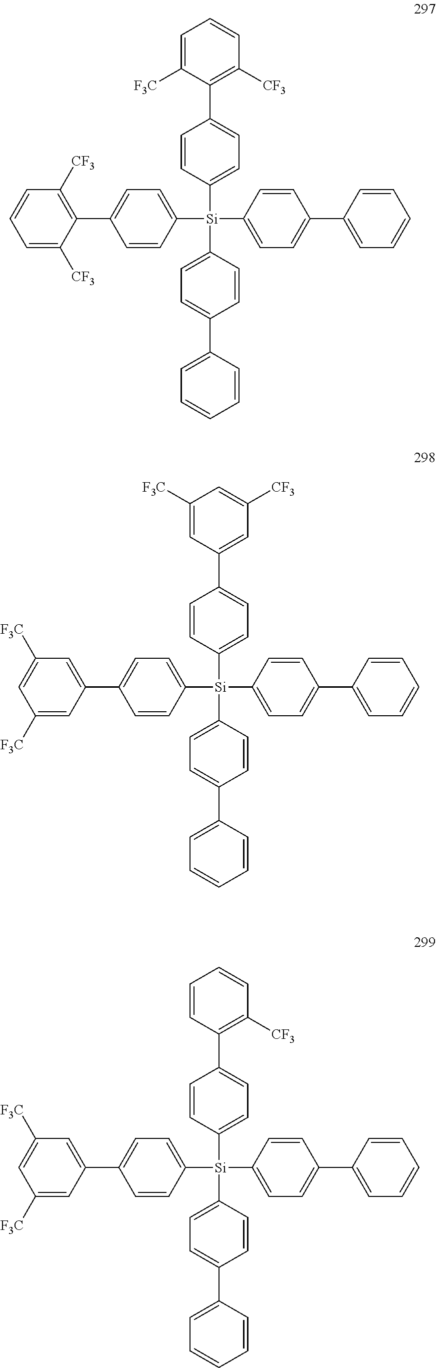 [SMILES: FC(F)(F)c1cccc(C(F)(F)F)c1-c1ccc([Si](c2ccc(-c3ccccc3)cc2)(c2ccc(-c3ccccc3)cc2)c2ccc(-c3c(C(F)(F)F)cccc3C(F)(F)F)cc2)cc1 ; FC(F)(F)c1cc(-c2ccc([Si](c3ccc(-c4ccccc4)cc3)(c3ccc(-c4ccccc4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4ccccc4)cc3)c3ccc(-c4ccccc4C(F)(F)F)cc3)cc2)cc(C(F)(F)F)c1]

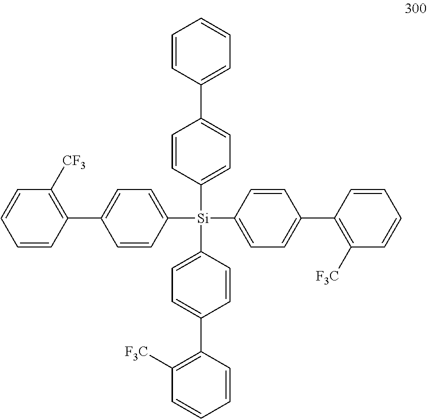 [SMILES: FC(F)(F)c1ccccc1-c1ccc([Si](c2ccc(-c3ccccc3)cc2)(c2ccc(-c3ccccc3C(F)(F)F)cc2)c2ccc(-c3ccccc3C(F)(F)F)cc2)cc1]

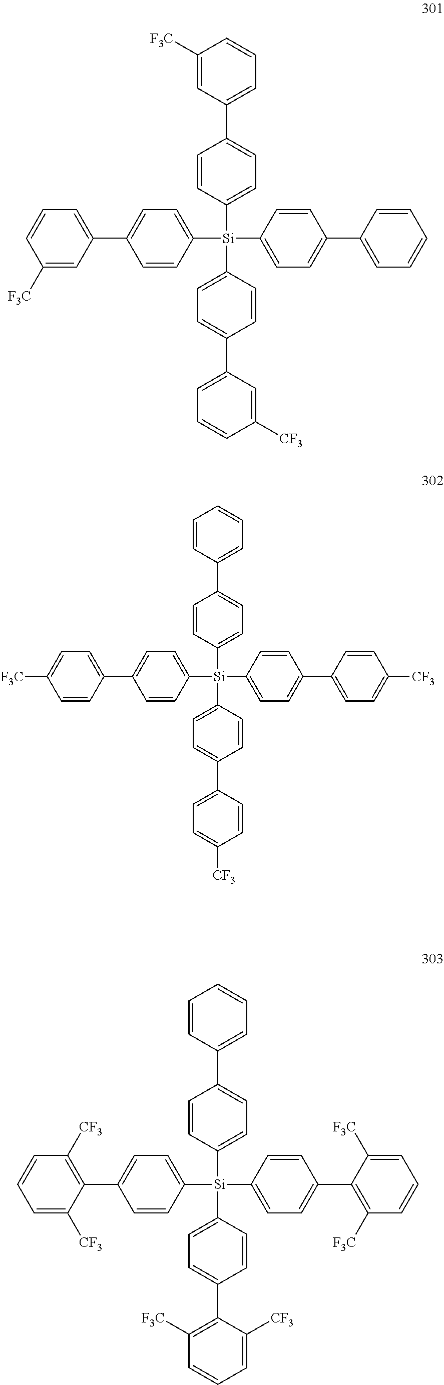 [SMILES: FC(F)(F)c1cccc(-c2ccc([Si](c3ccc(-c4ccccc4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccc(-c4ccccc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccc(-c3ccccc3)cc2)(c2ccc(-c3c(C(F)(F)F)cccc3C(F)(F)F)cc2)c2ccc(-c3c(C(F)(F)F)cccc3C(F)(F)F)cc2)cc1]

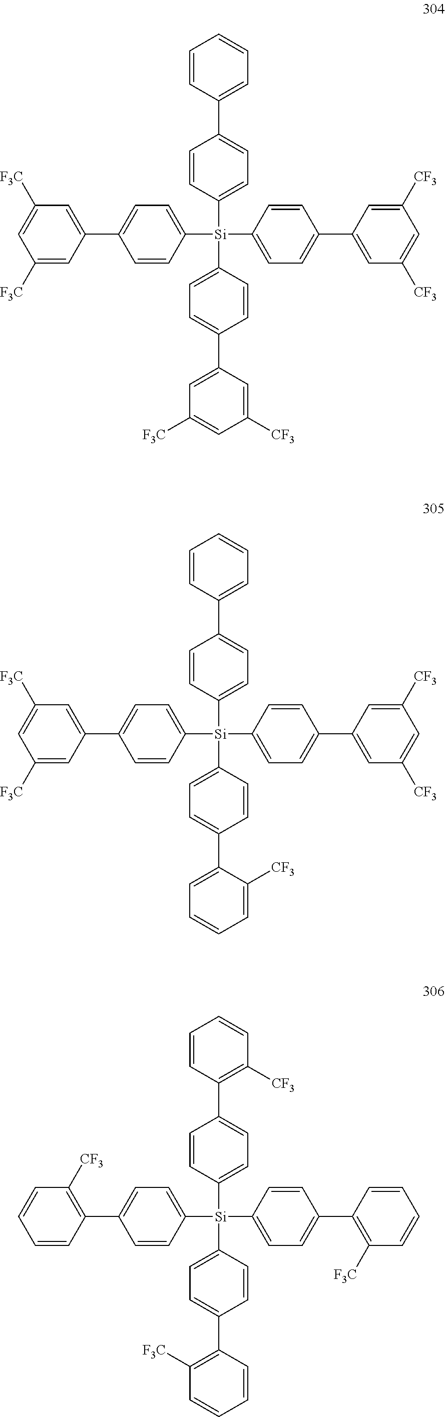 [SMILES: FC(F)(F)c1cc(-c2ccc([Si](c3ccc(-c4ccccc4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4ccccc4)cc3)(c3ccc(-c4cc(C(F)(F)F)cc(C(F)(F)F)c4)cc3)c3ccc(-c4ccccc4C(F)(F)F)cc3)cc2)cc(C(F)(F)F)c1 ; FC(F)(F)c1ccccc1-c1ccc([Si](c2ccc(-c3ccccc3C(F)(F)F)cc2)(c2ccc(-c3ccccc3C(F)(F)F)cc2)c2ccc(-c3ccccc3C(F)(F)F)cc2)cc1]

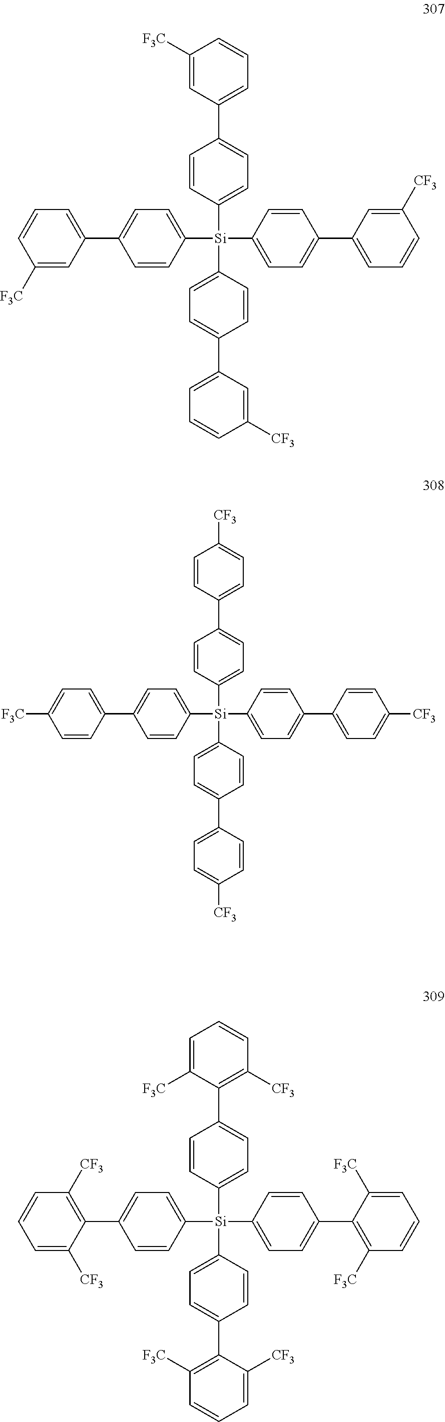 [SMILES: FC(F)(F)c1cccc(-c2ccc([Si](c3ccc(-c4cccc(C(F)(F)F)c4)cc3)(c3ccc(-c4cccc(C(F)(F)F)c4)cc3)c3ccc(-c4cccc(C(F)(F)F)c4)cc3)cc2)c1 ; FC(F)(F)c1ccc(-c2ccc([Si](c3ccc(-c4ccc(C(F)(F)F)cc4)cc3)(c3ccc(-c4ccc(C(F)(F)F)cc4)cc3)c3ccc(-c4ccc(C(F)(F)F)cc4)cc3)cc2)cc1 ; FC(F)(F)c1cccc(C(F)(F)F)c1-c1ccc([Si](c2ccc(-c3c(C(F)(F)F)cccc3C(F)(F)F)cc2)(c2ccc(-c3c(C(F)(F)F)cccc3C(F)(F)F)cc2)c2ccc(-c3c(C(F)(F)F)cccc3C(F)(F)F)cc2)cc1]

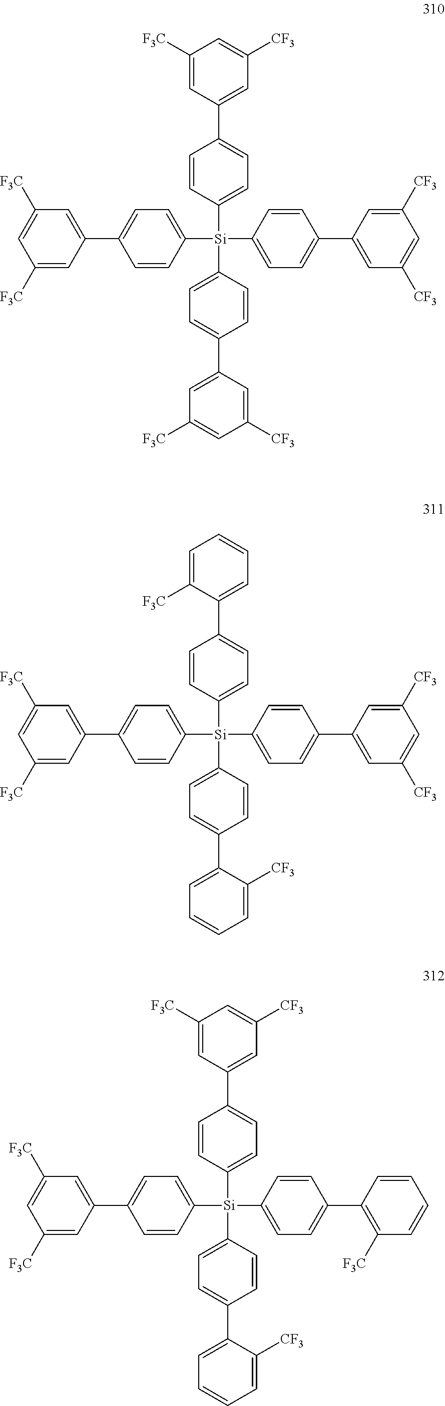 [SMILES: FC(F)(F)c1cc(-c2ccc([Si](c3ccc(-c4cc(C(F)(F)F)cc(C(F)(F)F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1 ; FC(F)(F)c1cc(-c2ccc([Si](c3ccc(-c4cc(C(F)(F)F)cc(C(F)(F)F)c4)cc3)(c3ccc(-c4ccccc4C(F)(F)F)cc3)c3ccc(-c4ccccc4C(F)(F)F)cc3)cc2)cc(C(F)(F)F)c1]

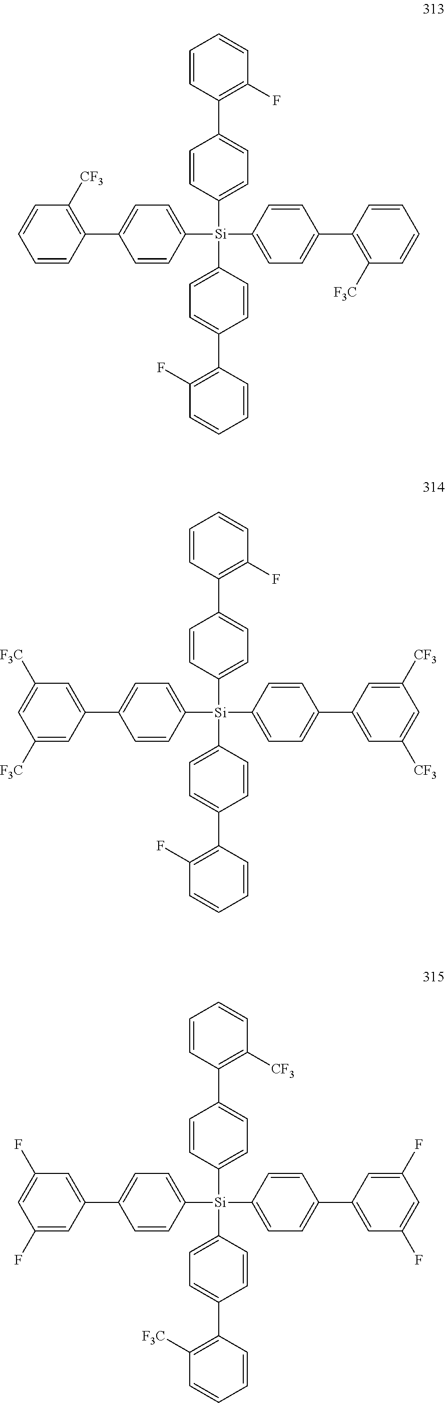 [SMILES: Fc1ccccc1-c1ccc([Si](c2ccc(-c3ccccc3F)cc2)(c2ccc(-c3ccccc3C(F)(F)F)cc2)c2ccc(-c3ccccc3C(F)(F)F)cc2)cc1 ; Fc1ccccc1-c1ccc([Si](c2ccc(-c3cc(C(F)(F)F)cc(C(F)(F)F)c3)cc2)(c2ccc(-c3cc(C(F)(F)F)cc(C(F)(F)F)c3)cc2)c2ccc(-c3ccccc3F)cc2)cc1 ; Fc1cc(F)cc(-c2ccc([Si](c3ccc(-c4cc(F)cc(F)c4)cc3)(c3ccc(-c4ccccc4C(F)(F)F)cc3)c3ccc(-c4ccccc4C(F)(F)F)cc3)cc2)c1]

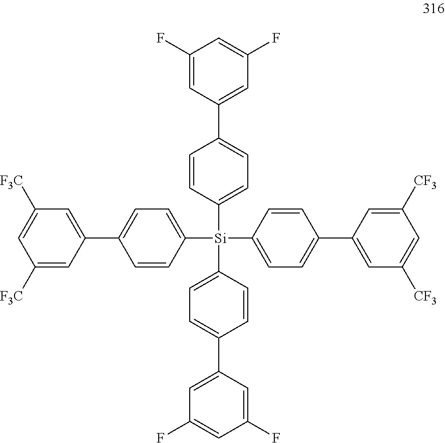 [SMILES: Fc1cc(F)cc(-c2ccc([Si](c3ccc(-c4cc(F)cc(F)c4)cc3)(c3ccc(-c4cc(C(F)(F)F)cc(C(F)(F)F)c4)cc3)c3ccc(-c4cc(C(F)(F)F)cc(C(F)(F)F)c4)cc3)cc2)c1]

5. An organic light-emitting device comprising a first electrode, a second electrode, and an organic layer having one or more layers disposed between the first electrode and the second electrode,
further comprising a light efficiency improving layer (capping layer) formed on at least one side opposite to the organic layer at the top or bottom of the first electrode and the second electrode,
wherein the light efficiency improving layer comprises the organic compound represented by Formula I above.

6. The organic light-emitting device of claim 5, wherein the light efficiency improving layer is formed on at least one of the bottom of the first electrode and the top of the second electrode.
